SEQUENCE LISTING (13 sequences)

SEQ ID NO: 1 - DNA, 18 bp, Artificial Sequence
  forward primer
gaggagatcaggcccaag

SEQ ID NO: 2 - DNA, 20 bp, Artificial Sequence
  reverse primer
cgttcaccacgtcatagtag

SEQ ID NO: 3 - DNA, 26 bp, Artificial Sequence
  sequence after ligation into vector
aagggcatcatcaccatcaccactga

SEQ ID NO: 4 - protein, 283 aa, Artificial Sequence
  predicted sequence of SYKKD expressed protein
MALEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKLARADENYYKAQTHGKWPVKWYAPECINYYFKSSKSDVWSFGVLMWEAFSYGQKPYRGMKGSEVTAMLEKGERMGCPAGCPREMYDLMNLCWTYDVENRPGFAAVELRLRNYYYDVVN

SEQ ID NO: 5 - protein, 292 aa, Artificial Sequence
  SYK
MALEEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKLARADENYYKAQTHGKWPVKWYAPECINYYFKSSKSDVWSFGVLMWEAFSYGQKPYRGMKGSEVTAMLEKGERMGCPAGCPREMYDLMNLCWTYDVENRPGFAAVELRLRNYYYDVVVNEGHHHHHH

SEQ ID NO: 6 - protein, 283 aa, human SYK; identical to SEQ ID NO: 4

SEQ ID NO: 7 - protein, 280 aa, pig SYK
EEIRPKEVYLDRKLLTLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEECNFVHRDLAARNVLLVTQHYAKISDFGLSKALRADENYYKAQTHGKWPVKWYAPECINYYKFSSKSDVWSFGVLMWEAFSYGQKPYRGMKGSEVSAMLEKGERMGCPPGCPREMYELMTLCWTYDVENRPGFVAVELRLRNYYYDVVN

SEQ ID NO: 8 - protein, 280 aa, rat SYK
EEIRPKEVYLDRKLLTLEDNELGSGNFGTVKKGYYQMKKVVKTVAVKILKNEANDPALKDELLAEANVMQQLDNPYIVRMIGICEAESWMLVMEMAAWGPLNKYLQQNRHIKDKNIIELVHQVSMGMKYLEESNFVHRDLAARNVLLVTQHYAKISDFGLSKALRADENYYKAQTHGKWPVKWYAPECINYFKFSSKSDVWSFGVLMWEAFSYGQKPYRGMKGSEVTAMLEKGERMGCPPGCPREMYDLMFLCWTYDVENRPGFAAVELRLRNYYYDVVN

SEQ ID NO: 9 - protein, 280 aa, rat SYKb
EEIRPKEVYLDRKLLTLEDNELGSGNFGTVKKGYYQMKKVVKTVAVKILKNEANDPALKDELLAEANVMQQLDNPYIVRMIGICEAESWMLVMEMAAWGPLNKYLQQNRHIKDKNIIELVHQVSMGMKYLEESNFVHRDLAARNVLLVTQHYAKISDFGLSKALRADENYYKAQTHGKWPVKWYAPECINYFKFSSKSDVWSFGVLMWEAFSYGQKPYRGMKGSEVTAMLEKGERMGCPPGCPREMYDLMNLCWTYDVENRPGFAAVELRLRNYYYDVVN

SEQ ID NO: 10 - protein, 280 aa, mouse SYK
EEIRPKEVYLDRKLLTLEDNELGSGNFGTVKKGYYQMKKVVKTVAVKILKNEANDPALKDELLAEANVMQQLDNPYIVRMIGICEAESWMLVMEMADVGPLNKYLQQNRHIKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPPGCPREMYDLMNLCWTYDVENRPGFAAVELRLRNYYYDVVN

SEQ ID NO: 11 - protein, 273 aa, T. rubripes SYK
DELRSSTVDRSQKLILEDGELGSGNFGTVKKGYIQMRKTEKSVAVKILKNDDNASVREEMLREANVMQQLDNPYIVRMIGICEAESLMLVMELADLGPLHKFLQKHKQISMKNITELVHQVSMGMRYLEEHKFVHRDLAARNVLLVTQHYAKISDFGLSKAVAEEKNYYKAKGHGKWPVKWYAPECINYFKFSSKSDVWSFGVLMWEAFSYGQKPYKGMKGNEVMQMIESGQRMDAPVNCPSEMYDLMACWTYKVEGRPEFTVVEPREYYYDI

SEQ ID NO: 12 - protein, 280 aa, human Zap70
EELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLAS

SEQ ID NO: 13 - protein, 280 aa, mouse Zap70
EELKDKKLFLKRENLLVADIELGCGNFGSVRQGVYRMRKKQIDVAIKVLKQGTEKADKDEMMREAQIMHQLDNPYIVRLIGVCQAEALMLVMEMAGGGPLHKFLLGKKEEIPVSNVAELLHQVAMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPQVLDFIKQGKRMECPPECPPEMYALMSDCWIYKWEDRPDFLTVEQRMRNYYYSPAS

(19) United States
(12) Patent Application Publication
(10) Pub. No.: US 2004/0253178 A1
(43) Pub. Date: Dec. 16, 2004

(54) Crystals and structures of spleen tyrosine kinase SYKKD

(76) Inventors: Shane Atwell, Encinitas, CA (US); Jorg Hendle, San Diego, CA (US); Sean Grant Buchanan, Encinitas, CA (US); Ingeborg Feil, San Diego, CA (US); Marijane Russell, Solana Beach, CA (US); John Badger, San Diego, CA (US); Masaki Tomimoto, San Diego, CA (US); Michael J. Sauder, Carlsbad, CA (US)

Correspondence Address: Structural GenomiX, Inc., 10505 Roselle Street, San Diego, CA, 92121, US

(21) Appl. No.: 10/738,575

(22) Filed: Dec. 17, 2003

Related U.S. Application Data
(60) Provisional application No. 60/435,476, filed on Dec. 20, 2002.

(51) Int. Cl. G06F 19/00; G01N 33/48; G01N 33/50

(52) U.S. Cl. 424/1.69; 530/350; 702/19

(57) ABSTRACT

The present invention provides machine readable media embedded with the three-dimensional molecular structure coordinates of SYKKD and subsets thereof, including binding pockets, methods of using the structure to identify and design affecters, including inhibitors and activator, mutants of SYKKD, and compounds and compositions that affect SYK activity.

### INTRODUCTION

This application claims benefit of priority from U.S. Provisional Patent Application 60/435,476, filed Dec. 20, 2002, which is hereby incorporated by reference as if fully set forth.

The present invention concerns crystalline forms of polypeptides that correspond to the kinase domain of spleen tyrosine kinase (SYK), methods of obtaining such crystals, and to the high-resolution X-ray diffraction structures and molecular structure coordinates obtained therefrom. The crystals of the invention and the atomic structural information obtained therefrom are useful, for example, for solving the crystal and solution structures of related and unrelated proteins, for screening for, identifying, and/or designing protein analogues and modified proteins, and for screening for, identifying and/or designing compounds that bind to and/or modulate a biological activity of SYK, including inhibitors and activators of SYK activity.

### BACKGROUND OF THE INVENTION

The present invention describes the 3-dimensional structure of the kinase domain of Homo sapiens SYK(SYKKD). SYK is a member of the SYK family of family of tyrosine protein kinases, a family of cytoplasmic tyrosine kinases characterized by the presence of two SH2 domains amino terminal to a single kinase domain. Homologs of the SYK family of protein kinases have been identified in a number of species, including human ZAP-70. The 3 dimensional structure of SYK may be useful, for example, for identifying novel therapeutic compounds that can modulate protein kinase activity, and for treatment of conditions mediated by human signal transduction kinase activity such as cancer, allergy, inflammation, and asthma.

Examples of homologs and orthologs of SYK among various species may be found in, for example, FIG. 3.

SYK has been reported to be involved in several cellular signalling events: immunoreceptor signaling, integrin signalling, and G protein-coupled receptor signalling. It is known to be expressed foremost in hematopoietic cells as wells as fibroblasts, epithelial cells, hepatocytes, neuronal cells, endothelial cells and mast cells. SYK is involved in hematopoietic responses such as proliferation, for example, SYK inhibitors have been suggested as modulators of thrombin-induced ASM cell proliferation; differentiation; and phagocytosis; and has been demonstrated to be important in nonhematopoietic cells as well, such as in fibroblasts, epithelial cells, breast tissue, hepatocytes, neuronal cells, and vascular endothelial cells. SYK has been implicated as playing a critical role in endothelial cell functions, including morphogenesis cell growth, migration, and survival, and as contributing to maintaining vascular integrity in vivo. Yanagi, S. et al., Biochem. Biophys. Res. Comm. 288:495-498 (2001).

Both SYK antisense and specific inhibitors have been shown to have some activity in asthma models and SYK is thought to be a target for the treatment of asthma and other airway diseases, as well as for allergies, inflammation, and autoimmunity. SYK has also been suggested as a target for the development of agonists in cancer therapy, due to its role in cell growth.

Knowledge of the 3-D structures of target proteins provides an important basis for structure-based approaches to drug design by defining the topographies of the complementary surfaces of ligands and their protein targets. Therefore, knowledge of the structure of the SYKKD protein described in the present invention may be useful in the identification, design, or development of novel and specific modulators of protein kinase activity as well as diagnostic and pharmaceutical compounds useful for disorders associated with aberrant SYK expression or activity. Knowledge of the structure may also be useful for gene therapy. The structural coordinates may be used, for example, to engineer more stable or other modified SYKs. The ability to obtain the molecular structure coordinates of SYKKD has not previously been realized.

Citation of documents herein is not intended as an admission that any is pertinent prior art. All statements as to the date or representation as to the contents of documents is based on the information available to the applicant and does not constitute any admission as to the correctness of the dates or contents of the documents.

### SUMMARY OF THE INVENTION

The present invention provides crystalline SYKKD, its molecular structure in atomic detail, homologs and mutants of the structure, methods of using the structure to identify and design compounds that modulate the activity of SYK, methods of preparing identified and/or designed compounds, methods of affecting cell growth and/or viability, and thus treating diseases or conditions, by modulating SYK activity, and methods of identifying and designing mutant SYKs. Knowledge of the structure of SYKKD may be useful in the development of novel compounds regulating immunoreceptor signaling, integrin signaling, G-protein-coupled receptor signaling, cell proliferation, cell migration, differentiation, cytoskeletal organization, gene expression, cell cycle progression, cell death, and mast cell signalling, including IgE signalling and histamine release in mast cells, and eosinophil activation. Knowledge of the structure of SYKKD may also be used to model the structure of kinases with related ligand binding sites, such as, for example, ZAP-70.

By “SYK activity” is meant SYK kinase activity, binding activity, imunogenicity, or any enzymatic activity of the SYK protein, or the SYK kinase domain alone. Thus, SYK activity may be assayed, where appropriate, using all or a portion of the entire SYK molecule. For example, the SYK kinase domain alone may be used in kinase, binding, immunogenicity, or other SYK enzymatic activities. Similarly, a modulator, inhibitor, or activator of SYK protein may also be a modulator, inhibitor, or activator of the SYK kinase domain, and modulation, inhibition or activation of SYK activity may be assayed by assaying the modulation, inhibition, or activation of SYK kinase domain activity. Also, where SYKKD activity is assayed, portions of the SYK molecule in addition to the SYKKD may be used in the assay. Thus, for example, where the present invention describes assaying modulation, inhibition, or activation of SYKKD, instead, an assay can be performed to determine modulation, inhibition, or activation of SYK.

Thus, in one aspect, the invention provides purified SYKKD, and methods of purifying SYKKD. SYKKD may be sufficiently pure such that it can be used to prepare diffraction quality crystals. For ease of obtaining diffraction quality crystals, the purified SYKKD may be predominantly, or entirely, of one phosphorylation state.

Thus, in one aspect, the invention provides a crystal comprising SYK or SYKKD peptides in preferrred crystalline form. In some embodiments of the invention the crystal is diffraction quality. The crystals of the invention include, for example, crystals of wild type SYKKD, crystals of mutated SYKKD, native crystals, heavy-atom derivative crystals, and crystals of SYKKD homologs or SYKKD mutants, such as, but not limited to, selenomethionine or selenocysteine mutants, mutants comprising conservative alterations in amino acid residues, and truncated or extended mutants.

The crystals of the invention also include co-crystals, in which crystallized SYKKD is in association with one or more compounds, including but not limited to, cofactors, ligands, substrates, substrate analogs, inhibitors, activators, agonists, antagonists, modulators, allosteric effectors, etc., to form a crystalline co-complex. Such compounds may or may not bind a catalytic or active site of SYKKD within the crystal. Alternatively, such compounds stably interact with another binding pocket of SYKKD within the crystal. The co-crystals may be native co-crystals, in which the co-complex is substantially pure, or they may be heavy-atom derivative co-crystals, in which the co-complex is in association with one or more heavy-metal atoms.

In other embodiments, the crystals of the invention are of sufficient quality to permit the determination of the three-dimensional X-ray diffraction structure of the crystalline polypeptide to high resolution, for example, to a resolution of better than 3 Å, or, at least 1 Å and up to about 3 Å, and more typically a resolution of greater than 1.5 Å and up to 2 Å or about 2 Å, or 2.5 Å or about 2.5 Å.

In some embodiments, the crystals are characterized by a unit cell of a=38.8 Å+/−2%, b=84.3 Å+/−2%, c=40.3 Å+/−2%, α=90°, β=99.57°+/−2% γ=90°, and a space group of P 1 21 1; or by a unit cell of a=39.8 Å+/−2%, b=84.7 Å+/−2%, c=90.2 Å+/−2%, α=90°, β=90°, γ=90°, and a space group of P 21 21 21.

In some embodiments, the crystals are characterized by a unit cell of a=40.3 Å+/−2%, b=84.7 Å+/−2%, c=90.9 Å+/−2%, α=90°, β=90°, γ=90°, and a group of P 21 21 21. In some embodiments, the crystals are characterized by a unit cell of a=39.8 Å+/−2%, b=85.8 Å+/−2%, c=90.5 Å+/−2%, α=90°, β=90°, γ90°, and a space group of P 21 21 21.

The invention also provides methods of making the crystals of the invention. Generally, crystals of the invention are grown by dissolving substantially pure polypeptide in an aqueous buffer that includes a precipitant at a concentration just below that necessary to precipitate the polypeptide. Water is then removed by controlled evaporation to produce precipitating conditions, which are maintained until the crystal forms and the size of the crystal is appropriate.

Co-crystals of the invention are prepared by soaking a native crystal prepared according to the above method in a liquor comprising the compound of the desired co-complex. Alternatively, the co-crystals may be prepared by co-crystallizing the polypeptide in the presence of the compound according to the method discussed above.

Heavy-atom derivative crystals of the invention may be prepared by soaking native crystals or co-crystals prepared according to the above method in a liquor comprising a salt of a heavy atom or an organometallic compound. Alternatively, heavy-atom derivative crystals may be prepared by crystallizing a polypeptide comprising modified amino acids, for example, selenomethionine and/or selenocysteine residues according to the methods described above for preparing native crystals.

In yet another embodiment of the present invention, a method is provided for determining the three-dimensional structure of a SYKKD crystal, comprising the steps of providing a crystal of the present invention; and analyzing the crystal by x-ray diffraction to determine the three-dimensional structure. Stated differently, the invention provides for the production of three-dimensional structural information (or “data”) from the crystals of the invention. Such information may be in the form of structural coordinates that define the three-dimensional structure of SYKKD in a crystal and/or co-crystal. Alternatively, the structural coordinates may define the three-dimensional structure of a portion of SYKKD in the crystal. Non-limiting examples of portions of SYKKD include the catalytic or active site, and a binding pocket. The structural coordinate information may include other structural information, such as vector representations of the molecular structures coordinates, and be stored or compiled in the form of a database, optionally in electronic form.

The invention thus provides methods of producing a computer readable database comprising the three-dimensional molecular structural coordinates of binding pocket of SYKKD, said methods comprising obtaining three-dimensional structural coordinates defining SYKKD or a binding pocket of SYKKD, from a crystal of SYKKD; and introducing said structural coordinates into a computer to produce a database containing the molecular structural coordinates of SYKKD or said binding pocket. The invention also provides databases produced by such methods.

In an alternative embodiment, the invention provides for the use of identifiers of structural information to be all or part of the information defining the three-dimensional structure of SYKKD so that all or part of the actual structural information need not be present. For example, and without limiting the invention, identifiers which reference structural coordinates defining a three-dimensional structure, substructure or shape may be used in place of the actual coordinate information. Such reference structural information is optionally stored separately from the identifiers used to define the three-dimensional structure of SYKKD. A non-limiting example is the use of an identifier for an alpha helix structure in place of the coordinates of the helical structure.

In another aspect, the invention provides computer machine-readable media embedded with the three-dimensional structural information obtained from the crystals of the invention, or portions or substrates thereof. The invention also provides methods for the introduction of the structural information into a computer readable medium, optionally as a computer readable database. The types of machine- or computer-readable media into which the structural information is embedded typically include magnetic tape, floppy discs, hard disc storage media, optical discs, CD-ROM, electrical storage media such as RAM or ROM, and hybrids of any of these storage media. Such media further include paper that can be read by a scanning device and converted into a three-dimensional structure with, for example, optical character recognition (OCR) software. In one example, the sheet of paper presents the molecular structure coordinates of crystalline polypeptide of the invention that are converted into, for example, a spread sheet by OCR software. The machine-readable media of the invention may further comprise additional information that is useful for representing the three-dimensional structure, including, but not limited to, thermal parameters, chain identifiers, and connectivity information.

Various machine-readable media are provided in the present invention. In one aspect, a machine-readable medium is provided that is embedded with information defining a three-dimensional structural representation of any of the crystals of the present invention, or a fragment or portion thereof. The information may be in the form of molecular structure coordinates, such as, for example, those of FIGS. 4-7. Alternatively, the information may include an identifier used to reference a particular three dimensional structure, substructure or shape. The machine-readable medium may be embedded with the molecular structure coordinates of a protein molecule comprising a SYKKD active site, active site homolog, binding pocket or binding pocket homolog. The various machine-readable media of the present invention may also comprise data corresponding to a molecule comprising a SYKKD binding pocket or binding pocket homolog in association with a compound or molecule bound to the protein, such as in a co-crystal.

The molecular structure coordinates and machine-readable media of the invention have a variety of uses. For example, the coordinates are useful for solving the three-dimensional X-ray diffraction and/or solution structures of other proteins, including mutant SYKKD, co-complexes comprising SYKKD, and unrelated proteins, to high resolution. Structural information may also be used in a variety of molecular modeling and computer-based screening applications to, for example, intelligently design mutants of the crystallized SYKKD that have altered biological activity and to computationally design and identify compounds that bind the polypeptide or a portion or fragment of the polypeptide, such as a subunit, a domain or an active site. Such compounds may be used directly or as lead compounds in pharmaceutical efforts to identify compounds that affect SYKKD activity. Compounds that bind to the polypeptide, or to a portion or fragment thereof may be used as, for example, antimicrobial agents.

The invention thus provides methods of producing a computer readable database comprising a representation of a compound capable of binding a binding pocket of SYKKD, said methods comprising introducing into a computer program a computer readable database comprising structural coordinates which may be used to produce a three dimensional representation of SYKKD, generating a three-dimensional representation of a binding pocket of SYKKD in said computer program, superimposing a three-dimensional model of at least one binding test compound on said representation of the binding pocket, assessing whether said test compound model fits spatially into the binding pocket of SYKKD and storing a representation of a compound that fits into the binding pocket into a computer readable database. The database used to store the representation of a compound may be the same or different from that used to store the structural coordinates of SYKKD. The invention further provides for the electronic transmission of any structural information resulting from the practice of the invention, such as by telephonic, computer implemented, microwave mediated, and satellite mediated means as non-limiting examples.

As described above, the molecular structure coordinates and/or machine-readable media associated with SYKKD structure may also be used in the production of three-dimensional structural information (or “data”) of a compound capable of binding SYKKD. Such information may be in the form of structural coordinates that define the three-dimensional structure of a compound, optionally in combination or with reference to structural components of SYKKD. In some embodiments, the structure coordinates of the compound are determined and presented (or represented) relative to the structure coordinates of the protein. Alternatively, identifiers of structural information are used to represent all or part of the information defining the three-dimensional structure of a compound so that all or part of the actual structural information need not be present. For example, and without limiting the invention, if the structural information of a compound includes a region defining a pyrophosphate (or pyrophosphate mimetic) moiety, the structural coordinates of pyrophosphate may be substituted by an identifier representing the structure of pyrophosphate, such as the name, chemical formula or other chemical representation. Any compound capable of binding SYKKD may be represented by chemical name, chemical or molecular formula, chemical structure, and/or other identifying information. As a non-limiting example, the compound CH₃CH₂OH can be represented by names such as ethanol or ethyl alcohol, abbreviations such as EtOH, chemical or molecular formulas such as CH₃CH₂OH or C₂H₅OH or C₂H₆O, and/or by structural representations in two or three dimensions. Non-limiting examples of the latter include Fisher projections, electron density maps and representations, space filling models, and the following:

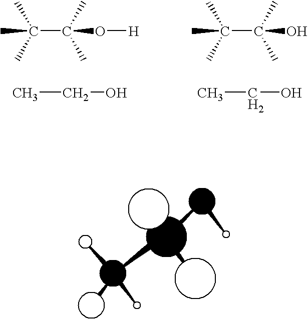

Non-limiting examples of other identifying information include Chemical Abstract Service (CAS) Registry numbers and physical or chemical properties indicative of the compound (such as, but not limited to, NMR spectra, IR spectra, MS spectra, GC profiles, and melting point). Of course the structures of a portion of a compound (e.g. a substructure) can be similarly identified by reference to any of the above used to identify a compound as a whole.

To produce structural information of a compound capable of binding SYKKD, the invention provides for the use of a variety of methods, including a) the superimposition of structures of known compounds on the structure of SYKKD or a portion thereof, b) the determination of a “pharmacophore” structure which binds SYKKD, and c) the determination of substructure(s) of compounds, wherein the substructure(s) interact with SYKKD. The structural coordinate information may include other structural information, such as vector representations of the molecular structures coordinates, and be stored or compiled in the form of a database, optionally in electronic form. With respect to a), the invention includes the computational screening of a three-dimensional structural representation of SYKKD or a portion thereof, or a molecule comprising a SYKKD binding pocket or binding pocket homolog, with a plurality of chemical compounds and chemical entities. Alternatively, the present invention provides a method of identifying at least one compound that potentially binds to SYKKD, comprising, constructing a three-dimensional structure of a protein molecule comprising a SYKKD binding pocket or binding pocket homolog, or constructing a three-dimensional structure of a molecule comprising a SYKKD binding pocket, and computationally screening a plurality of compounds using the constructed structure, and identifying at least one compound that computationally binds to the structure. In one aspect, the method further comprises determining whether the compound binds SYKKD.

With respect to b) the invention includes the computational screening of a plurality of chemical compounds to determine which compound(s), or portion(s) thereof, fit a pharmacophore determined as fitting within a SYKKD binding pocket. Stated differently, the structures of chemical compounds may be screened to identify which compound(s), or portion(s) thereof, is encompassed by the parameters of an identified pharmacophore. As used herein, “pharmacophore” refers to the structural characteristics determined as necessary for a chemical moiety to fit or bind a SYKKD binding pocket. A non-limiting example of a pharmacophore is a description of the electronic characteristics necessary for interaction with a binding site. These characteristics may be representations of the ground and excited state wave functions of a pharmacophore, including specification of known expansions of such functions. Representations of a pharmacophore contain the chemical moieties, and/or atoms thereof, within the pharmacophore as well as their electronic characteristics and their three dimensional arrangement in space. Other representations may also be used because different chemical moieties may have similar characteristics. A non-limiting example is seen in the case of a —SH moiety at a particular position, which has similar characteristics to a —OH moiety at the same position. Chemical moieties that may be substituted for each other within a pharmacophore are referred to as “homologous”.

The present invention thus provides methods for producing a computer readable database comprising a representation of a compound capable of binding a binding pocket of SYKKD, said methods comprising introducing into a computer program a computer readable database comprising structural coordinates which may be used to produce a three dimensional representation of SYKKD, determining a pharmacophore that fits within said binding pocket, computationally screening a plurality of compounds to determine which compound(s) or portion(s) thereof fit said pharmacophore, and storing a representation of said compound(s) or portion(s) thereof into a computer readable database. The database may be the same or different from that used to store the structural coordinates of SYKKD. Determination of a pharmacophore that fits may be performed by any means known in the art.

With respect to c) the invention includes the computational screening of a plurality of chemical compounds to determine which compounds comprise a substructure that interacts with SYKKD. The invention thus provides methods of producing a computer readable database comprising a representation of a compound capable of binding a binding pocket of SYKKD, said methods comprising introducing into a computer program a computer readable database comprising structural coordinates which may be used to produce a three dimensional representation of SYKKD, determining a chemical moiety that interacts with said binding pocket, computationally screening a plurality of compounds to determine which compound(s) comprise said moiety as a substructure of said compound(s), and storing a representation of said compound(s) and/or said moiety into a computer readable database which may be the same or different from that used to store the structural coordinates of SYKKD.

In one embodiment of the invention, the particulars of which may be used in combination with the other embodiments of the invention, a method is provided for producing structural information of a compound capable of binding SYKKD by selecting at least one compound that potentially binds to SYKKD. The method comprises constructing a three-dimensional structure of SYKKD having structure coordinates selected from the group consisting of the structure coordinates of the crystals of the present invention, the structure coordinates of FIGS. 4-7, and the structure coordinates of a protein having a root mean square deviation of the alpha carbon atoms of up to about 1.5 Å, preferably up to about 1.25 Å, preferably up to about 1 Å, preferably up to about 0.75 Å, preferably up to about 0.5 Å, and preferably up to about 0.25 Å, when compared to the structure coordinates of FIGS. 4-7, or a portion thereof, or constructing a three-dimensional structure of a molecule comprising a SYKKD binding pocket or binding pocket homolog; and selecting at least one compound which potentially binds SYKKD; wherein the selecting is performed with the aid of the constructed structure of SYKKD.

It is anticipated that in some cases, upon binding a compound, the conformation of the protein may be altered. Useful compounds may bind to this altered conformational form. Thus, included within the scope of the present invention are methods of producing structural information of a compound capable of binding SYKKD by selecting compounds that potentially bind to a SYKKD molecule or homolog where the molecule or homolog comprises an amino acid sequence that is at least 50%, preferably at least 60%, more preferably at least 70%, more preferably at least 80%, and more preferably at least 90% identical to the amino acid sequence of FIG. 2, using, for example, a PSI BLAST search, such as, but not limited to version 2.2.2 (Altschul, S.F., et al., Nuc. Acids Rec. 25:3389-3402, 1997). Preferably at least 50%, more preferably at least 70% of the sequence is aligned in this analysis and where at least 50%, more preferably 60%, more preferably 70%, more preferably 80%, and most preferably 90% of the amino acids of the molecule or homolog have structure coordinates selected from the group consisting of the structure coordinates of the crystals of the present invention, the structure coordinates of FIGS. 4-7, and the structure coordinates of a protein having a root mean square deviation of the alpha carbon atoms of up to about 1.5 Å, preferably up to about 1.25 Å, preferably up to about 1 Å, preferably up to about 0.75 Å, preferably up to about 0.5 Å, and preferably up to about 0.25 Å, when compared to the structure coordinates of FIGS. 4-7, or a portion thereof, or constructing a three dimensional structure of a molecule comprising a SYK binding pocket or binding pocket homolog; and selecting at least one compound which potentially binds SYK; wherein the selecting is performed with the aid of the constructed structure. The selected compounds thus provide information concerning the structure of compounds that bind SYK.

Once produced, structural information of a compound capable of binding SYK may be stored in machine-readable form as described above for SYK structural information.

In yet another aspect of the present invention, a method is provided of identifying a modulator of SYK by rational drug design, comprising; designing a potential modulator of SYK that forms covalent or non-covalent bonds with amino acids in a binding pocket of SYK based on the molecular structure coordinates of the crystals of the present invention, or based on the molecular structure coordinates of a molecule comprising a SYK binding pocket or binding pocket homolog; synthesizing the modulator; and determining whether the potential modulator affects the activity of SYK. The binding pocket may comprise the active site of SYK. The binding pocket may instead comprise an allosteric binding pocket of SYK. A modulator may be, for example, an inhibitor, an activator, or an allosteric modulator of SYK.

Other methods of designing modulators of SYK include, for example, a method for identifying a modulator of SYK activity comprising: providing a computer modeling program with a three dimensional conformation for a molecule that comprises a binding pocket of SYK, or binding pocket homolog; providing a said computer modeling program with a set of structure coordinates of a chemical entity; using said computer modeling program to evaluate the potential binding or interfering interactions between the chemical entity and said binding pocket, or binding pocket homolog; and determining whether said chemical entity potentially binds to or interferes with said molecule; wherein binding to the molecule is indicative of potential modulation, including, for example, inhibition of SYK activity.

In another embodiment, a method is provided for designing a modulator of SYK activity comprising: providing a computer modeling program with a set of structure coordinates, or a three dimensional conformation derived therefrom, for a molecule that comprises a binding pocket of SYK, or binding pocket homolog; providing a said computer modeling program with a set of structure coordinates, or a three dimensional conformation derived therefrom, of a chemical entity; using said computer modeling program to evaluate the potential binding or interfering interactions between the chemical entity and said binding pocket, or binding pocket homolog; computationally modifying the structure coordinates or three dimensional conformation of said chemical entity; and determining whether said modified chemical entity potentially binds to or interferes with said molecule; wherein binding to the molecule is indicative of potential modulation of SYK activity.

In other aspects, determining whether the chemical entity potentially binds to said molecule comprises performing a fitting operation between the chemical entity and a binding pocket, or binding pocket homolog, of the molecule or molecular complex; and computationally analyzing the results of the fitting operation to quantify the association between, or the interference with, the chemical entity and the binding pocket, or binding pocket homolog. In a further embodiment, the method further comprises screening a library of chemical entities.

The SYK modulator may also be designed de novo. Thus, the present invention also provides a method for designing a modulator of SYK, comprising: providing a computer modeling program with a set of structure coordinates, or a three dimensional conformation derived therefrom, for a molecule that comprises a binding pocket having the structure coordinates of the binding pocket of SYK, or a binding pocket homolog; computationally building a chemical entity represented by set of structure coordinates; and determining whether the chemical entity is a modulator expected to bind to or interfere with the molecule wherein binding to the molecule is indicative of potential modulation of SYK activity. In other embodiments, determining whether the chemical entity potentially binds to said molecule comprises performing a fitting operation between the chemical entity and a binding pocket of the molecule or molecular complex, or a binding pocket homolog; and computationally analyzing the results of the fitting operation to quantify the association between, or the interference with, the chemical entity and the binding pocket, or a binding pocket homolog.

In yet other embodiments, once a modulator is computationally designed or identified, the potential modulator may be supplied or synthesized, then assayed to determine whether it inhibits SYK activity. The molecular structure coordinates and/or machine-readable media associated with the SYK structure and/or a compound capable of binding SYKKD may be used in the production of compounds capable of binding SYK. Methods for the production of such compounds include the preparation of an initial compound containing chemical groups most likely to bind or interact with residues of SYKKD based upon the molecular structure coordinates of SYKKD and/or a compound capable of binding it. Such an initial compound may also be viewed as a scaffold comprising one or more reactive moieties (chemical groups) that are capable of binding or interacting with SYK residues. The initial compound may be further optimized for binding to SYK by introduction of additional chemical groups for increased interactions with SYKKD residues. An initial compound may thus comprise reactive groups which may be used to introduce one or more additional chemical groups into the compound. The introduction of additional groups may also be at positions of an initial compound that do not result in interactions with SYK residues, but rather improve other characteristics of the compound, such as, but not limited to, stability against degradation, handling or storage, solubility in hydrophilic and hydrophobic environments, and overall charge dynamics of the compound.

The present invention also provides modulators of SYK activity identified, designed, or made according to any of the methods of the present invention, as well as pharmaceutical compositions comprising such modulators. Pharmaceutical compositions may be in the form of a salt, and may further comprise a pharmaceutically acceptable carrier. A modulator can be identified or confirmed as an activator or inhibitor by contacting a protein that comprises a SYK active site or binding pocket with said modulator and determining whether it activates or inhibits the activity of the protein. The activity may be SYK activity. A naturally occurring SYK protein may also be used in such methods.

Also provided in the present invention is a method of modulating SYK activity comprising contacting SYK with a modulator designed or identified according to the present invention. Methods include methods of treating a disease or condition associated with inappropriate SYK activity comprising the method of administering by, for example, contacting cells of an individual with a SYK modulator designed or identified according to the present invention. The term “inappropriate activity” refers to SYK activity that is higher or lower than that in normal cells.

The molecular structure coordinates and/or machine-readable media of the invention may also be used in identification of active sites and binding pockets of SYKKD. Methods for the identification of such sites and pockets are known in the art. The techniques include the use of sequence comparisons, such as that shown in FIG. 3, to identify regions of homology or conserved substitutions which define conserved structure among different forms of SYKKD. The techniques may also include comparisons of structure with other proteins with the same activities as SYK to identify the structural components (e.g. amino acid residues and/or their arrangement in three dimensions) of the active sites and binding pockets.

In another embodiment of the present invention, a method is provided for producing a mutant of SYK, having an altered property relative to SYK, comprising, a) constructing a three-dimensional structure of SYKKD having structure coordinates selected from the group consisting of the structure coordinates of the crystals of the present invention, the structure coordinates of FIGS. 4-7, and the structure coordinates of a protein having a root mean square deviation of the alpha carbon atoms of the protein of up to about 1.5 Å, preferably up to about 1.25 Å, preferably up to about 1 Å, preferably up to about 0.75 Å, preferably up to about 0.5 Å, and preferably up to about 0.25 Å, when compared to the structure coordinates of FIGS. 4-7; b) using modeling methods to identify in the three-dimensional structure at least one structural part of the SYKKD molecule wherein an alteration in the structural part is predicted to result in the altered property; c) providing a nucleic acid molecule having a modified sequence that encodes a deletion, insertion, or substitution of one or more amino acids at a position corresponding to the structural part; and d) expressing the nucleic acid molecule to produce the mutant; wherein the mutant has at least one altered property relative to the parent. The mutant may, for example, have altered SYK activity. The altered SYK activity may be, for example, altered binding activity, altered enzymatic activity, and altered immunogenicity, such as, for example, where an epitope of the protein is altered because of the mutation. The mutation that alters the epitope may be, for example, within the region of the protein that comprises the epitope. Or, the mutation may be, for example, at a site outside of the epitope region, yet causes a conformational change in the epitope region. Those of ordinary skill in the art will recognize that the region that contains the epitope may comprise either contiguous or non-contiguous amino acids.

Also provided in the present invention is a method for obtaining structural information about a molecule or a molecular complex of unknown structure comprising: crystallizing the molecule or molecular complex; generating an x-ray diffraction pattern from the crystallized molecule or molecular complex; and using a molecular replacement method to interpret the structure of said molecule; wherein said molecular replacement method uses the structure coordinates of FIGS. 4-7, or structure coordinates having a root mean square deviation for the alpha-carbon atoms of said structure coordinates of up to about 2.0 Å, preferably up to about 1.75 Å, preferably up to about 1.5 Å, preferably up to about 1.25 Å, preferably up to about 1.0 Å, preferably up to about 0.75 Å, the structure coordinates of the binding pocket of FIGS. 4-7, or a binding pocket homolog. The coordinates of the resulting structure are stored in a computer readable database as described herein.

In another aspect of the invention, a method is provided of using the SYKKD structure coordinates, or the SYKKD binding site, active site, or accessory binding site structure coordinates as an anti-target in rational drug design. When designing compounds that modulate a protein target's activity, it is often desirable to increase specificity for the target and reduce side effects. The protein structure information is useful to design compounds that do not bind to, interact with, or modulate the activity of the protein. Thus, one aspect of the present invention comprises the use of anti-target structures to assist in selecting a compound that modulates the target, but does not modulate SYK, or does not modulate SYK in sufficient amount to cause a detrimental side affect.

The target may, for example, be another kinase. The target may be another SYK family kinase, for example, ZAP70.

Thus, in one aspect of the invention, a method is provided of identifying a compound that modulates the activity of a target protein, comprising: a) introducing into a computer program information derived from structural coordinates defining an active site conformation of a target protein molecule based upon three-dimensional structure determination, wherein said program utilizes or displays the three-dimensional structure thereof; b) generating a three-dimensional representation of the active site cavity of said target protein in said computer program; c) superimposing a model of a test compound on the model of said active site of said target protein; d) assessing whether said test compound model fits spatially into the active site of said target protein; e) generating a three-dimensional representation of a binding pocket of a SYKKD protein in a computer program; f) superimposing a model of said test compound on the model of said binding pocket of said SYKKD protein; and g) assessing whether said test compound model fits spatially into said binding pocket of said SYKKD protein.

The binding pocket of the SYKKD protein may be, for example, an active site or an accessory binding site. Said target protein may be a kinase. The test compound model may or may not fit spatially into the binding pocket of said SYKKD protein. The method may further comprise performing a fitting operation to computationally analyze the association between the test compound and the SYKKD protein. The test compound may bind with greater efficiency to the target protein than to the SYKKD protein; the test compound may, for example, not bind to the SYKKD protein.

In yet another aspect of the invention, a method is provided for homology modeling of a SYKKD homolog comprising: aligning the amino acid sequence of a SYKKD homolog with an amino acid sequence of SYKKD; incorporating the sequence of the SYKKD homolog into a model of the structure of SYKKD, wherein said model has the same structure coordinates as the structure coordinates of FIGS. 4-7, or wherein the structure coordinates of said model's alpha-carbon atoms have a root mean square deviation from the structure coordinates of FIGS. 4-7 of up to about 2.0 Å, preferably up to about 1.75 Å, preferably up to about 1.5 Å, preferably up to about 1.25 Å, preferably up to about 1.0 Å, and preferably up to about 0.75 Å, to yield a preliminary model of said homolog; subjecting the preliminary model to energy minimization to yield an energy minimized model; and remodeling regions of the energy minimized model where stereochemistry restraints are violated to yield a final model of said homolog.

The invention also provides SYKKD in crystalline form, as well as a computer or machine readable medium containing information that reflects the three dimensional structure of such crystals and/or compounds that interact with them. Also provided is a method of producing a computer readable database containing the three-dimensional molecular structure coordinates of a compound capable of binding the active site or binding pocket of a SYKKD but not another protein molecule. Such a method comprises a) introducing into a computer program information concerning the structure of SYKKD; b) generating a three-dimensional representation of the active site or binding pocket of SYKKD in said computer program; c) superimposing a three-dimensional model of at least one binding test compound on said representation of the active site or binding pocket; d) assessing whether said test compound model fits spatially into the active site or binding pocket of SYKKD; e) assessing whether a compound that fits will fit a three-dimensional model of another protein, the structural coordinates of which are also introduced into said computer program and used to generate a three-dimensional representation of the other protein; and f) storing the three-dimensional molecular structure coordinates of a model that does not fit the other protein into a computer readable database. An alternative form of such a method produces a computer readable database containing the three-dimensional molecular structural coordinates of a compound capable of specifically binding the active site or binding pocket of SYKKD, said method comprising introducing into a computer program a computer readable database containing the structural coordinates of SYKKD, generating a three-dimensional representation of the active site or binding pocket of SYKKD in said computer program, superimposing a three-dimensional model of at least one binding test compound on said representation of the active site or binding pocket, assessing whether said test compound model fits spatially into the active site or binding pocket of SYKKD, assessing whether a compound that fits will fit a three-dimensional model of another protein, the structural coordinates of which are also introduced into said computer program and used to generate a three-dimensional representation of the other protein, and storing the three-dimensional molecular structural coordinates of a model that does not fit the other protein into a computer readable database. Conversely, such methods may be used to determine that compounds identified as binding other proteins do not bind SYKKD. Thus, such methods may use SYKKD as an anti-target, to identify compounds that do not bind SYKKD.

The invention also provides methods comprising the production of a co-crystal of a compound and SYKKD. Such co-crystals may be used in a variety of ways, including the determination of structural coordinates of the compound and/or SYKKD, or a binding pocket thereof, in the co-crystal. Such coordinates may be introduced and/or stored in a computer readable database in accordance with the present invention for further use. The invention thus provides methods of producing a computer readable database comprising a representation of a binding pocket of SYKKD in a co-crystal with a compound, said methods comprising preparing a binding test compound represented in a computer readable database produced by any method described herein, forming a co-crystal of said compound with a protein comprising a binding pocket of SYKKD, obtaining the structural coordinates of said binding pocket in said co-crystal, and introducing the structural coordinates of said binding pocket or said co-crystal into a computer-readable database. The invention further provides for a combination of such methods with rational compound design by providing methods of producing a computer readable database comprising a representation of a binding pocket of SYKKD in a co-crystal with a compound rationally designed to be capable of binding said binding pocket, said methods comprising preparing a binding test compound represented in a computer readable database produced by any method described herein, forming a co-crystal of said compound with a protein comprising a binding pocket of SYKKD, obtaining the structural coordinates of said binding pocket in said co-crystal, and introducing the structural coordinates of said binding pocket or said co-crystal into a computer-readable database.

The invention is illustrated by way of the present application, including working examples demonstrating the purification and the crystallization of SYKKD, the characterization of crystals, the collection of diffraction data, and the determination and analysis of the three-dimensional structure of SYKKD.

### BRIEF DESCRIPTION OF THE FIGURES

FIG. 1 provides a ribbon diagram of the structure of SYKKD.

FIG. 2 provides the predicted amino acid sequence of the SYKKD expressed protein used to obtain the crystals and structural coordinates of the present invention. Note that this amino acid sequence may comprise amino acids encoded by the ORF, as well as other amino acids encoded by the expression vector. Further information regarding sequence changes, if any, may be found in the examples.

FIG. 3 (A-C) provides a sequence alignment of SYKKD from various species. Homologs were identified with PSI-BLAST 2.2.2 using the Genbank non-redundant database. DbClustal was used to create the multiple alignment. ESPript was used to generate the PostScript version of the alignment. The species is identified along with the Genbank gi number (in parenthesis). The secondary structure of SYKKD was calculated by STRIDE. References: Frishman, D; Argos, P. “STRIDE: Knowledge-based protein secondary structure assignment.” Protein, 23:566-79, 1995; Thompson, J. D.; Plewniak, F; Thierry J; Poch O. “DbClustal: Rapid and reliable global multiple alignments of the protein sequences detected by database searches.” Nucleic Acids Research, 28:2919-26, 2000; Gouet, P; Courcelle, E; Stuart D I; Metoz, F. “ESPript: analysis of multiple sequence alignments in PostScript.” Bioinformatics, 15:305-08, 1999). Active site residues are indicated by a blackened oval.

The top line indicates various alpha helices and beta sheets calculated from the Homo sapiens structure. In this sequence alignment, highly conserved residues are indicated by a box. Strictly conserved residues are highlighted by inverse shading (white on black).

FIG. 4 (A-HH) provides the molecular structure coordinates of SYKKD.

FIG. 5 (A-JJ) provides the molecular structure coordinates of SYKKD in a different space group from the SYKKD represented by the coordinates of FIG. 4, 5, 6 and 7.

FIG. 6 (A-LL) provides the molecular structure coordinates of a SYKKD-staurosporine co-crystal.

FIG. 7 (A-MM) provides the molecular structure coordinates of a SYKKD co-crystal.

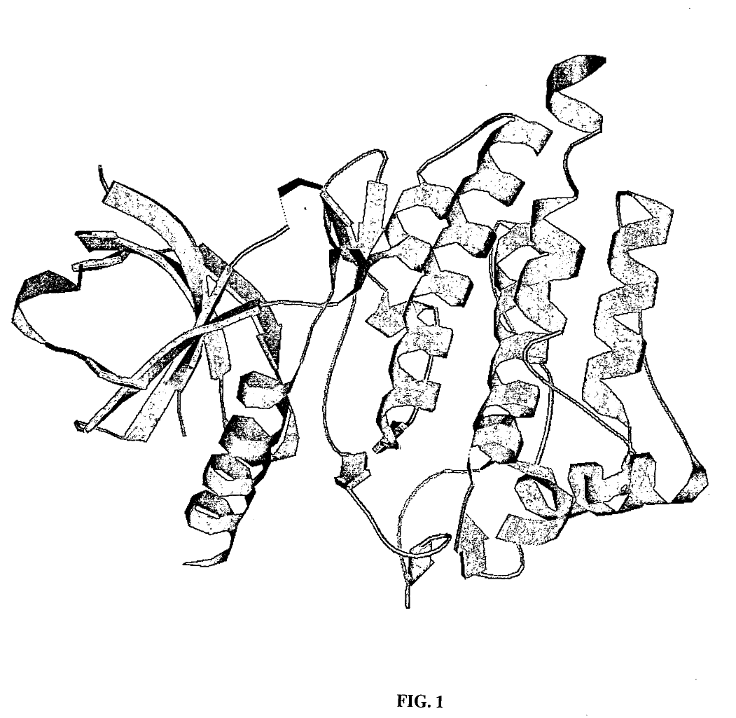

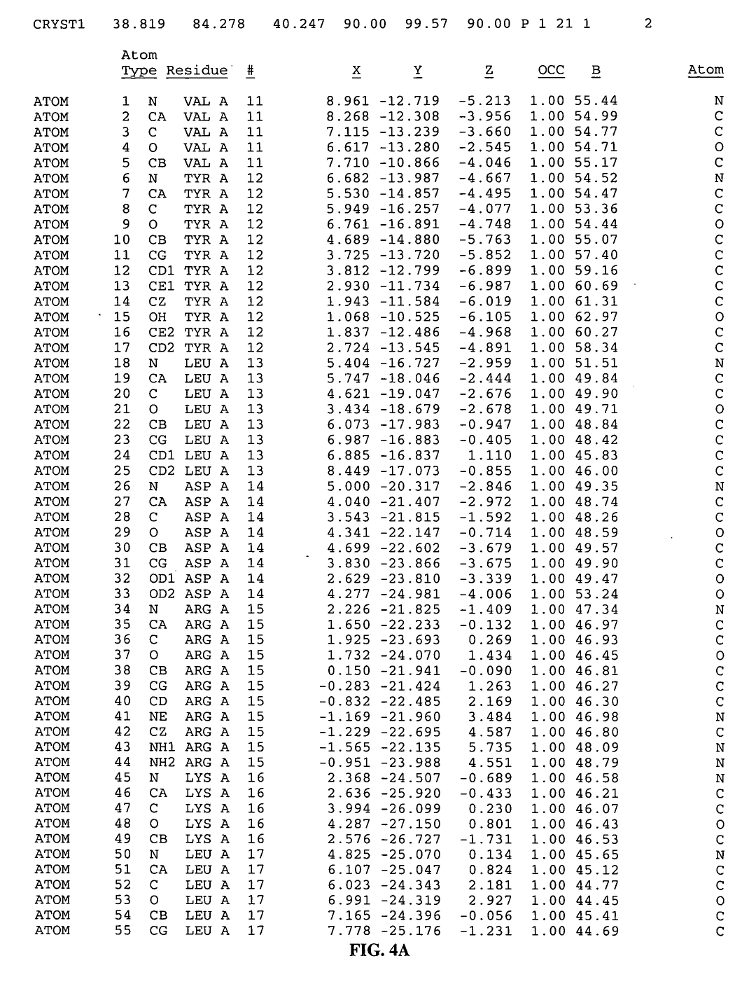

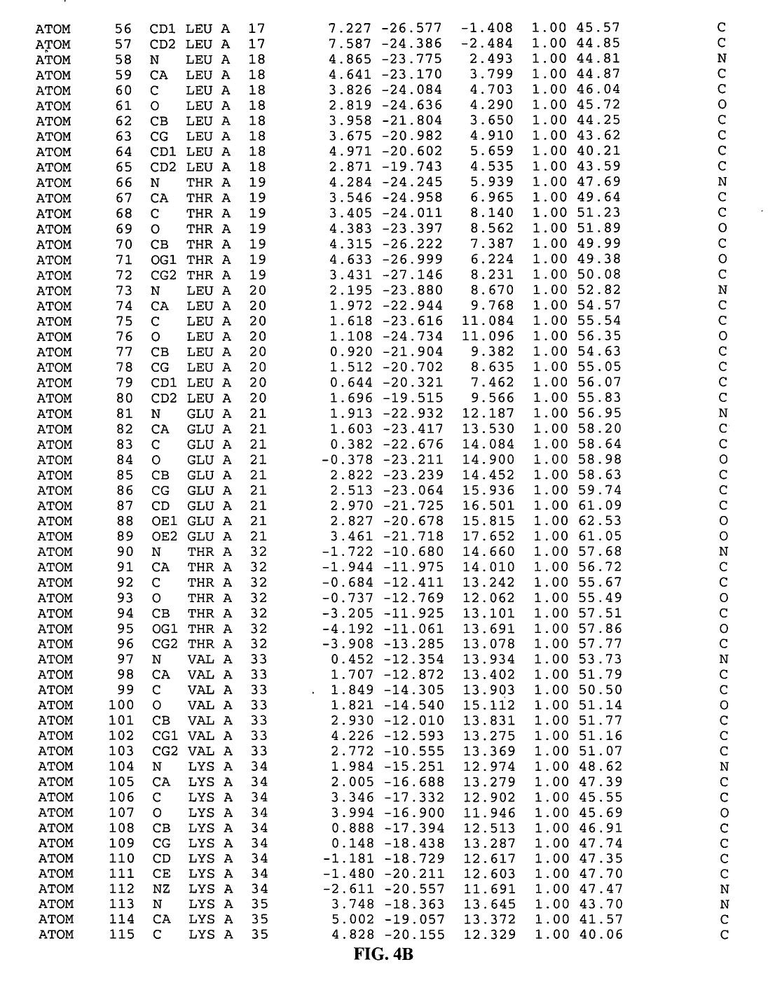

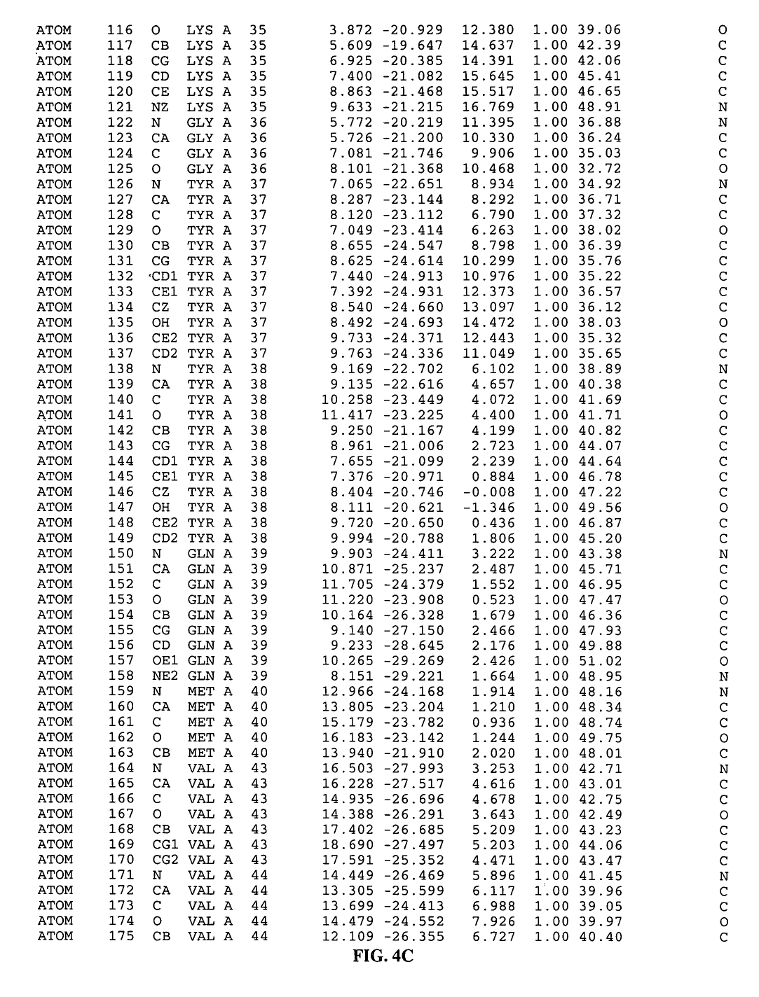

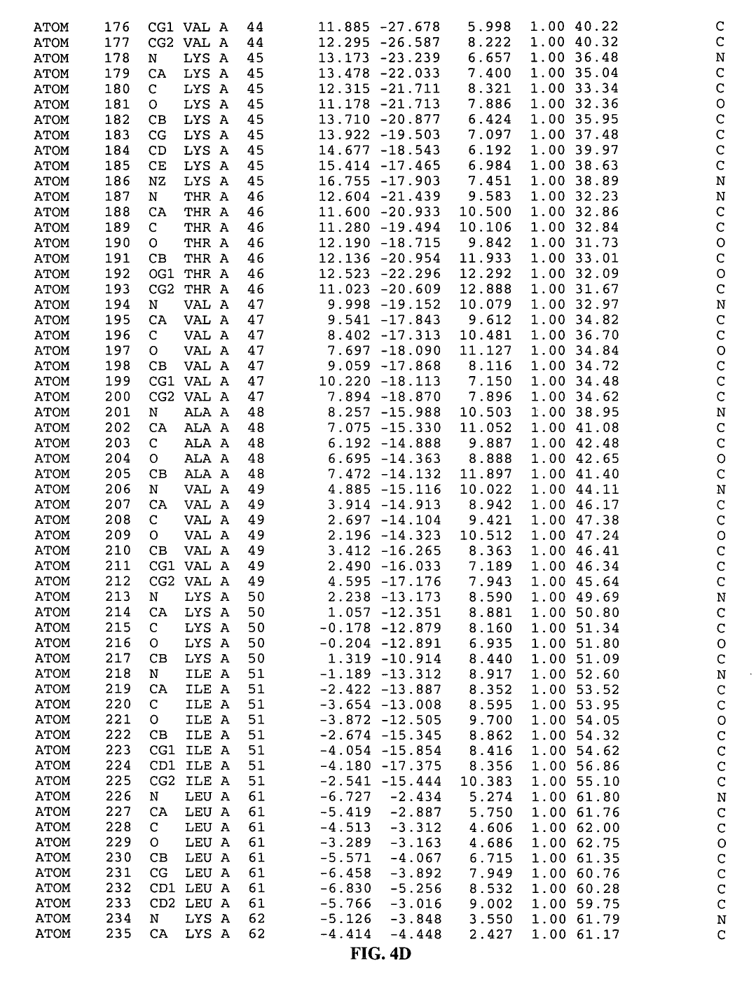

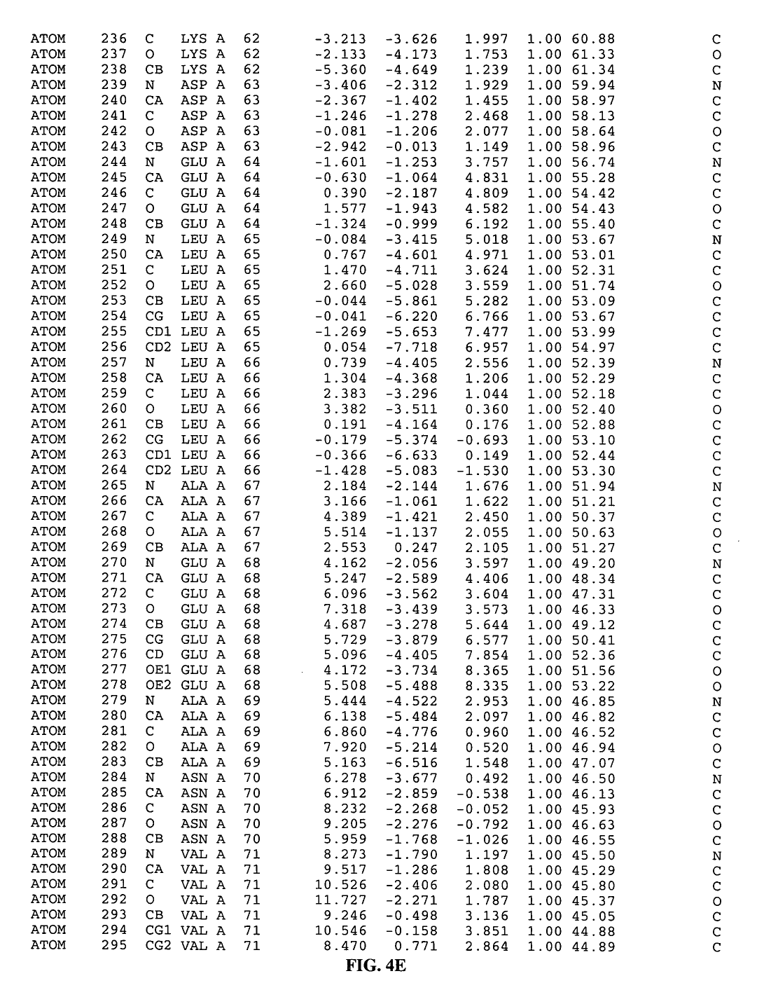

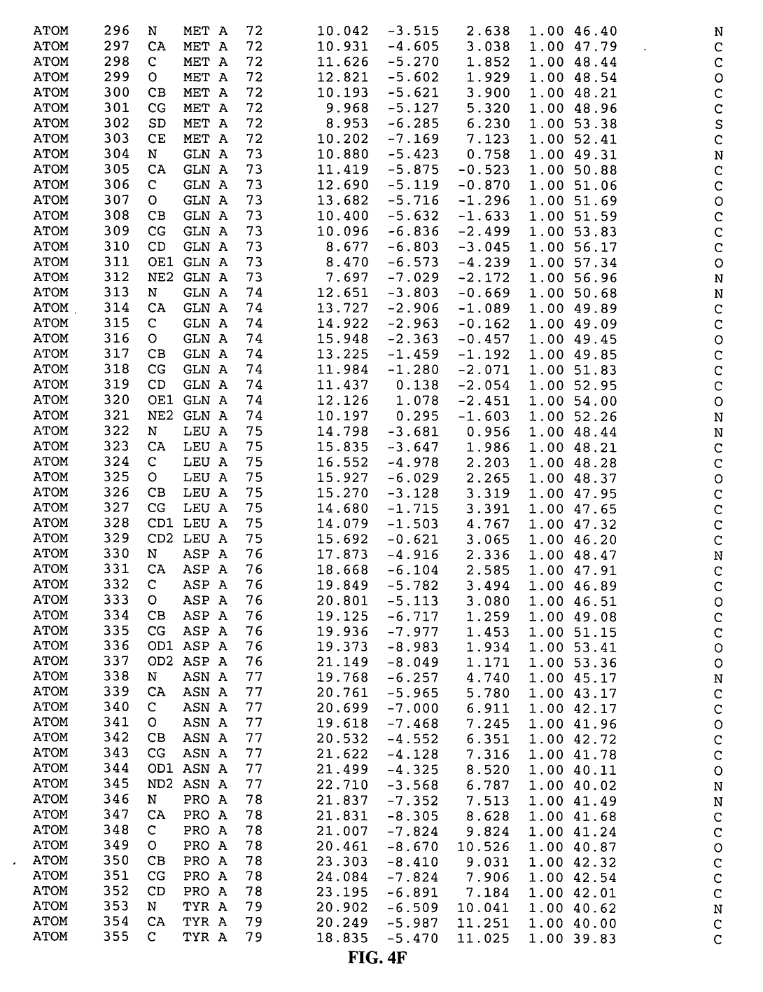

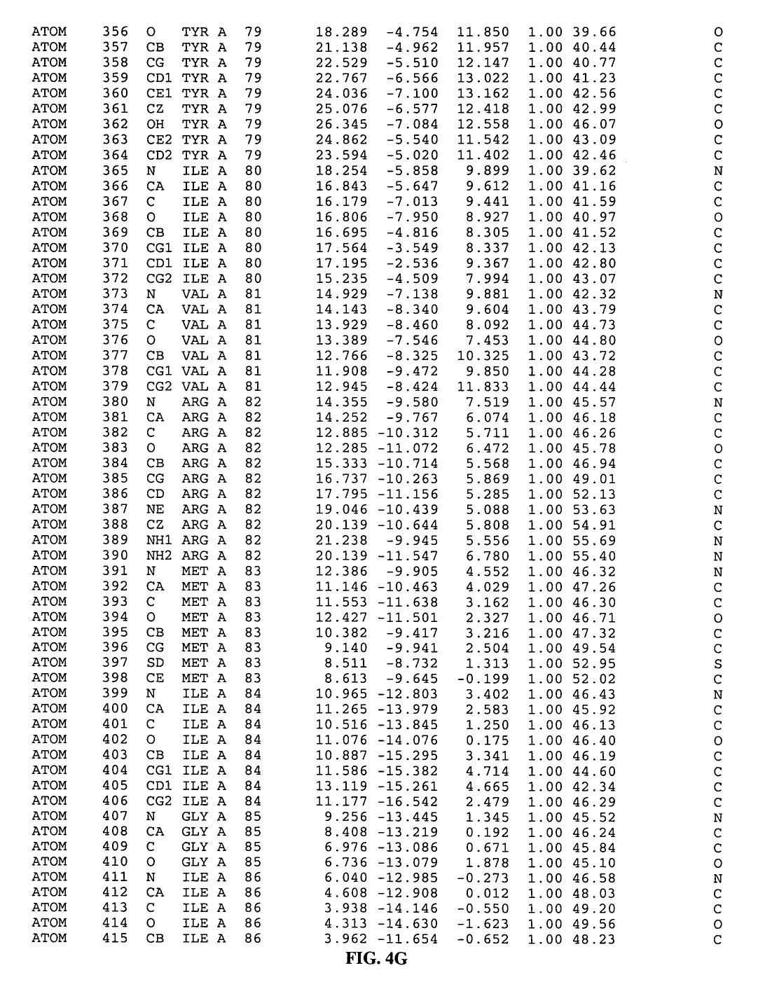

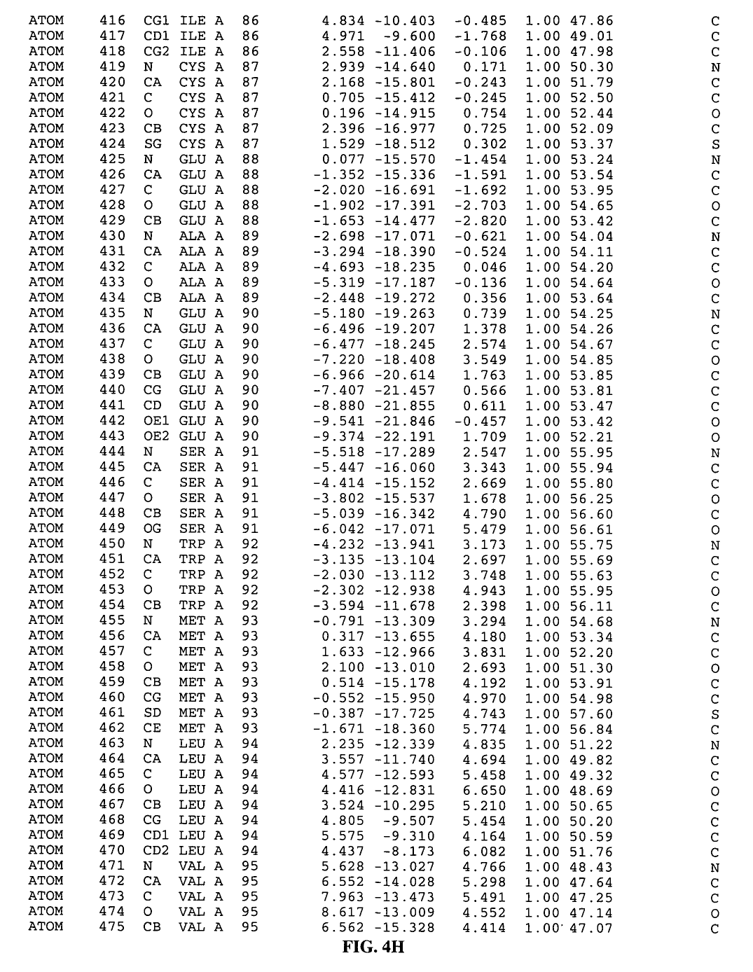

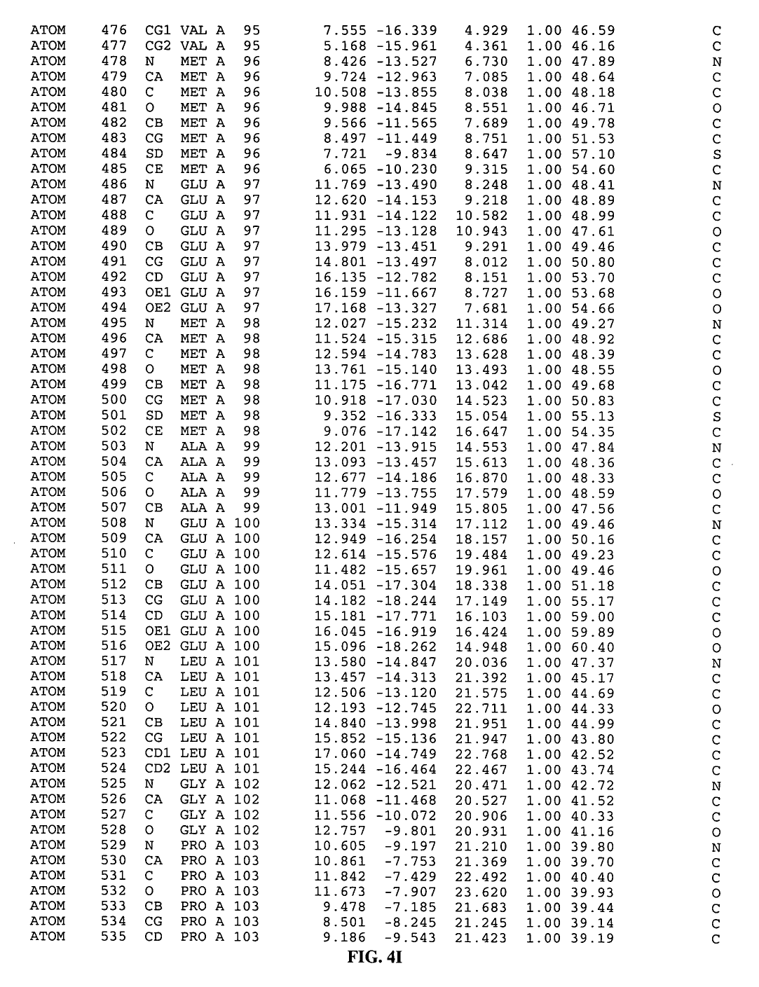

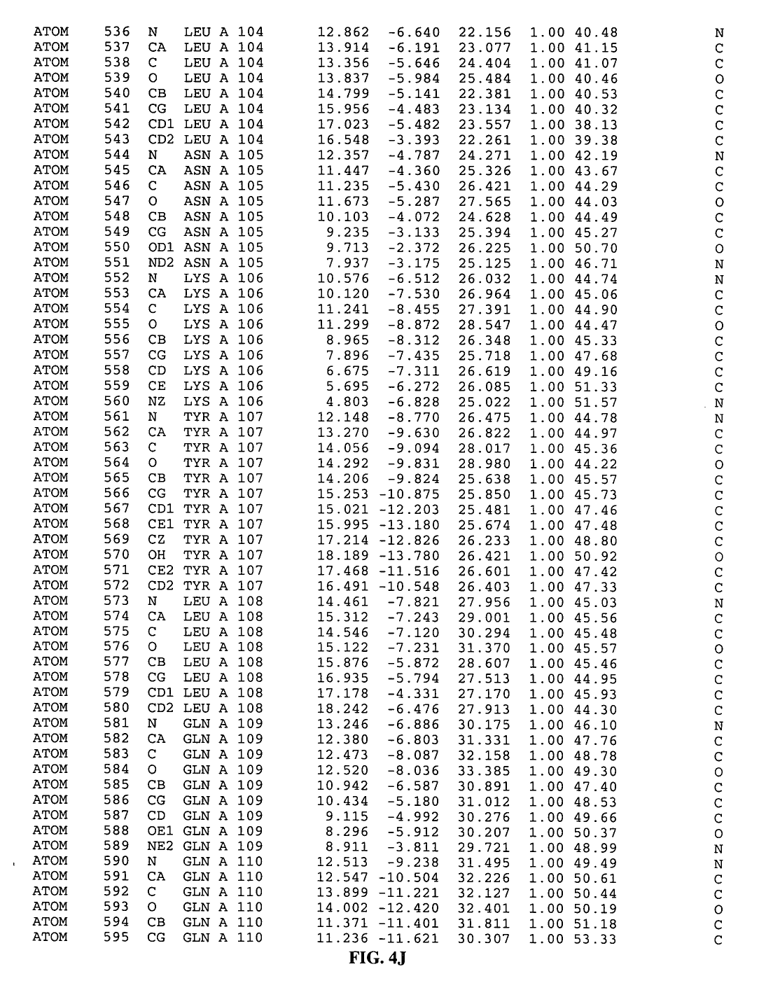

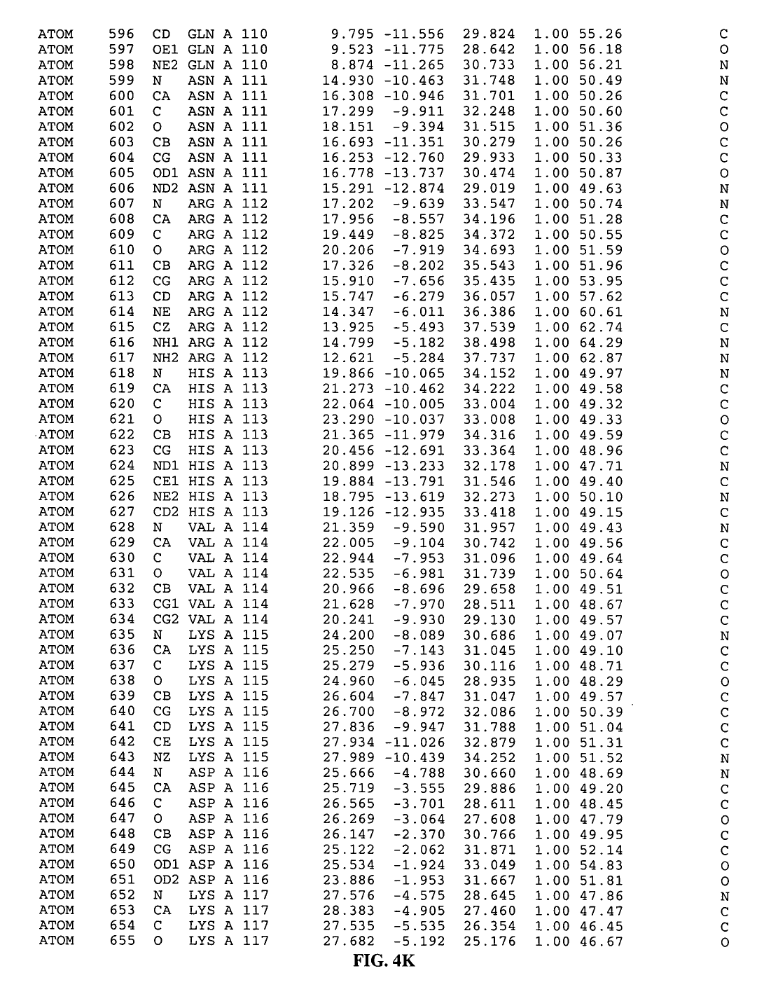

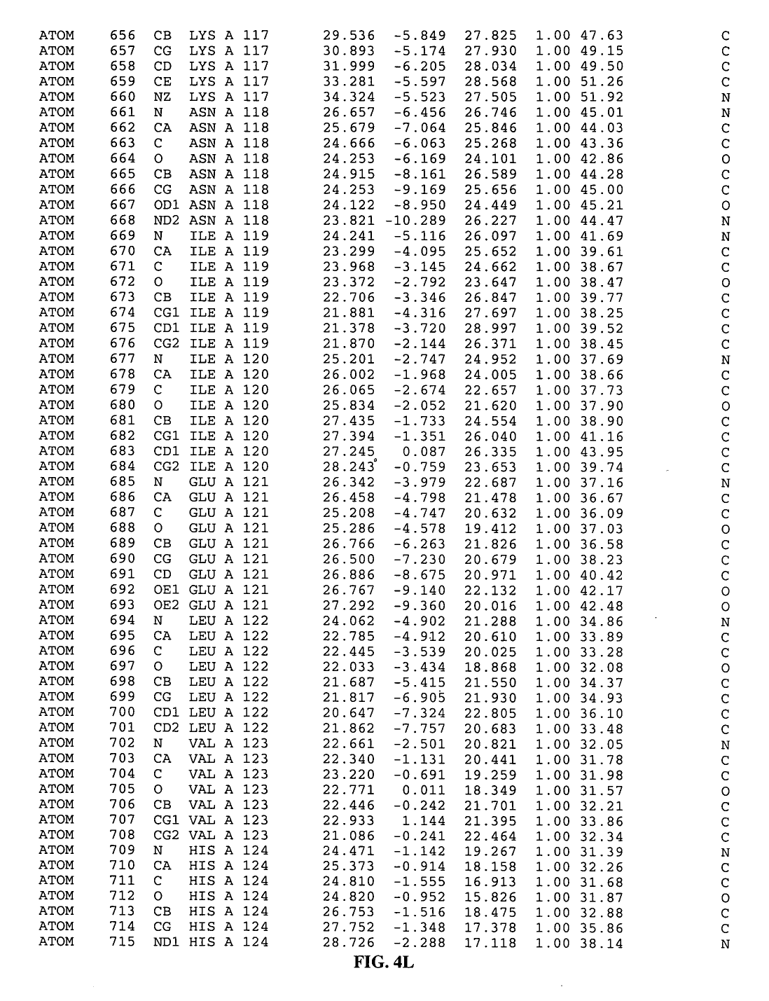

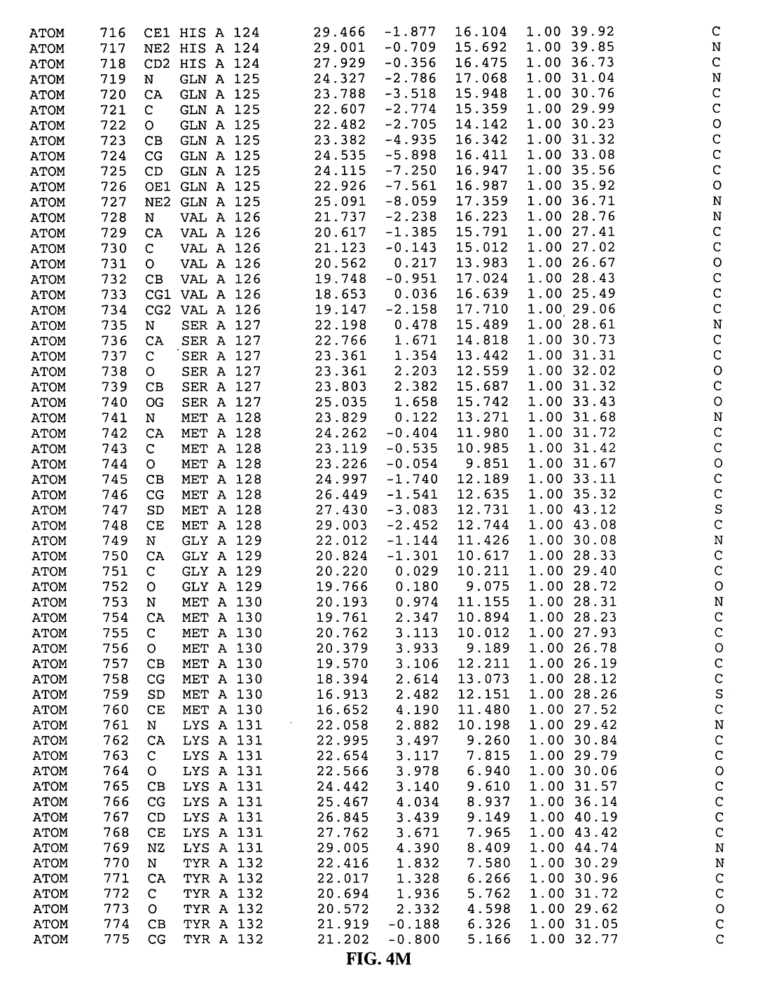

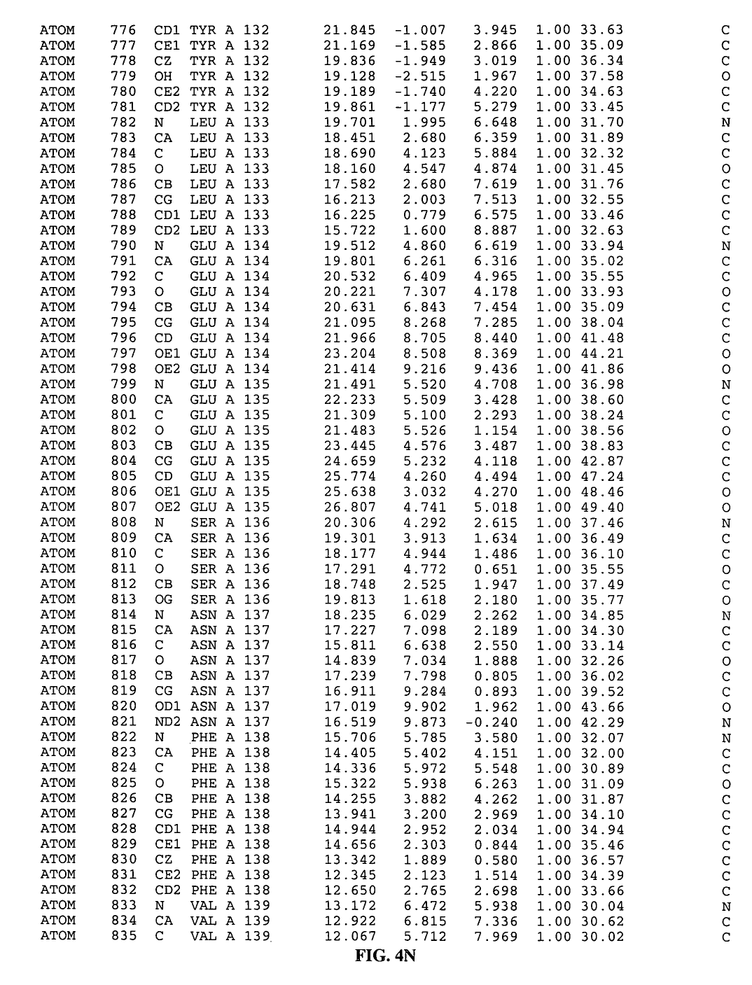

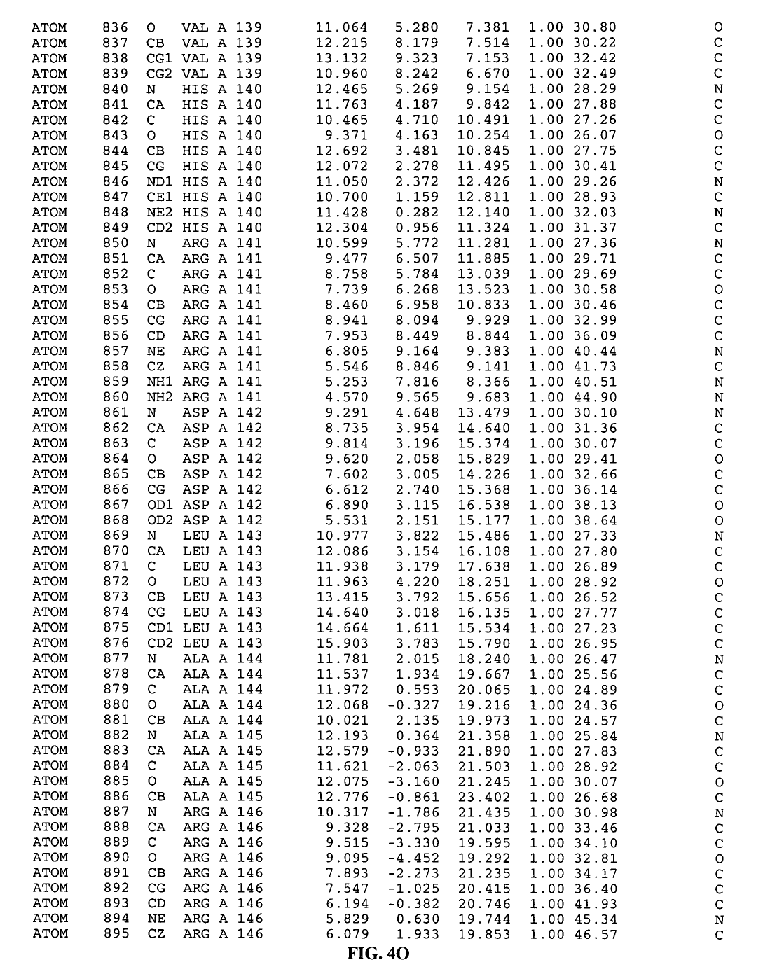

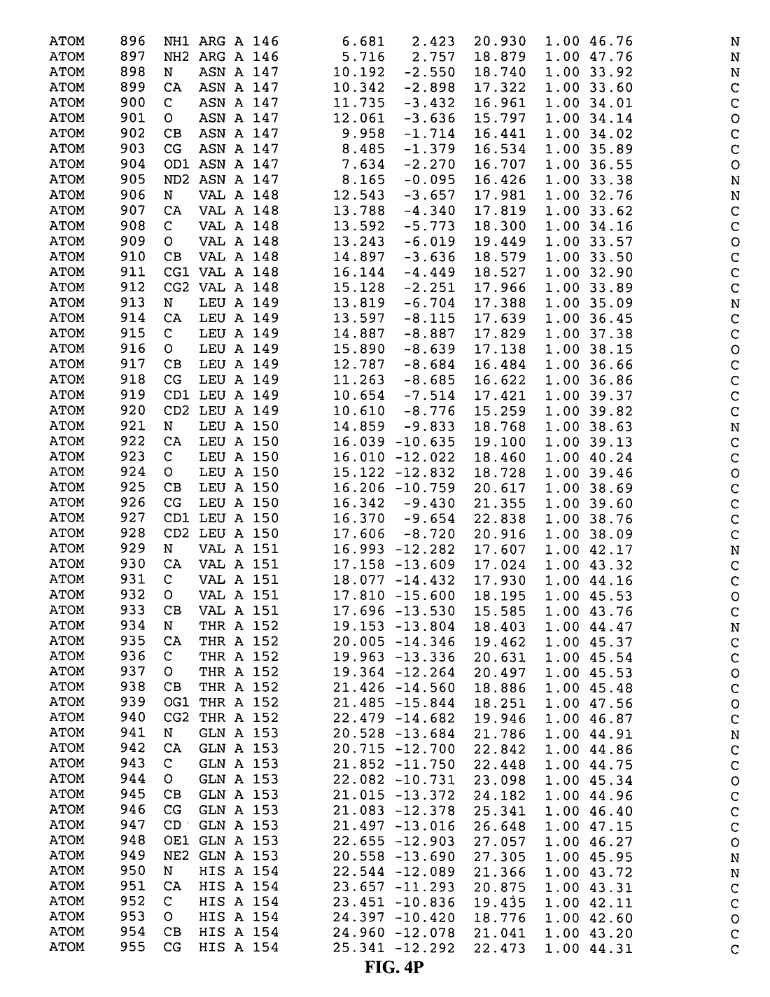

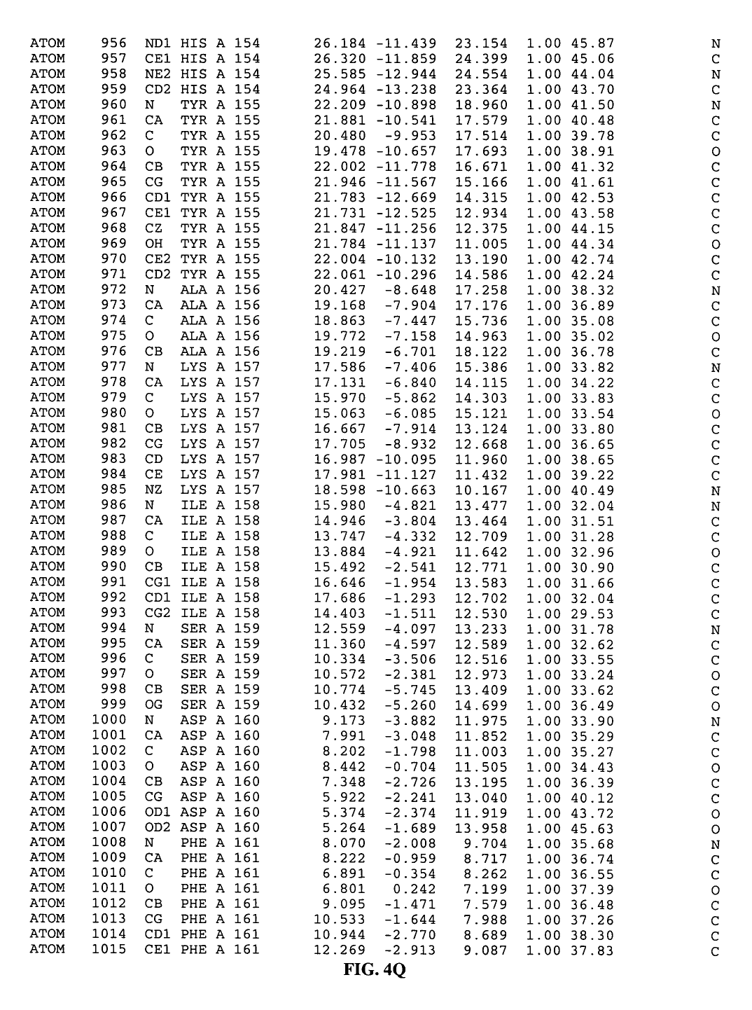

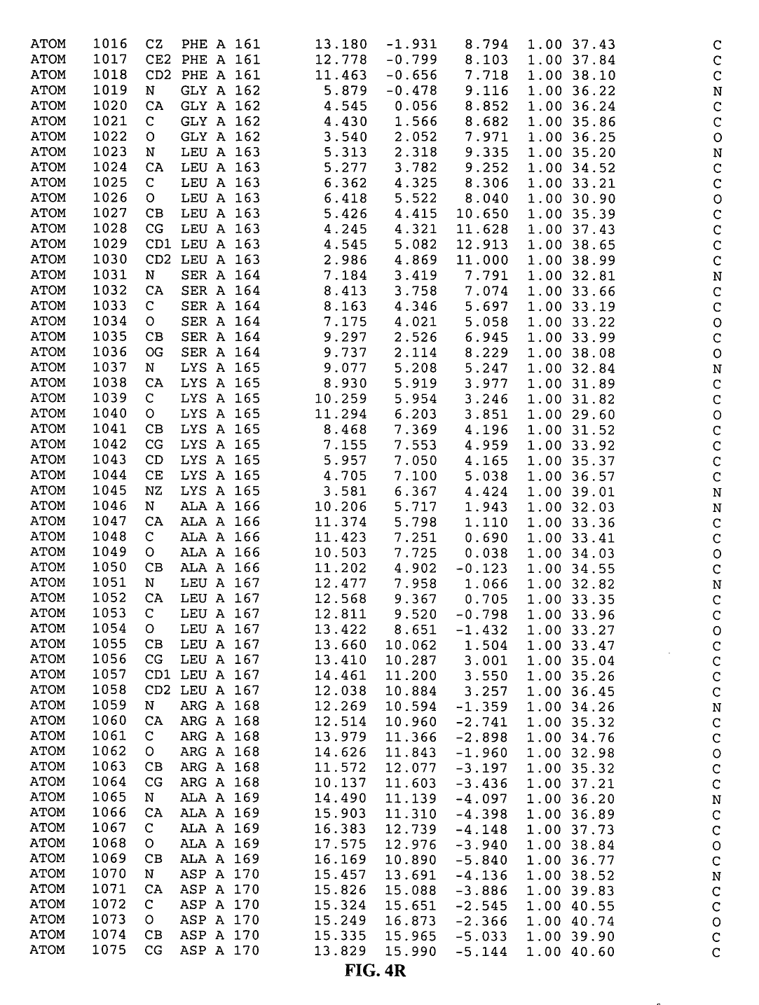

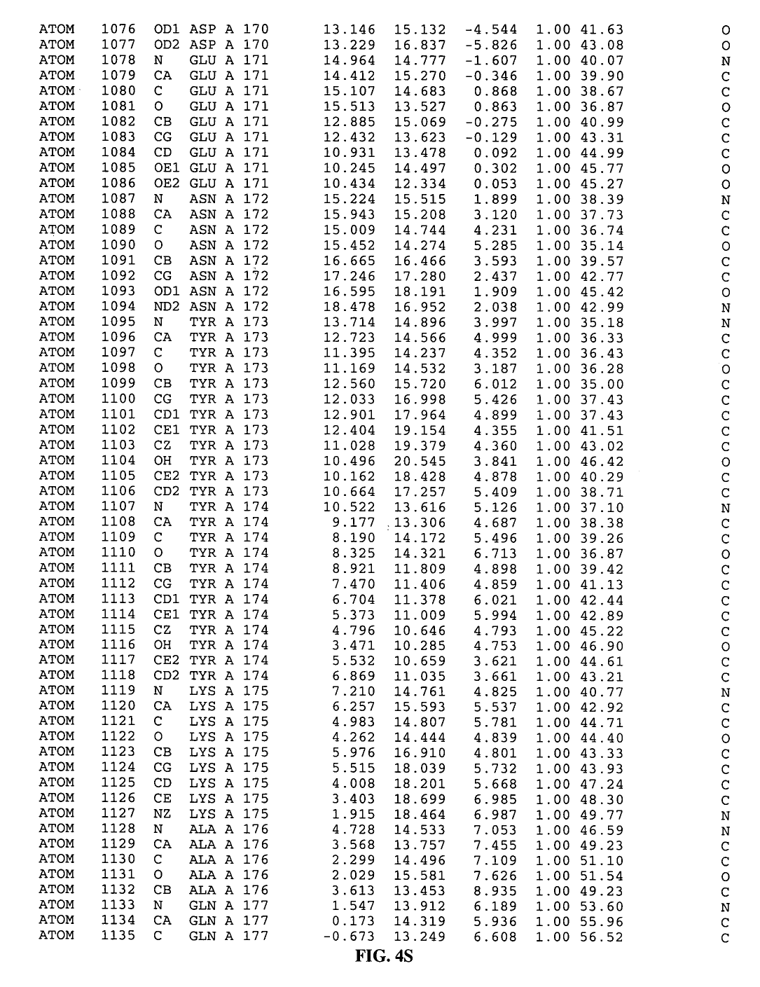

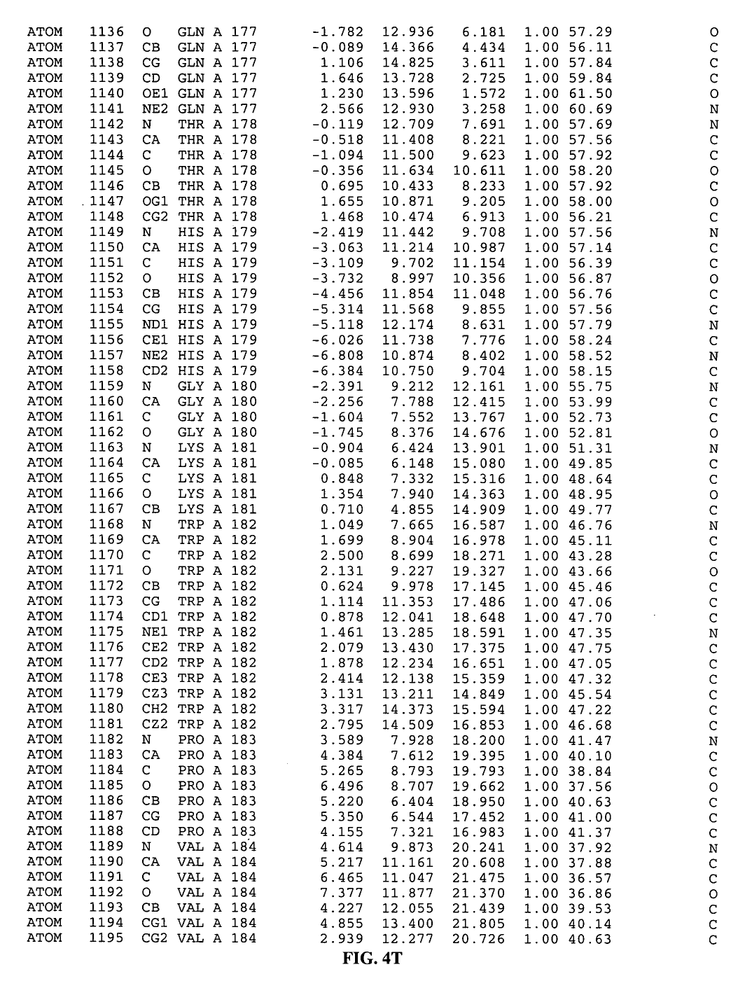

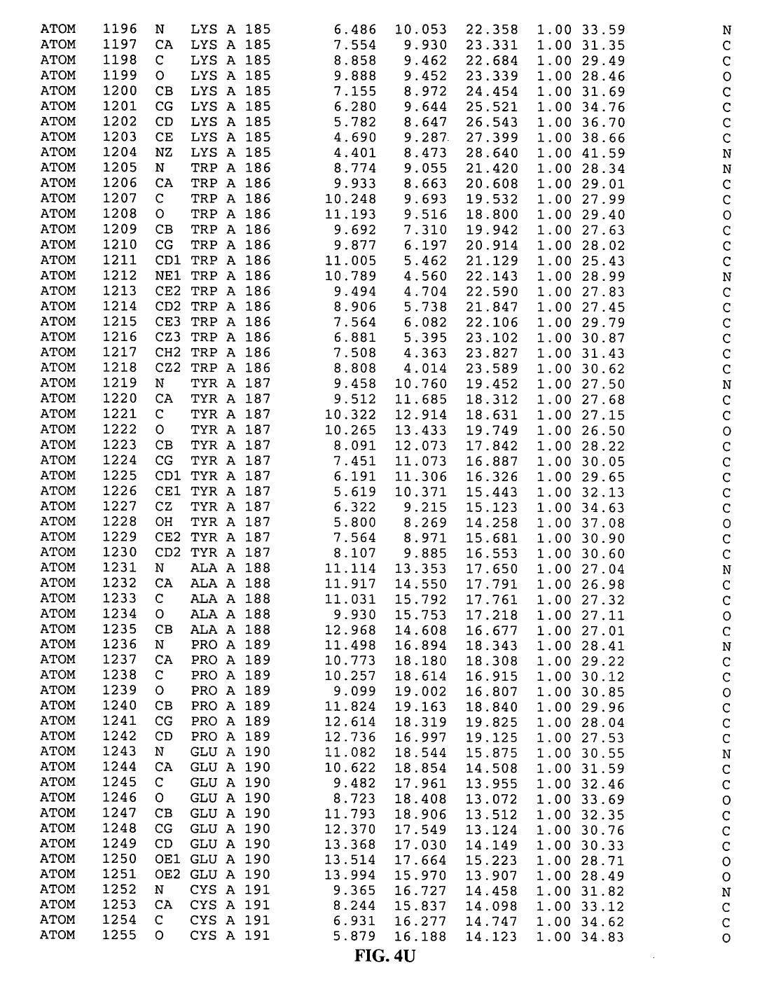

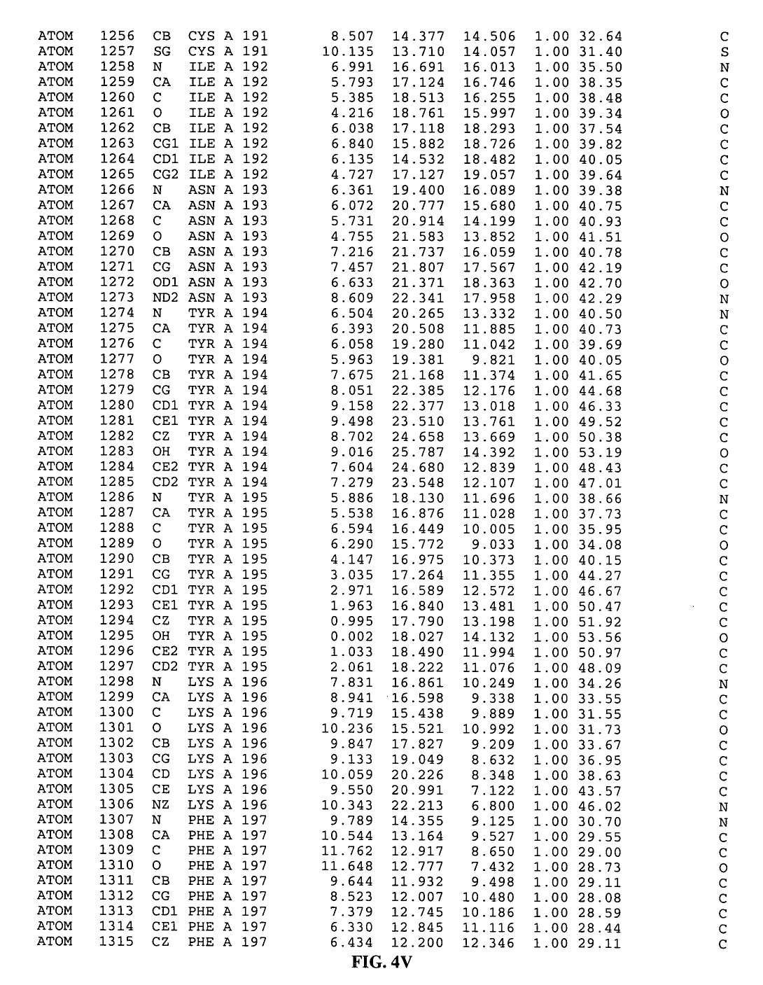

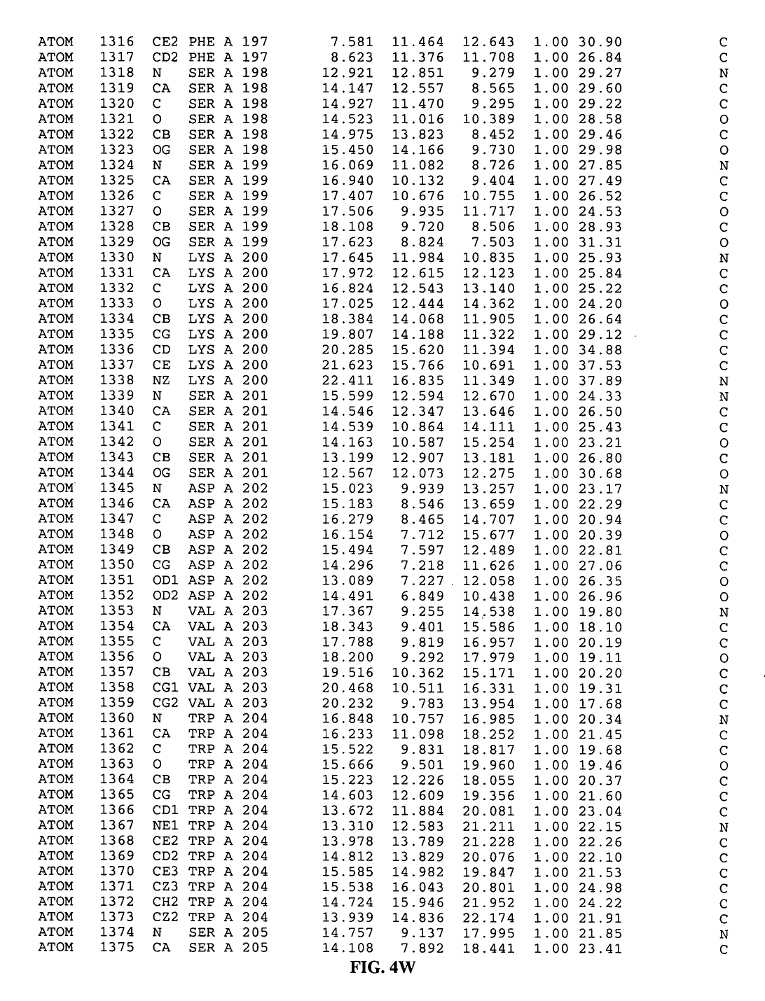

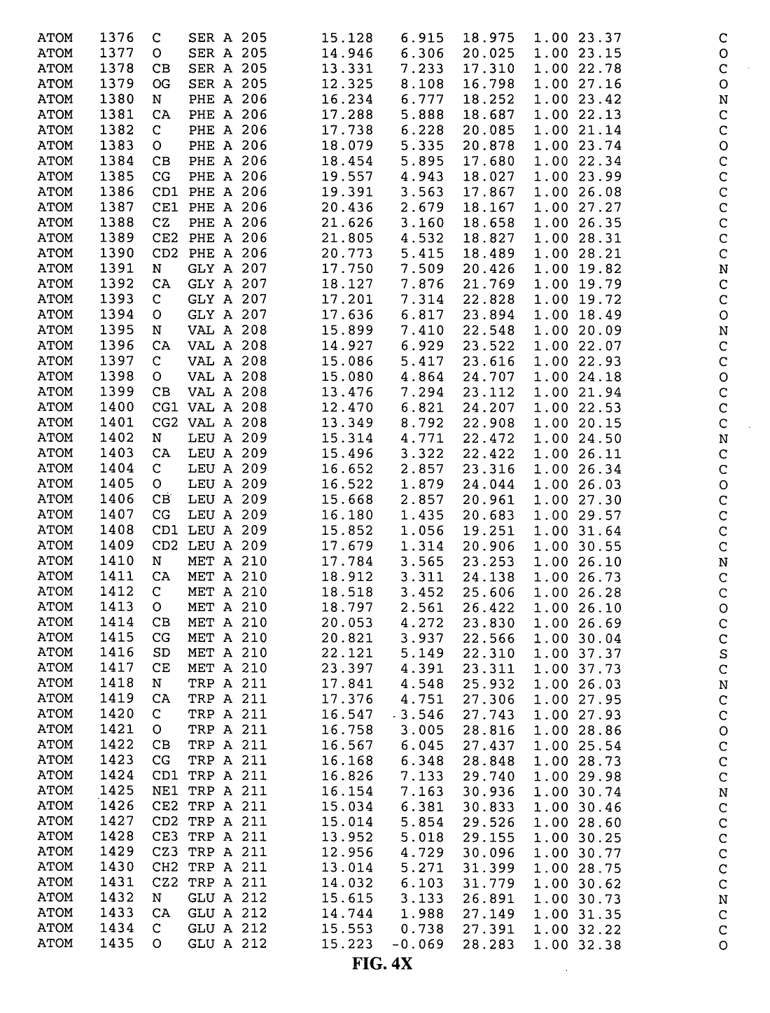

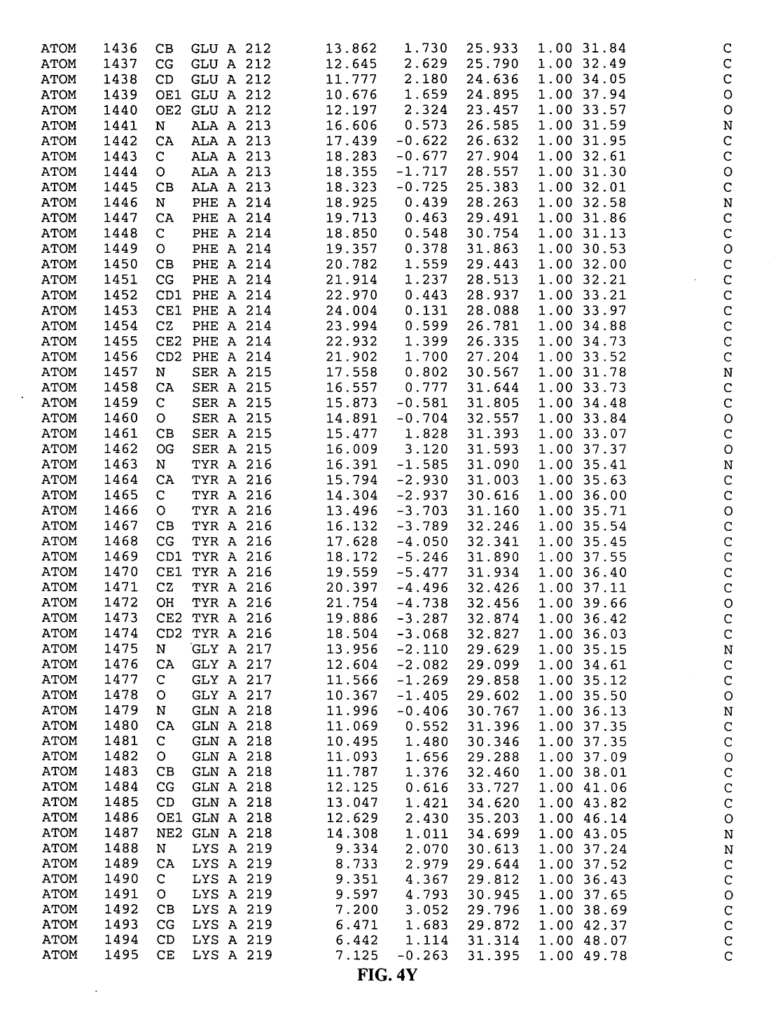

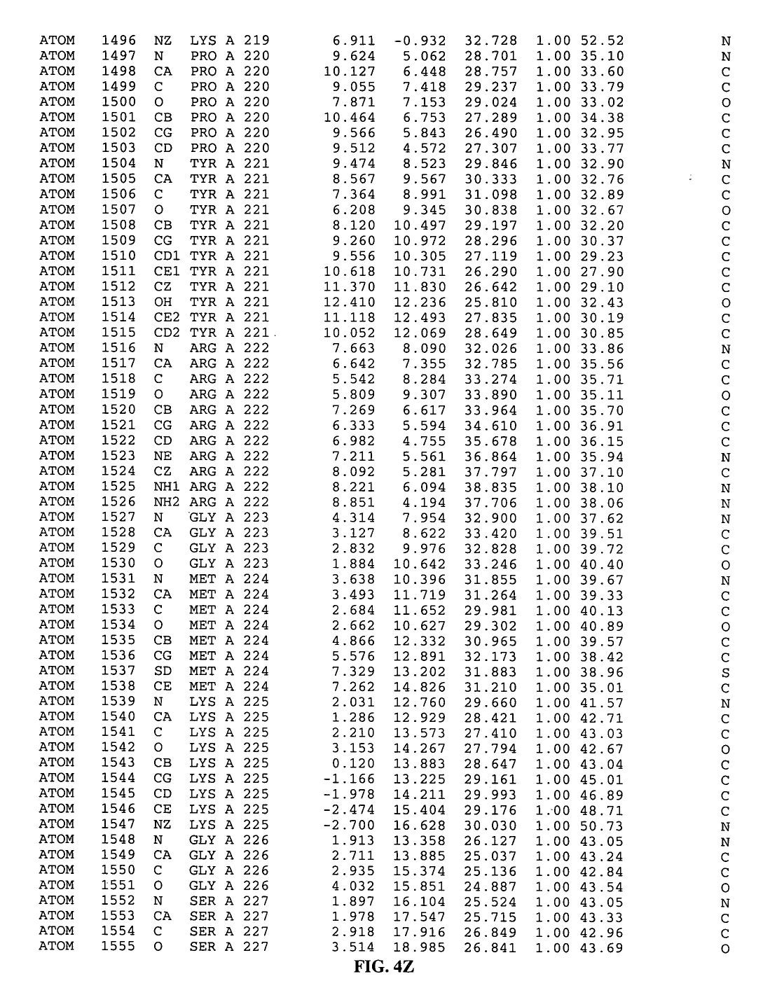

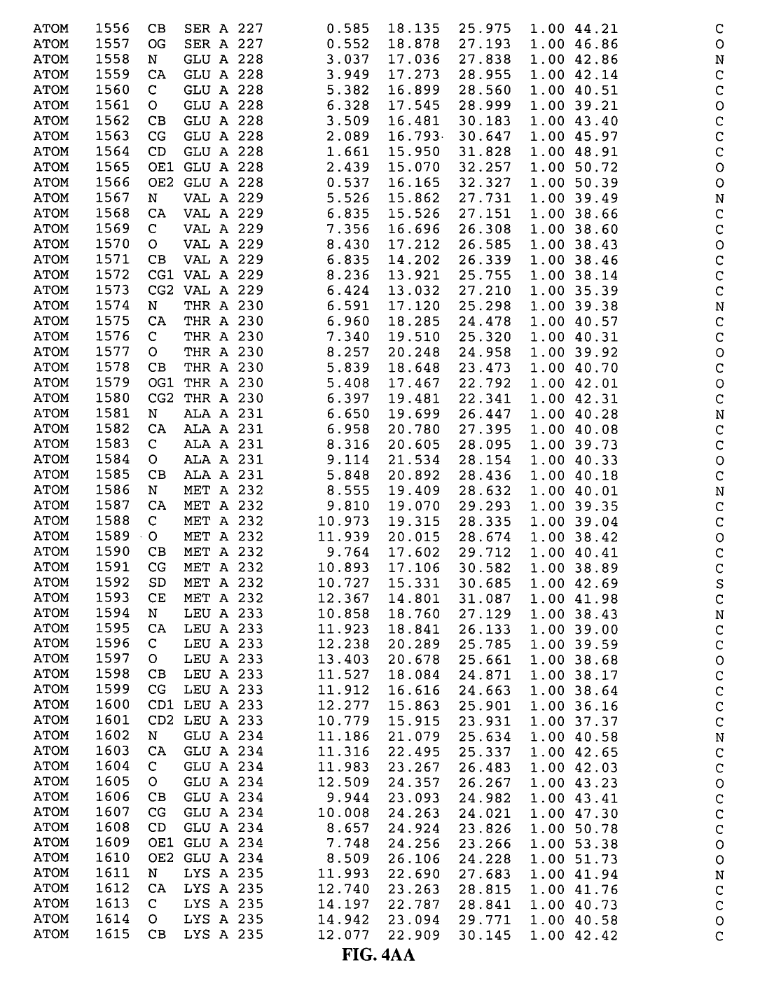

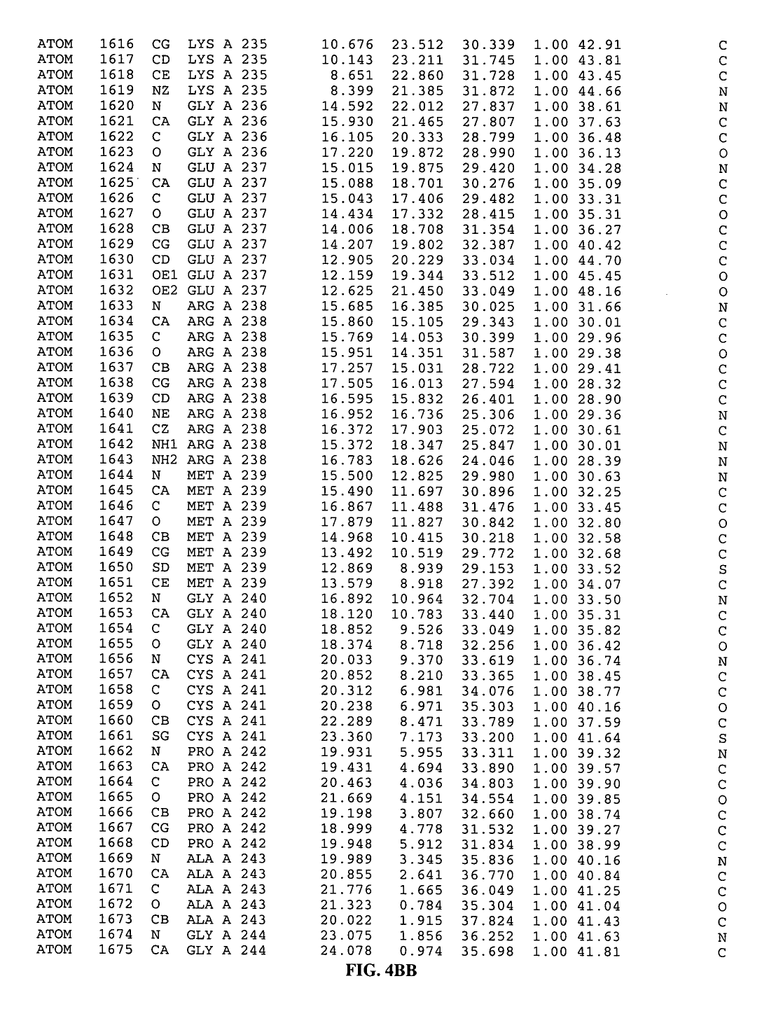

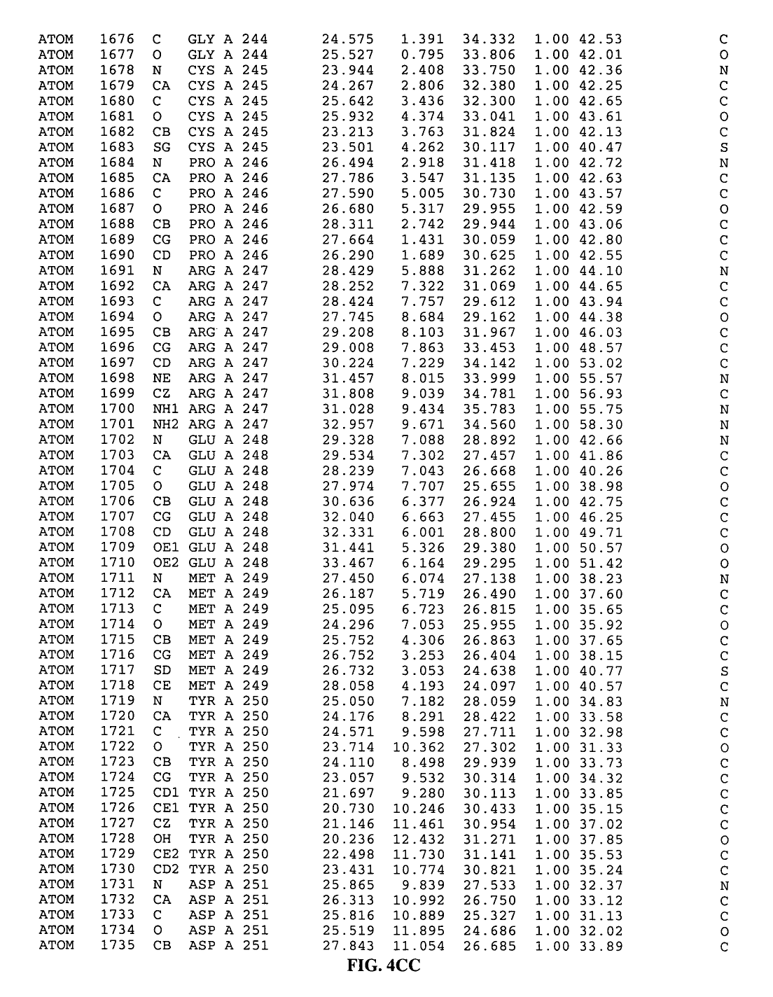

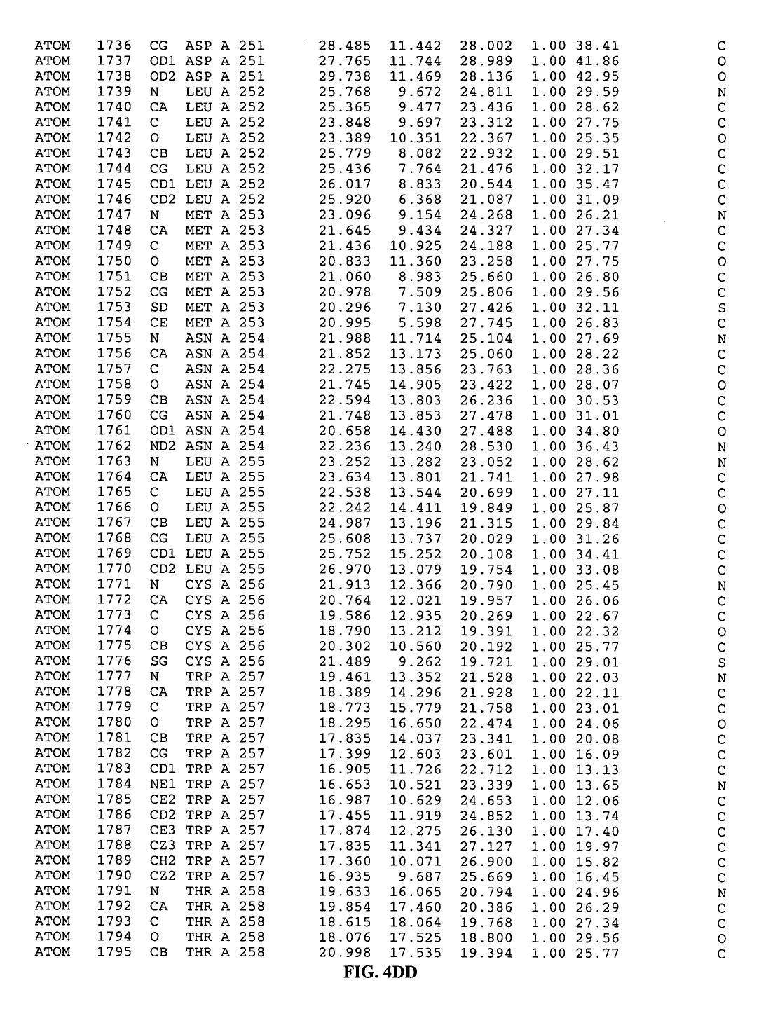

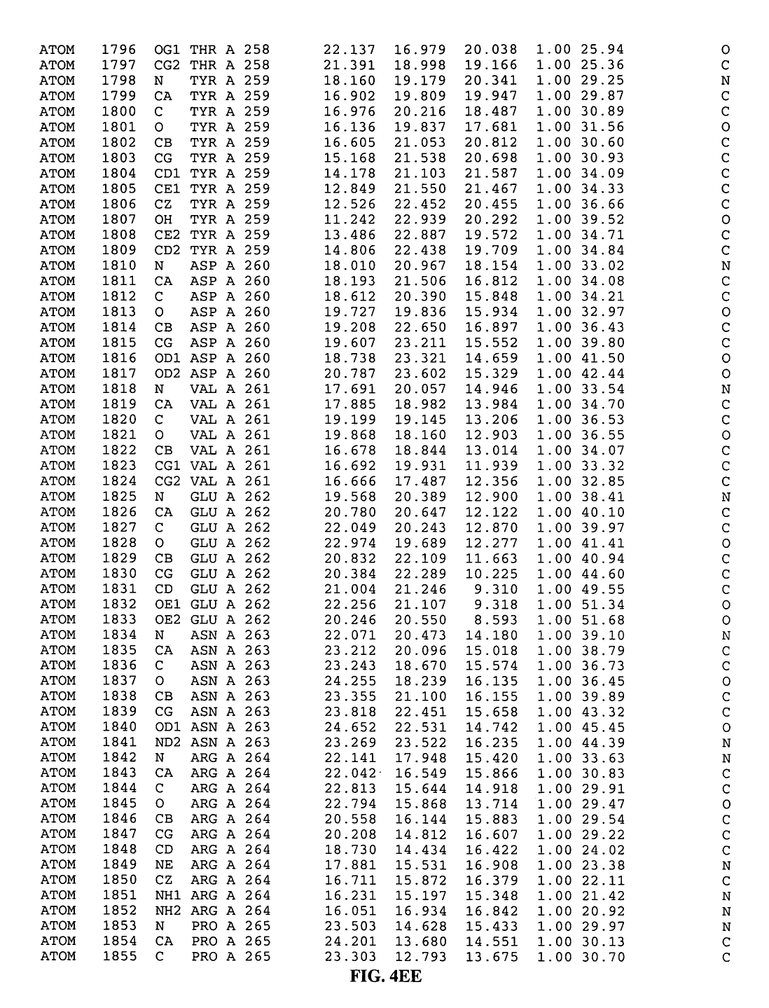

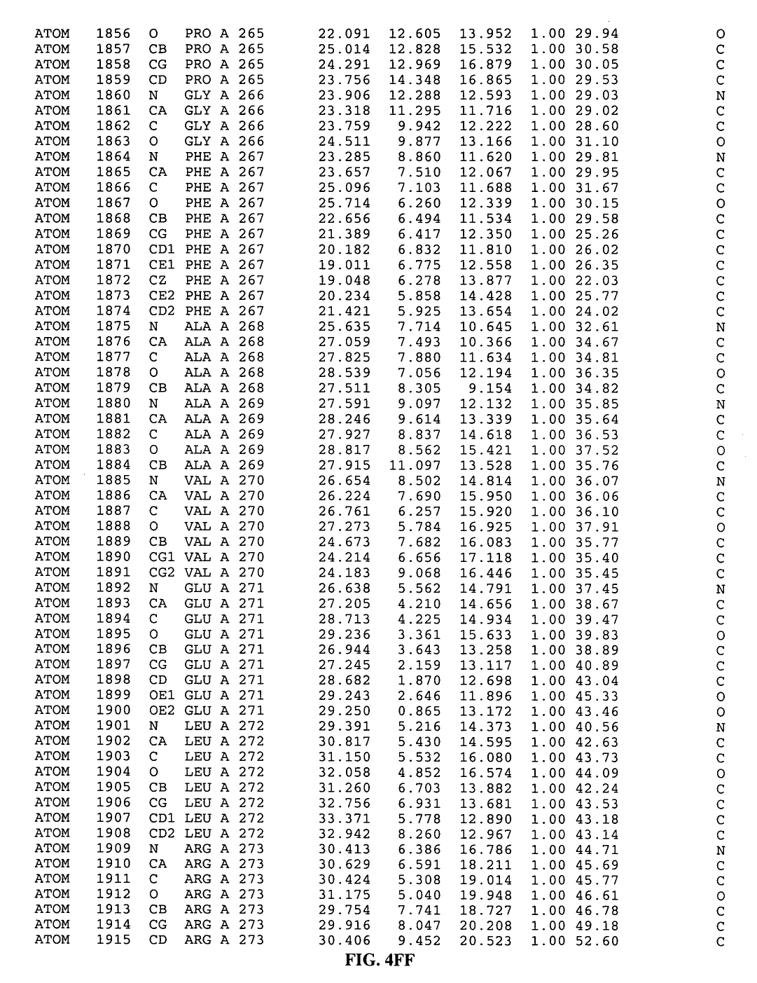

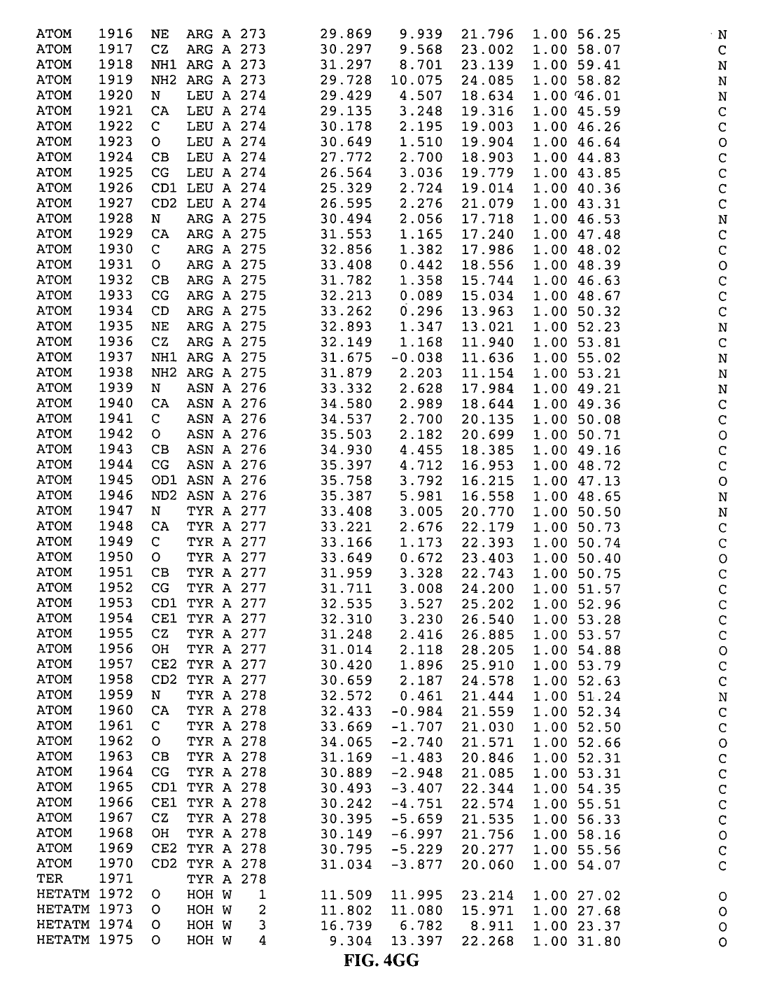

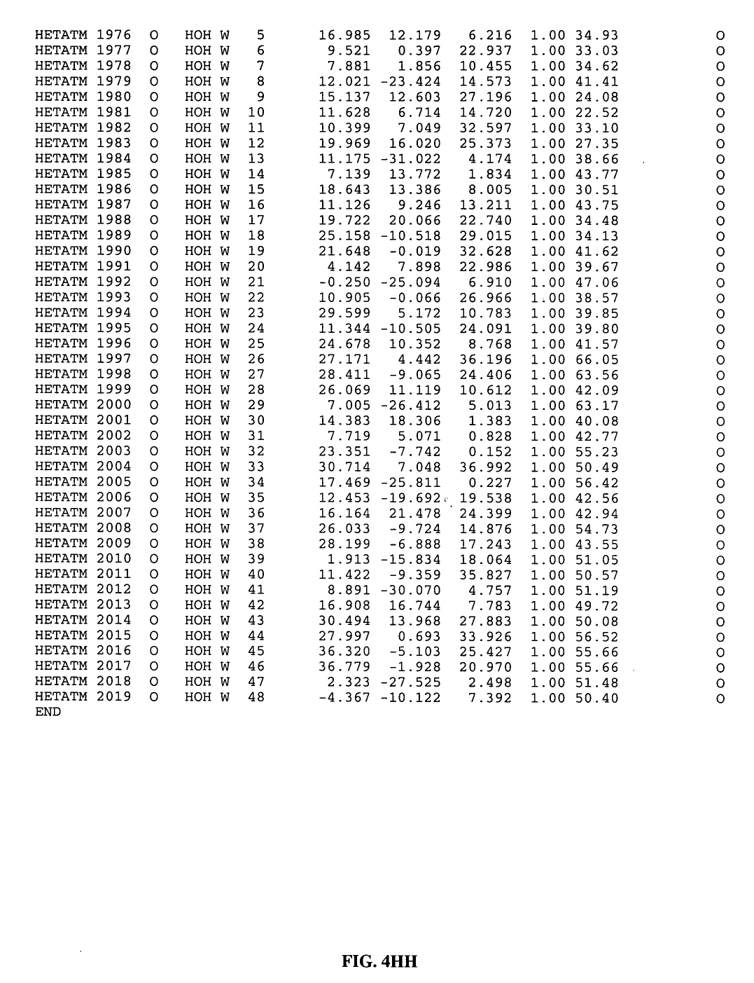

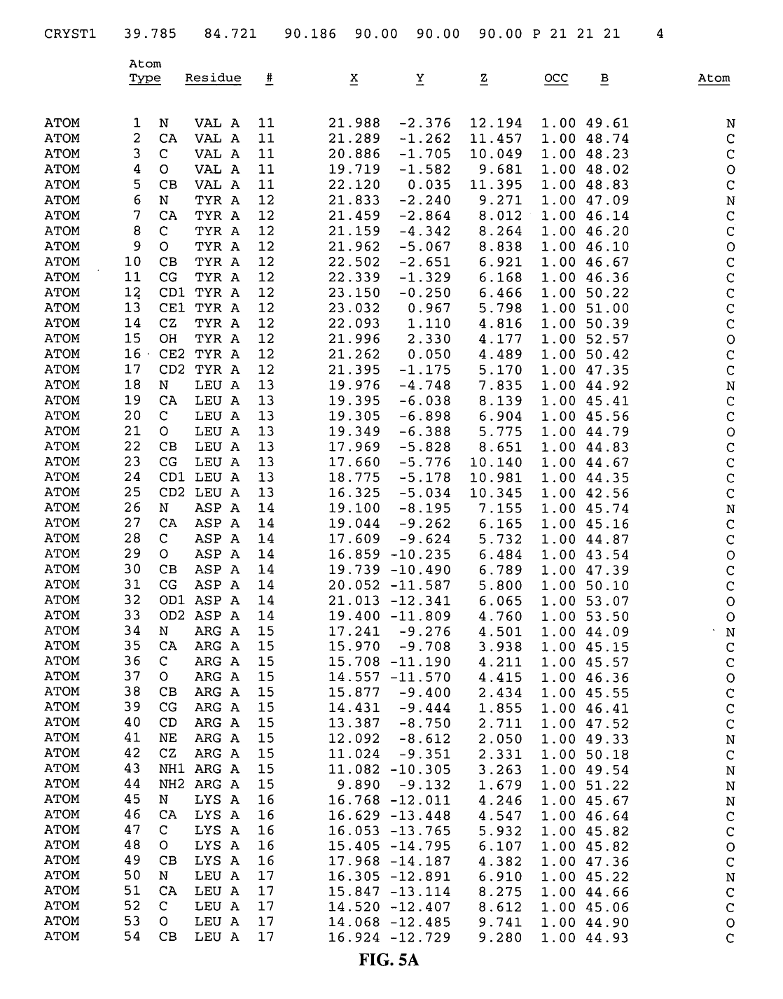

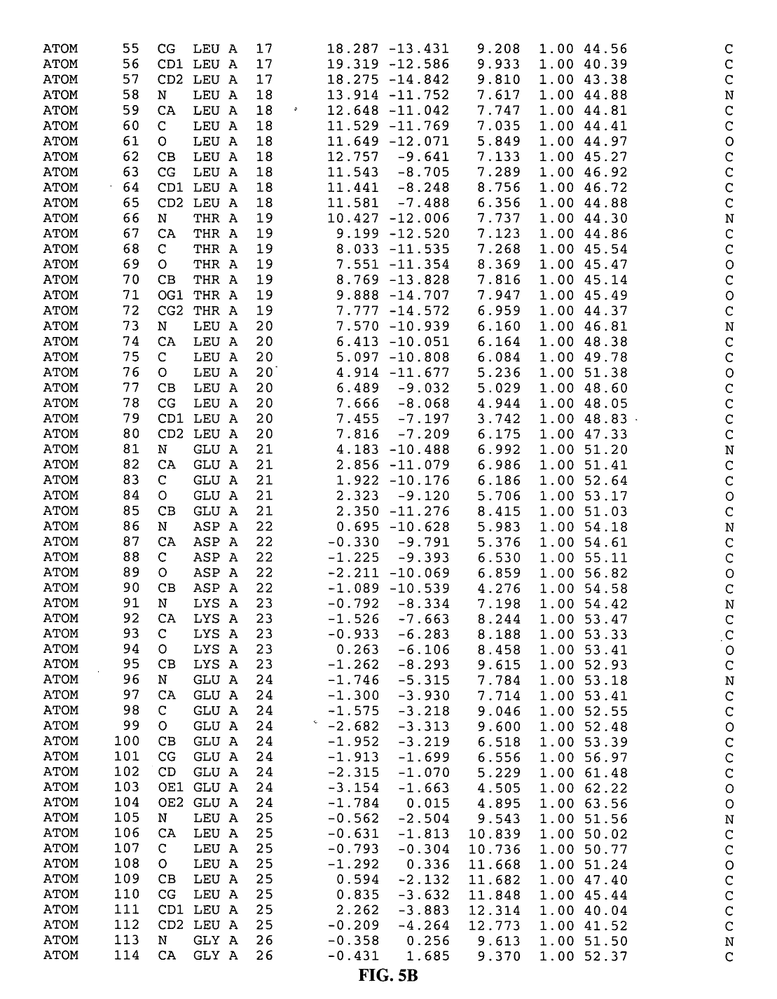

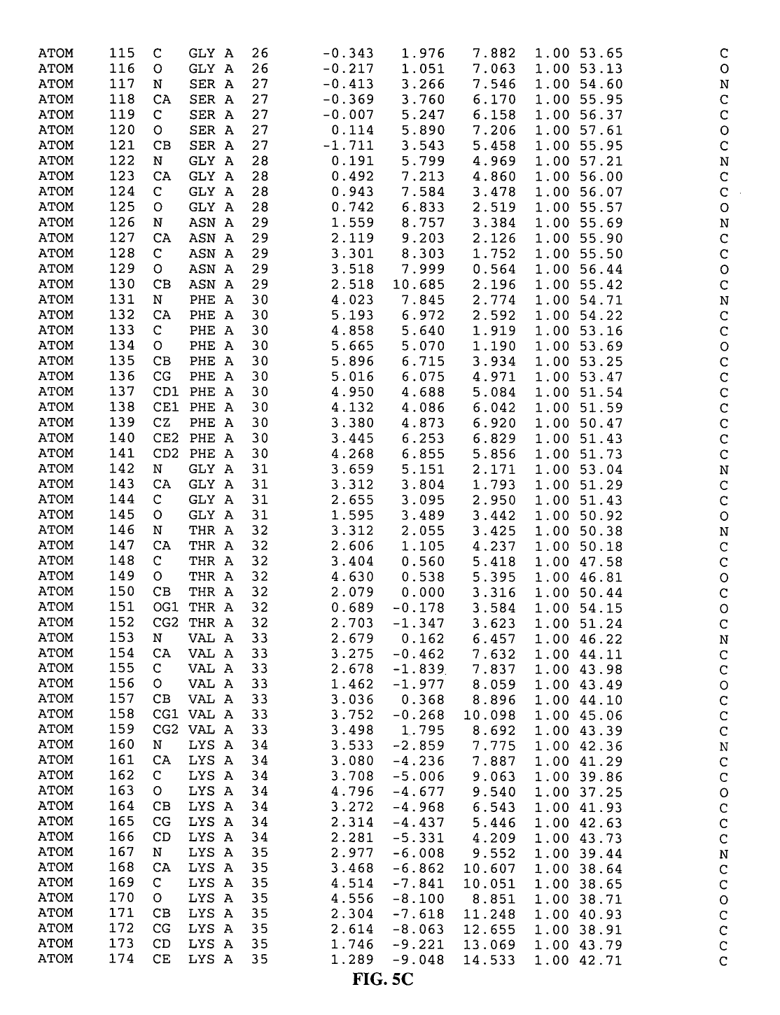

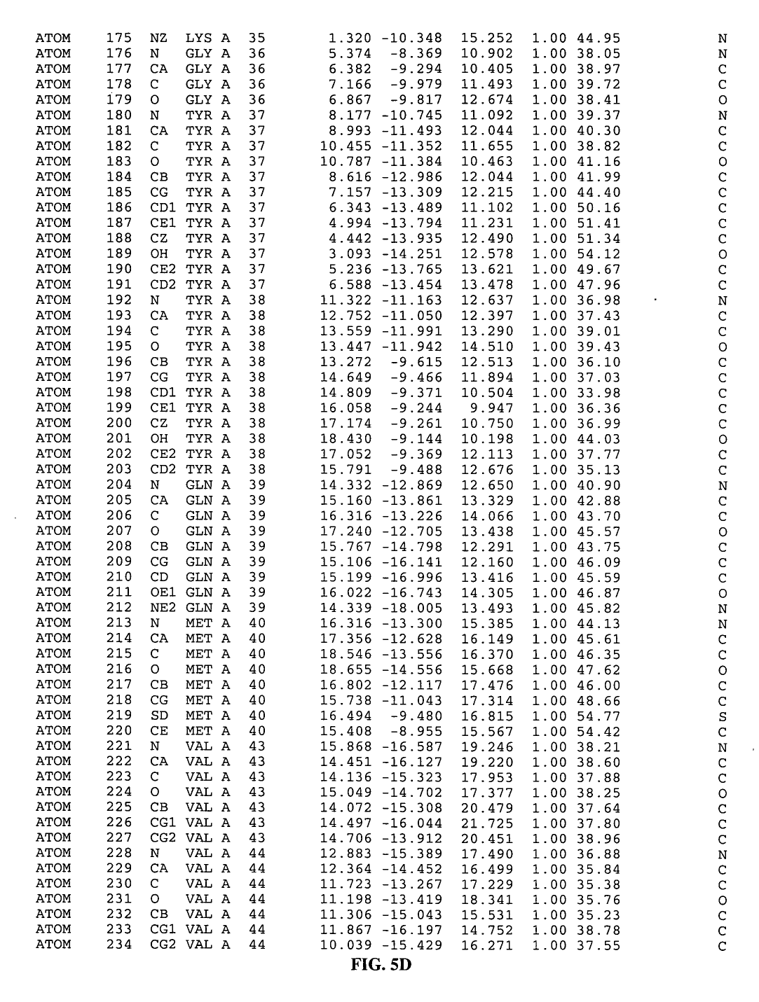

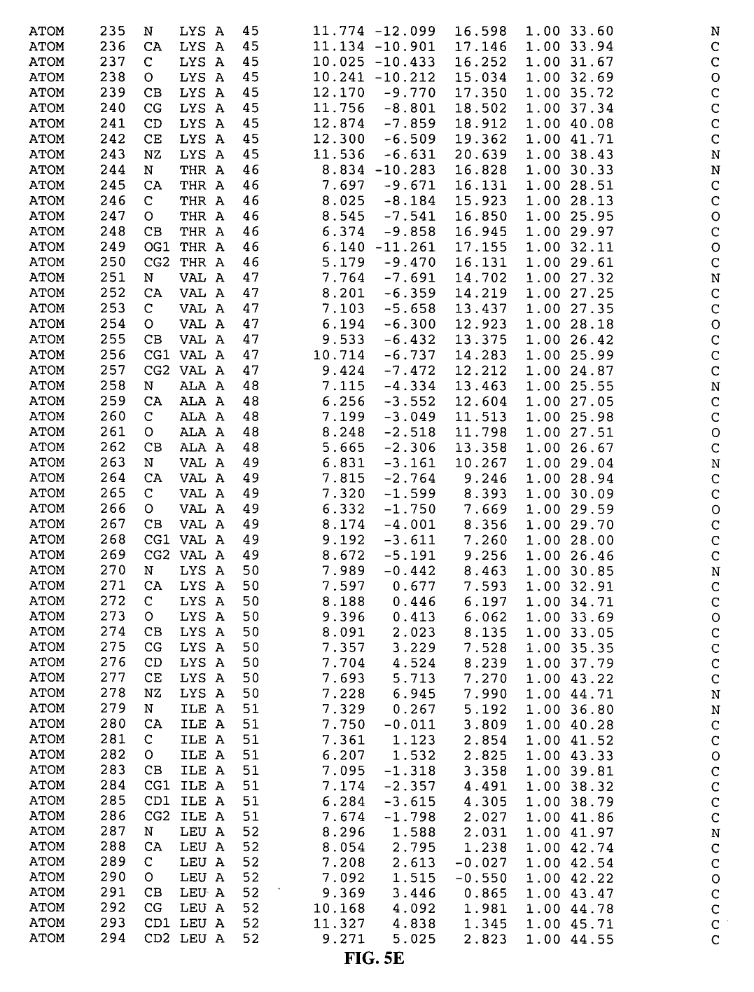

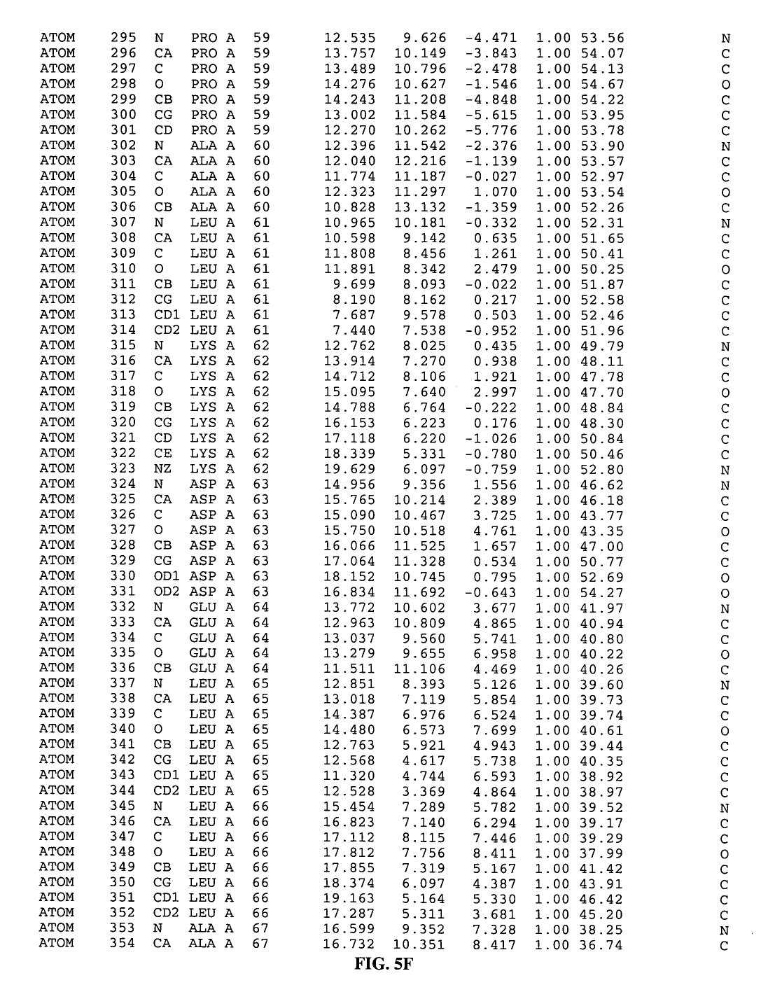

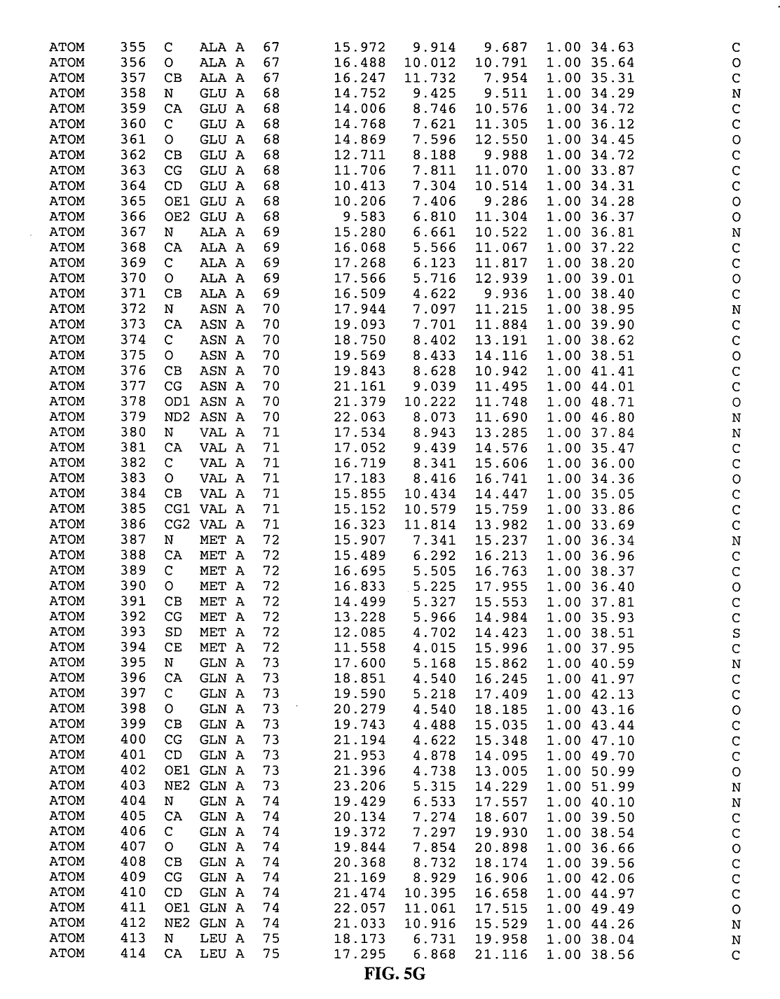

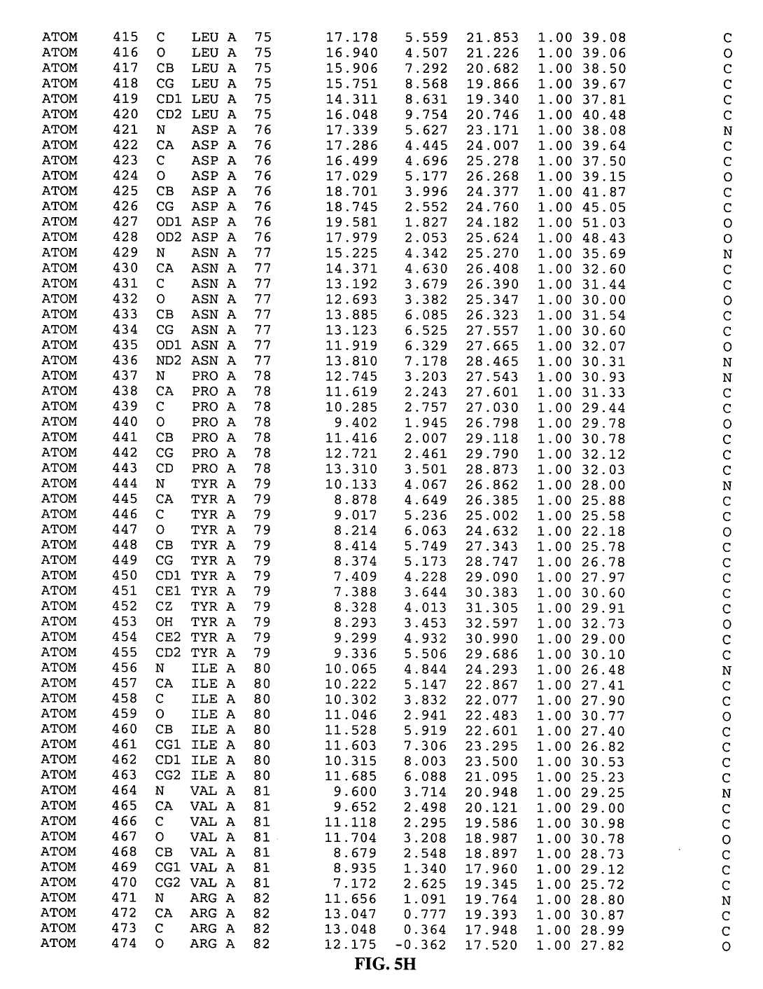

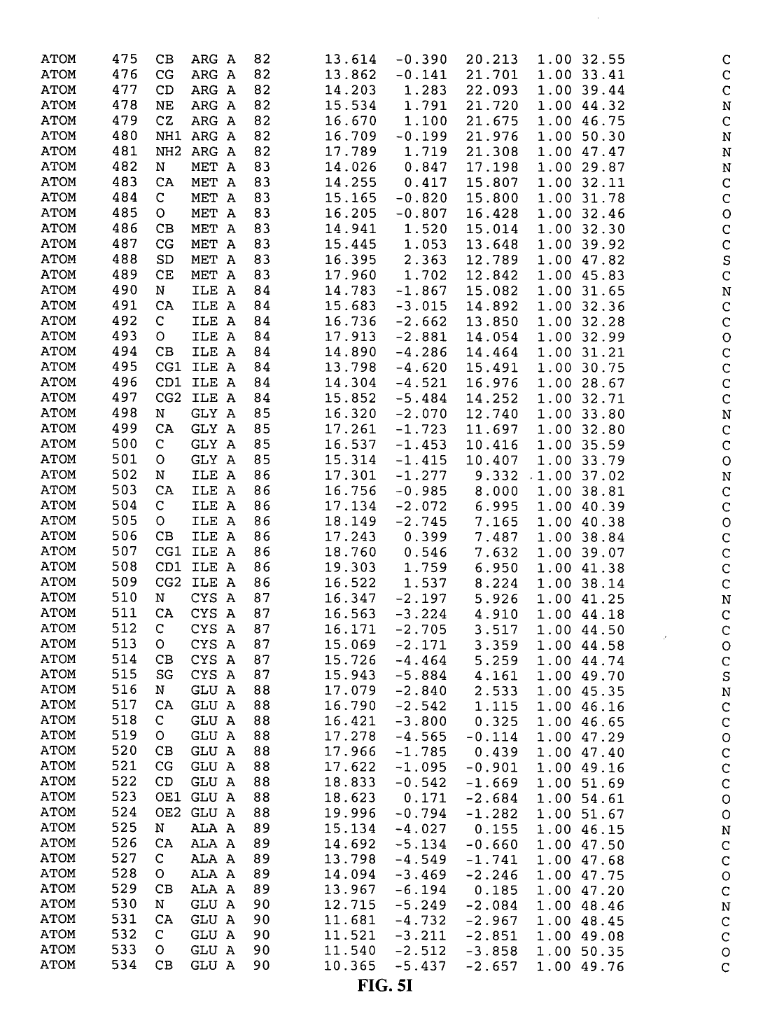

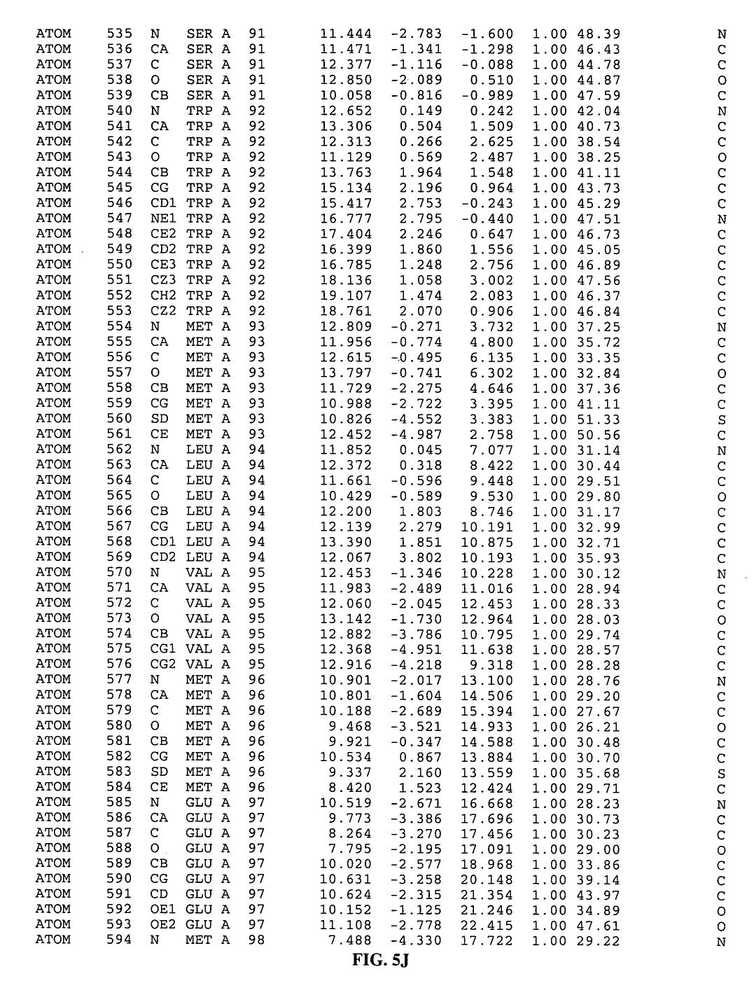

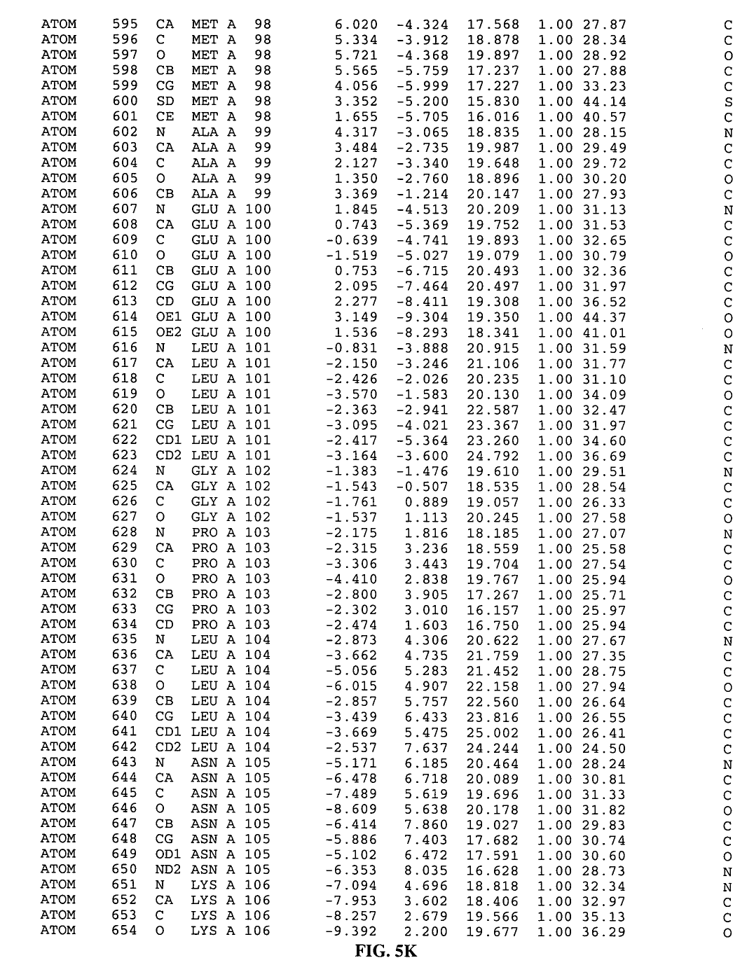

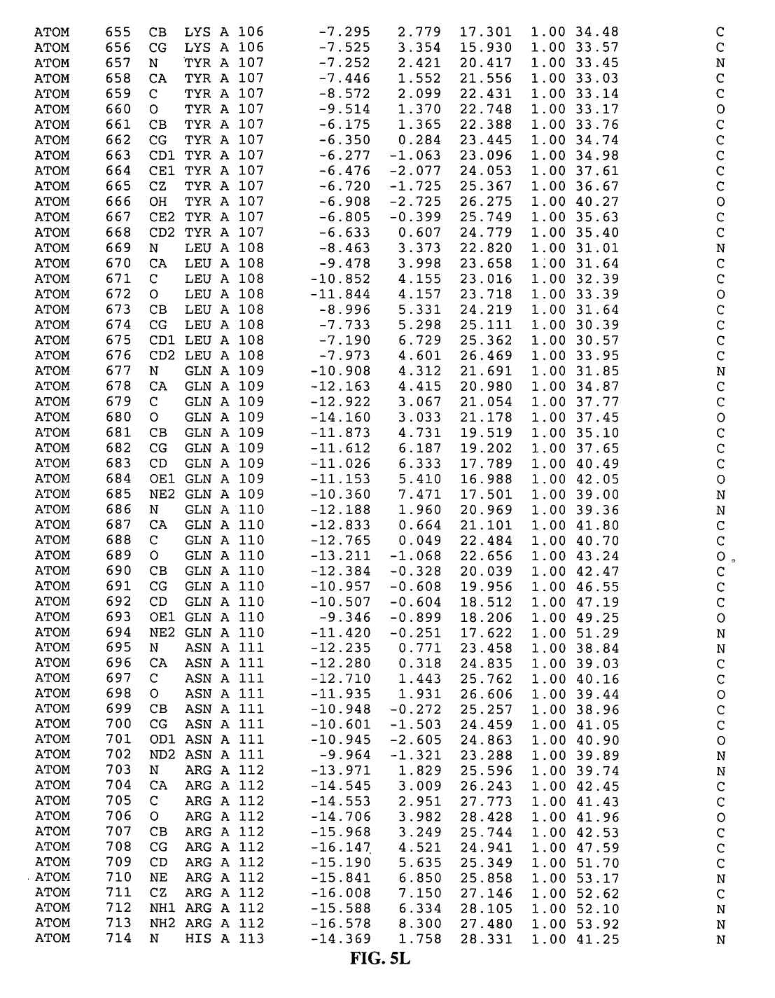

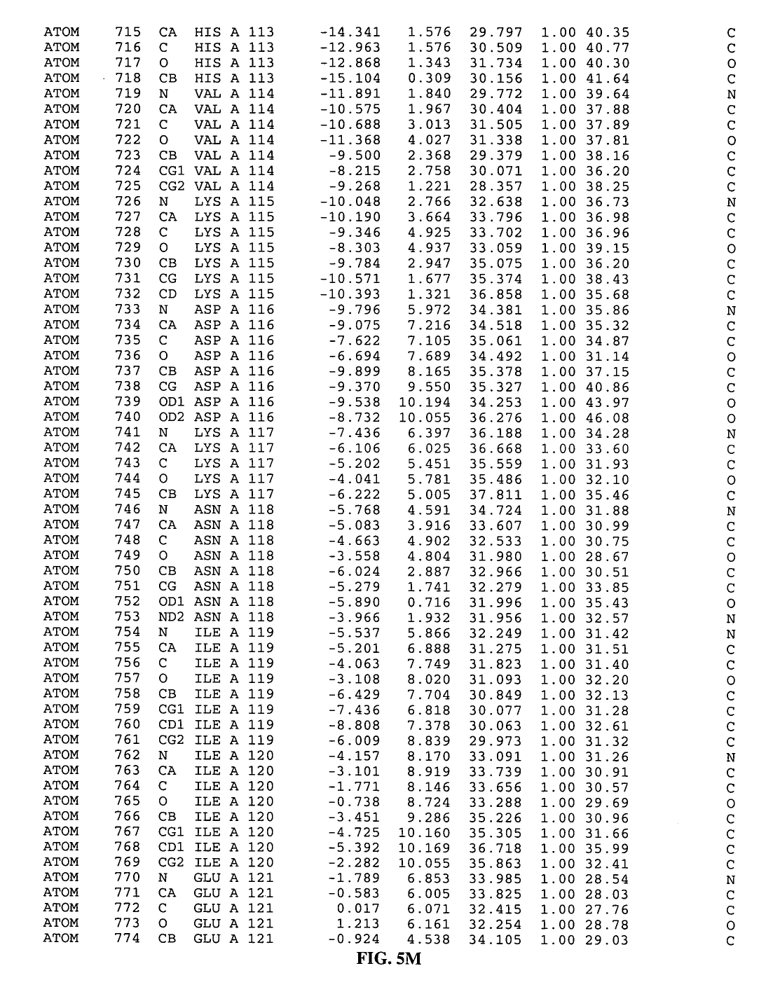

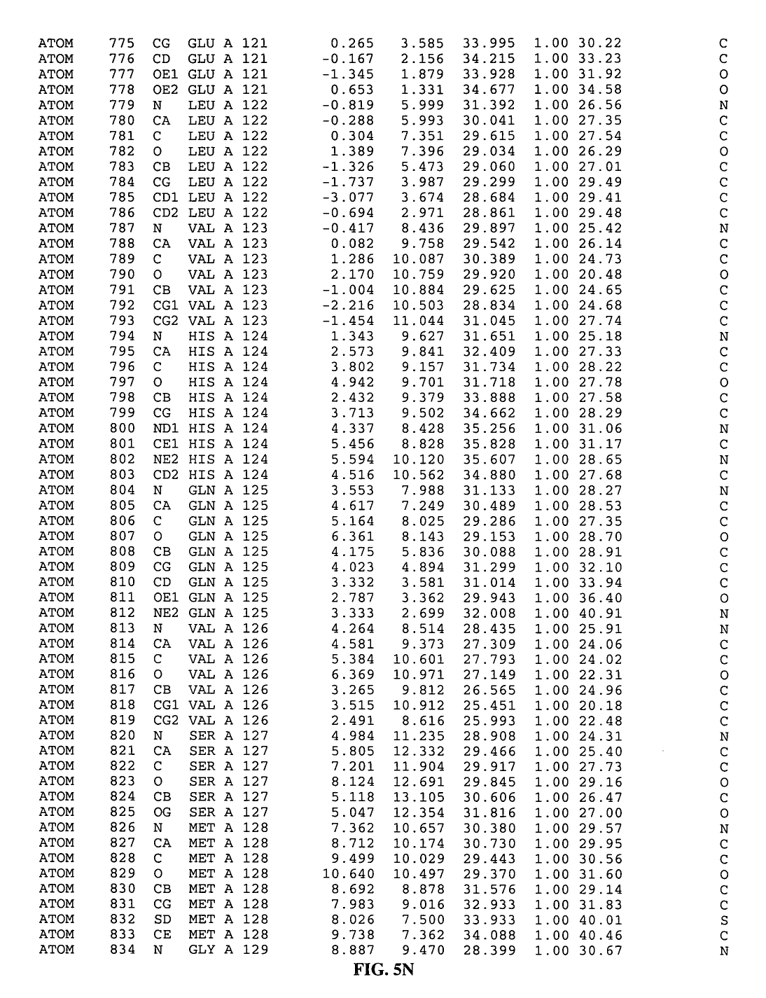

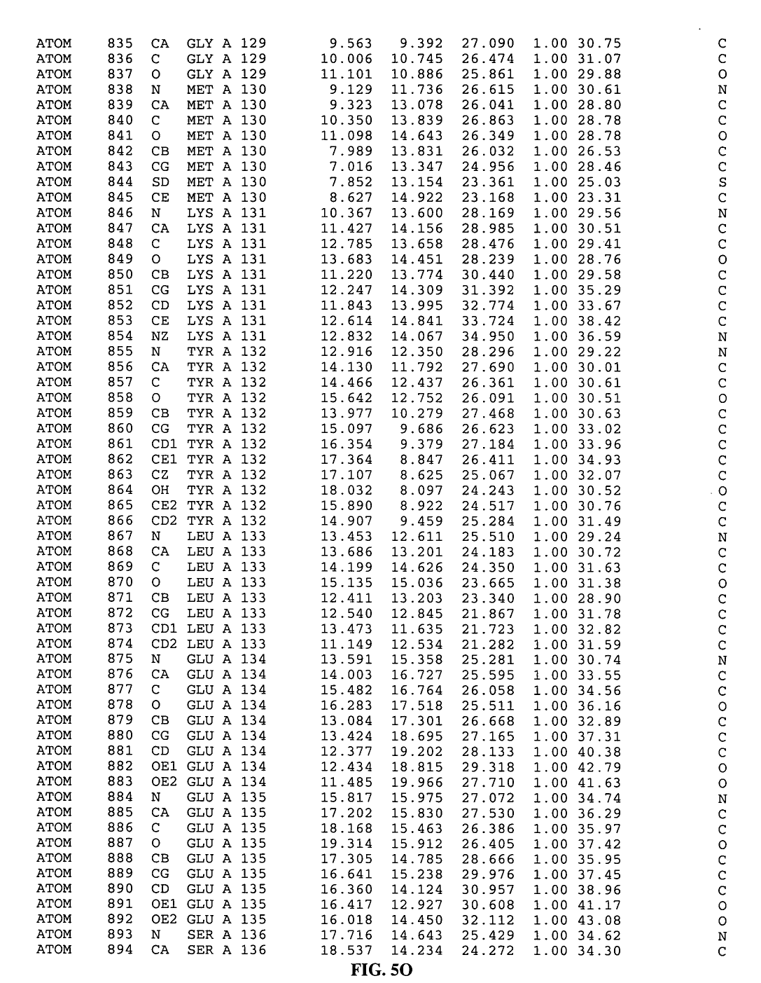

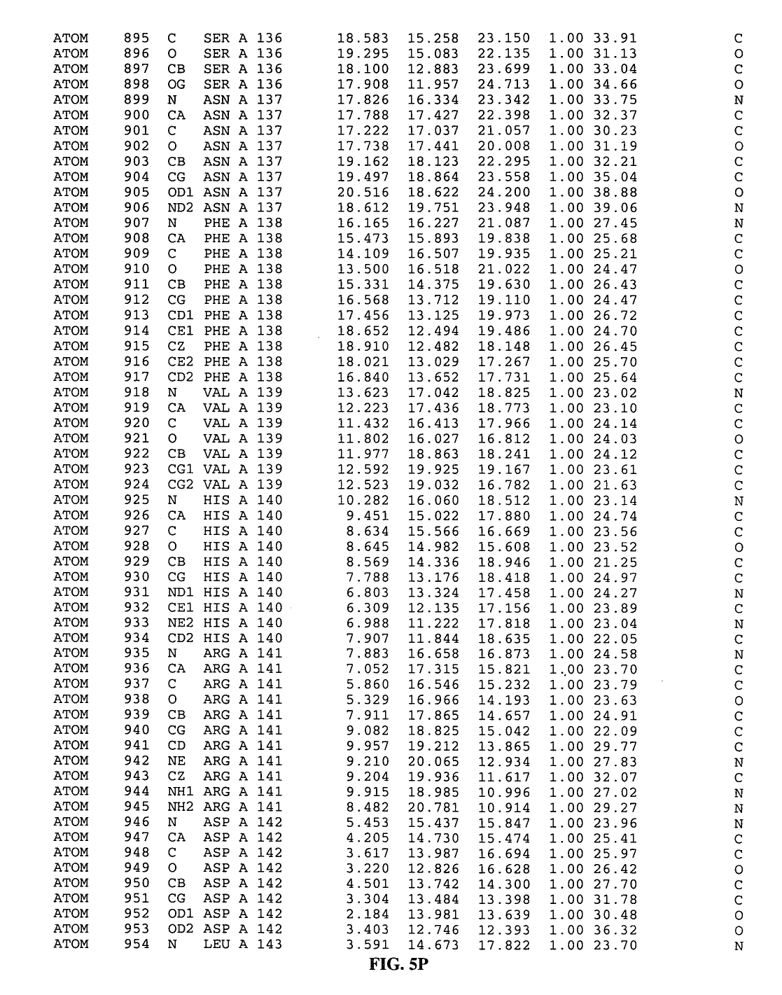

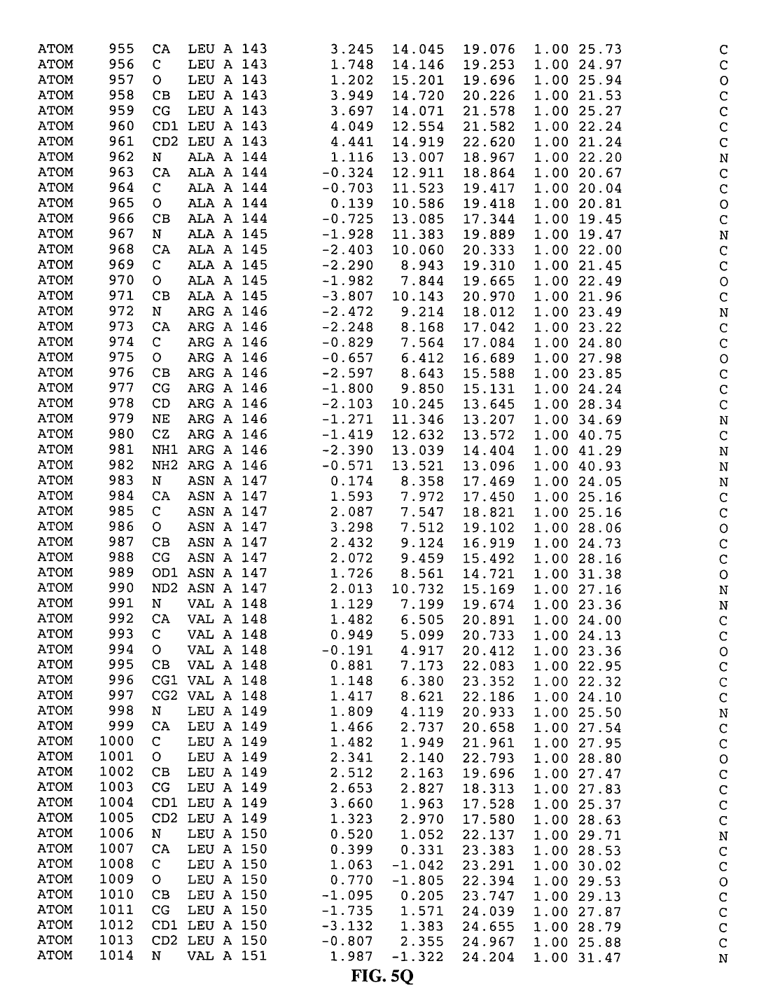

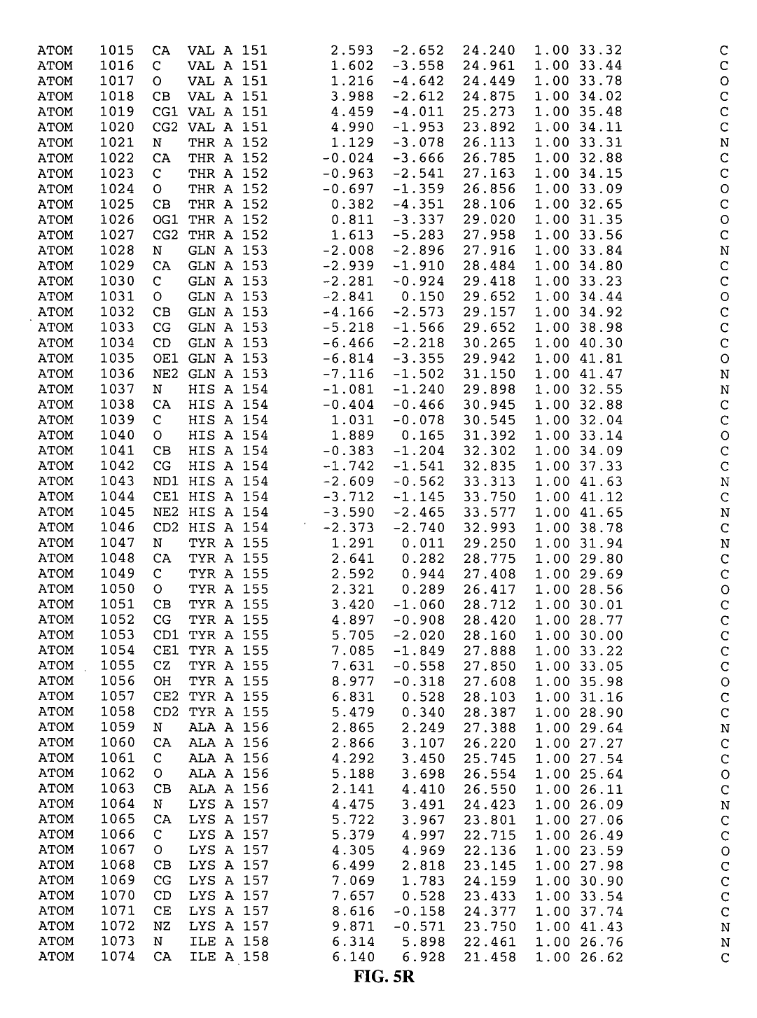

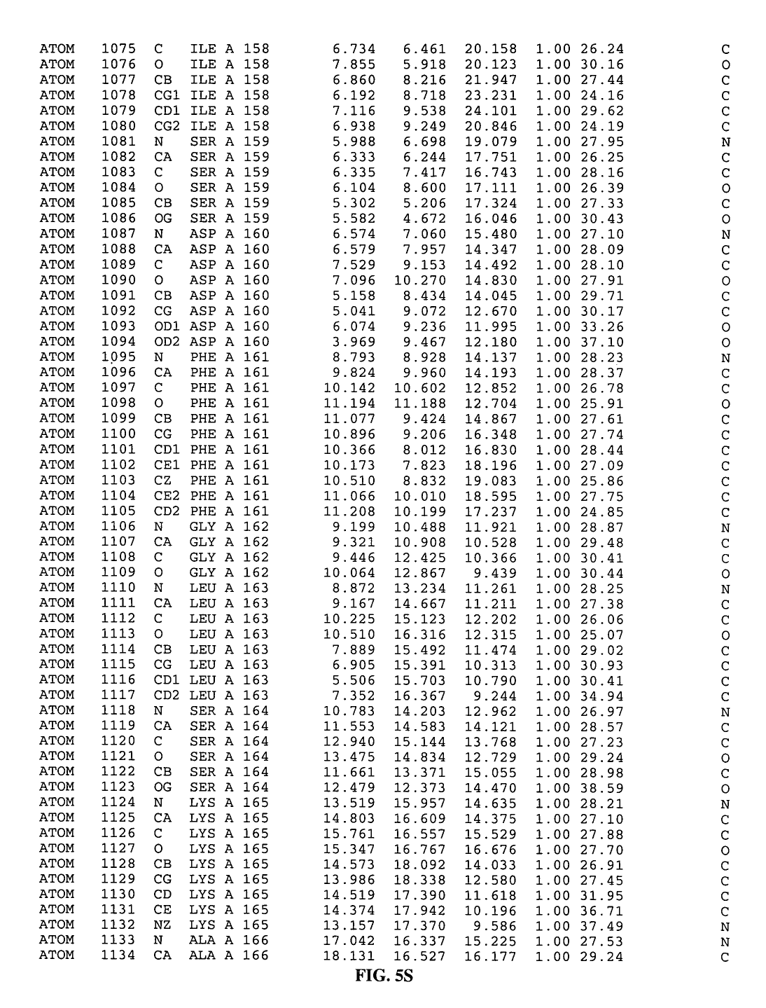

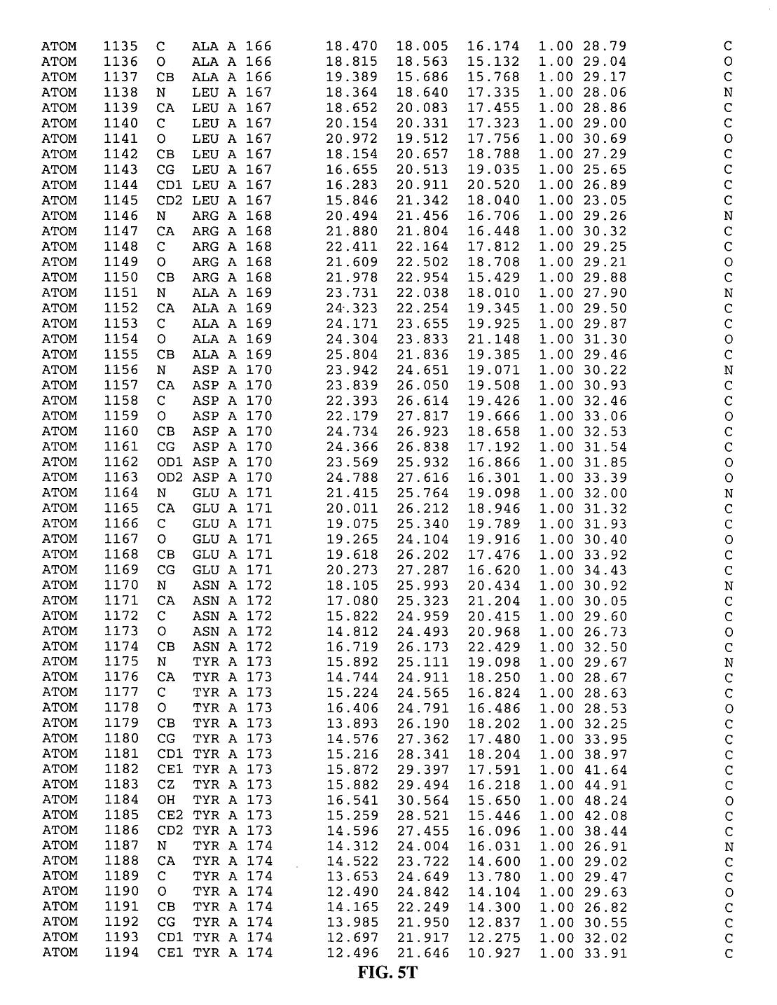

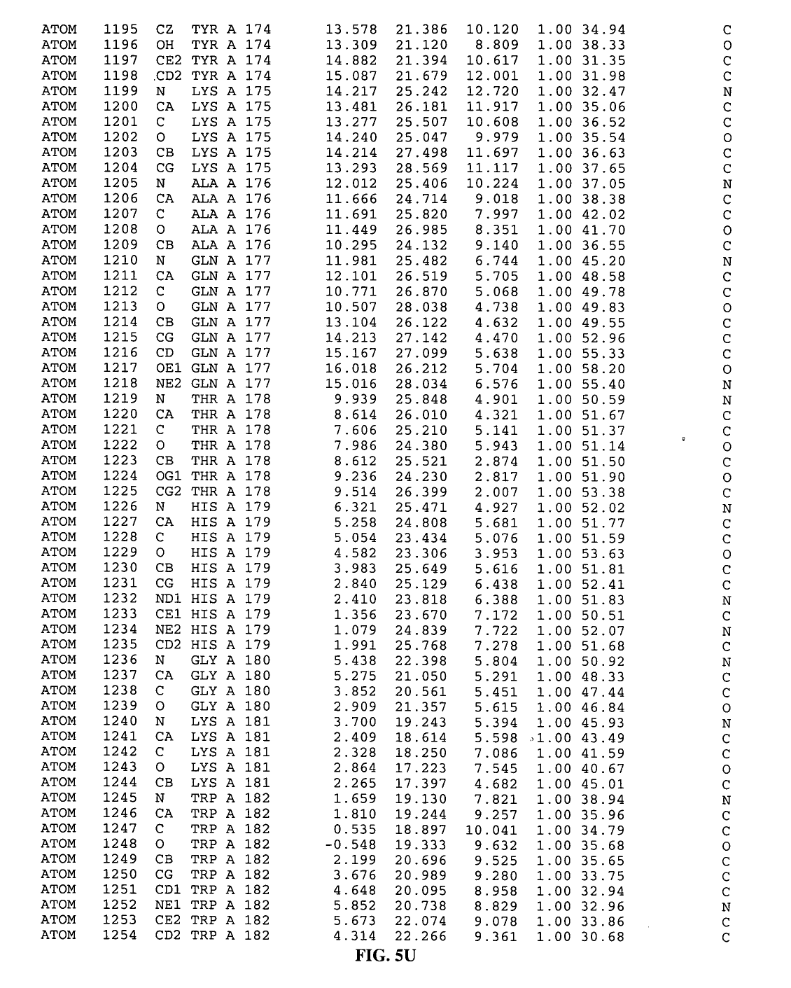

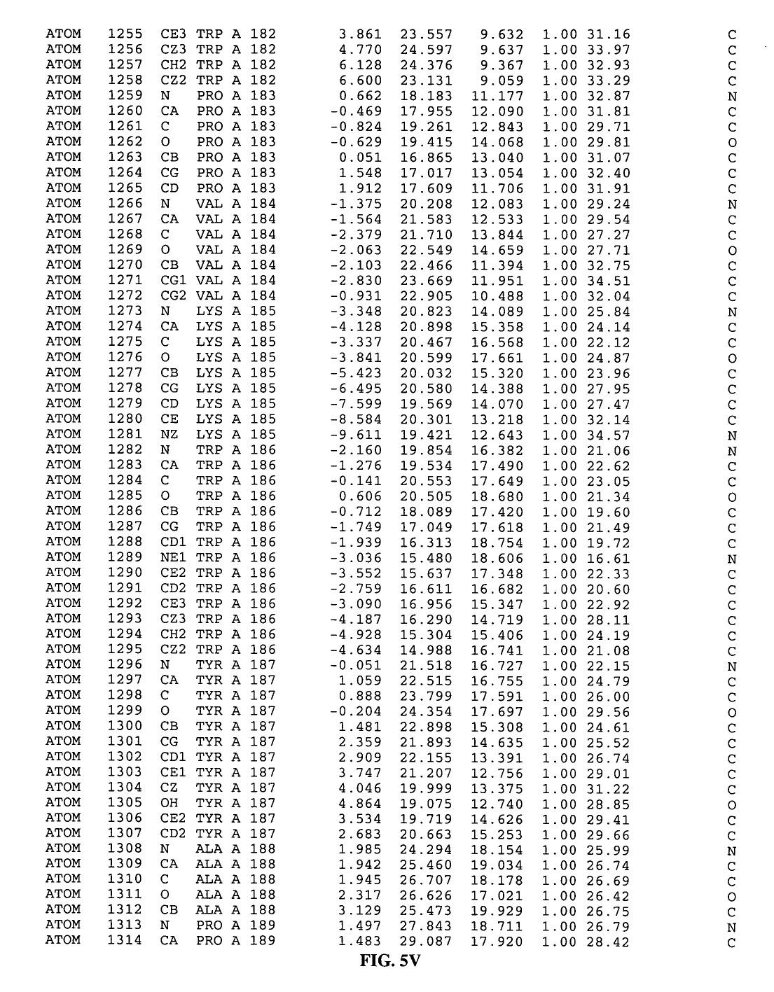

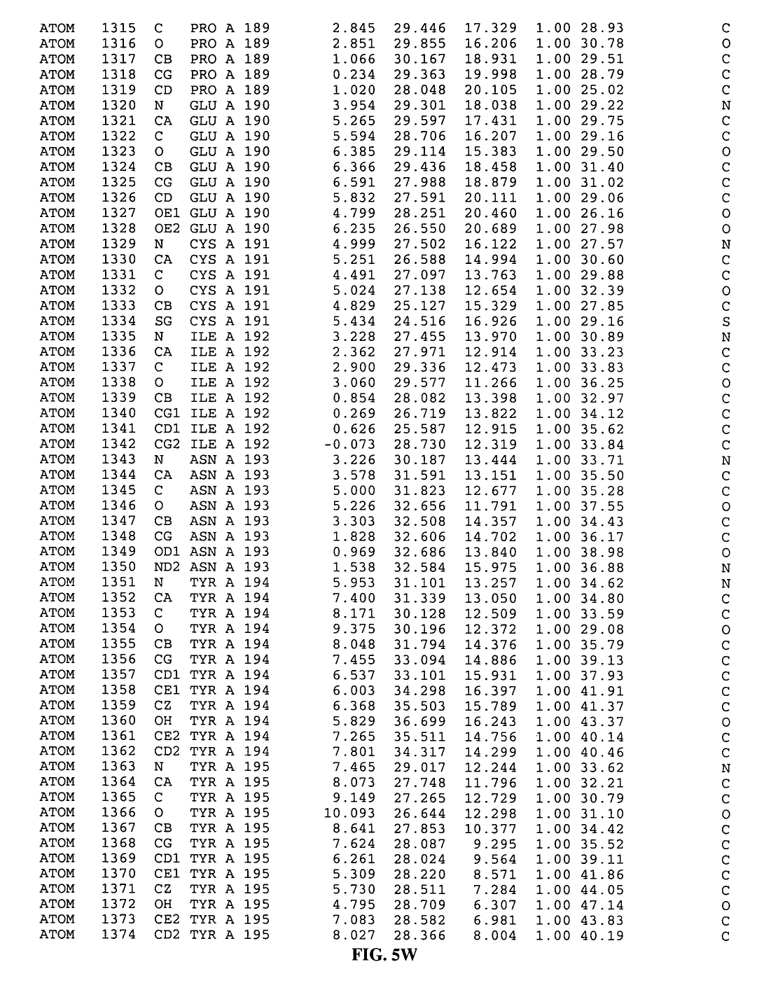

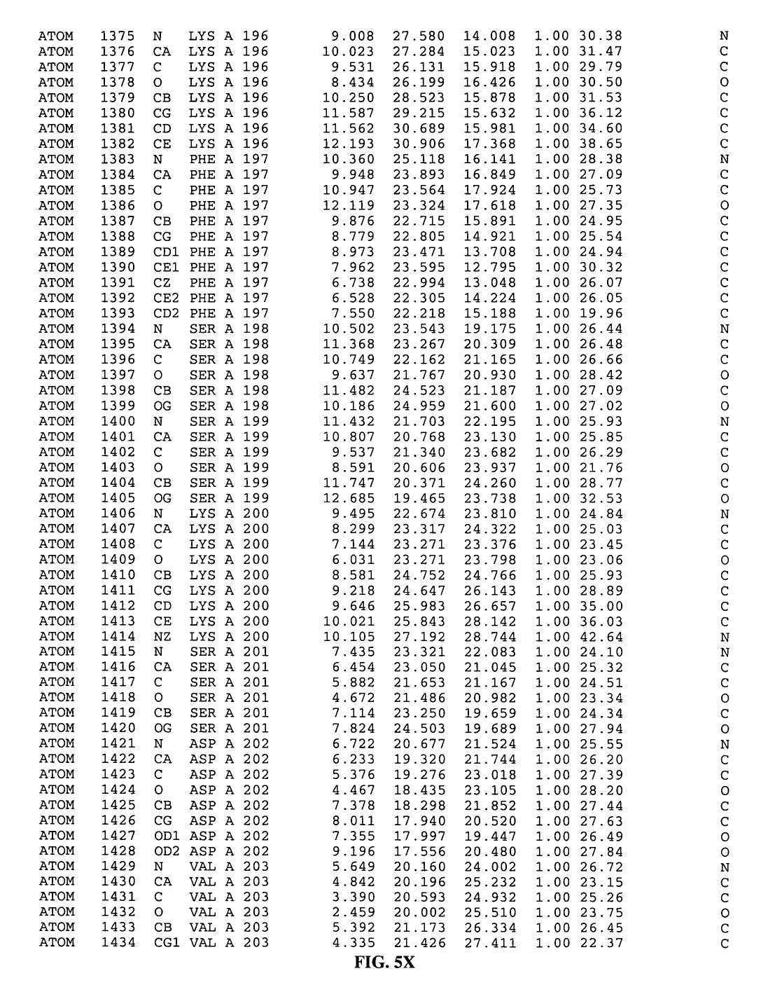

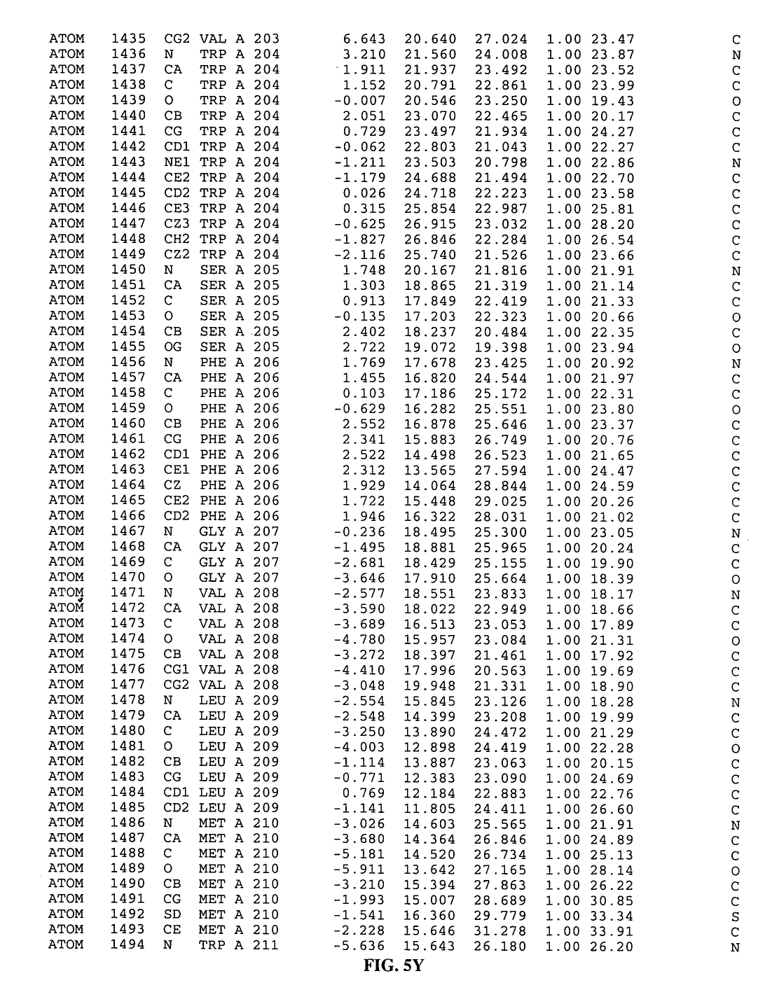

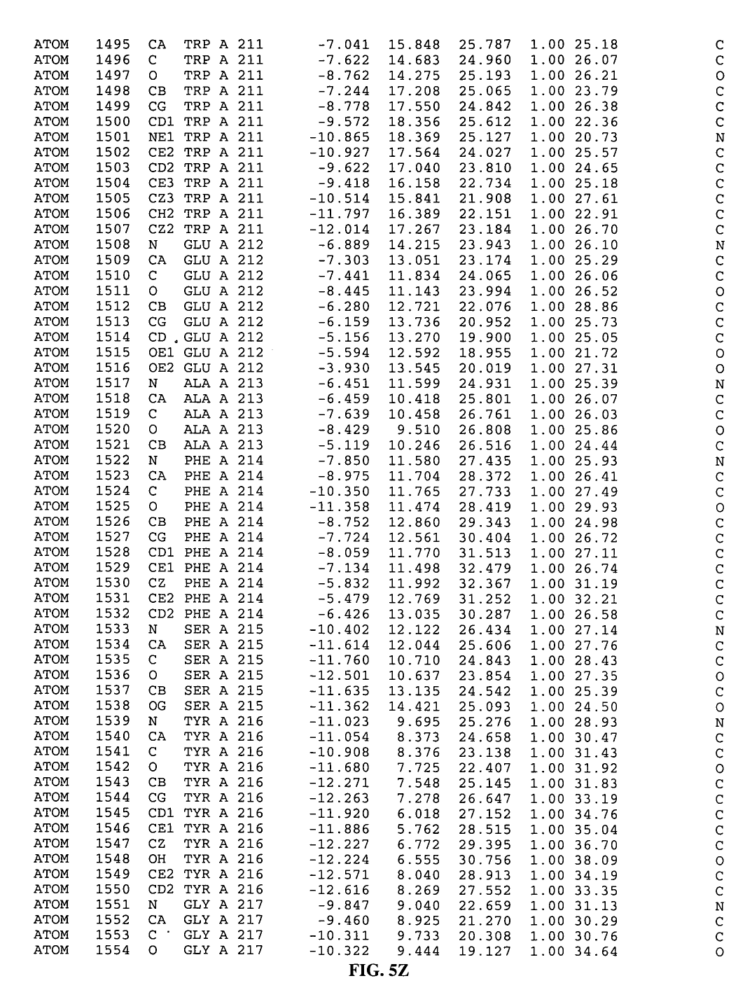

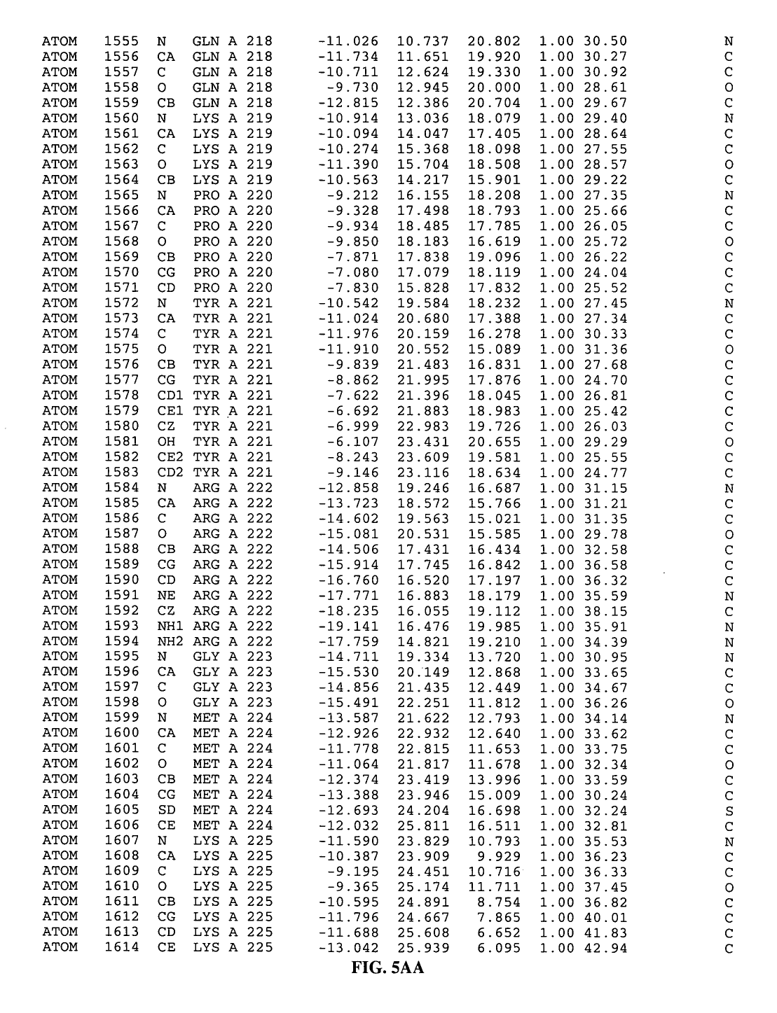

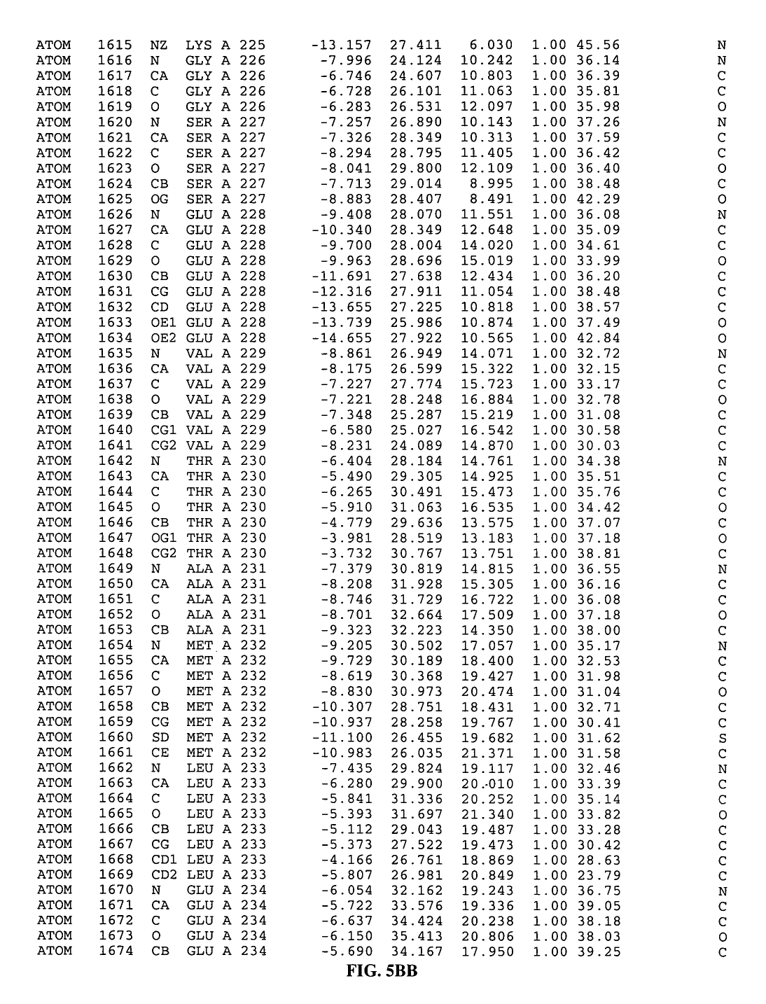

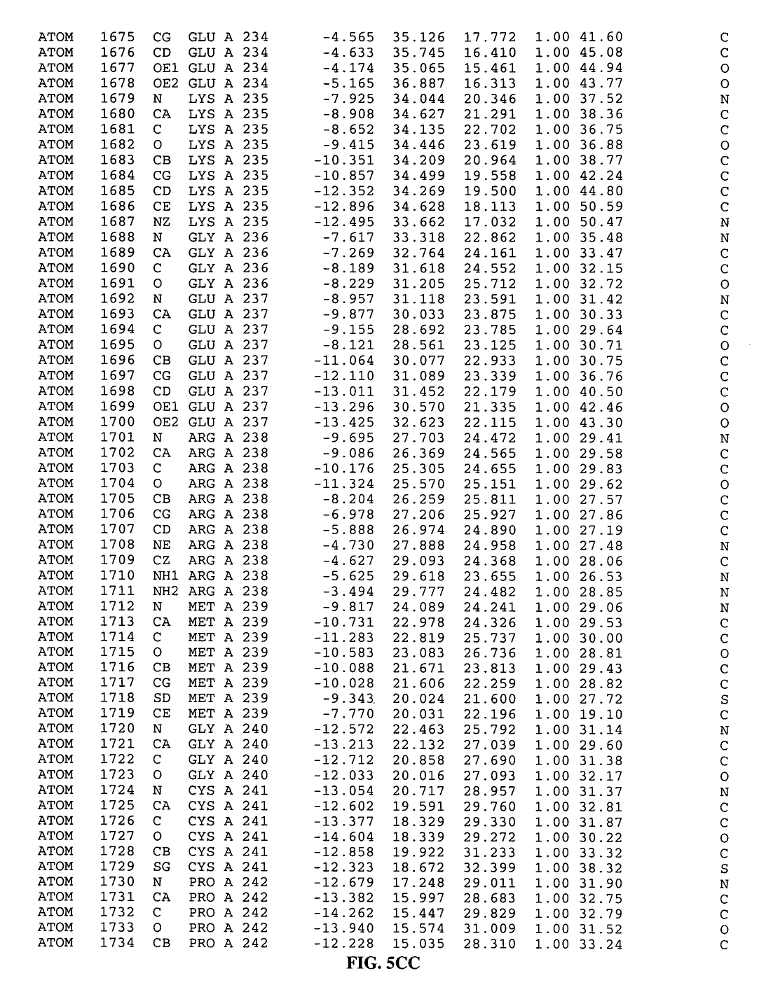

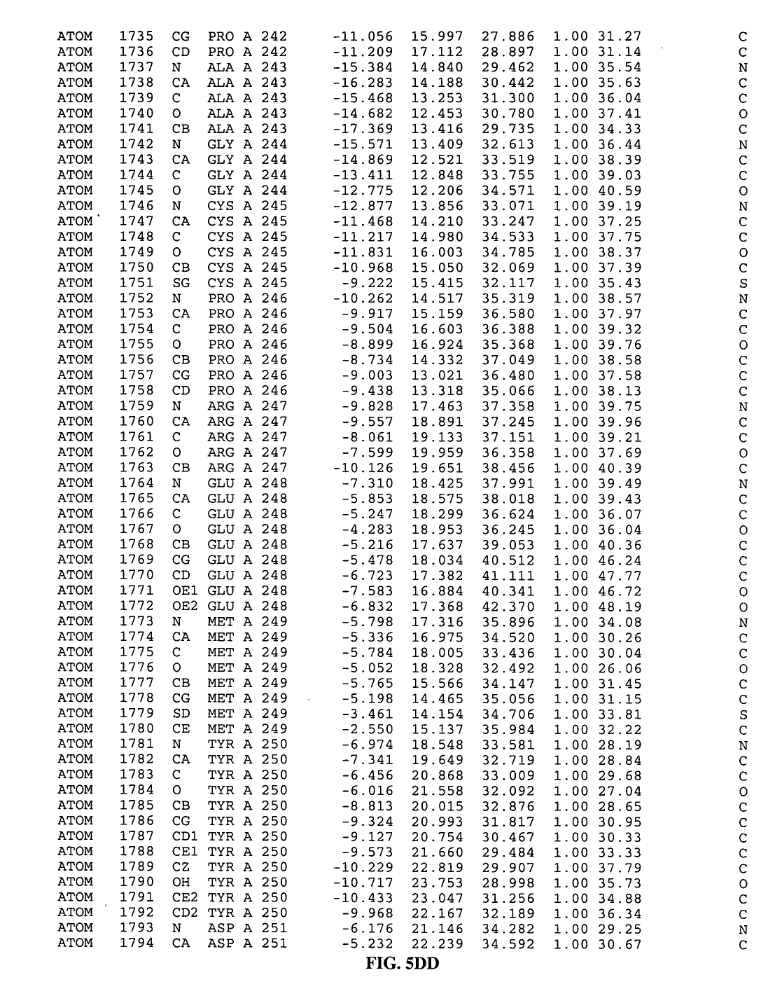

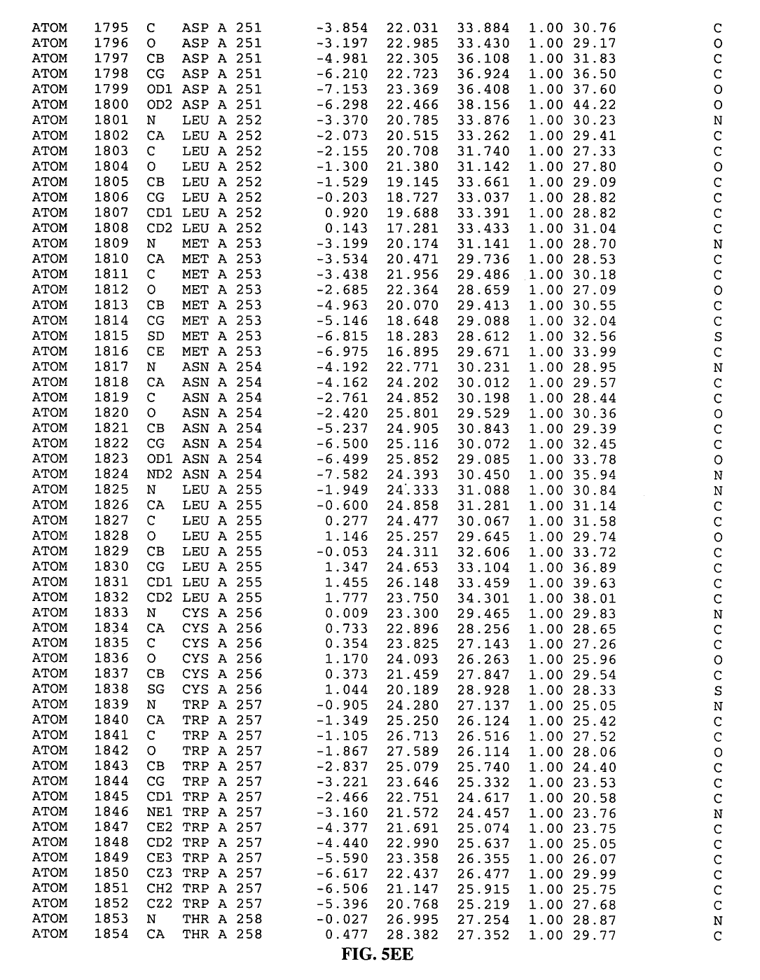

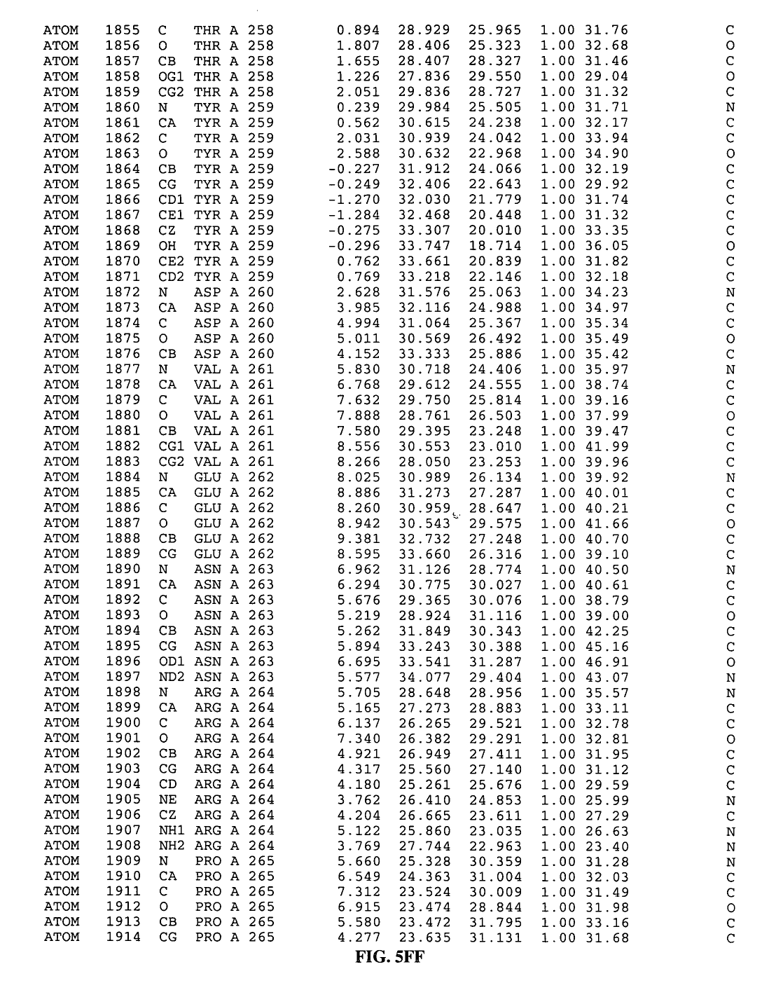

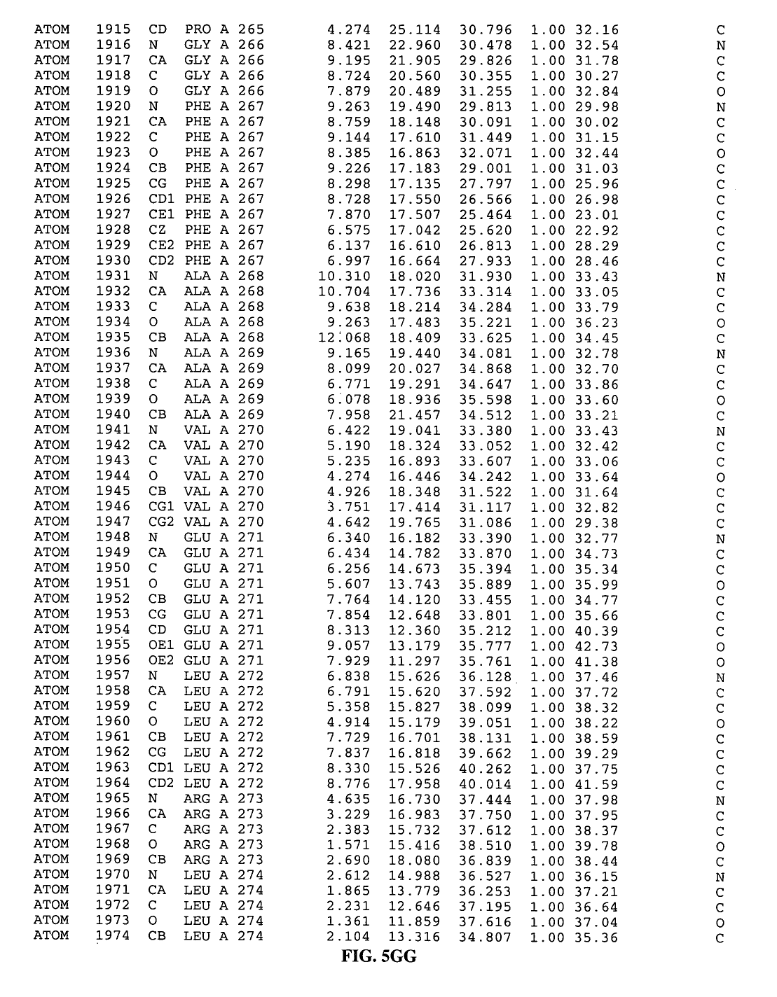

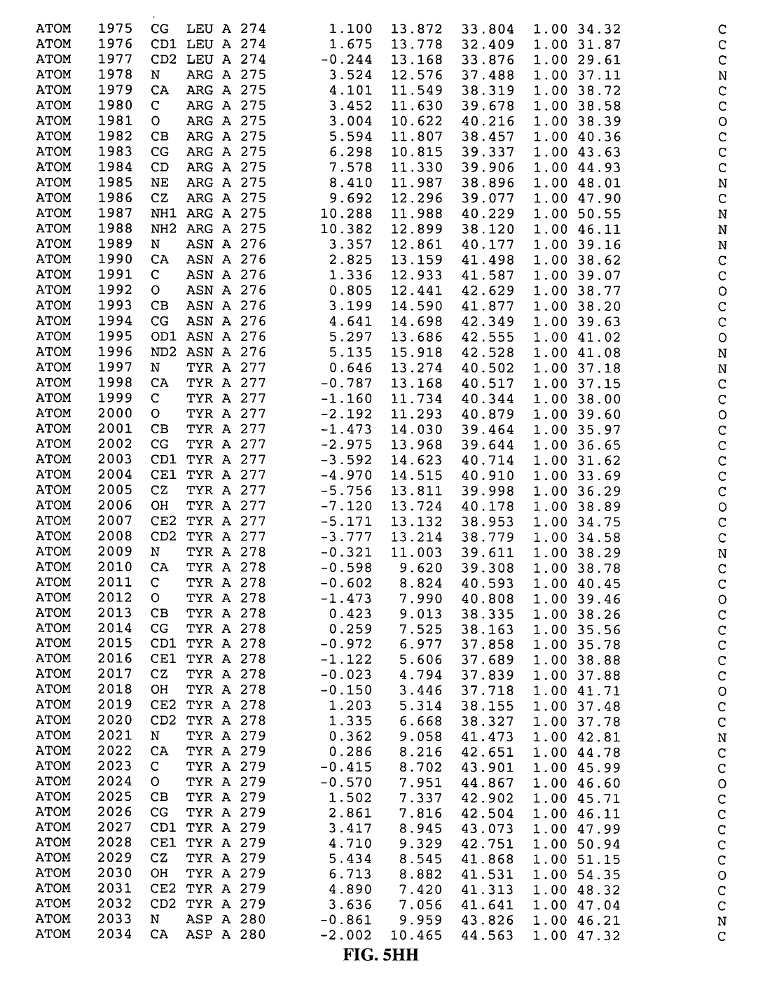

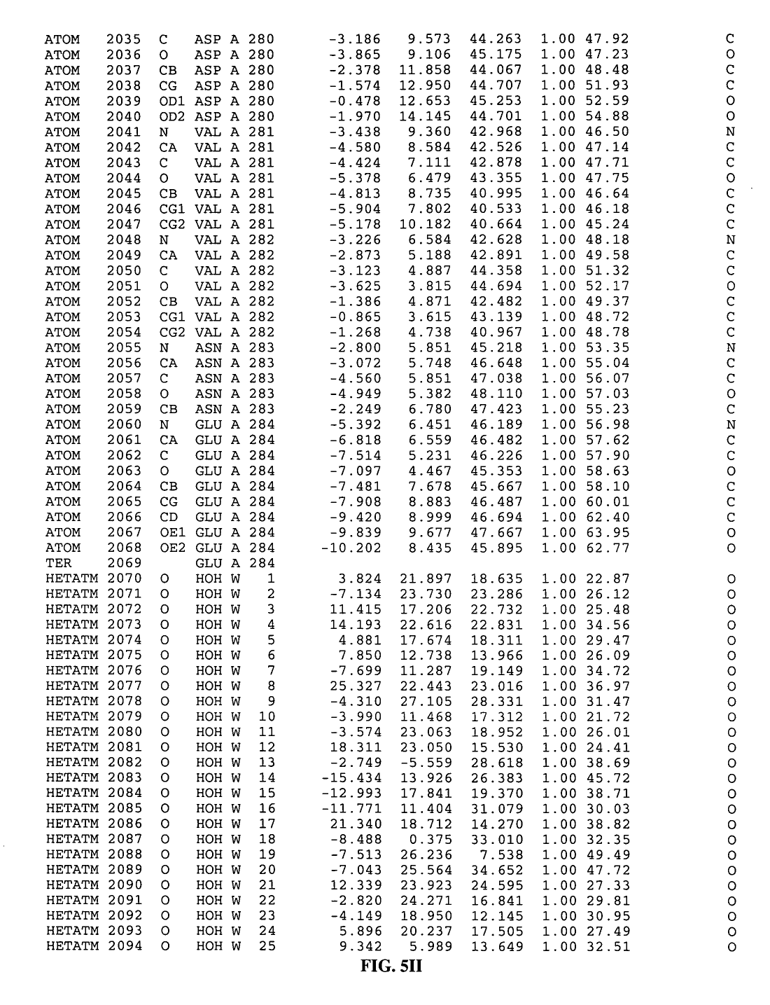

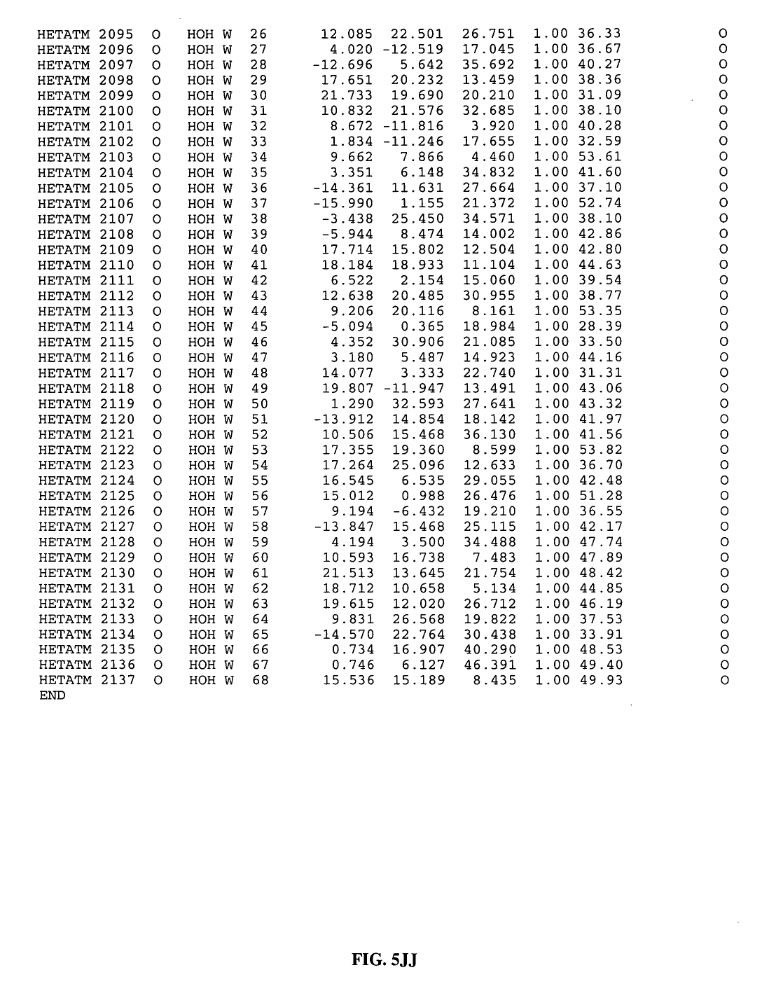

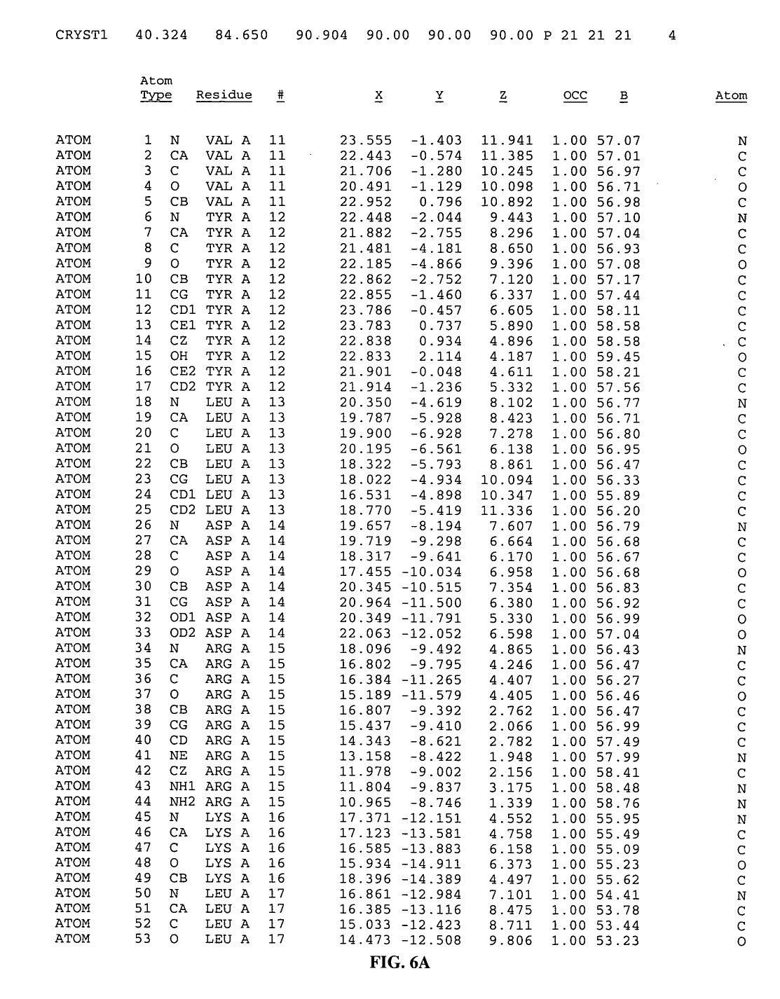

The following abbreviations are used in FIGS. 4, 5, 6, and 7.

“Atom Type” and “Atom” refer to the individual atom whose coordinates are provided, with and without indicating the position of the atom in the amino acid residue, respectively. The first letter in the column refers to the element.

HETATM refers to atomic coordinates within non-standard HET groups, such as prosthetic groups, inhibitors, solvent molecules, and ions for which coordinates are supplied. HETATMS include residues that are a) not one of the standard amino acids, including, for example, SeMet and SeCys, b) not one of the nucleic acids (C, G, A, T, U, and I), c) not one of the modified versions of nucleic acids (+C, +G, +A, +T, +U, and +I), and d) not an unknown amino acid or nucleic acid where UNK is used to indicate the unknown residue name.

“Residue” refers to the amino acid residue.

“#” refers to the residue number, starting from the N-terminal amino acid. The number designations of each amino acid residues reflect the position predicted in the expressed protein, including the His tag and the initial methionine.

“X, Y and Z” provide the Cartesian coordinates of the atom.

“B” is a thermal factor that measures movement of the atom around its atomic center.

“OCC” refers to occupancy, and represents the percentage of time the atom type occupies the particular coordinate. OCC values range from 0 to 1, with 1 being 100%.

Structure coordinates for SYKKD according to FIG. 4, 5, 6 or 7 may be modified by mathematical manipulation. Such manipulations include, but are not limited to, crystallographic permutations of the raw structure coordinates, fractionalization of the raw structure coordinates, integer additions or subtractions to sets of the raw structure coordinates, inversion of the raw structure coordinates, and any combination of the above.

FIG. 8 is a schematic of a SYKKD-cloning vector.

FIG. 9 is a schematic of a SYKKD-baculovirus expression destination vector.

FIG. 10 is a schematic representation of a pyruvate kinase-lactate dehydrogenase (PK/LDH) coupled enzyme assay.

FIG. 11 is a graph of dose response data for the PK/LDH coupled SYK enzyme assay and the kinase inhibitors piceatannol and staurosporine. Piceatannol is a plate alkaloid and a known selective SYK inhibitor with a published IC50 of 10 μM (Wang, B. H., et al. 1998. Planta Med. 64) in close agreement with the measurement shown here of 16 μM.

### ABBREVIATIONS

The amino acid notations used herein for the twenty genetically encoded amino acids are:

| | One-Letter | Three-Letter |
|---|---|---|
| Amino Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

As used herein, unless specifically delineated otherwise, the three-letter amino acid abbreviations designate amino acids in the L-configuration. Amino acids in the D-configuration are preceded with a “D-.” For example, Arg designates L-arginine and D-Arg designates D-arginine. Likewise, the capital one-letter abbreviations refer to amino acids in the L-configuration. Lower-case one-letter abbreviations designate amino acids in the D-configuration. For example, “R” designates L-arginine and “r” designates D-arginine.

Unless noted otherwise, when polypeptide sequences are presented as a series of one-letter and/or three-letter abbreviations, the sequences are presented in the N→C direction, in accordance with common practice.

Definitions

As used herein, the following terms shall have the following meanings:

“Genetically Encoded Amino Acid” refers to the twenty amino acids that are defined by genetic codons. The genetically encoded amino acids are glycine and the L-isomers of alanine, valine, leucine, isoleucine, serine, methionine, threonine, phenylalanine, tyrosine, tryptophan, cysteine, proline, histidine, aspartic acid, asparagine, glutamic acid, glutamine, arginine and lysine.

“Non-Genetically Encoded Amino Acid” refers to amino acids that are not defined by genetic codons. Non-genetically encoded amino acids include derivatives or analogs of the genetically-encoded amino acids that are capable of being enzymatically incorporated into nascent polypeptides using conventional expression systems, such as selenomethionine (SeMet) and selenocysteine (SeCys); isomers of the genetically-encoded amino acids that are not capable of being enzymatically incorporated into nascent polypeptides using conventional expression systems, such as D-isomers of the genetically-encoded amino acids; L- and D-isomers of naturally occurring α-amino acids that are not defined by genetic codons, such as α-aminoisobutyric acid (Aib); L- and D-isomers of synthetic α-amino acids that are not defined by genetic codons; and other amino acids such as β-amino acids,y-amino acids, etc. In addition to the D-isomers of the genetically-encoded amino acids, common non-genetically encoded amino acids include, but are not limited to norleucine (Nle), penicillamine (Pen), N-methylvaline (MeVal), homocysteine (hCys), homoserine (hSer), 2,3-diaminobutyric acid (Dab) and ornithine (Orn). Additional exemplary non-genetically encoded amino acids are found, for example, in Practical Handbook of Biochemistry and Molecular Biology, Fasman, Ed., CRC Press, Inc., Boca Raton, Fla., pp. 3-76, 1989, and the various references cited therein.

“Hydrophilic Amino Acid” refers to an amino acid having a side chain exhibiting a hydrophobicity of up to about zero according to the normalized consensus hydrophobicity scale of Eisenberg et al., J. Mol. Biol. 179:125-42, 1984. Genetically encoded hydrophilic amino acids include Thr (T), Ser (S), His (H), Glu (E), Asn (N), Gln (Q), Asp (D), Lys (K) and Arg (R). Non-genetically encoded hydrophilic amino acids include the D-isomers of the above-listed genetically-encoded amino acids, ornithine (Orn), 2,3-diaminobutyric acid (Dab) and homoserine (hSer).

“Acidic Amino Acid” refers to a hydrophilic amino acid having a side chain pK value of up to about 7 under physiological conditions. Acidic amino acids typically have negatively charged side chains at physiological pH due to loss of a hydrogen ion. Genetically encoded acidic amino acids include Glu (E) and Asp (D). Non-genetically encoded acidic amino acids include D-Glu (e) and D-Asp (d).

“Basic Amino Acid” refers to a hydrophilic amino acid having a side chain pK value of greater than 7 under physiological conditions. Basic amino acids typically have positively charged side chains at physiological pH due to association with hydronium ion. Genetically encoded basic amino acids include His (H), Arg (R) and Lys (K). Non-genetically encoded basic amino acids include the D-isomers of the above-listed genetically-encoded amino acids, ornithine (Orn) and 2,3-diaminobutyric acid (Dab).

“Polar Amino Acid” refers to a hydrophilic amino acid having a side chain that is uncharged at physiological pH, but which comprises at least one covalent bond in which the pair of electrons shared in common by two atoms is held more closely by one of the atoms. Genetically encoded polar amino acids include Asn (N), Gln (Q), Ser (S), and Thr (T). Non-genetically encoded polar amino acids include the D-isomers of the above-listed genetically-encoded amino acids and homoserine (hSer).

“Hydrophobic Amino Acid” refers to an amino acid having a side chain exhibiting a hydrophobicity of greater than zero according to the normalized consensus hydrophobicity scale of Eisenberg et al., J. Mol. Biol. 179:125-42, 1984. Genetically encoded hydrophobic amino acids include Pro (P), Ile (I), Phe (F), Val (V), Leu (L), Trp (W), Met (M), Ala (A), Gly (G) and Tyr (Y). Non-genetically encoded hydrophobic amino acids include the D-isomers of the above-listed genetically-encoded amino acids, norleucine (Nle) and N-methyl valine (MeVal).

“Aromatic Amino Acid” refers to a hydrophobic amino acid having a side chain comprising at least one aromatic or heteroaromatic ring. The aromatic or heteroaromatic ring may contain one or more substituents such as —OH, —SH, —CN, —F, —Cl, —Br, —I, —NO₂, —NO, —NH₂, —NHR, —NRR, —C(O)R, —C(O)OH, —C(O)OR, —C(O)NH₂, —C(O)NHR, —C(O)NRR and the like where each R is independently (C₁-C₆) alkyl, (C₁-C₆) alkenyl, or (C₁-C₆) alkynyl. Genetically encoded aromatic amino acids include Phe (F), Tyr (Y), Trp (W) and His (H). Non-genetically encoded aromatic amino acids include the D-isomers of the above-listed genetically-encoded amino acids.

“Apolar Amino Acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded apolar amino acids include Leu (L), Val (V), Ile (I), Met (M), Gly (G) and Ala (A). Non-genetically encoded apolar amino acids include the D-isomers of the above-listed genetically-encoded amino acids, norleucine (Nle) and N-methyl valine (MeVal).

“Aliphatic Amino Acid” refers to a hydrophobic amino acid having an aliphatic hydrocarbon side chain. Genetically encoded aliphatic amino acids include Ala (A), Val (V), Leu (L) and Ile (I). Non-genetically encoded aliphatic amino acids include the D-isomers of the above-listed genetically-encoded amino acids, norleucine (Nle) and N-methyl valine (MeVal).

“Helix-Breaking Amino Acid” refers to those amino acids that have a propensity to disrupt the structure of α-helices when contained at internal positions within the helix. Amino acid residues exhibiting helix-breaking properties are well-known in the art (see, e.g., Chou & Fasman, Ann. Rev. Biochem. 47:251-76, 1978) and include Pro (P), D-Pro (p), Gly (G) and potentially all D-amino acids (when contained in an L-polypeptide; conversely, L-amino acids disrupt helical structure when contained in a D-polypeptide).

“Cysteine-like Amino Acid” refers to an amino acid having a side chain capable of participating in a disulfide linkage. Thus, cysteine-like amino acids generally have a side chain containing at least one thiol (—SH) group. Cysteine-like amino acids are unusual in that they can form disulfide bridges with other cysteine-like amino acids. The ability of Cys (C) residues and other cysteine-like amino acids to exist in a polypeptide in either the reduced free -SH or oxidized disulfide-bridged form affects whether they contribute net hydrophobic or hydrophilic character to a polypeptide. Thus, while Cys (C) exhibits a hydrophobicity of 0.29 according to the consensus scale of Eisenberg (Eisenberg, 1984, supra), it is to be understood that for purposes of the present invention Cys (C) is categorized as a polar hydrophilic amino acid, notwithstanding the general classifications defined above. Other cysteine-like amino acids are similarly categorized as polar hydrophilic amino acids. Typical cysteine-like residues include, for example, penicillamine (Pen), homocysteine (hCys), etc.

As will be appreciated by those of skill in the art, the above-defined classes or categories are not mutually exclusive. Thus, amino acids having side chains exhibiting two or more physical-chemical properties can be included in multiple categories. For example, amino acid side chains having aromatic groups that are further substituted with polar substituents, such as Tyr (Y), may exhibit both aromatic hydrophobic properties and polar or hydrophilic properties, and could therefore be included in both the aromatic and polar categories. Typically, amino acids will be categorized in the class or classes that most closely define their net physical-chemical properties. The appropriate categorization of any amino acid will be apparent to those of skill in the art.

Other amino acid residues not specifically mentioned herein can be readily categorized based on their observed physical and chemical properties in light of the definitions provided herein.

“Wild-type SYKKD” refers to a polypeptide having an amino acid sequence that corresponds to the amino acid sequence of a naturally-occurring SYKKD, and wherein said polypeptide, when compared to SYKKD, has an rmsd of its backbone atoms of less than 2 Å.

“Homo sapiens SYKKD” refers to a polypeptide having an amino acid sequence that corresponds identically to the wild-type SYKKD from Homo sapiens.

By “or” is meant one, or another member of a group, or more than one member. For example, A, B, or C, may indicate any of the following: A alone; B alone; C alone; A and B;B and C;A and C; A, B, and C.

“Association” refers to the status of two or more molecules that are in close proximity to each other. The two molecules may be associated non-covalently, for example, by hydrogen-bonding, van der Waals, electrostatic or hydrophobic interactions, or covalently.

“Co-Complex” refers to a polypeptide in association with one or more compounds. Such compounds include, by way of example and not limitation, cofactors, ligands, substrates, substrate analogues, inhibitors, allosteric affecters, etc. Lead compounds for designing SYK inhibitors include, but are not restricted to, ATP, β-amido ATP, staurosporine, and derivatives and analogs thereof. A co-complex may also refer to a computer represented, or in silica generated association between a peptide and a compound. An “unliganded” form of a protein structure, or structural coordinates thereof, refers to the coordinates of the native form of a protein structure, or the apostructure, not a co-complex. A “liganded” form refers to the coordinates of a protein or peptide that is part of a co-complex. Unliganded forms include peptides and proteins associated with various ions, such as manganese, zinc, and magnesium, as well as with water. Liganded forms include peptides associated with natural substrates, non-natural substrates, inhibitors, substrate analogs, agonists or antagonists, proteins, co-factors and small molecules, as well as, optionally, in addition, various ions or water.

“Mutant” refers to a polypeptide characterized by an amino acid sequence that differs from the wild-type sequence by the substitution of at least one amino acid residue of the wild-type sequence with a different amino acid residue and/or by the addition and/or deletion of one or more amino acid residues to or from the wild-type sequence. The additions and/or deletions can be from an internal region of the wild-type sequence and/or at either or both of the N- or C-termini. A mutant polypeptide may have substantially the same three-dimensional structure as the corresponding wild-type polypeptide. A mutant may have, but need not have, SYK activity. A mutant may display biological activity that is substantially similar to that of the wild-type SYKKD. By “substantially similar biological activity” is meant that the mutant displays biological activity that is within 1% to 10,000% of the biological activity of the wild-type polypeptide, for example, within 25% to 5,000%, and, for example, within 50% to 500%, or 75% to 200% of the biological activity of the wild-type polypeptide, using assays known to those of ordinary skill in the art for that particular class of polypeptides. Mutants may also decrease or eliminate SYKKD activity. Mutants may be synthesized according to any method known to those skilled in the art, including, but not limited to, those methods of expressing SYKKD molecules described herein.

“Active Site” refers to a site in SYKKD that associates with the substrate for SYK activity. This site may include, for example, residues involved in catalysis, as well as residues involved in binding a substrate. Inhibitors may bind to the residues of the active site. In SYKKD, the active site includes one or more of the following amino acid residues: Val33, Ala48, Lys50, Val81, Leu94, Met96, Glu97, Met98, Ala99, Glu100, Gly102, Pro103, Arg146, Asn147, Leu149 and Ser159. Preferably, the active site comprises Glu97, Met98 and Ala99. Preferably, the active site further comprises Ala48 and Leul49, preferably the active site further comprises Val33, Lys50, Val81, Leu94, Met96, Glu100, Gly102, Pro103, Arg146, Asn147 and Ser159. Preferably the active site further comprises Leu25, Lys106, and Glu68. Amino acid residue numbers presented herein refer to the sequence of FIGS. 4, 5, 6, or 7. In SYKKD, the active site may, for example, comprise Glu68, Val71, Met72, Lys106, Val139, His140, Asp142, Asp160 and Phe161.

In SYKKD, the active site may, for example, comprise Asp160, Phe161, His140, Val139, Met96, Val71, Met72, Glu68, Ser159, Met98, Leu94, Leu149, Ala99, and Gly102.

“Binding Pocket” refers to a region in SYK which associates with a ligand such as a natural substrate, non-natural substrate, inhibitor, substrate analog, agonist or antagonist, protein, co-factor or small molecule, as well as, optionally, in addition, various ions or water, and/or has an internal cavity sufficient to bind a small molecule and may be used as a target for binding drugs. The term includes the active site but is not limited thereby.

“Accessory Binding Pocket” refers to a binding pocket in SYKKD other than that of the “active site.” An accessory binding pocket in SYK comprises, for example, Phe30, Val33, Lys50, Glu68, Ser159 and Aspl60.

“Conservative Mutant” refers to a mutant in which at least one amino acid residue from the wild-type sequence is substituted with a different amino acid residue that has similar physical and chemical properties, i.e., an amino acid residue that is a member of the same class or category, as defined above. For example, a conservative mutant may be a polypeptide that differs in amino acid sequence from the wild-type sequence by the substitution of a specific aromatic Phe (F) residue with an aromatic Tyr (Y) or Trp (W) residue.

“Non-Conservative Mutant” refers to a mutant in which at least one amino acid residue from the wild-type sequence is substituted with a different amino acid residue that has dissimilar physical and/or chemical properties, i.e., an amino acid residue that is a member of a different class or category, as defined above. For example, a non-conservative mutant may be a polypeptide that differs in amino acid sequence from the wild-type sequence by the substitution of an acidic Glu (E) residue with a basic Arg (R), Lys (K) or Orn residue.

“Deletion Mutant” refers to a mutant having an amino acid sequence that differs from the wild-type sequence by the deletion of one or more amino acid residues from the wild-type sequence. The residues may be deleted from internal regions of the wild-type sequence and/or from one or both termini.

“Truncated Mutant” refers to a deletion mutant in which the deleted residues are from the N- and/or C-terminus of the wild-type sequence.

“Extended Mutant” refers to a mutant in which additional residues are added to the N- and/or C-terminus of the wild-type sequence.

“Methionine mutant” refers to (1) a mutant in which at least one methionine residue of the wild-type sequence is replaced with another residue, such as with an aliphatic residue, such as an Ala (A), Leu (L), or Ile (I) residue; or (2) a mutant in which a non-methionine residue, such as an aliphatic residue, such as an Ala (A), Leu (L) or Ile (I) residue, of the wild-type sequence is replaced with a methionine residue.

“Selenomethionine mutant” refers to (1) a mutant which includes at least one selenomethionine (SeMet) residue, typically by substitution of a Met residue of the wild-type sequence with a SeMet residue, or by addition of one or more SeMet residues at one or both termini, or (2) a methionine mutant in which at least one Met residue is substituted with a SeMet residue. In some embodiments, each Met residue is substituted with a SeMet residue.

“Cysteine mutant” refers to a mutant in which at least one cysteine residue of the wild-type sequence is replaced with another residue, such as with a Ser (S) residue.

“Serine mutant” refers to a mutant in which at least one serine residue of the wild-type sequence is replaced with another residue, such as with a cysteine residue.

“Selenocysteine mutant” refers to (1) a mutant which includes at least one selenocysteine (SeCys) residue, typically by substitution of a Cys residue of the wild-type sequence with a SeCys residue, or by addition of one or more SeCys residues at one or both termini, or (2) a cysteine mutant in which at least one Cys residue is substituted with a SeCys residue. In some embodiments, SeCys mutants are those in which each Cys residue is substituted with a SeCys residue.

“Homolog” refers to a polypeptide having at least 30%, preferably at least 40%, preferably at least 50%, preferably at least 60%, preferably at least 70%, more preferably at least 80%, and most preferably at least 90% amino acid sequence identity or having a BLAST E-value of 1×10⁻⁶ over at least 100 amino acids (Altschul et al., Nucleic Acids Res., 25:3389-402, 1997) with SYKKD or any functional domain of SYKKD.

“Crystal” refers to a composition comprising a polypeptide in crystalline form. The term “crystal” includes native crystals, heavy-atom derivative crystals and co-crystals, as defined herein.

“Native Crystal” refers to a crystal wherein the polypeptide is substantially pure. As used herein, native crystals do not include crystals of polypeptides comprising amino acids that are modified with heavy atoms, such as crystals of selenomethionine mutants, selenocysteine mutants, etc.

“Heavy-atom Derivative Crystal” refers to a crystal wherein the polypeptide is in association with one or more heavy-metal atoms. As used herein, heavy-atom derivative crystals include native crystals into which a heavy metal atom is soaked, as well as crystals of selenomethionine mutants and selenocysteine mutants.

“Co-Crystal” refers to a composition comprising a co-complex, as defined above, in crystalline form. Co-crystals include native co-crystals and heavy-atom derivative co-crystals.

“Apo-crystal” refers to a crystal wherein the polypeptide is substantially pure and substantially free of compounds that might form a co-complex with the polypeptide such as cofactors, ligands, substrates, substrate analogues, inhibitors, allosteric affecters, etc.

“Diffraction Quality Crystal” refers to a crystal that is well-ordered and of a sufficient size, i.e., at least 10 m, at least 50 μm, or at least 100 μm in its smallest dimension such that it produces measurable diffraction to at least 3 Å resolution, preferably to at least 2 Å resolution, and most preferably to at least 1.5 Å resolution or lower. Diffraction quality crystals include native crystals, heavy-atom derivative crystals, and co-crystals.

“Unit Cell” refers to the smallest and simplest volume element (i.e., parallelepiped-shaped block) of a crystal that is completely representative of the unit or pattern of the crystal, such that the entire crystal can be generated by translation of the unit cell. The dimensions of the unit cell are defined by six numbers: dimensions a, b and c and the angles are defined as α, β, and γ (Blundell et al., Protein Crystallography, 83-84, Academic Press. 1976). A crystal is an efficiently packed array of many unit cells.

“Triclinic Unit Cell” refers to a unit cell in which a≠b≠c and α≠β≠γ.

“Monoclinic Unit Cell” refers to a unit cell in which a≠b≠c; α=γ=90°; and β>90°.

“Hexagonal Unit Cell” refers to a unit cell in which a=b≠c; α=β=90°; and γ=120°.

“Orthorhombic Unit Cell” refers to a unit cell in which a≠b≠c; and α=β=γ=90°.

“Tetragonal Unit Cell” refers to a unit cell in which a=b≠c; and α=β=γ=90°.

“Trigonal/Rhombohedral Unit Cell” refers to a unit cell in which a=b=c; and α=β=γ90°.

“Trigonal/Hexagonal Unit Cell” refers to a unit cell in which a=b≠c; α=β=90°; and γ=120°.

“Cubic Unit Cell” refers to a unit cell in which a=b=c; and α=β=γ=90°.

“Crystal Lattice” refers to the array of points defined by the vertices of packed unit cells.

“Space Group” refers to the set of symmetry operations of a unit cell. In a space group designation (e.g., C2) the capital letter indicates the lattice type and the other symbols represent symmetry operations that can be carried out on the unit cell without changing its appearance.

“Asymmetric Unit” refers to the largest aggregate of molecules in the unit cell that possesses no symmetry elements that are part of the space group symmetry, but that can be juxtaposed on other identical entities by symmetry operations.

“Crystallographically-Related Dimer (or oligomer)” refers to a dimer (or oligomer, such as, for example, a trimer or a tetramer) of two (or more) molecules wherein the symmetry axes or planes that relate the two (or more) molecules comprising the dimer (or oligomer) coincide with the symmetry axes or planes of the crystal lattice.

“Non-Crystallographically-Related Dimer (or oligomer)” refers to a dimer (or oligomer, such as, for example, a trimer or a tetramer) of two (or more) molecules wherein the symmetry axes or planes that relate the two (or more) molecules comprising the dimer (or oligomer) do not coincide with the symmetry axes or planes of the crystal lattice.

“Isomorphous Replacement” refers to the method of using heavy-atom derivative crystals to obtain the phase information necessary to elucidate the three-dimensional structure of a crystallized polypeptide (Blundell et al., Protein Crystallography, Academic Press, esp. pp. 151-64, 1976; Methods in Enzymology 276:361-557, Academic Press, 1997). The phrase “heavy-atom derivatization” is synonymous with “isomorphous replacement.”

“Multi-Wavelength Anomalous Dispersion or MAD” refers to a crystallographic technique in which X-ray diffraction data are collected at several different wavelengths from a single heavy-atom derivative crystal, wherein the heavy atom has absorption edges near the energy of incoming X-ray radiation. The resonance between X-rays and electron orbitals leads to differences in X-ray scattering from absorption of the X-rays (known as anomalous scattering) and permits the locations of the heavy atoms to be identified, which in turn provides phase information for a crystal of a polypeptide. A detailed discussion of MAD analysis can be found in Hendrickson, Trans. Am. Crystallogr. Assoc., 21:11, 1985; Hendrickson et al., EMBO J. 9:1665, 1990; and Hendrickson, Science, 254:51-58, 1991.

“Single Wavelength Anomalous Dispersion or SAD” refers to a crystallographic technique in which X-ray diffraction data are collected at a single wavelength from a single native or heavy-atom derivative crystal, and phase information is extracted using anomalous scattering information from atoms such as sulfur or chlorine in the native crystal or from the heavy atoms in the heavy-atom derivative crystal. The wavelength of X-rays used to collect data for this phasing technique needs to be close to the absorption edge of the anomalous scatterer. A detailed discussion of SAD analysis can be found in Brodersen, et al., Acta Cryst., D56:431-41, 2000.

“Single Isomorphous Replacement With Anomalous Scattering or SIRAS” refers to a crystallographic technique that combines isomorphous replacement and anomalous scattering techniques to provide phase information for a crystal of a polypeptide. X-ray diffraction data are collected at a single wavelength, usually from a single heavy-atom derivative crystal. Phase information obtained only from the location of the heavy atoms in a single heavy-atom derivative crystal leads to an ambiguity in the phase angle, which is resolved using anomalous scattering from the heavy atoms. Phase information is therefore extracted from both the location of the heavy atoms and from anomalous scattering of the heavy atoms. A detailed discussion of SIRAS analysis can be found in North, Acta Cryst. 18:212-16, 1965; Matthews, Acta Cryst., 20:82-86, 1966.

“Molecular Replacement” refers to the method using the structure coordinates of a known polypeptide to calculate initial phases for a new crystal of a polypeptide whose structure coordinates are unknown. This is done by orienting and positioning a polypeptide whose structure coordinates are known within the unit cell of the new crystal. Phases are then calculated from the oriented and positioned polypeptide and combined with observed amplitudes to provide an approximate Fourier synthesis of the structure of the polypeptides comprising the new crystal. The model is then refined to provide a refined set of structure coordinates for the new crystal (Lattman, Methods in Enzymology, 115:55-77, 1985; Rossmann, “The Molecular Replacement Method,” Int. Sci. Rev. Ser. No. 13, Gordon & Breach, New York, 1972; Methods in Enzymology, Vols. 276, 277 (Academic Press, San Diego 1997)). Molecular replacement may be used, for example, to determine the structure coordinates of a crystalline mutant or homolog of SYKKD using the structure coordinates of SYKKD.

“Structure coordinates” refers to mathematical coordinates derived from mathematical equations related to the patterns obtained on diffraction of a monochromatic beam of X-rays by the atoms (scattering centers) of a SYKKD in crystal form. The diffraction data are used to calculate an electron density map of the repeating unit of the crystal. The electron density maps are used to establish the positions of the individual atoms within the unit cell of the crystal.

“Having substantially the same three-dimensional structure” refers to a polypeptide that is characterized by a set of molecular structure coordinates that have a root mean square deviation (r.m.s.d.) of up to about or equal to 1.5 Å, preferably 1.25 Å, preferably 1 Å, and preferably 0.5 Å, and preferably 0.25 Å, when superimposed onto the molecular structure coordinates of FIGS. 4-7 when at least 50% to 100% of the C-alpha atoms of the coordinates are included in the superposition. The program MOE may be used to compare two structures (Chemical Computing Group, Inc., Montreal, Canada). Where structure coordinates are not available for a particular amino acid residue(s), those coordinates are not included in the calculation.

“α-C” or “α-carbon” or “CA” as used herein, “α-C” or “α-carbon” refer to the alpha carbon of an amino acid residue.

“α-helix” refers to the conformation of a polypeptide chain in the form of a spiral chain of amino acids stabilized by hydrogen bonds.

The term “β-sheet” refers to the conformation of a polypeptide chain stretched into an extended zig-zag conformation. Portions of polypeptide chains that run “parallel” all run in the same direction. Where polypeptide chains are “antiparallel,” neighboring chains run in opposite directions from each other. The term “run” refers to the N to COOH direction of the polypeptide chain.

### DETAILED DESCRIPTION OF THE INVENTION

Crystalline SYK

Both native and heavy-atom derivative crystals, such as those obtained from selenium methionine derivative SYKKD may be used to obtain the molecular structure coordinates of the present invention.

The SYK comprising the crystals of the invention can be isolated from any bacterial, plant, or animal source in which SYK is present. Within the scope of the present invention are proteins that are homologous to SYK that are derived from any biological kingdom. The SYK may be derived from a mammalian source, such as, for example, Homo sapiens. The crystals may comprise wild-type SYK or mutants of wild-type SYK. Mutants of wild-type SYK are obtained by replacing at least one amino acid residue in the sequence of the wild-type SYK with a different amino acid residue, or by adding or deleting one or more amino acid residues within the wild-type sequence and/or at the N-and/or C-terminus of the wild-type SYK. The mutants may, but not necessarily, crystallize under crystallization conditions that are substantially similar to those used to crystallize the wild-type SYK.

The types of mutants contemplated by this invention include, but are not limited to, conservative mutants, non-conservative mutants, deletion mutants, truncated mutants, extended mutants, methionine mutants, selenomethionine mutants, cysteine mutants and selenocysteine mutants. A mutant may have, but need not display, SYK activity. A mutant, for example, may display biological activity that is substantially similar to that of the wild-type polypeptide. Methionine, selenomethione, cysteine, and selenocysteine mutants are particularly useful for producing heavy-atom derivative crystals, as described in detail, below.

It will be recognized by one of skill in the art that the types of mutants contemplated herein are not mutually exclusive; that is, for example, a polypeptide having a conservative mutation in one amino acid may in addition have a truncation of residues at the N-terminus, and several Ala, Leu, or Ile→Met mutations.

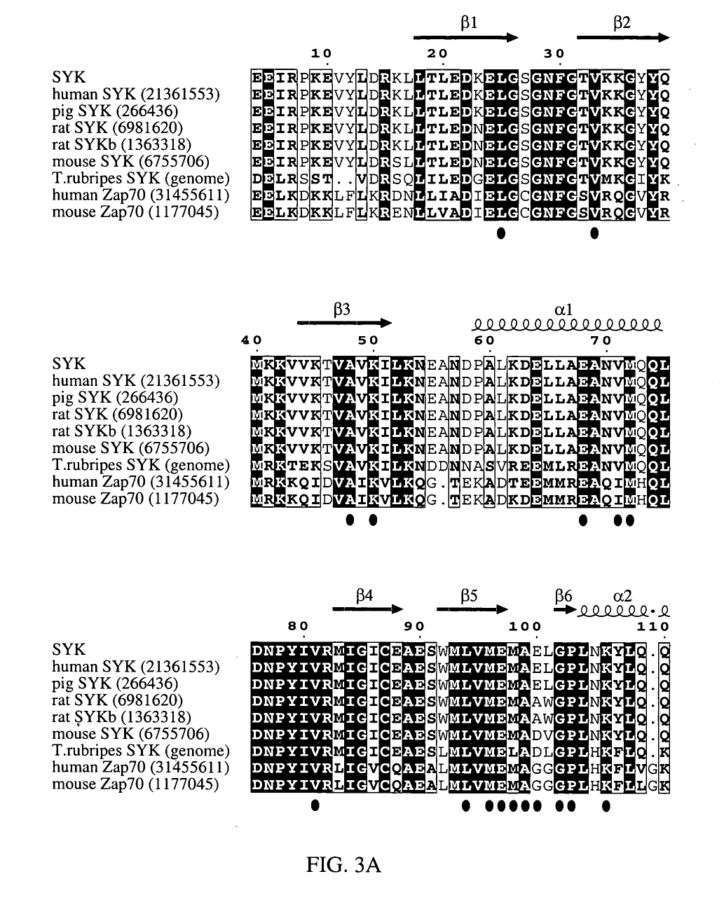

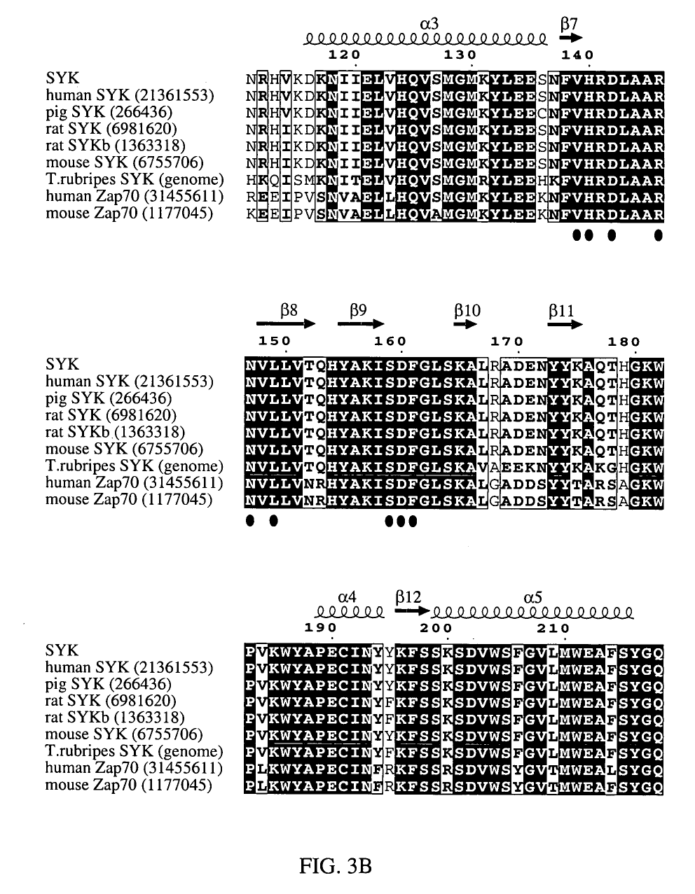

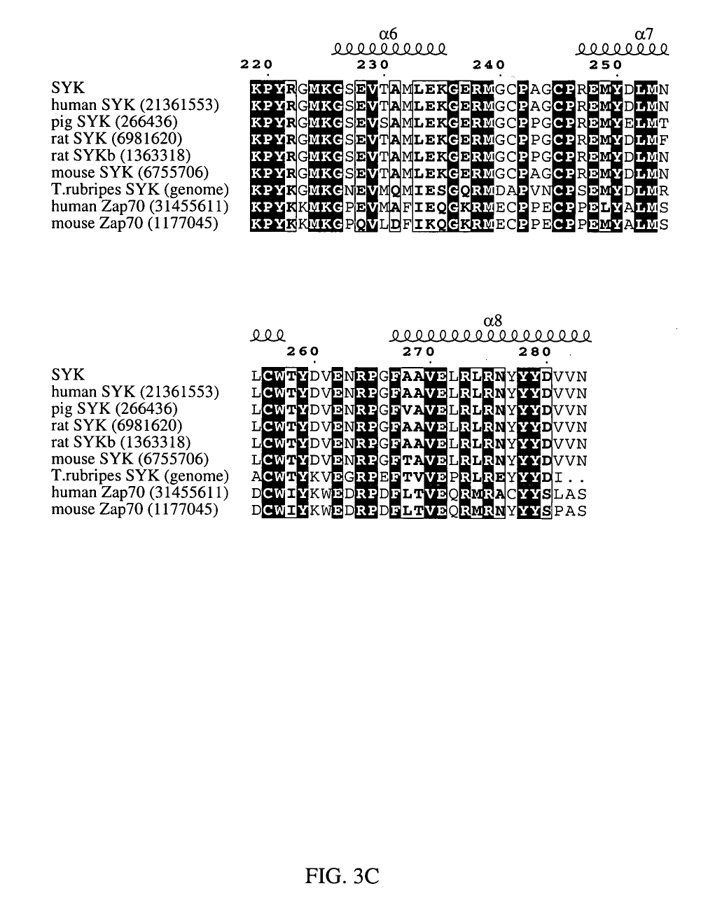

Sequence alignments of polypeptides in a protein family or of homologous polypeptide domains can be used to identify potential amino acid residues in the polypeptide sequence that are candidates for mutation. Identifying mutations that do not significantly interfere with the three-dimensional structure of SYK and/or that do not deleteriously affect, and that may even enhance, the activity of SYK will depend, in part, on the region where the mutation occurs. In highly variable regions of the molecule, such as those shown in FIG. 3, non-conservative substitutions as well as conservative substitutions may be tolerated without significantly disrupting the folding, the three-dimensional structure and/or the biological activity of the molecule. In highly conserved regions, or regions containing significant secondary structure, such as those regions shown in FIG. 3, conservative amino acid substitutions may be tolerated.

Conservative amino acid substitutions are well known in the art, and include substitutions made on the basis of a similarity in polarity, charge, solubility, hydrophobicity and/or the hydrophilicity of the amino acid residues involved. Typical conservative substitutions are those in which the amino acid is substituted with a different amino acid that is a member of the same class or category, as those classes are defined herein. Thus, typical conservative substitutions include aromatic to aromatic, apolar to apolar, aliphatic to aliphatic, acidic to acidic, basic to basic, polar to polar, etc. Other conservative amino acid substitutions are well known in the art. It will be recognized by those of skill in the art that generally, a total of 20% or fewer, typically 10% or fewer, most usually 5% or fewer, of the amino acids in the wild-type polypeptide sequence can be conservatively substituted with other amino acids without deleteriously affecting the biological activity, the folding, and/or the three-dimensional structure of the molecule, provided that such substitutions do not involve residues that are critical for activity, for example, critical binding pocket residues.

In some embodiments, it may be desirable to make mutations in the active site of a protein, e.g., to reduce or completely eliminate protein activity. For example, it may be desirable to mutate important residues in the active site of a protease in order to reduce or eliminate protease activity and to avoid autolysis in solution or in a crystal. Thus, for example, in aspartyl proteases, the active site Asp residue may be mutated to an Ala or Asn residue to reduce protease activity. The active site Ser residue in serine proteases may be mutated to an Ala, Cys or Thr residue to reduce or eliminate protease activity. Similarly, the activity of a cysteine protease may be reduced or eliminated by mutating the active site Cys residue to an Ala, Ser or Thr residue. Other mutations that will reduce or completely eliminate the activity of a particular protein will be apparent to those of skill in the art.

The amino acid residue Cys (C) is unusual in that it can form disulfide bridges with other Cys (C) residues or other sulfhydryls, such as, for example, sulfhydryl-containing amino acids (“cysteine-like amino acids”). The ability of Cys (C) residues and other cysteine-like amino acids to exist in a polypeptide in either the reduced free —SH or oxidized disulfide-bridged form affects whether Cys (C) residues contribute net hydrophobic or hydrophilic character to a polypeptide. While Cys (C) exhibits a hydrophobicity of 0.29 according to the consensus scale of Eisenberg (Eisenberg et al., J. Mol. Biol. 179:125-42, 1984), it is to be understood that for purposes of the present invention Cys (C) is categorized as a polar hydrophilic amino acid, notwithstanding the general classifications defined above. For example, Cys residues that are known to participate in disulfide bridges are not substituted or are conservatively substituted with other cysteine-like amino acids' so that the residue can participate in a disulfide bridge. Typical cysteine-like residues include, for example, Pen, hCys, etc. Substitutions for Cys residues that interfere with crystallization are discussed infra.

The structural coordinates of a binding pocket and/or of the protein may be used, for example, to engineer new molecules. These new molecules may be expressed in cells, for example, in plant cells using, for example, gene transformation, to improve nutrient yields in plant crops or to use plants to produce new molecules.

While in most instances the amino acids of SYKKD will be substituted with genetically-encoded amino acids, in certain circumstances mutants may include non-genetically encoded amino acids. For example, non-encoded derivatives of certain encoded amino acids, such as SeMet and/or SeCys, may be incorporated into the polypeptide chain using biological expression systems (such SeMet and SeCys mutants are described in more detail, infra).

Alternatively, in instances where the mutant will be prepared in whole or in part by chemical synthesis, virtually any non-encoded amino acids may be used, ranging from D-isomers of the genetically encoded amino acids to non-encoded naturally-occurring natural and synthetic amino acids.

Conservative amino acid substitutions for many of the commonly known non-genetically encoded amino acids are well known in the art. Conservative substitutions for other non-encoded amino acids can be determined based on their physical properties as compared to the properties of the genetically encoded amino acids.

Those of ordinary skill in the art will recognize that substitutions, additions, and/or deletions that do not substantially alter the three dimensional structure of SYKKD and that, for example, do not substantially alter the three dimensional structure of the SYKKD binding pocket or pockets discussed in the present application, are within the scope of the present invention. Such substitutions, additions, and/or deletions may be useful, for example, to provide convenient cloning sites in cDNA encoding SYK, to aid in its purification, or to aid in obtaining crystallization.

These substitutions, deletions and/or additions include, but are not limited to, His tags, intein-containing self-cleaving tags, maltose binding protein fusions, glutathione S-transferase protein fusions, antibody fusions, green fluorescent protein fusions, signal peptide fusions, biotin accepting peptide fusions, tags that contain protease cleavage sites, and the like. Mutations may also be introduced into a polypeptide sequence where there are residues, e.g., cysteine residues that interfere with crystallization. These cysteine residues can be substituted with an appropriate amino acid that does not readily form covalent bonds with other amino acid residues under crystallization conditions; e.g., by substituting the cysteine with Ala, Ser or Gly. Any cysteine located in a non-helical or non-stranded segment, based on secondary structure assignments, are good candidates for replacement.

Mutants within the scope of the invention may or may not have SYK activity. Amino acid substitutions, additions and/or deletions that might alter or inhibit SYK activity are within the scope of the present invention. These mutants can be used in their crystalline form, or the molecular structure coordinates obtained therefrom, for example, to determine SYK structure and/or to provide phase information to aid the determination of the three-dimensional X-ray structures of other related or non-related crystalline polypeptides.

The heavy-atom derivative crystals from which the molecular structure coordinates of the invention are obtained generally comprise a crystalline SYKKD polypeptide in association with one or more heavy atoms, such as, for example, Xe, Kr, Br, I, or a heavy metal atom. The polypeptide may correspond to a wild-type or a mutant SYKKD, which may optionally be in co-complex with one or more molecules, as previously described. There are various types of heavy-atom derivatives of polypeptides: heavy-atom derivatives resulting from exposure of the protein to a heavy atom in solution, wherein crystals are grown in medium comprising the heavy atom, or in crystalline form, wherein the heavy atom diffuses into the crystal, heavy-atom derivatives wherein the polypeptide comprises heavy-atom containing amino acids, e.g., selenomethionine and/or selenocysteine, and heavy atom derivatives where the heavy atom is forced in under pressure, such as, for example, in a xenon chamber.

In practice, heavy-atom derivatives of the first type can be formed by soaking a native crystal in a solution comprising heavy metal atom salts, or organometallic compounds, e.g., lead chloride, gold thiomalate, ethylmercurithiosalicylic acid-sodium salt (thimerosal), uranyl acetate, platinum tetrachloride, osmium tetraoxide, zinc sulfate, and cobalt hexamine, which can diffuse through the crystal and bind to the crystalline polypeptide.

Heavy-atom derivatives of this type can also be formed by adding to a crystallization solution comprising the polypeptide to be crystallized, an amount of a heavy metal atom salt, which may associate with the protein and be incorporated into the crystal. The location(s) of the bound heavy metal atom(s) can be determined by X-ray diffraction analysis of the crystal. This information, in turn, is used to generate the phase information needed to construct the three-dimensional structure of the protein.

Heavy-atom derivative crystals may also be prepared from polypeptides that include one or more SeMet and/or SeCys residues (SeMet and/or SeCys mutants). Such selenocysteine or selenomethionine mutants may be made from wild-type or mutant SYKKD by expression of SYKKD-encoding cDNAs in auxotrophic E. coli strains (Hendrickson et al., EMBO J. 9(5):1665-72, 1990). In this method, the wild-type or mutant SYKKD cDNA may be expressed in a host organism on a growth medium depleted of either natural cysteine or methionine (or both) but enriched in selenocysteine or selenomethionine (or both). Alternatively, selenocysteine or selenomethionine mutants may be made using nonauxotrophic E. coli strains, e.g., by inhibiting methionine biosynthesis in these strains with high concentrations of Ile, Lys, Phe, Leu, Val or Thr and then providing selenomethionine in the medium (Doublié, Methods in Enzymology, 276:523-30, 1997). Furthermore, selenocysteine can be selectively incorporated into polypeptides by exploiting the prokaryotic and eukaryotic mechanisms for selenocysteine incorporation into certain classes of proteins in vivo, as described in U.S. Pat. No. 5,700,660 to Leonard et al. (filed Jun. 7, 1995). One of skill in the art will recognize that selenocysteine may, for example, not incorporated in place of cysteine residues that form disulfide bridges, as these may be important for maintaining the three-dimensional structure of the protein and may, for example, not be eliminated. One of skill in the art will further recognize that, in order to obtain accurate phase information, approximately one selenium atom should be incorporated for every 140 amino acid residues of the polypeptide chain. The number of selenium atoms incorporated into the polypeptide chain can be conveniently controlled by designing a Met or Cys mutant having an appropriate number of Met and/or Cys residues, as described more fully below.

In some instances, the polypeptide to be crystallized may not contain cysteine or methionine residues. Therefore, if selenomethionine and/or selenocysteine mutants are to be used to obtain heavy-atom derivative crystals, methionine and/or cysteine residues may be introduced into the polypeptide chain. Likewise, Cys residues must be introduced into the polypeptide chain if the use of a cysteine-binding heavy metal, such as mercury, is contemplated for production of a heavy-atom derivative crystal.

Such mutations are, for example, introduced into the polypeptide sequence at sites that will not disturb the overall protein fold. For example, a residue that is conserved among many members of the protein family or that is thought to be involved in maintaining its activity or structural integrity, as determined by, e.g., sequence alignments, should not be mutated to a Met or Cys. In addition, conservative mutations, such as Ser to Cys, or Leu or Ile to Met, are, for example, introduced. One additional consideration is that, in order for a heavy-atom derivative crystal to provide phase information for structure determination, the location of the heavy atom(s) in the crystal unit cell must be determinable and provide phase information. Therefore, a mutation is, for example, not introduced into a portion of the protein that is likely to be mobile, e.g., at, or within 1-5 residues of, the N- and C-termini, or within loops.

Conversely, if there are too many methionine and/or cysteine residues in a polypeptide sequence, over-incorporation of the selenium-containing side chains can lead to the inability of the polypeptide to fold and/or crystallize, and may potentially lead to complications in solving the crystal structure. In this case, methionine and/or cysteine mutants are prepared by substituting one or more of these Met and/or Cys residues with another residue. The considerations for these substitutions are the same as those discussed above for mutations that introduce methionine and/or cysteine residues into the polypeptide. Specifically, the Met and/or Cys residues are, for example, conservatively substituted with Leu/Ile and Ser, respectively.

As DNA encoding cysteine and methionine mutants can be used in the methods described above for obtaining SeCys and SeMet heavy-atom derivative crystals, the preferred Cys or Met mutant will have one Cys or Met residue for every 140 amino acids.

Production of Polypeptides

The native and mutated SYKKD or SYK polypeptides described herein may be chemically synthesized in whole or part using techniques that are well known in the art (see, e.g., Creighton, Proteins: Structures and Molecular Principles, W.H. Freeman & Co., NY, 1983).

Gene expression systems may be used for the synthesis of native and mutated polypeptides. Expression vectors containing the native or mutated polypeptide coding sequence and appropriate transcriptional/translational control signals, that are known to those skilled in the art may be constructed. These methods include in vitro recombinant DNA techniques, synthetic techniques and in vivo recombination/genetic recombination. See, for example, the techniques described in Sambrook et al., Molecular Cloning: A Laboratory Manual, Cold Spring Harbor Laboratory, NY, 2001, and Ausubel et al., Current Protocols in Molecular Biology, Greene Publishing Associates and Wiley Interscience, NY, 1989.

Host-expression vector systems may be used to express SYKKD or SYK. These include, but are not limited to, microorganisms such as bacteria transformed with recombinant bacteriophage DNA, plasmid DNA or cosmid DNA expression vectors containing the coding sequence; yeast transformed with recombinant yeast expression vectors containing the coding sequence; insect cell systems infected with recombinant virus expression vectors (e.g., baculovirus) containing the coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the coding sequence; or animal cell systems. The protein may also be expressed in human gene therapy systems, including, for example, expressing the protein to augment the amount of the protein in an individual, or to express an engineered therapeutic protein. The expression elements of these systems vary in their strength and specificities.

Specifically designed vectors allow the shuttling of DNA between hosts such as bacteria-yeast or bacteria-animal cells. An appropriately constructed expression vector may contain: an origin of replication for autonomous replication in host cells, one or more selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one that causes mRNAs to be initiated at high frequency.

The expression vector may also comprise various elements that affect transcription and translation, including, for example, constitutive and inducible promoters. These elements are often host and/or vector dependent. For example, when cloning in bacterial systems, inducible promoters such as the T7 promoter, pL of bacteriophage λ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, mammalian promoters (e.g., metallothionein promoter) or mammalian viral promoters, (e.g., adenovirus late promoter; vaccinia virus 7.5K promoter; SV40 promoter; bovine papilloma virus promoter; and Epstein-Barr virus promoter) may be used.

Various methods may be used to introduce the vector into host cells, for example, transformation, transfection, infection, protoplast fusion, and electroporation. The expression vector-containing cells are clonally propagated and individually analyzed to determine whether they produce the appropriate polypeptides. Various selection methods, including, for example, antibiotic resistance, may be used to identify host cells that have been transformed. Identification of polypeptide expressing host cell clones may be done by several means, including but not limited to immunological reactivity with anti-SYKKD or SYK antibodies, and the presence of host cell-associated activity.

Expression of cDNA may also be performed using in vitro produced synthetic mRNA. Synthetic mRNA can be efficiently translated in various cell-free systems, including but not limited to wheat germ extracts and reticulocyte extracts, as well as efficiently translated in cell-based systems, including, but not limited, to microinjection into frog oocytes.

To determine the cDNA sequence(s) that yields optimal levels of activity and/or protein, modified CDNA molecules are constructed. A non-limiting example of a modified CDNA is where the codon usage in the CDNA has been optimized for the host cell in which the CDNA will be expressed. Host cells are transformed with the cDNA molecules and the levels of SYKKD or SYK RNA and/or protein are measured.

Levels of SYK or SYKKD protein in host cells are quantitated by a variety of methods such as immunoaffinity and/or ligand affinity techniques, SYK or SYKKD-specific affinity beads or specific antibodies are used to isolate 35S-methionine labeled or unlabeled protein. Labeled or unlabeled protein is analyzed by SDS-PAGE. Unlabeled protein is detected by Western blotting, ELISA or RIA employing specific antibodies.

Following expression of SYK or SYKKD in a recombinant host cell, polypeptides may be recovered to provide the protein in active form. Several purification procedures are available and suitable for use. Recombinant SYK SYKKD may be purified from cell lysates or from conditioned culture media, by various combinations of, or individual application of, fractionation, or chromatography steps that are known in the art.

In addition, recombinant SYK or SYKKD can be separated from other cellular proteins by use of an immuno-affinity column made with monoclonal or polyclonal antibodies specific for full length nascent protein or polypeptide fragments thereof. Other affinity based purification techniques known in the art may also be used.

Alternatively, the polypeptides may be recovered from a host cell in an unfolded, inactive form, e.g., from inclusion bodies of bacteria. Proteins recovered in this form may be solubilized using a denaturant, e.g., guanidinium hydrochloride, and then refolded into an active form using methods known to those skilled in the art, such as dialysis.

Crystallization Of Polypeptides And Characterization Of Crystal

Various methods known in the art may be used to produce the native and heavy-atom derivative crystals of the present invention. Methods include, but are not limited to, batch, liquid bridge, dialysis, and vapor diffusion (see, e.g., McPherson, Crystallization of Biological Macromolecules, Cold Spring Harbor Press, New York, 1998; McPherson, Eur. J. Biochem. 189:1-23, 1990; Weber, Adv. Protein Chem. 41:1-36, 1991; Methods in Enzymology 276:13-22, 100-110; 131-143, Academic Press, San Diego, 1997).

Generally, native crystals are grown by dissolving substantially pure polypeptide in an aqueous buffer containing a precipitant at a concentration just below that necessary to precipitate the protein. Examples of precipitants include, but are not limited to, polyethylene glycol, ammonium sulfate, 2-methyl-2,4-pentanediol, sodium citrate, sodium chloride, glycerol, isopropanol, lithium sulfate, sodium acetate, sodium formate, potassium sodium tartrate, ethanol, hexanediol, ethylene glycol, dioxane, t-butanol and combinations thereof. Water is removed by controlled evaporation to produce precipitating conditions, which are maintained until crystal growth ceases.

In one embodiment, native crystals are grown by vapor diffusion in hanging drops or sitting drops (McPherson, Preparation and Analysis of Protein Crystals, John Wiley, New York, 1982; McPherson, Eur. J. Biochem. 189:1-23, 1990). Generally, up to about 25 μl, or up to about 5 μl, 3 μl, or 2 μl, of substantially pure polypeptide solution is mixed with a volume of reservoir solution. The ratio may vary according to biophysical conditions, for example, the ratio of protein volume: reservoir volume in the drop may be 1:1, giving a precipitant concentration about half that required for crystallization. Those of ordinary skill in the art recognize that the drop and reservoir volumes may be varied within certain biophysical conditions and still allow crystallization. In the sitting drop method, the polypeptide/precipitant solution is allowed to equilibrate in a closed container with a larger aqueous reservoir having a precipitant concentration optimal for producing crystals. In the hanging drop method, the polypeptide solution mixed with reservoir solution is suspended as a droplet underneath, for example, a coverslip, which is sealed onto the top of the reservoir. For both methods, the sealed container is allowed to stand, usually, for example, for up to 2-6 weeks, until crystals grow. It is preferable to check the drop periodically to determine if a crystal has formed. One way of viewing the drop is using, for example, a microscope. One method of checking the drop, for high throughput purposes, includes methods that may be found in, for example, U.S. Utility patent application Ser. No. 10/042,929, filed Oct. 18, 2001, entitled “Apparatus and Method for Identification of Crystals By In-situ X-Ray Diffraction.” Such methods include, for example, using an automated apparatus comprising a crystal growing incubator, an X-ray source adjacent to the crystal growing incubator, where the X-ray source is configured to irradiate the crystalline material grown in the crystal growing incubator, and an X-ray detector configured to detect the presence of the diffracted X-rays from crystalline material grown in the incubator. In more preferred methods, a charge coupled video camera is included in the detector system.

Those having skill in the art will recognize that the above-described crystallization conditions can be varied. Such variations may be used alone or in combination, and may include various volumes of protein solution and reservoir solution known to those of ordinary skill in the art. Other buffer solutions may be used such as Tris, imidazole, or MOPS buffer, so long as the desired pH range is maintained, and the chemical composition of the buffer is compatible with crystal formation.

Heavy-atom derivative crystals can be obtained by soaking native crystals in mother liquor containing salts of heavy metal atoms and can also be obtained from SeMet and/or SeCys mutants, as described above for native crystals.

Mutant proteins may crystallize under slightly different crystallization conditions than wild-type protein, or under very different crystallization conditions, depending on the nature of the mutation, and its location in the protein. For example, a non-conservative mutation may result in alteration of the hydrophilicity of the mutant, which may in turn make the mutant protein either more soluble or less soluble than the wild-type protein. Typically, if a protein becomes more hydrophilic as a result of a mutation, it will be more soluble than the wild-type protein in an aqueous solution and a higher precipitant concentration will be needed to cause it to crystallize. Conversely, if a protein becomes less hydrophilic as a result of a mutation, it will be less soluble in an aqueous solution and a lower precipitant concentration will be needed to cause it to crystallize. If the mutation happens to be in a region of the protein involved in crystal lattice contacts, crystallization conditions may be affected in more unpredictable ways.

Characterization of Crystals

The dimensions of a unit cell of a crystal are defined by six numbers, the lengths of three unique edges, a, b, and c, and three unique angles α, β, and γ. The type of unit cell that comprises a crystal is dependent on the values of these variables, as discussed above.

When a crystal is exposed to an X-ray beam, the electrons of the molecules in the crystal diffract the beam such that there is a sphere of diffracted X-rays around the crystal. The angle at which diffracted beams emerge from the crystal can be computed by treating diffraction as if it were reflection from sets of equivalent, parallel planes of atoms in a crystal (Bragg's Law). The most obvious sets of planes in a crystal lattice are those that are parallel to the faces of the unit cell. These and other sets of planes can be drawn through the lattice points. Each set of planes is identified by three indices, hkl. The h index gives the number of parts into which the a edge of the unit cell is cut, the k index gives the number of parts into which the b edge of the unit cell is cut, and the l index gives the number of parts into which the c edge of the unit cell is cut by the set of hkl planes. Thus, for example, the 235 planes cut the a edge of each unit cell into halves, the b edge of each unit cell into thirds, and the c edge of each unit cell into fifths. Planes that are parallel to the bc face of the unit cell are the 100 planes; planes that are parallel to the ac face of the unit cell are the 010 planes; and planes that are parallel to the ab face of the unit cell are the 001 planes.

When a detector is placed in the path of the diffracted X-rays, in effect cutting into the sphere of diffraction, a series of spots, or reflections, may be recorded of a still crystal (not rotated) to produce a “still” diffraction pattern. Each reflection is the result of X-rays reflecting off one set of parallel planes, and is characterized by an intensity, which is related to the distribution of molecules in the unit cell, and hkl indices, which correspond to the parallel planes from which the beam producing that spot was reflected. If the crystal is rotated about an axis perpendicular to the X-ray beam, a large number of reflections are recorded on the detector, resulting in a diffraction pattern.

The unit cell dimensions and space group of a crystal can be determined from its diffraction pattern. First, the spacing of reflections is inversely proportional to the lengths of the edges of the unit cell. Therefore, if a diffraction pattern is recorded when the X-ray beam is perpendicular to a face of the unit cell, two of the unit cell dimensions may be deduced from the spacing of the reflections in the x and y directions of the detector, the crystal-to-detector distance, and the wavelength of the X-rays. Those of skill in the art will appreciate that, in order to obtain all three unit cell dimensions, the crystal must be rotated such that the X-ray beam is perpendicular to another face of the unit cell. Second, the angles of a unit cell can be determined by the angles between lines of spots on the diffraction pattern. Third, the absence of certain reflections and the repetitive nature of the diffraction pattern, which may be evident by visual inspection, indicate the internal symmetry, or space group, of the crystal. Therefore, a crystal may be characterized by its unit cell and space group, as well as by its diffraction pattern.

Once the dimensions of the unit cell are determined, the likely number of polypeptides in the asymmetric unit can be deduced from the size of the polypeptide, the density of the average protein, and the typical solvent content of a protein crystal, which is usually in the range of 30-70% of the unit cell volume (Matthews, J. Mol. Biol. 33(2):491-97, 1968). Collection of Data and Determination of Structure Solutions

The diffraction pattern is related to the three-dimensional shape of the molecule by a Fourier transform. The process of determining the solution is in essence a re-focusing of the diffracted X-rays to produce a three-dimensional image of the molecule in the crystal. Since re-focusing of X-rays cannot be done with a lens at this time, it is done via mathematical operations.

The sphere of diffraction has symmetry that depends on the internal symmetry of the crystal, which means that certain orientations of the crystal will produce the same set of reflections. Thus, a crystal with high symmetry has a more repetitive diffraction pattern, and there are fewer unique reflections that need to be recorded in order to have a complete representation of the diffraction. The goal of data collection, a dataset, is a set of consistently measured, indexed intensities for as many reflections as possible. A complete dataset is collected if at least 80%, preferably at least 90%, most preferably at least 95% of unique reflections are recorded. In one embodiment, a complete dataset is collected using one crystal. In another embodiment, a complete dataset is collected using more than one crystal of the same type.

Sources of X-rays include, but are not limited to, a rotating anode X-ray generator such as a Rigaku RU-200, a micro source or mini-source, a sealed-beam source, or a beam line at a synchrotron light source, such as the Advanced Photon Source at Argonne National Laboratory. Suitable detectors for recording diffraction patterns include, but are not limited to, X-ray sensitive film, multiwire area detectors, image plates coated with phosphorus, and CCD cameras. Typically, the detector and the X-ray beam remain stationary, so that, in order to record diffraction from different parts of the crystal's sphere of diffraction, the crystal itself is moved via an automated system of moveable circles called a goniostat.

One of the biggest problems in data collection, particularly from macromolecular crystals having a high solvent content, is the rapid degradation of the crystal in the X-ray beam. In order to slow the degradation, data is often collected from a crystal at liquid nitrogen temperatures. In order for a crystal to survive the initial exposure to liquid nitrogen, the formation of ice within the crystal may be prevented by the use of a cryoprotectant. Suitable cryoprotectants include, but are not limited to, low molecular weight polyethylene glycols, ethylene glycol, sucrose, glycerol, xylitol, and combinations thereof. Crystals may be soaked in a solution comprising the one or more cryoprotectants prior to exposure to liquid nitrogen, or the one or more cryoprotectants may be added to the crystallization solution. Data collection at liquid nitrogen temperatures may allow the collection of an entire dataset from one crystal.

Once a dataset is collected, the information is used to determine the three-dimensional structure of the molecule in the crystal. This phase information may be acquired by methods described below in order to perform a Fourier transform on the diffraction pattern to obtain the three-dimensional structure of the molecule in the crystal. It is the determination of phase information that in effect refocuses X-rays to produce the image of the molecule.

One method of obtaining phase information is by isomorphous replacement, in which heavy-atom derivative crystals are used. In this method, the positions of heavy atoms bound to the molecules in the heavy-atom derivative crystal are determined, and this information is then used to obtain the phase information necessary to elucidate the three-dimensional structure of a native crystal (Blundell et al., Protein Crystallography, Academic Press, 1976).

Another method of obtaining phase information is by molecular replacement, which is a method of calculating initial phases for a new crystal of a polypeptide whose structure coordinates are unknown by orienting and positioning a polypeptide whose structure coordinates are known within the unit cell of the new crystal so as to best account for the observed diffraction pattern of the new crystal. Phases are then calculated from the oriented and positioned polypeptide and combined with observed amplitudes to provide an approximate Fourier synthesis of the structure of the molecules comprising the new crystal (Lattman, Methods in Enzymology 115:55-77, 1985; Rossmann, “The Molecular Replacement Method,” Int. Sci. Rev. Ser. No. 13, Gordon & Breach, New York, 1972).

A third method of phase determination is multi-wavelength anomalous diffraction or MAD. In this method, X-ray diffraction data are collected at several different wavelengths from a single crystal containing at least one heavy atom with absorption edges near the energy of incoming X-ray radiation. The resonance between X-rays and electron orbitals leads to differences in X-ray scattering that permits the locations of the heavy atoms to be identified, which in turn provides phase information for a crystal of a polypeptide. A detailed discussion of MAD analysis can be found in Hendrickson, Trans. Am. Crystallogr. Assoc., 21:11, 1985; Hendrickson etal., EMBO J. 9:1665, 1990; and Hendrickson, Science, 254:51-58, 1991).

A fourth method of determining phase information is single wavelength anomalous dispersion or SAD. In this technique, X-ray diffraction data are collected at a single wavelength from a single native or heavy-atom derivative crystal, and phase information is extracted using anomalous scattering information from atoms such as sulfur or chlorine in the native crystal or from the heavy atoms in the heavy-atom derivative crystal. The wavelength of X-rays used to collect data for this phasing technique need not be close to the absorption edge of the anomalous scatterer. A detailed discussion of SAD analysis can be found in Brodersen, et al., Acta Cryst., D56:431-41, 2000.

A fifth method of determining phase information is single isomorphous replacement with anomalous scattering or SIRAS. SIRAS combines isomorphous replacement and anomalous scattering techniques to provide phase information for a crystal of a polypeptide. X-ray diffraction data are collected at a single wavelength, usually from both a native and a single heavy-atom derivative crystal. Phase information obtained only from the location of the heavy atoms in a single heavy-atom derivative crystal leads to an ambiguity in the phase angle, which is resolved using anomalous scattering from the heavy atoms. Phase information is extracted from both the location of the heavy atoms and from anomalous scattering of the heavy atoms. A detailed discussion of SIRAS analysis can be found in North, Acta Cryst. 18:212-16, 1965; Matthews, Acta Cryst. 20:82-86, 1966; Methods in Enzymology 276:530-37, 1997.

Once phase information is obtained, it is combined with the diffraction data to produce an electron density map, an image of the electron clouds surrounding the atoms that constitute the molecules in the unit cell. The higher the resolution of the data, the more distinguishable the features of the electron density map, because atoms that are closer together are resolvable. A model of the macromolecule is then built into the electron density map with the aid of a computer, using as a guide all available information, such as the polypeptide sequence and the established rules of molecular structure and stereochemistry. Interpreting the electron density map is a process of finding the chemically reasonable conformation that fits the map precisely.

After a model is generated, a structure is refined. Refinement is the process of minimizing the function φ, which is the difference between observed and calculated intensity values (measured by an R-factor), and which is a function of the position, temperature factor, and occupancy of each non-hydrogen atom in the model. This usually involves alternate cycles of real space refinement, i.e., calculation of electron density maps and model building, and reciprocal space refinement, i.e., computational attempts to improve the agreement between the original intensity data and intensity data generated from each successive model. Refinement ends when the function φ converges on a minimum wherein the model fits the electron density map and is stereochemically and conformationally reasonable. During the last stages of refinement, ordered solvent molecules are added to the structure.

Structures of SYKKD

The present invention provides, for the first time, the high-resolution three-dimensional structures and molecular structure coordinates of crystalline SYKKD as determined by X-ray crystallography.

Contemplated within the scope of the present invention are any set of structure coordinates obtained for crystals of SYKKD, whether native crystals, heavy-atom derivative crystals or co-crystals, that have a root mean square deviation (“r.m.s.d.”) of up to about or equal to 1.5 Å, preferably 1.25 Å, preferably 1 Å, preferably 1.75 Å, and preferably 0.5 Å when superimposed, using backbone atoms (N, C-α, C and O), or using C-α atoms, on the structure coordinates listed in FIGS. 4-7 are considered to be within the scope of the present invention when at least 50% to 100% of the backbone atoms of SYKKD are included in the superposition. The amino acid numbers in FIGS. 4-7 reflect the amino acid position in the expressed protein used to obtain the crystals of the present invention. Those of ordinary skill in the art may align the sequence with other sequences of SYKKD to, if desired, correlate the amino acid residue number. Thus, the “sequence of FIG. 4, 5, 6, or 7” relates to the amino acid number designations, for the amino acid sequence, and not specifically the structural coordinates of FIGS. 4-7.

Structure Coordinates

The molecular structure coordinates can be used in molecular modeling and design, as described more fully below. The present invention encompasses the structure coordinates and other information, e.g., amino acid sequence, connectivity tables, vector-based representations, temperature factors, etc., used to generate the three-dimensional structure of the polypeptide for use in the software programs described below and other software programs.

The invention includes methods of producing computer readable databases comprising the three-dimensional molecular structure coordinates of certain molecules, including, for example, the SYKKD structure coordinates, the structure coordinates of binding pockets or active sites of SYKKD, or structure coordinates of compounds capable of binding to SYKKD. The databases of the present invention may comprise any number of sets of molecular structure coordinates for any number of molecules, including, for examples, structure coordinates of one molecule. In other embodiments, the databases of the present invention may comprise structure coordinates of a compound or compounds that have been identified by virtual screening to bind to a SYK binding pocket, or other representations of such compounds such as, for example, a graphic representation or a name. By “database” is meant a collection of retrievable data. The invention encompasses machine readable media embedded with or containing information regarding the three-dimensional structure of a crystalline polypeptide and/or model, such as, for example, its molecular structure coordinates, described herein, or with subunits, domains, and/or, portions thereof such as, for example, portions comprising active sites, accessory binding sites, and/or binding pockets in either liganded or unliganded forms. Alternatively, the information may be that of identifiers which represent specific structures found in a protein. As used herein, “machine readable medium” refers to any medium that can be read and accessed directly by a computer or scanner. Such media may take many forms, including but not limited to, non-volatile, volatile and transmission media. Non-volatile media, i.e., media that can retain information in the absence of power, includes a ROM. Volatile media, i.e., media that cannot retain information in the absence of power, includes a main memory. Transmission media includes coaxial cables, copper wire and fiber optics, including the wires that comprise the bus. Transmission media can also take the form of carrier waves; i.e., electromagnetic waves that can be modulated, as in frequency, amplitude or phase, to transmit information signals. Additionally, transmission media can take the form of acoustic or light waves, such as those generated during radio wave and infrared data communications.

Such media also include, but are not limited to: magnetic storage media, such as floppy discs, flexible discs, hard disc storage medium and magnetic tape; optical storage media such as optical discs or CD-ROM; electrical storage media such as RAM or ROM, PROM (i.e., programmable read only memory), EPROM (i.e., erasable programmable read only memory), including FLASH-EPROM, any other memory chip or cartridge, carrier waves, or any other medium from which a processor can retrieve information, and hybrids of these categories such as magnetic/optical storage media. Such media further include paper on which is recorded a representation of the molecular structure coordinates, e.g., Cartesian coordinates, that can be read by a scanning device and converted into a format readily accessed by a computer or by any of the software programs described herein by, for example, optical character recognition (OCR) software. Such media also include physical media with patterns of holes, such as, for example, punch cards, and paper tape.

A variety of data storage structures are available for creating a computer readable medium having recorded thereon the molecular structure coordinates of the invention or portions thereof and/or X-ray diffraction data. The choice of the data storage structure will generally be based on the means chosen to access the stored information. In addition, a variety of data processor programs and formats can be used to store the sequence and X-ray data information on a computer readable medium. Such formats include, but are not limited to, macromolecular Crystallographic Information File (“mmCIF”) and Protein Data Bank (“PDB”) format (Research Collaboratory for Structural Bioinformatics; www.rcsb.org; Cambridge Crystallographic Data Centre format (www.ccdc.can.ac.uk/support/csd_doc/volume3/z323.html); Structure-data (“SD”) file format (MDL Information Systems, Inc.; Dalby, et al., J. Chem. Inf. Comp. Sci., 32:244-55, 1992; and line-notation, e.g., as used in SMILES (Weininger, J. Chem. Inf. Comp. Sci. 28:31-36, 1988). Methods of converting between various formats read by different computer software will be readily apparent to those of skill in the art, e.g., BABEL (v. 1.06, Walters & Stahl, ©1992, 1993, 1994; www.brunel.ac.uk/departments/chem/babel.htm). All format representations of the polypeptide coordinates described herein, or portions thereof, are contemplated by the present invention. By providing computer readable medium having stored thereon the atomic coordinates of the invention, one of skill in the art can routinely access the atomic coordinates of the invention, or portions thereof, and related information for use in modeling and design programs, described in detail below.

A computer may be used to display the structure coordinates or the three-dimensional representation of the protein or peptide structures, or portions thereof, such as, for example, portions comprising active sites, accessory binding sites, and/or binding pockets, in either liganded or unliganded form, of the present invention. The term “computer” includes, but is not limited to, mainframe computers, personal computers, portable laptop computers, and personal data assistants (“PDAs”) which can store data and independently run one or more applications, i.e., programs. The computer may include, for example, a machine readable storage medium of the present invention, a working memory for storing instructions for processing the machine-readable data encoded in the machine readable storage medium, a central processing unit operably coupled to the working memory and to the machine readable storage medium for processing the machine readable information, and a display operably coupled to the central processing unit for displaying the structure coordinates or the three-dimensional representation. The information contained in the machine-readable medium may be in the form of, for example, X-ray diffraction data, structure coordinates, electron density maps, or ribbon structures. The information may also include such data for co-complexes between a compound and a protein or peptide of the present invention.

The computers of the present invention may also include, for example, a central processing unit, a working memory which may be, for example, random-access memory (RAM) or “core memory,” mass storage memory (for example, one or more disk drives or CD-ROM drives), one or more cathode-ray tube (“CRT”) display terminals or one or more LCD displays, one or more keyboards, one or more input lines, and one or more output lines, all of which are interconnected by a conventional bi-directional system bus. Machine-readable data of the present invention may be inputted and/or outputted through a modem or modems connected by a telephone line or a dedicated data line (either of which may include, for example, wireless modes of communication). The input hardware may also (or instead) comprise CD-ROM drives or disk drives. Other examples of input devices are a keyboard, a mouse, a trackball, a finger pad, or cursor direction keys. Output hardware may also be implemented by conventional devices. For example, output hardware may include a CRT, or any other display terminal, a printer, or a disk drive. The CPU coordinates the use of the various input and output devices, coordinates data accesses from mass storage and accesses to and from working memory, and determines the order of data processing steps. The computer may use various software programs to process the data of the present invention. Examples of many of these types of software are discussed throughout the present application.

Those of skill in the art will recognize that a set of structure coordinates is a relative set of points that define a shape in three dimensions. Therefore, two different sets of coordinates could define the identical or a similar shape. Also, minor changes in the individual coordinates may have very little effect on the peptide's shape. Minor changes in the overall structure may have very little to no effect, for example, on the binding pocket, and would not be expected to significantly alter the nature of compounds that might associate with the binding pocket.

Although Cartesian coordinates are important and convenient representations of the three-dimensional structure of a polypeptide, other representations of the structure are also useful. Therefore, the three-dimensional structure of a polypeptide, as discussed herein, includes not only the Cartesian coordinate representation, but also all alternative representations of the three-dimensional distribution of atoms. For example, atomic coordinates may be represented as a Z-matrix, wherein a first atom of the protein is chosen, a second atom is placed at a defined distance from the first atom, and a third atom is placed at a defined distance from the second atom so that it makes a defined angle with the first atom. Each subsequent atom is placed at a defined distance from a previously placed atom with a specified angle with respect to the third atom, and at a specified torsion angle with respect to a fourth atom. Atomic coordinates may also be represented as a Patterson function, wherein all interatomic vectors are drawn and are then placed with their tails at the origin. This representation is particularly useful for locating heavy atoms in a unit cell. In addition, atomic coordinates may be represented as a series of vectors having magnitude and direction and drawn from a chosen origin to each atom in the polypeptide structure. Furthermore, the positions of atoms in a three-dimensional structure may be represented as fractions of the unit cell (fractional coordinates), or in spherical polar coordinates.

Additional information, such as thermal parameters, which measure the motion of each atom in the structure, chain identifiers, which identify the particular chain of a multi-chain protein in which an atom is located, and connectivity information, which indicates to which atoms a particular atom is bonded, is also useful for representing a three-dimensional molecular structure.

The structural information of a compound that binds a SYKKD of the invention may be similarly stored and transmitted as described above for structural information of SYKKD.

Uses of the Molecular Structure Coordinates

Structure information, typically in the form of molecular structure coordinates, can be used in a variety of computational or computer-based methods to, for example, design, screen for, and/or identify compounds that bind the crystallized polypeptide or a portion or fragment thereof, or to intelligently design mutants that have altered biological properties.

When designing or identifying compounds that may associate with a given protein, binding pockets are often analyzed. The term “binding pocket,” refers to a region of a protein that, because of its shape, likely associates with a chemical entity or compound. A binding pocket may be the same as an active site. A binding pocket of a protein is usually involved in associating with the protein's natural ligands or substrates, and is often the basis for the protein's activity. A binding pocket may refer to an active site. Many drugs act by associating with a binding pocket of a protein. A binding pocket may comprise amino acid residues that line the cleft of the pocket. Those of ordinary skill in the art will recognize that the numbering system used for other isoforms of SYKKD may be different, but that the corresponding amino acids may be determined with a homology software program known to those of ordinary skill in the art. A binding pocket homolog comprises amino acids having structure coordinates that have a root mean square deviation from structure coordinates, as indicated in FIGS. 4-7, of the binding pocket amino acids of up to about 1.5 Å, preferably up to about 1.25 Å, preferably up to about 1 Å, preferably up to about 0.75 Å, preferably up to about 0.5 Å, and preferably up to about 0.25 Å.

Where a binding pocket or regulatory site is said to comprise amino acids having particular structure coordinates, the amino acids comprise the same amino acid residues, or may comprise amino acids having similar properties, as shown in, for example, Table 1, and have either the same relative three-dimensional structure coordinates as FIGS. 4-7, or the group of amino acid residues named as part of the binding pocket have an rmsd of within 1.5 Å, preferably within 1.25 Å, preferably within 1 Å, preferably within 0.75 Å, preferably within 0.5 Å, and preferably within 0.25 Å of the structure coordinates of FIGS. 4-7. Preferably, when comparing the structure coordinates of the backbone atoms of the amino acid residues, the rmsd is within 1.5 Å, preferably within 1.25 Å, preferably within 1 Å, preferably within 0.75 Å, preferably within 0.5 Å, and more preferably within 0.25 Å.

Software applications are available to compare structures, or portions thereof, to determine if they are sufficiently similar to the structures of the invention such as DALI (Holm and Sander, J. Mol. Biol. 233:123-38, 1993; (See European Bioinformatics Institute site at www.ebi.ac.uk/); MOE; CE (Shindyalov, IN, Bourne, PE, “Protein Structure Alignment by Incremental Combinatorial Extension (CE) of the Optimal Path,” Protein Engineering, 11:739-47, 1998); and DEJAVU (Uppsala Software Factory; Kleywegt, G.S. & Jones, T.A., “Detecting Folding Motifs and Similarities in Protein Structure,” Methods in Enzymology, 277:525-45, 1997).

The crystals and structure coordinates obtained therefrom may be used for rational drug design to identify and/or design compounds that bind SYK as an approach towards developing new therapeutic agents. For example, a high resolution X-ray structure of, for example, a crystallized protein saturated with solvent, will often show the locations of ordered solvent molecules around the protein, and in particular at or near putative binding pockets of the protein. This information can then be used to design molecules that bind these sites, the compounds synthesized and tested for binding in biological assays (Travis, Science, 262:1374, 1993).

The structure may also be computationally screened with a plurality of molecules to determine their ability to bind to the SYKKD at various sites. Such compounds can be used as targets or leads in medicinal chemistry efforts to identify, for example, inhibitors of potential therapeutic importance (Travis, Science, 262:1374, 1993). The three dimensional structures of such compounds may be superimposed on a three dimensional representation of SYKKD or an active site or binding pocket thereof to assess whether the compound fits spatially into the representation and hence the protein. Structural information produced by such methods and concerning a compound that fits (or a fitting portion of such a compound) may be stored in a machine readable medium. Alternatively, one or more identifiers of a compound that fits, or a fitting portion thereof, may be stored in a machine readable medium. Examples of identifiers include chemical name or abbreviation, chemical or molecular formula, chemical structure, and/or other identifying information. As an non-limiting example, if the three dimensional structure of phenol is found to fit the active site of SYKKD, the structural information of phenol, or the portion that fits, may be stored for further use. Alternatively, an identifier of phenol, or of the portion that fits, such as the —OH group, may be stored for further use. Other identifying information for phenol may also be used to represent it. All storage of information concerning a compound that fits may optionally be in combination with one or more pieces of information concerning SYKKD.

In an analogous manner, the structure of SYKKD or an active site or binding pocket thereof can be used to computationally screen small molecule databases for chemical entities or compounds that can bind in whole, or in part, to SYK. In this screening, the quality of fit of such entities or compounds to the binding pocket may be judged either by shape complementarity or by estimated interaction energy (Meng, et al., J. Comp. Chem. 13:505-24, 1992).

In still another embodiment, compounds can be developed that are analogues of natural substrates, reaction intermediates or reaction products of SYK. The reaction intermediates of SYK can be deduced from the substrates, or reaction products in co-complex with SYKKD. The binding of substrates, reaction intermediates, and reaction products may change the conformation of the binding pocket, which provides additional information regarding binding patterns of potential ligands, activators, inhibitors, and the like. Such information is also useful to design improved analogues of known SYK inhibitors or to design novel classes of inhibitors based on the substrates, reaction intermediates, and reaction products of SYKKD and SYKKD-inhibitor co-complexes. This provides a novel route for designing SYKKD inhibitors with both high specificity and stability.

Another method of screening or designing compounds that associate with a binding pocket includes, for example, computationally designing a negative image of the binding pocket. This negative image may be used to identify a set of pharmacophores. A pharmacophore may be a description of functional groups and how they relate to each other in three-dimensional space. This set of pharmacophores can be used to design compounds and screen chemical databases for compounds that match with the pharmacophore(s). Compounds identified by this method may then be further evaluated computationally or experimentally for binding activity. Various computer programs may be used to create the negative image of the binding pocket, for example; GRID (Goodford, J. Med. Chem. 28:849-57, 1985; GRID is available from Oxford University, Oxford, UK); MCSS (Miranker & Karplus, Proteins: Structure, Function and Genetics 11:29-34, 1991; MCSS is available from Accelrys, Inc., San Diego, Calif.); LUDI (Bohm, J. Comp. Aid. Molec. Design 6:61-78, 1992; LUDI is available from Accelrys, Inc., San Diego, Calif.); DOCK (Kuntz et al.; J. Mol. Biol. 161:269-88, 1982; DOCK is available from University of California, San Francisco, Calif.); DOCKIT (Metaphorics, Mission Viejo, Calif.) and MOE. Other appropriate programs are desribed in, for example, Halperin, et al., Proteins 47(4): 409-43 (2002).

Thus, among the various embodiments of the present invention are methods of identifying, screening, and designing compounds that associate with a binding pocket of SYKKD.

The design of compounds that bind to and/or modulate SYK, for example that inhibit or activate SYK according to this invention generally involves consideration of two factors. First, the compound must be capable of physically and structurally associating, either covalently or non-covalently with SYK. For example, covalent interactions may be important for designing irreversible or suicide inhibitors of a protein. Non-covalent molecular interactions important in the association of SYK with the compound include hydrogen bonding, ionic interactions and van der Waals and hydrophobic interactions. Second, the compound must be able to assume a conformation and orientation in relation to the binding pocket, that allows it to associate with SYK. Although certain portions of the compound will not directly participate in this association with SYK, those portions may still influence the overall conformation of the molecule and may have a significant impact on potency. Conformational requirements include the overall three-dimensional structure and orientation of the chemical group or compound in relation to all or a portion of the binding pocket, or the spacing between functional groups of a compound comprising several chemical groups that directly interact with SYK.

Computer modeling techniques may be used to assess the potential modulating or binding effect of a chemical compound on SYKKD. If computer modeling indicates a strong interaction, the molecule may then be synthesized and tested for its ability to bind to SYK and affect (by inhibiting or activating) its activity.

Modulating or other binding compounds of SYK may be computationally evaluated and designed by means of a series of steps in which chemical groups or fragments are screened and selected for their ability to associate with the individual binding pockets or other areas of SYK. Several methods are available to screen chemical groups or fragments for their ability to associate with SYK. This process may begin by visual inspection of, for example, the active site on the computer screen based on the SYKKD coordinates. Selected fragments or chemical groups may then be positioned in a variety of orientations, or docked, within an individual binding pocket of SYKKD (Blaney, J. M. and Dixon, J. S., Perspectives in Drug Discovery and Design, 1:301, 1993). Manual docking may be accomplished using software such as Insight II (Accelrys, San Diego, Calif.) MOE; CE (Shindyalov, Ind., Bourne, P E, “Protein Structure Alignment by Incremental Combinatorial Extension (CE) of the Optimal Path,” Protein Engineering, 11:739-47, 1998); and SYBYL (Molecular Modeling Software, Tripos Associates, Inc., St. Louis, Mo., 1992), followed by energy minimization and molecular dynamics with standard molecular mechanics force fields, such as CHARMM (Brooks, et al., J. Comp. Chem. 4:187-217, 1983). More automated docking may be accomplished by using programs such as DOCK (Kuntz et al., J. Mol. Biol., 161:269-88, 1982; DOCK is available from University of California, San Francisco, Calif.); AUTODOCK (Goodsell & Olsen, Proteins: Structure, Function, and Genetics 8:195-202, 1990; AUTODOCK is available from Scripps Research Institute, La Jolla, Calif.); GOLD (Cambridge Crystallographic Data Centre (CCDC); Jones et al., J. Mol. Biol. 245:43-53, 1995); and FLEXX (Tripos, St. Louis, Mo.; Rarey, M., et al., J. Mol. Biol. 261:470-89, 1996); AMBER (Weiner, et al., J. Am. Chem. Soc. 106: 765-84, 1984) and C² MMFF (Merck Molecular Force Field; Accelrys, San Diego, Calif.). Other appropriate programs are described in, for example, Halperin, et al.

Specialized computer programs may also assist in the process of selecting fragments or chemical groups. These include DOCK; GOLD; LUDI; FLEXX (Tripos, St. Louis, Mo.; Rarey, M., et al., J. Mol. Biol. 261:470-89, 1996); and GLIDE (Eldridge, et al., J. Comput. Aided Mol. Des. 11:425-45, 1997; Schrödinger, Inc., New York). Other appropriate programs are described in, for example, Halperin, et al., (Portland, Org.).

Once suitable chemical groups or fragments have been selected, they can be assembled into a single compound or inhibitor. Assembly may proceed by visual inspection of the relationship of the fragments to each other in the three-dimensional image displayed on a computer screen in relation to the structure coordinates of SYKKD. This would be followed by manual model building using software such as SYBYL, (Tripos, St. Louis, Mo.); Insight II (Accelrys, San Diego, Calif.); and MOE (Chemical Computing Group, Inc., Montreal, Canada). Other appropriate program are described in, for example, Halperin, et al.

Useful programs to aid one of skill in the art in connecting the individual chemical groups or fragments include, for example:

1. CAVEAT (Bartlett et al., ‘CAVEAT: A Program to Facilitate the Structure-Derived Design of Biologically Active Molecules'. In Molecular Recognition in Chemical and Biological Problems’, Special Pub., Royal Chem. Soc. 78:182-96, 1989). CAVEAT is available from the University of California, Berkeley, Calif.

2. 3D Database systems such as ISIS or MACCS-3D (MDL Information Systems, San Leandro, Calif.). This area is reviewed in Martin, J. Med. Chem. 35:2145-54, 1992).

3. HOOK (Eisen et al., Proteins: Struct., Funct., Genet., 19:199-221, 1994) (available from Accelrys, Inc., San Diego, Calif.).

4. LUDI (Bohm, J. Comp. Aid. Molec. Design 6:61-78, 1992). LUDI is available from Accelrys, Inc., San Diego, Calif.

Instead of proceeding to build a SYK inhibitor in a step-wise fashion one fragment or chemical group at a time, as described above, SYK binding compounds may be designed as a whole or ‘de novo’ using either an empty active site or optionally including some portion(s) of a known inhibitor(s). These methods include, for example:

1. LUDI (Bohm, J. Comp. Aid. Molec. Design 6:61-78, 1992). LUDI is available from Accelrys, Inc., San Diego, Calif.

2. LEGEND (Nishibata & Itai, Tetrahedron, 47:8985, 1991). LEGEND is available from Accelrys, Inc., San Diego, Calif.

3. LeapFrog (available from Tripos, Inc., St. Louis, Mo.).

4. SPROUT (Gillet et al., J. Comput. Aided Mol. Design 7:127-53, 1993) (available from the University of Leeds, U.K.).

5. GenStar (Murcko, M. A. and Rotstein, S. H. J. Comput. Aided Mol. Des. 7:23-43, 1993).

6. GroupBuild (Rotstein, S. H., and Murcko, M. A., J. Med. Chem. 36:1700, 1993).

7. GrowMol (Rich, D. H. et al., Chimia, 51:45, 1997).

8. Grow (UpJohn; Moon J, Howe W, Proteins, 11:314-28, 1991).

9. SmoG (DeWitte, R. S., Abstr. Pap Am Chem. S. 214:6-Comp Part 1, Sept. 7, 1997; DeWitte, R. S. & Shakhnovich, E. I., J. Am. Chem. Soc. 118:11733-44, 1996).

10. LigBuilder (PDB (www.rcsb.org/pdb); Wang R, Ying G, Lai L, J. Mol. Model. 6: 498-516, 1998).

Other molecular modeling techniques may also be employed in accordance with this invention. See, e.g., Cohen et al., J. Med. Chem. 33:883-94, 1990. See also, Navia & Murcko, Current Opinions in Structural Biology 2:202-10, 1992; Balbes et al., Reviews in Computational Chemistry, 5:337-80, 1994, (Lipkowitz and Boyd, Eds.) (VCH, New York); Guida, Curr. Opin. Struct. Biol. 4:777-81, 1994.

During design and selection of compounds by the above methods, the efficiency with which that compound may bind to SYKKD may be tested and optimized by computational evaluation. For example, a compound that has been designed or selected to function as a SYK inhibitor may occupy a volume not overlapping the volume occupied by the active site residues when the native substrate is bound, however, those of ordinary skill in the art will recognize that there is some flexibility, allowing for rearrangement of the main chains and the side chains. In addition, one of ordinary skill may design compounds that could exploit protein rearrangement upon binding, such as, for example, resulting in an induced fit. An effective SYK inhibitor may demonstrate a relatively small difference in energy between its bound and free states (i.e., it must have a small deformation energy of binding and/or low conformational strain upon binding). Thus, the most efficient SYK inhibitors should, for example, be designed with a deformation energy of binding of not greater than 10 kcal/mol, for example, not greater than 7 kcal/mol, for example, not greater than 5 kcal/mol and, for example, not greater than 2 kcal/mol. SYK inhibitors may interact with the protein in more than one conformation that is similar in overall binding energy. In those cases, the deformation energy of binding is taken to be the difference between the energy of the free compound and the average energy of the conformations observed when the inhibitor binds to the enzyme.

A compound selected or designed for binding to SYKKD may be further computationally optimized so that in its bound state it would, for example, lack repulsive electrostatic interaction with the target protein. Non-complementary electrostatic interactions include repulsive charge-charge, dipole-dipole and charge-dipole interactions. Specifically, the sum of all electrostatic interactions between the inhibitor and the protein when the inhibitor is bound to it may make a neutral or favorable contribution to the enthalpy of binding.

Specific computer software is available in the art to evaluate compound deformation energy and electrostatic interaction. Examples of programs designed for such uses include: Gaussian 94, revision C (Frisch, Gaussian, Inc., Pittsburgh, Pa. ©1995); AMBER, version 7.1 (Kollman, University of California at San Francisco, ©2002); QUANTA/CHARMM (Accelrys, Inc., San Diego, Calif. ©1995); Insight II/Discover (Accelrys, Inc., San Diego, Calif. ©1995); DelPhi (Accelrys, Inc., San Diego, Calif. ©1995); and AMSOL (University of Minnesota) (Quantum Chemistry Program Exchange, Indiana University). These programs may be implemented, for instance, using a computer workstation, as are well known in the art, for example, a LINUX, SGI or Sun workstation. Other hardware systems and software packages will be known to those skilled in the art.

Once a SYKKD binding compound has been optimally selected or designed, as described above, substitutions may then be made in some of its atoms or chemical groups in order to improve or modify its binding properties. Generally, initial substitutions are conservative, i.e., the replacement group will have approximately the same size, shape, hydrophobicity and charge as the original group. One of skill in the art will understand that substitutions known in the art to alter conformation should be avoided. Such altered chemical compounds may then be analyzed for efficiency of binding to SYKKD by the same computer methods described in detail above. Methods of structure-based drug design are described in, for example, Klebe, G., J. Mol. Med. 78:269-81, 2000); Hol. W. G. J., Angewandte Chemie (Int'l Edition in English) 25:767-852, 1986; and Gane, P. J. and Dean, P. M., Current Opinion in Structural Biology, 10:401-04, 2000.

The present invention also provides means for the preparation of a compound the structure of which has been identified or designed, as described above, as binding SYKKD or an active site or binding pocket thereof. Where the compound is already known or designed, the synthesis thereof may readily proceed by means known in the art. Alternatively, compounds that match the structure of one or more pharmacophores as described above may be prepared by means known in the art. In an alternative embodiment, the production of a compound may proceed by introduction of one or more desired chemical groups by attachment to an initial compound which binds SYKKD or an active site or binding pocket thereof and which has, or has been modified to contain, one or more chemical moieties for attachment of one or more desired chemical groups. The initial compound may be viewed as a “scaffold” comprising at least one moiety capable of binding or associating with one or more residues of SYKKD or an active site or binding pocket thereof.

The initial compound may be a flexible or rigid “scaffold”, optionally containing a linker for introduction of additional chemical moieties. Various scaffold compounds can be used, including, but not limited to, aliphatic carbon chains, pyrrolidinones, sulfonamidopyrrolidinones, cycloalkanonedienes including cyclopentanonedienes, cyclohexanonedienes, and cyclopheptanonedienes, carbazoles, imidazoles, benzimidiazoles, pyridine, isoxazoles, isoxazolines, benzoxazinones, benzamidines, pyridinones and derivatives thereof. Other scaffolds are described in, for example, Klebe, G., J. Mol. Med. 78: 269-281 (2000); Maignan, S. and Mikol, V., Curr. Top. Med. Chem. 1: 161-174 (2001); and U.S. Pat. No. 5,756,466 to Bemis et al. The scaffold compound used may, for example, be one that comprises at least one moiety capable of binding or associating with one or more residues of SYKKD or an active site or binding pocket thereof.

Chemical moieties on the scaffold compound that permit attachment of one or more desired functional chemical groups may undergo conventional reactions by coupling, substitution, and electrophilic or nucleophilic displacement. For example, the moieties may be those already present on the compound or readily introduced. Alternatively, an variant of the scaffold compound comprising the moieties is utilized initially. As a non-limiting example, the moiety can be a leaving group which can readily be removed from the scaffold compound. Various moieties can be used, including but not limited to pyrophosphates, acetates, hydroxy groups, alkoxy groups, tosylates, brosylates, halogens, and the like. In another embodiment of the invention, the scaffold compound is synthesized from readily available starting materials using conventional techniques. (See e.g., U.S. Pat. No. 5,756,466 for general synthetic methods). Chemical groups are then introduced into the scaffold compound to increase the number of interactions with one or more residues of SYKKD or an active site or binding pocket thereof.

Because SYKKD may crystallize in more than one crystal form, the structure coordinates of SYKKD, or portions thereof, are particularly useful to solve the structure of those other crystal forms of SYKKD. They may also be used to solve the structure of SYKKD mutants, SYKKD co-complexes, or of the crystalline form of any other protein with significant amino acid sequence homology to any functional domain of SYKKD.

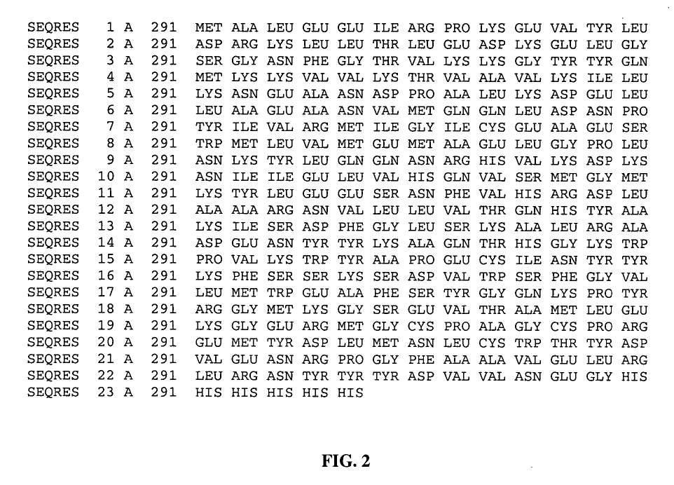

Homologs or mutants of SYKKD may, for example, have an amino acid sequence homology to the Homo sapiens amino acid sequence of FIG. 2 of greater than 60%, more preferred proteins have a greater than 70% sequence homology, more preferred proteins have a greater than 80% sequence homology, more preferred proteins have a greater than 90% sequence homology, and most preferred proteins have greater than 95% sequence homology. A protein domain, region, or binding pocket may have a level of amino acid sequence homology to the corresponding domain, region, or binding pocket amino acid sequence of Homo sapiens of FIG. 2 of greater than 60%, more preferred proteins have a greater than 70% sequence homology, more preferred proteins have a greater than 80% sequence homology, more preferred proteins have a greater than 90% sequence homology, and most preferred proteins have greater than 95% sequence homology. Percent homology may be determined using, for example, a PSI BLAST search, such as, but not limited to version 2.1.2 (Altschul, S.F., et al., Nuc. Acids Rec. 25:3389-3402, 1997).

One method that may be employed for this purpose is molecular replacement. In this method, the unknown crystal structure, whether it is another crystal form of SYKKD, a SYKKD mutant, or a SYKKD co-complex, or the crystal of some other protein with significant amino acid sequence homology to any functional domain of SYKKD, may be determined using phase information from the SYKKD structure coordinates. This method may provide an accurate three-dimensional structure for the unknown protein in the new crystal more quickly and efficiently than attempting to determine such information ab initio. In addition, in accordance with this invention, SYKKD mutants may be crystallized in co-complex with known SYKKD inhibitors. The crystal structures of a series of such complexes may then be solved by molecular replacement and compared with that of wild-type SYKKD. Potential sites for modification within the various binding pockets of the protein may thus be identified. This information provides an additional tool for determining the most efficient binding interactions, for example, increased hydrophobic interactions, between SYKKD and a chemical group or compound.

If an unknown crystal form has the same space group as and similar cell dimensions to the known SYKKD crystal form, then the phases derived from the known crystal form can be directly applied to the unknown crystal form, and in turn, an electron density map for the unknown crystal form can be calculated. Difference electron density maps can then be used to examine the differences between the unknown crystal form and the known crystal form. A difference electron density map is a subtraction of one electron density map, e.g., that derived from the known crystal form, from another electron density map, e.g., that derived from the unknown crystal form. Therefore, all similar features of the two electron density maps are eliminated in the subtraction and only the differences between the two structures remain. For example, if the unknown crystal form is of a SYKKD co-complex, then a difference electron density map between this map and the map derived from the native, uncomplexed crystal will ideally show only the electron density of the ligand. Similarly, if amino acid side chains have different conformations in the two crystal forms, then those differences will be highlighted by peaks (positive electron density) and valleys (negative electron density) in the difference electron density map, making the differences between the two crystal forms easy to detect. However, if the space groups and/or cell dimensions of the two crystal forms are different, then this approach will not work and molecular replacement must be used in order to derive phases for the unknown crystal form.

All of the complexes referred to above may be studied using well-known X-ray diffraction techniques and may be refined against data extending from about 500 Å to at least 3.0 Å or 1.5 Å, until the refinement has converged to limits accepted by those skilled in the art, such as, but not limited to, R=0.2, Rfree=0.25. This may be determined using computer software, such as X-PLOR, CNX, or refmac (part of the CCP4 suite; Collaborative Computational Project, Number 4, “The CCP4 Suite: Programs for Protein Crystallography,” Acta Cryst. D50, 760-63, 1994). See, e.g., Blundell et al., Protein Crystallography, Academic Press; Methods in Enzymology, Vols. 114 & 115, 1976; Wyckoff et al., eds., Academic Press, 1985; Methods in Enzymology, Vols. 276 and 277 (Carter & Sweet, eds., Academic Press 1997); “Application of Maximum Likelihood Refinement” G. Murshudov, A.Vagin and E.Dodson, (1996) in the Refinement of Protein Structures, Proceedings of Daresbury Study Weekend; G. N. Murshudov, A. A. Vagin and E. J. Dodson, Acta Cryst. D53, 240-55, 1997; G. N. Murshudov, A. Lebedev, A. A. Vagin, K. S. Wilson and E. J. Dodson, Acta Cryst. Section D55, 247-55, 1999. See, e.g., Blundell et al., Protein Crystallography, Academic Press; Methods in Enzymology, Vols. 114 & 115, 1976; Wyckoff et al., eds., Academic Press, Methods in Enzymology, Vols. 276 and 277, 1985 (Carter & Sweet, eds., Academic Press 1997). This information may thus be used to optimize known classes of SYK inhibitors, and more importantly, to design and synthesize novel classes of SYK inhibitors.

The structure coordinates of SYKKD mutants will also facilitate the identification of related proteins or enzymes analogous to SYK in function, structure or both, thereby further leading to novel therapeutic modes for treating or preventing diseases or disorders in which SYK activity is implicated.

Subsets of the molecular structure coordinates can be used in any of the above methods. Particularly useful subsets of the coordinates include, but are not limited to, coordinates of single domains, coordinates of residues lining an active site or binding pocket, coordinates of residues that participate in important protein-protein contacts at an interface, and alpha-carbon coordinates. For example, the coordinates of one domain of a protein that contains the active site may be used to design inhibitors that bind to that site, even though the protein is fully described by a larger set of atomic coordinates. Therefore, a set of atomic coordinates that define the entire polypeptide chain, although useful for many applications, do not necessarily need to be used for the methods described herein.

### EXAMPLES

### Example 1

### Determination of SYKKD Structure

The subsections below describe the production of a polypeptide comprising the Homo sapiens SYK, and the preparation and characterization of diffraction quality crystals and heavy-atom derivative crystals.

### Example 1.1

### Preparation of SYKKD Crystals

Human liver cDNA was synthesized using a standard cDNA synthesis kit following the manufacturers' instructions. The template for the cDNA synthesis was mRNA isolated from Hep G2 cells [ATCC HB-8065] using a standard RNA isolation kit. An open-reading frame for SYKKD was amplified from the human liver cDNA by the polymerase chain reaction (PCR) using the following primers:

Forward primer:
GAGGAGATCAGGCCCAAG
Reverse primer:
CGTTCACCACGTCATAGTAG

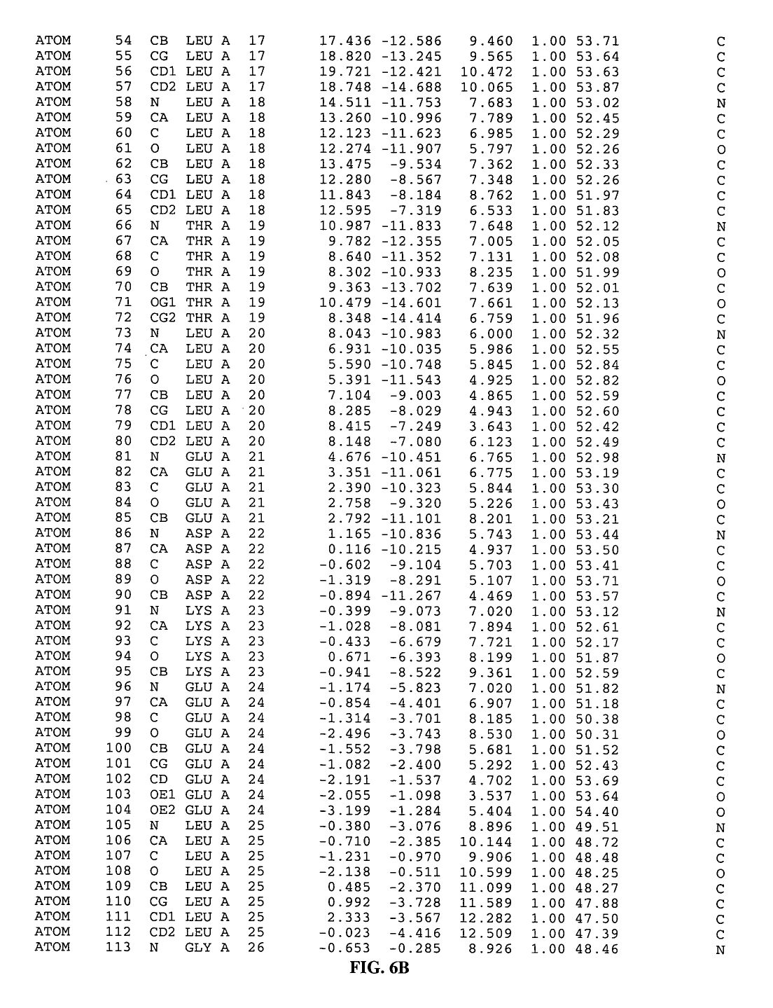

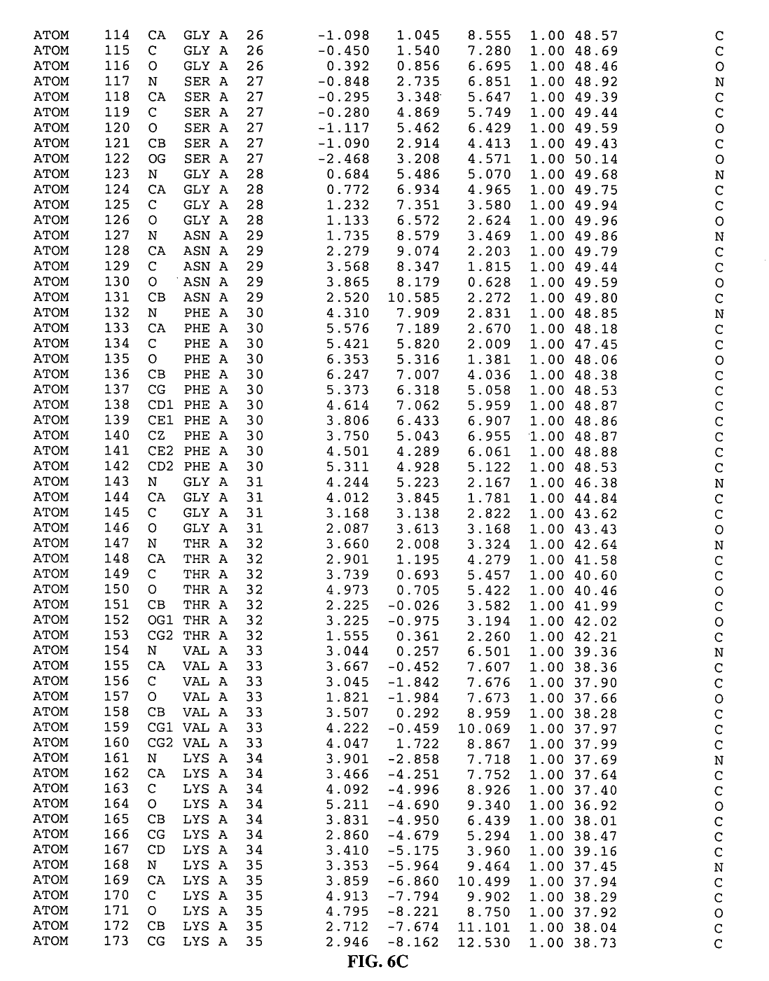

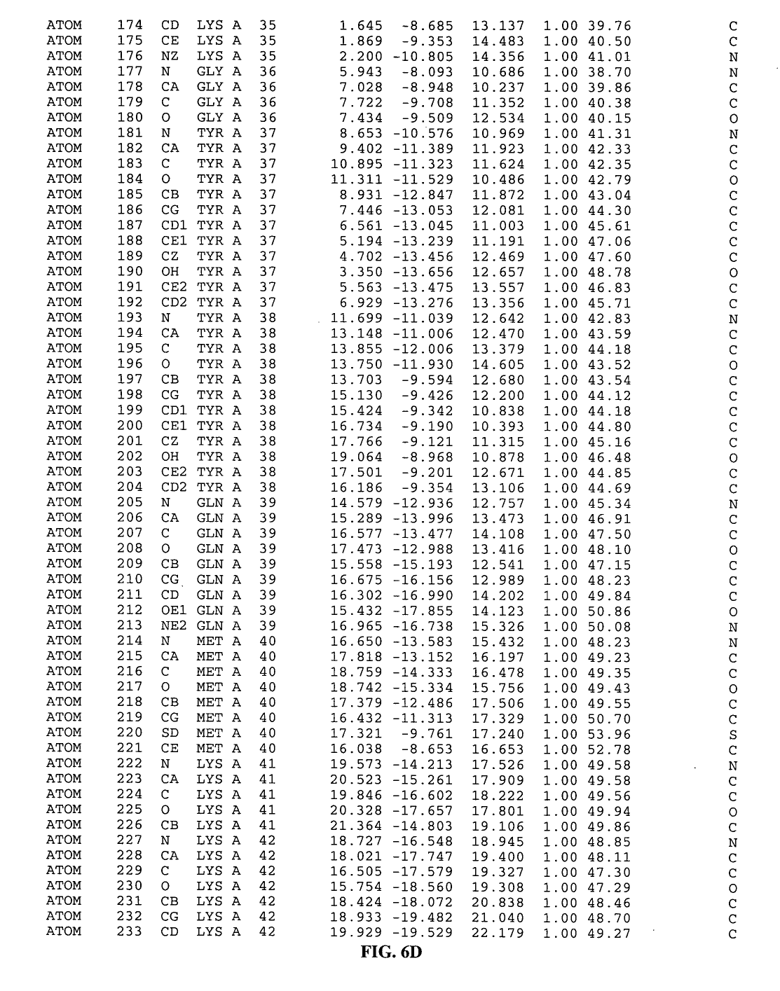

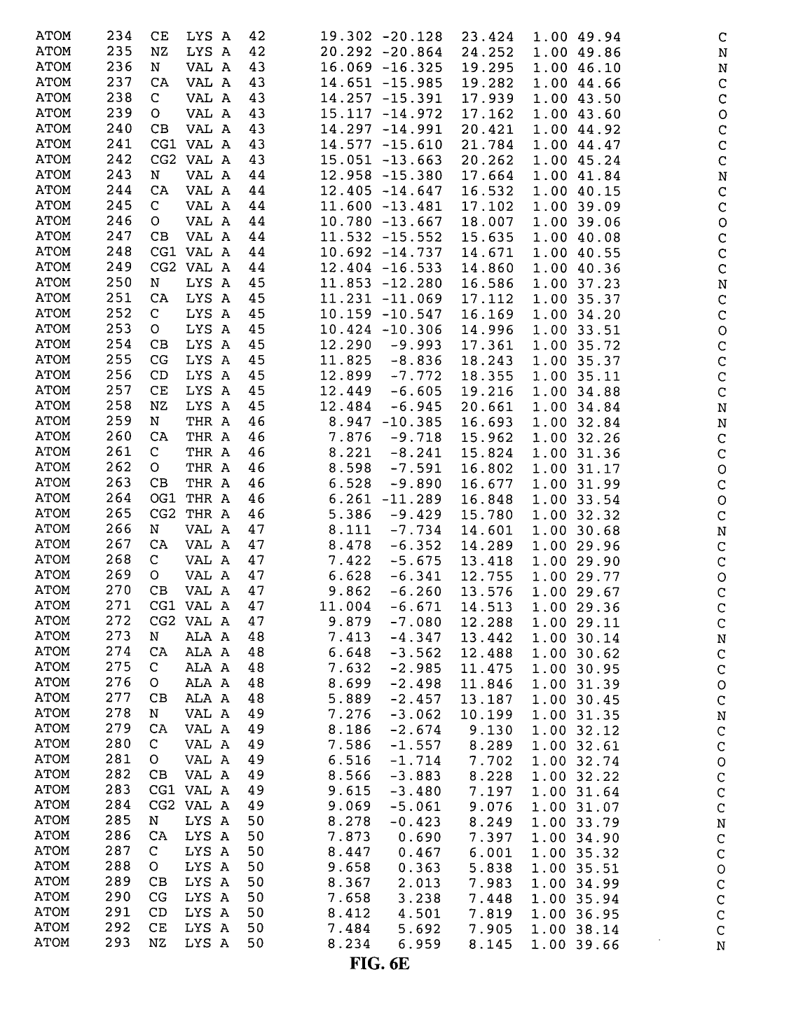

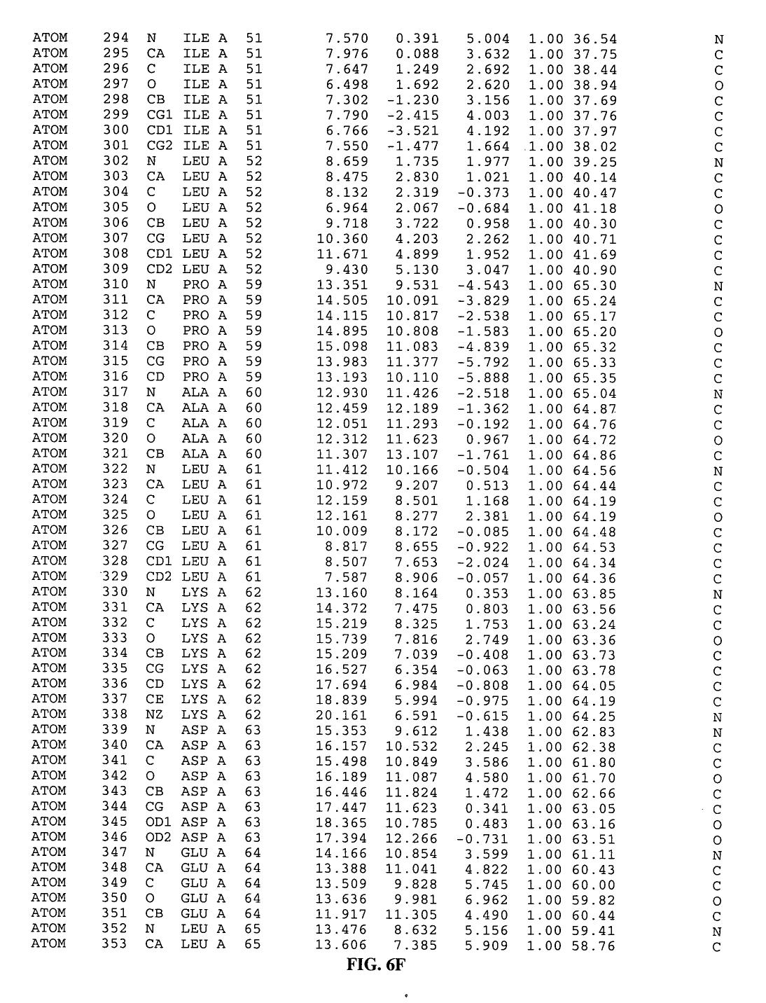

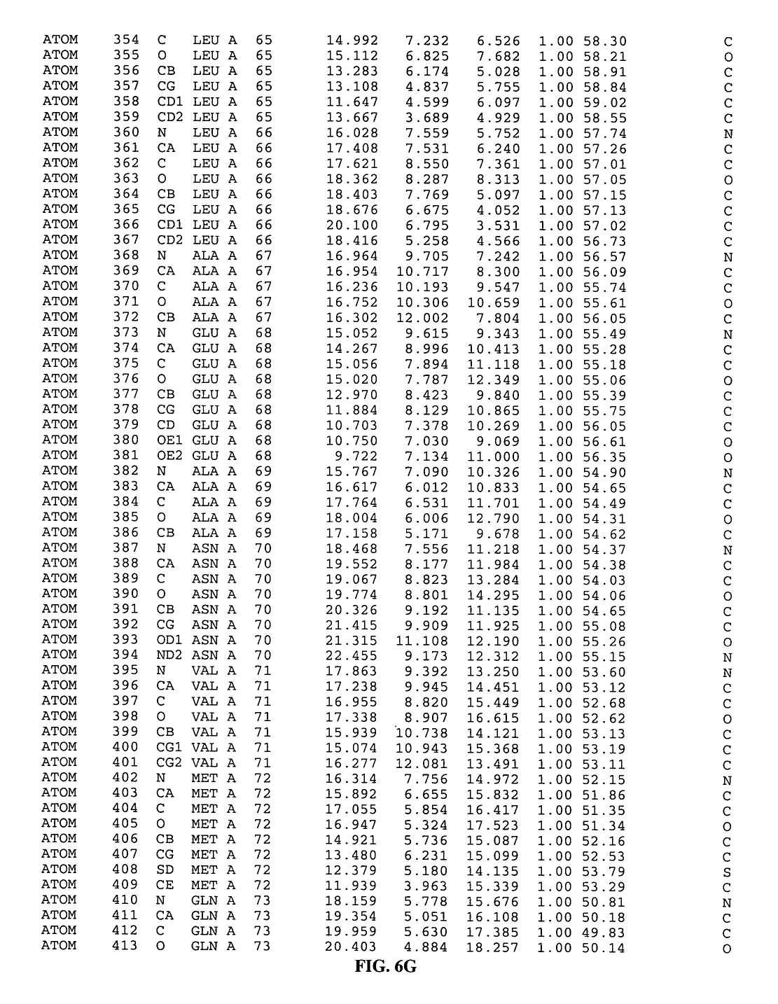

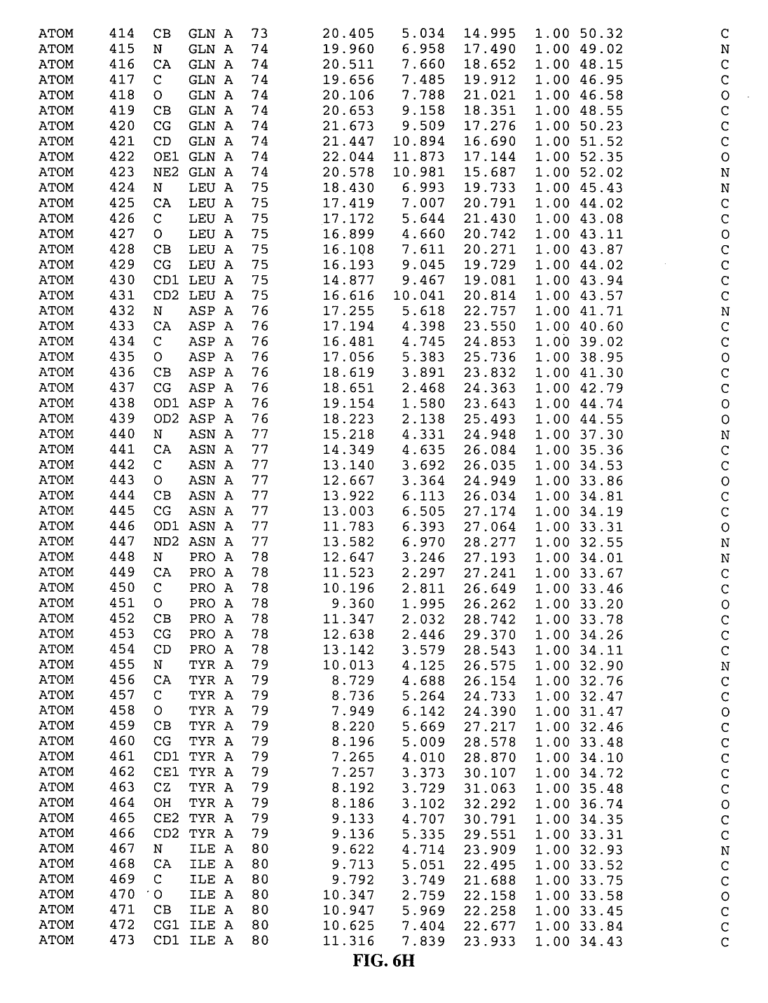

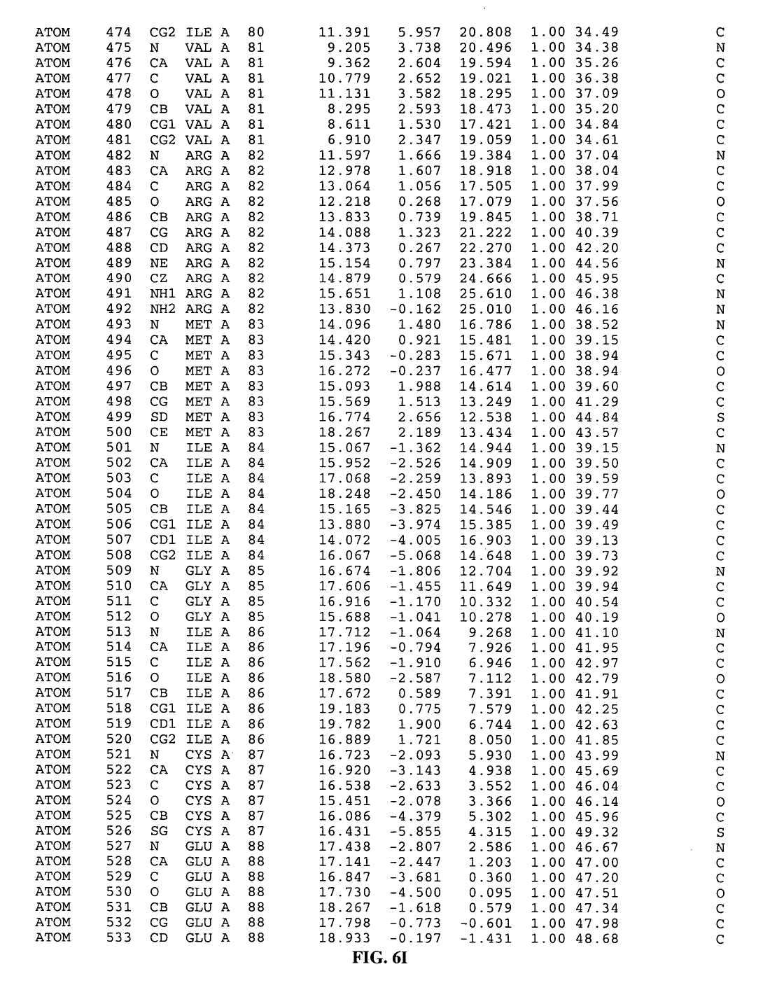

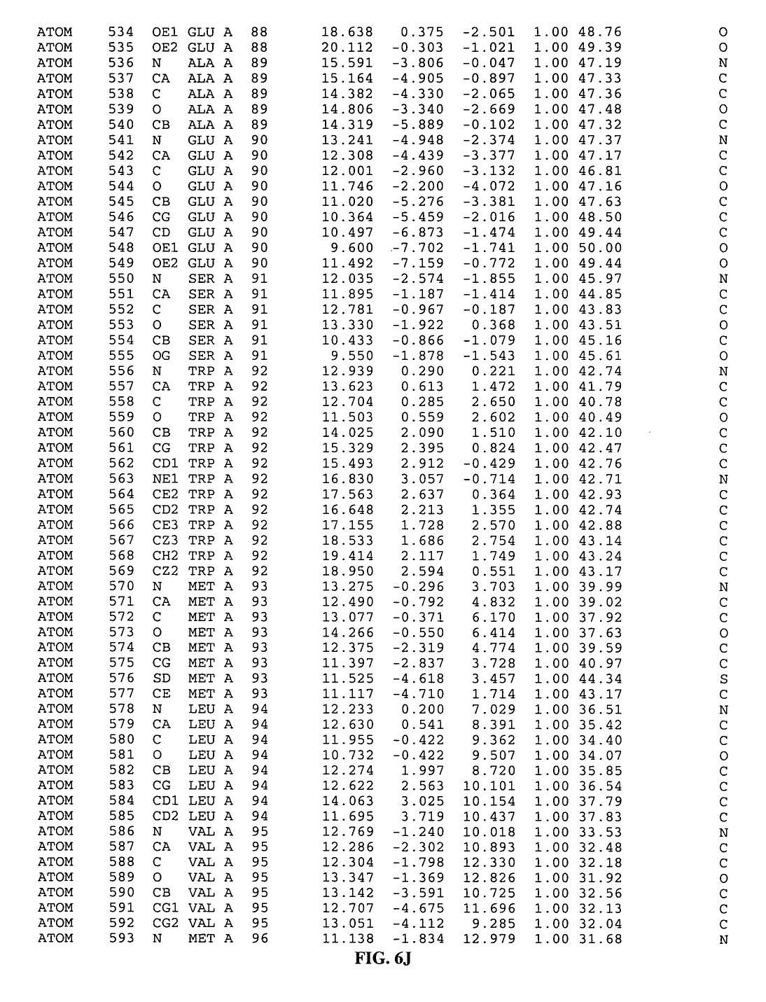

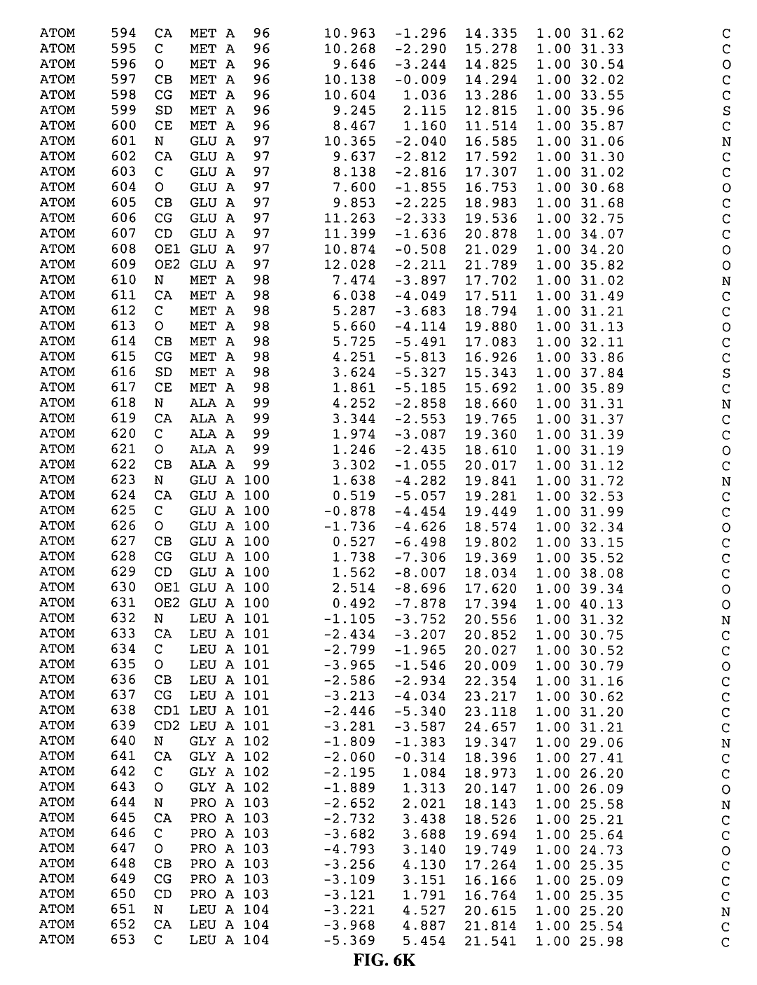

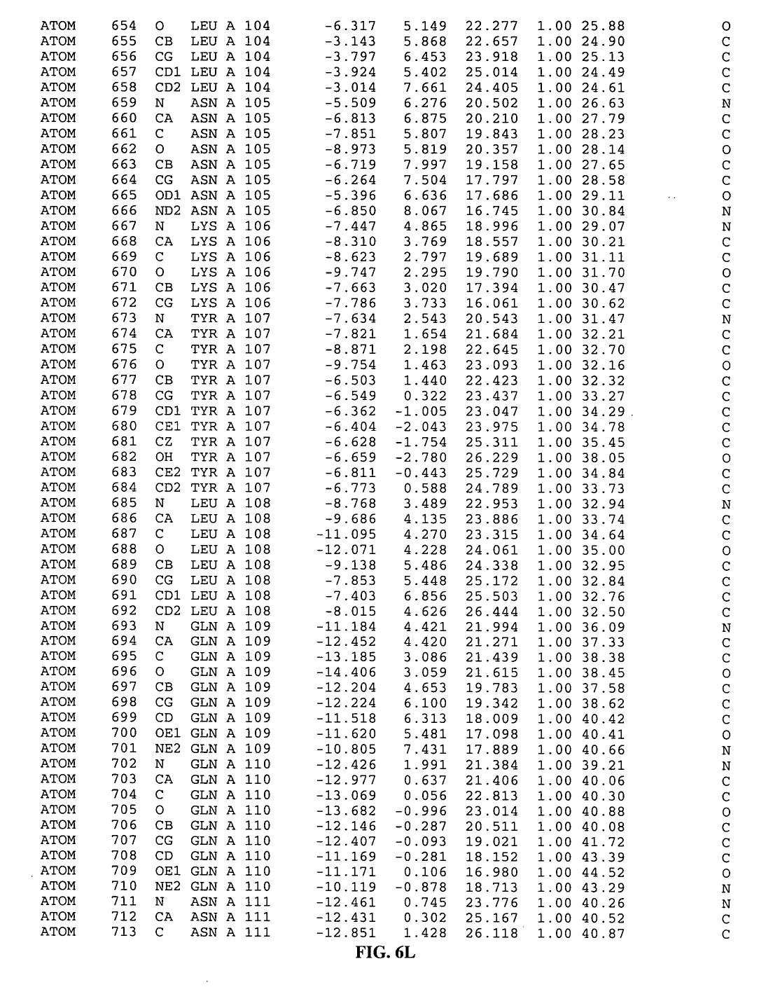

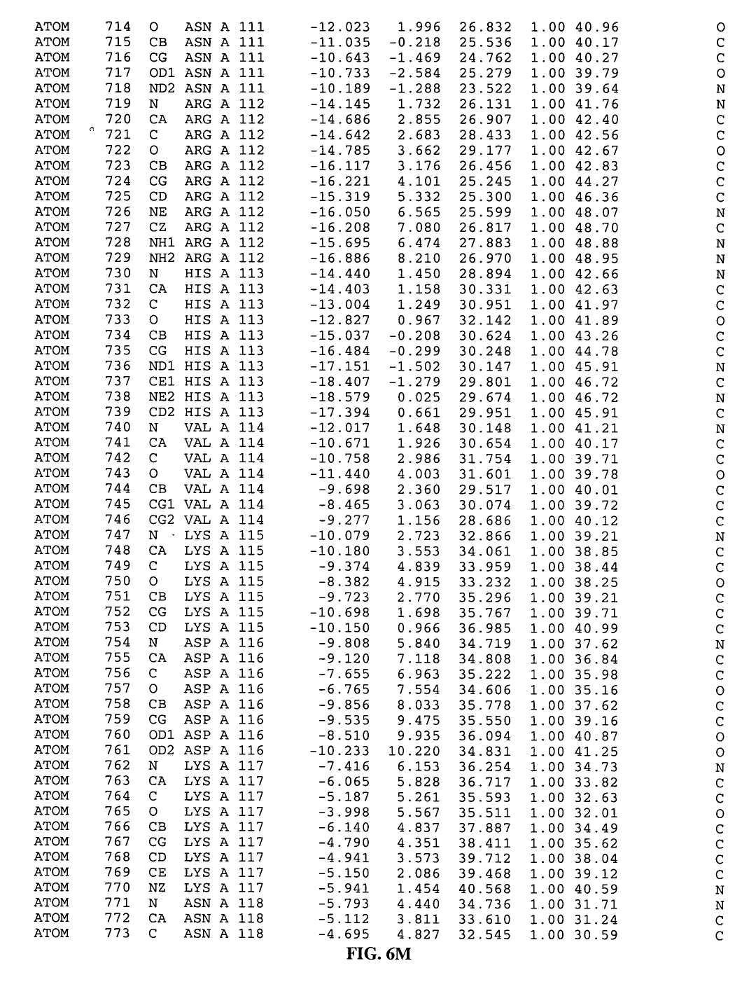

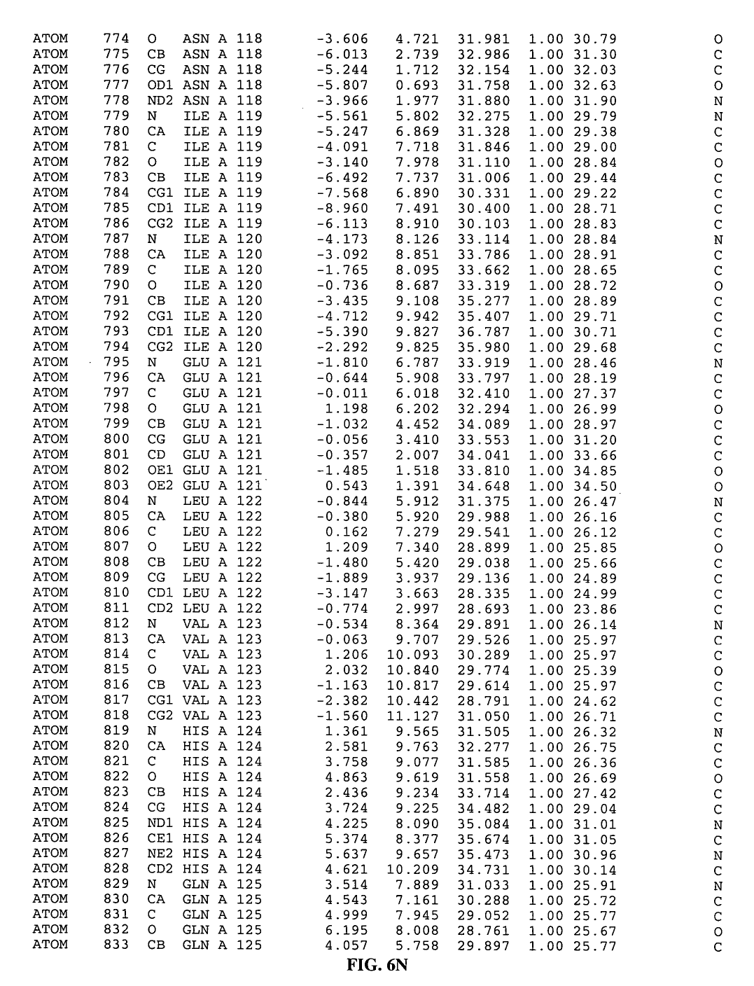

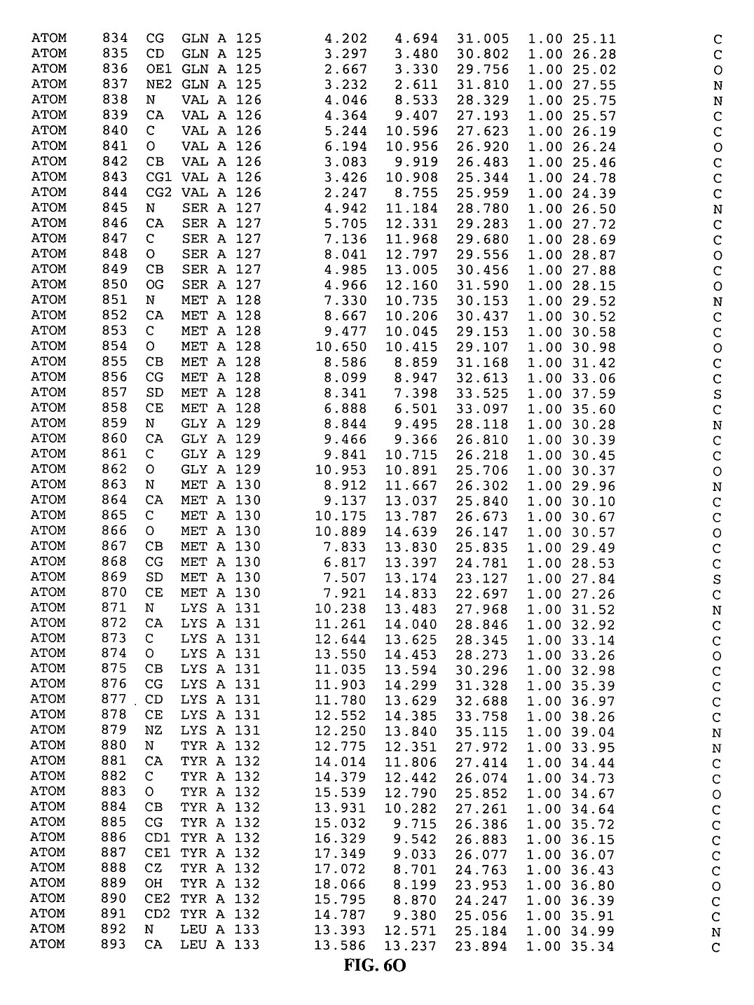

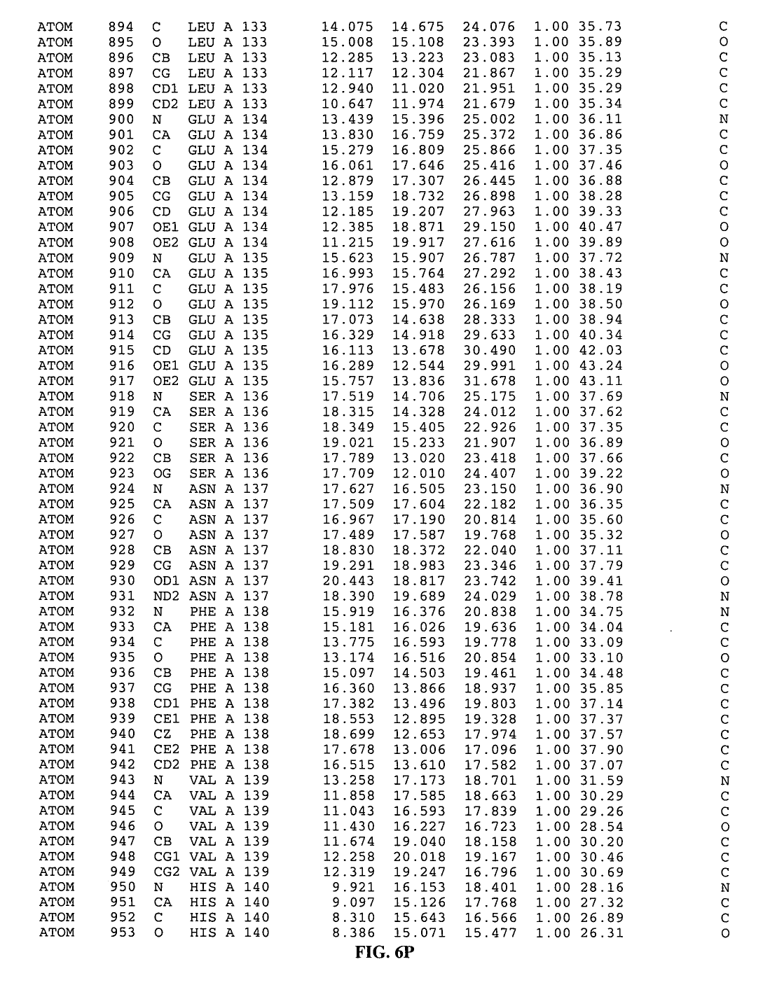

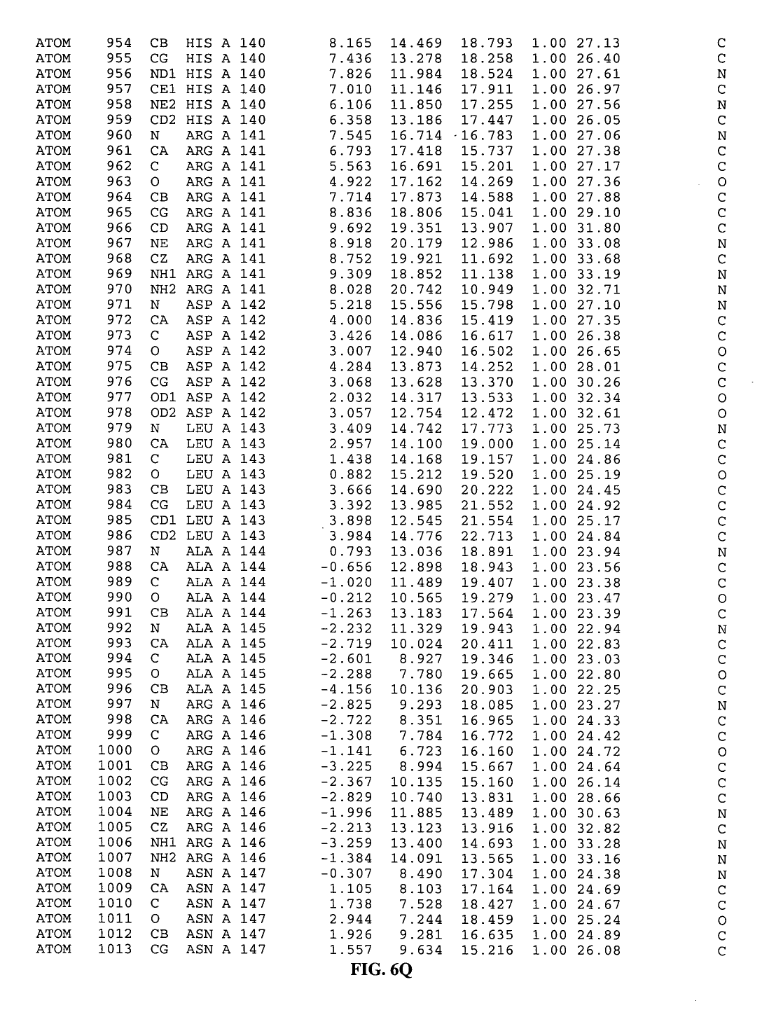

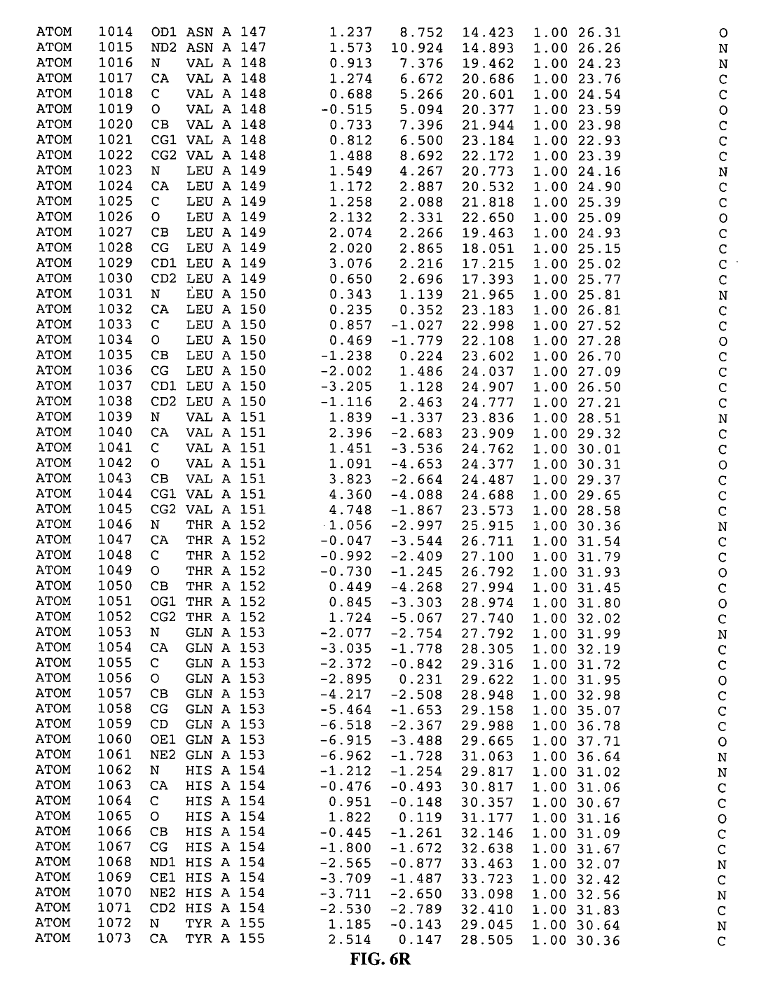

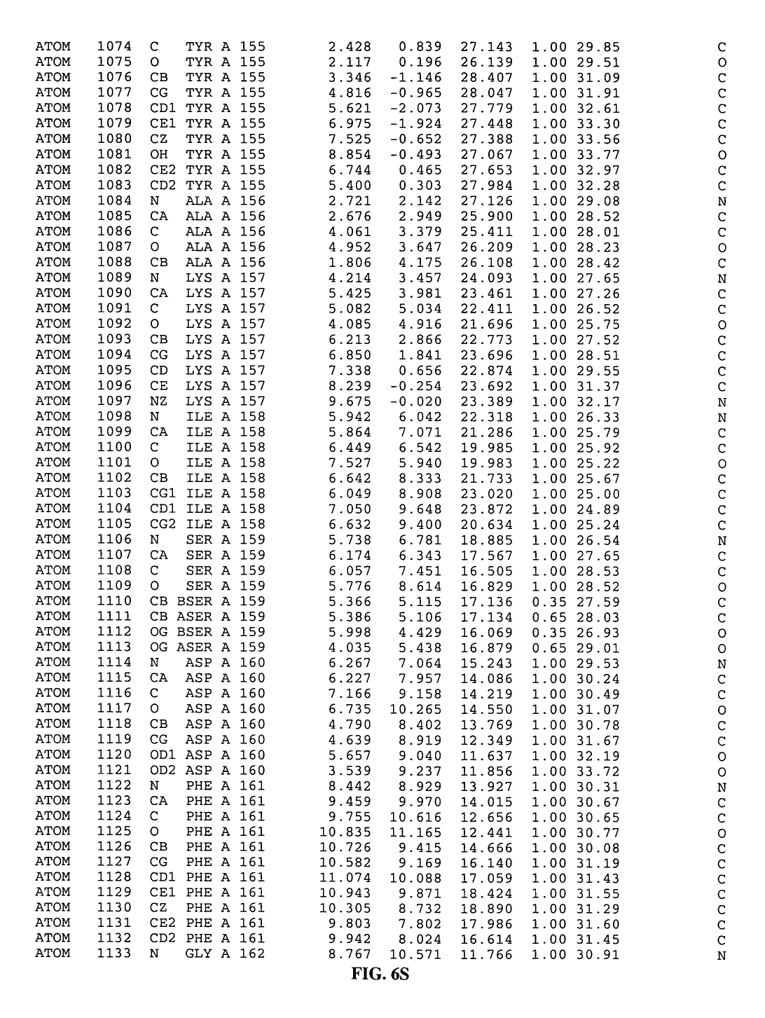

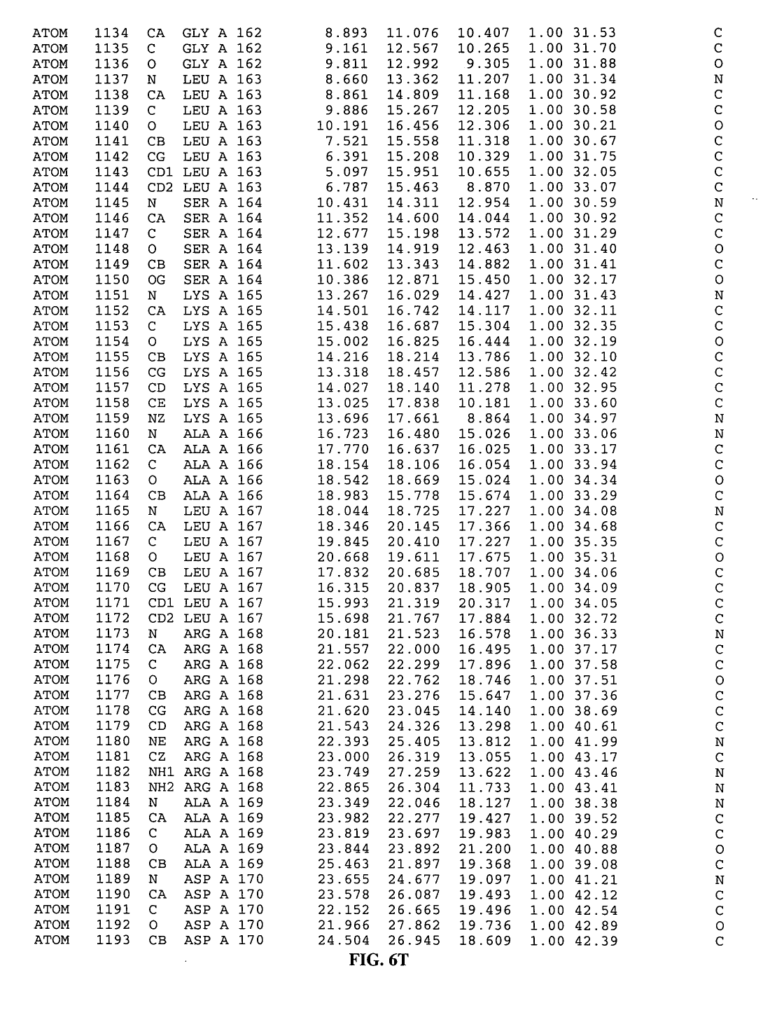

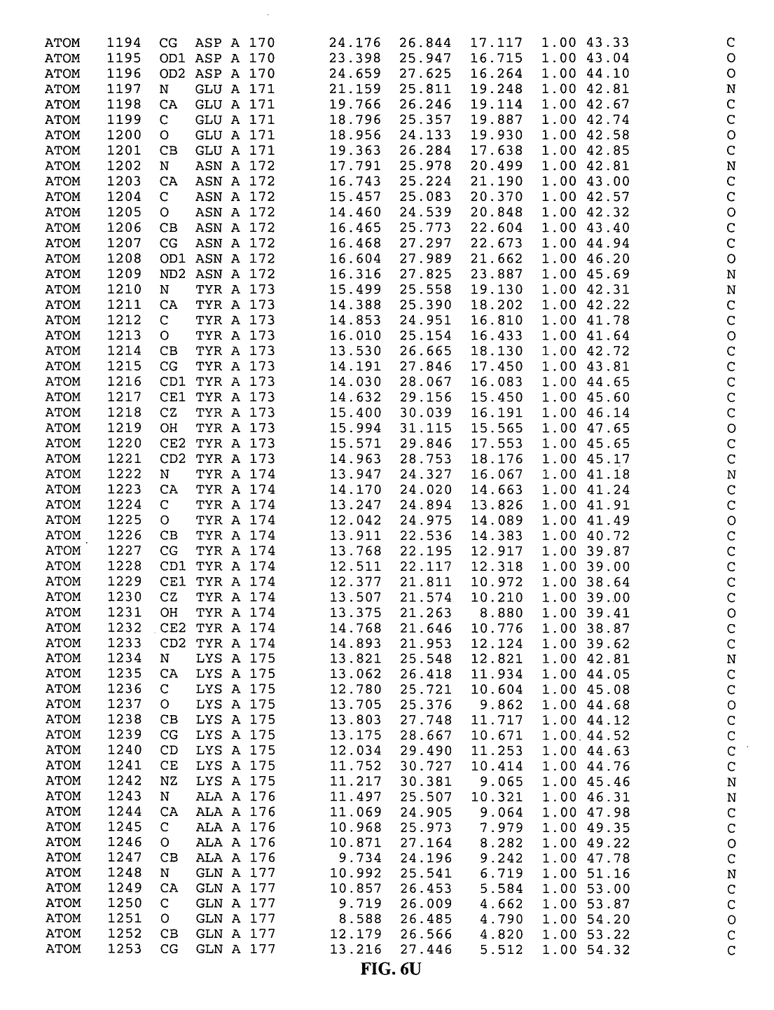

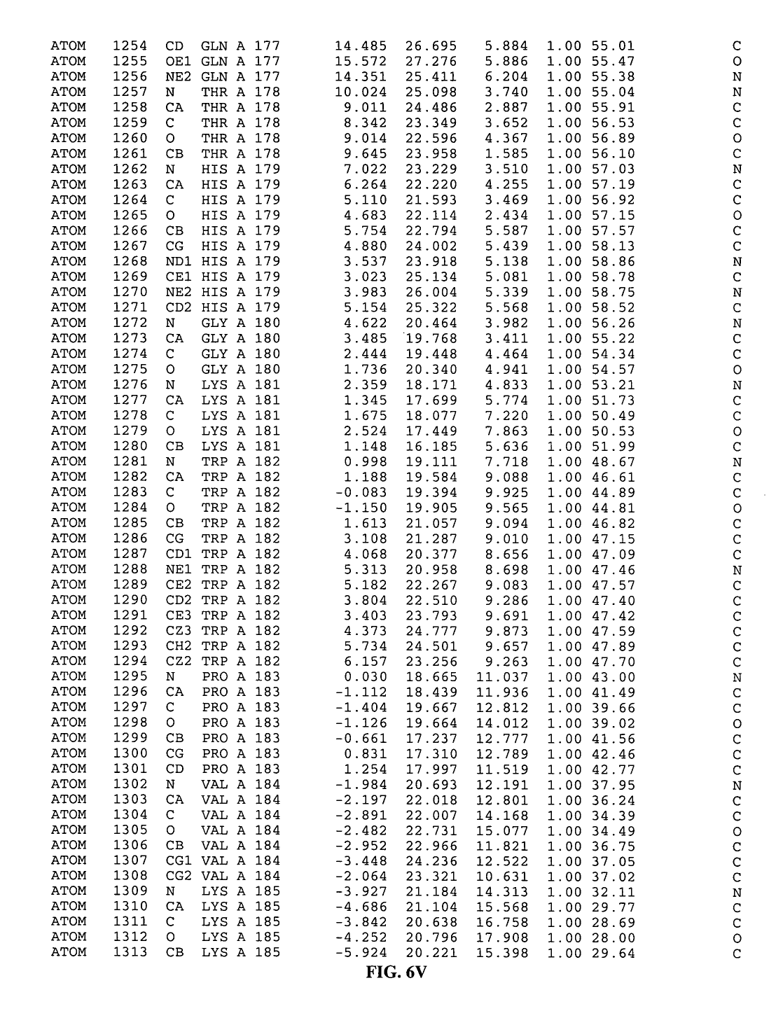

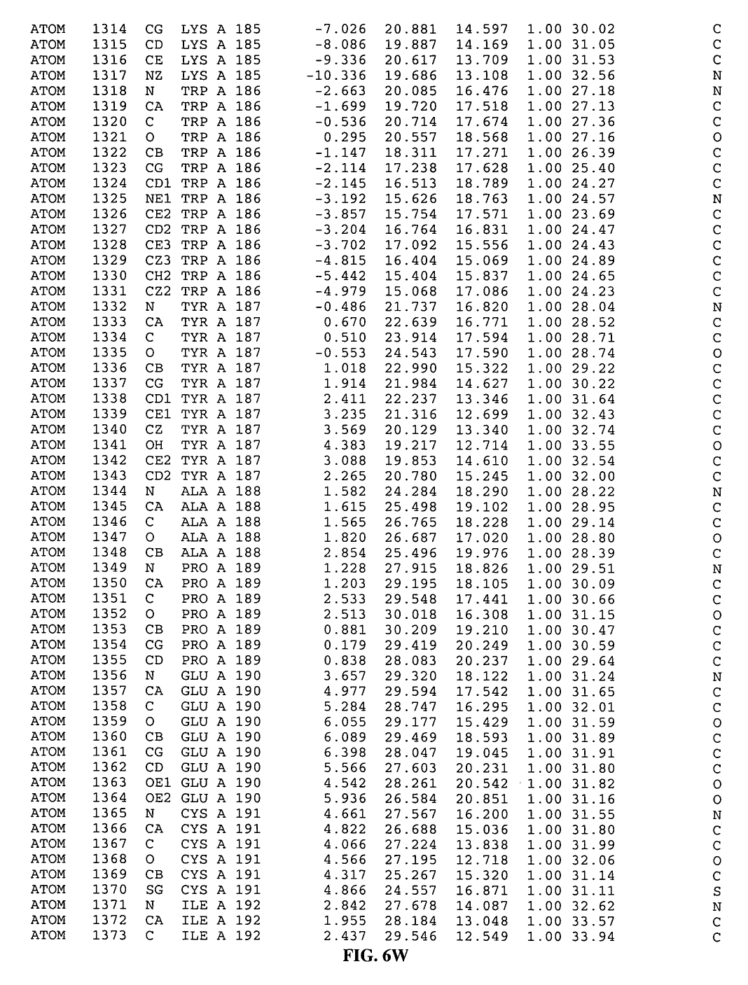

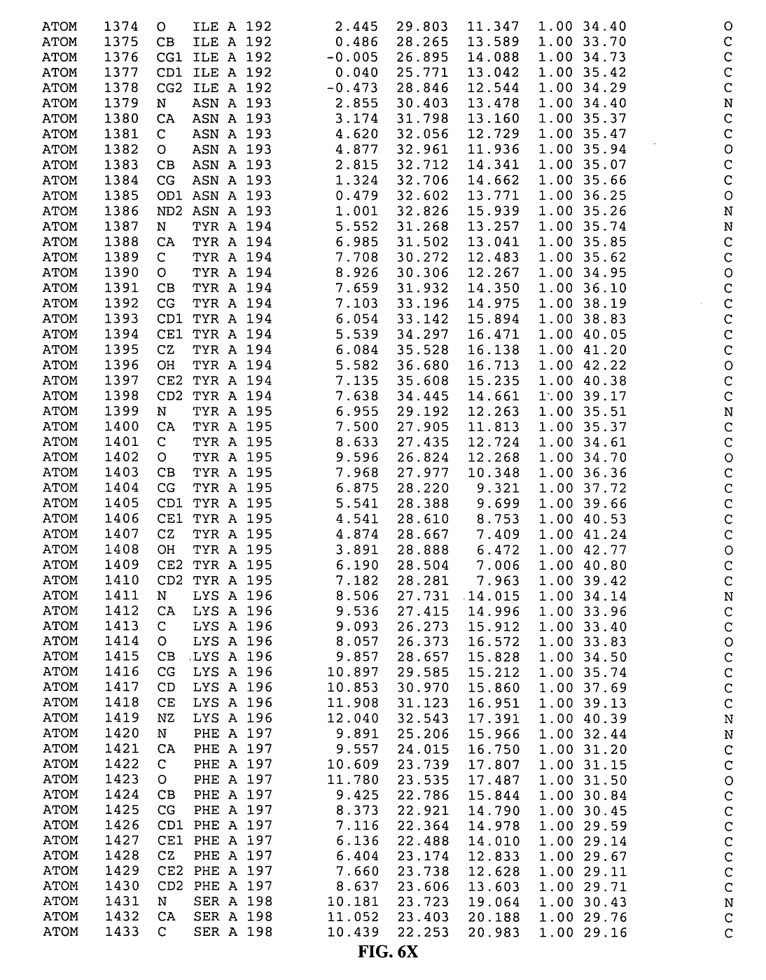

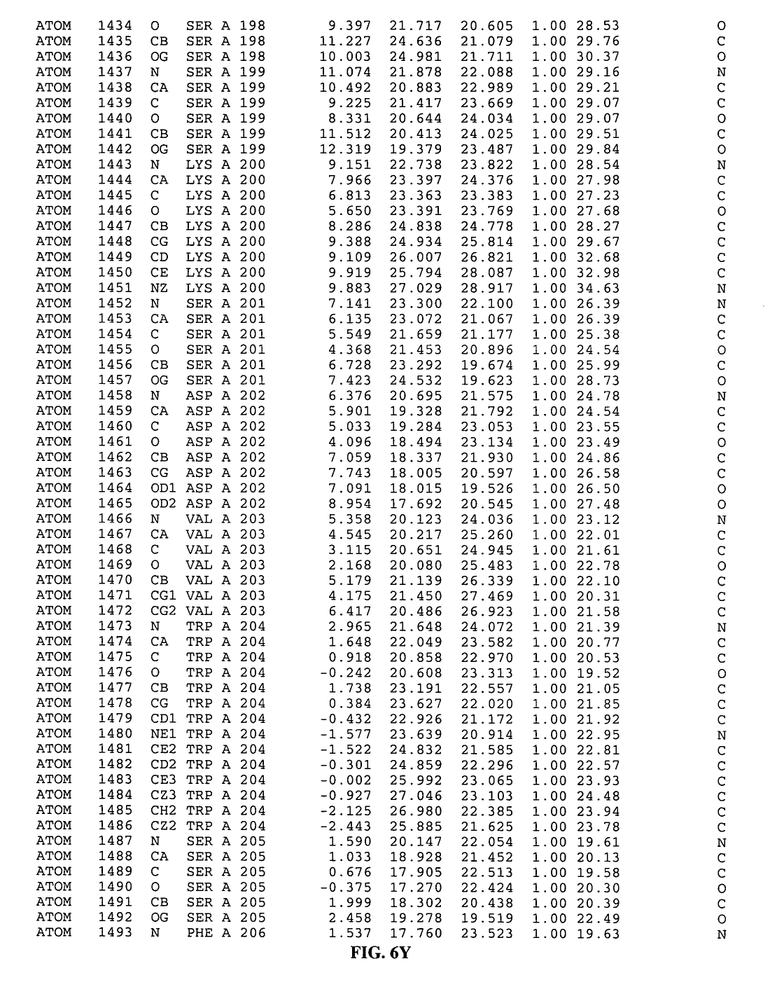

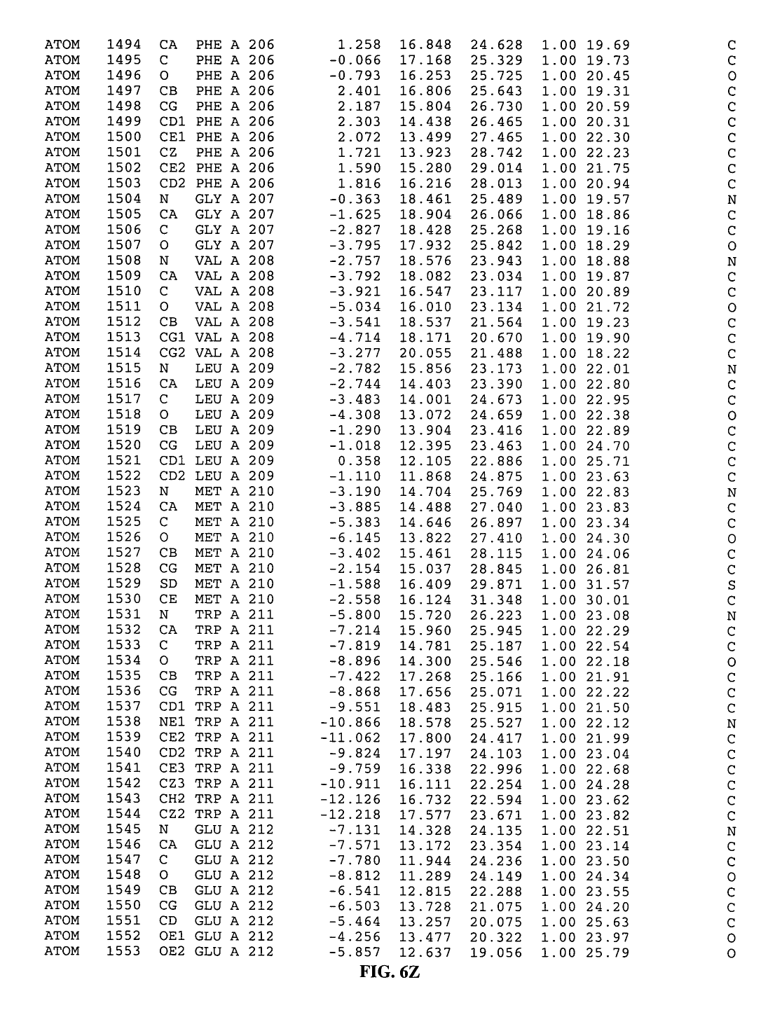

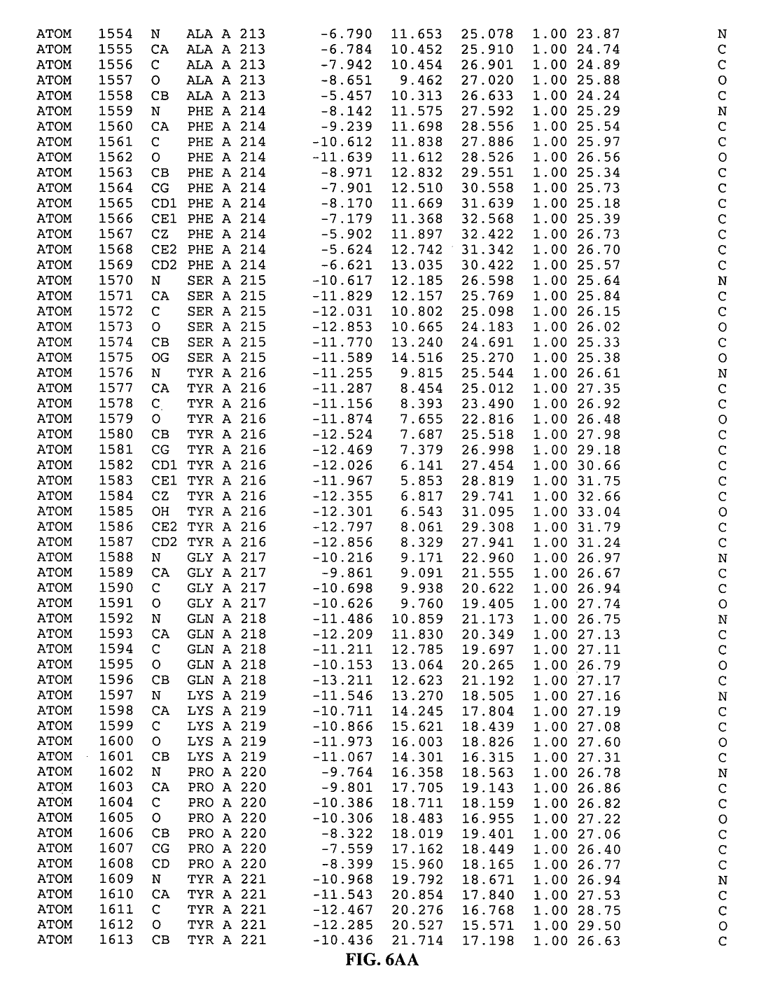

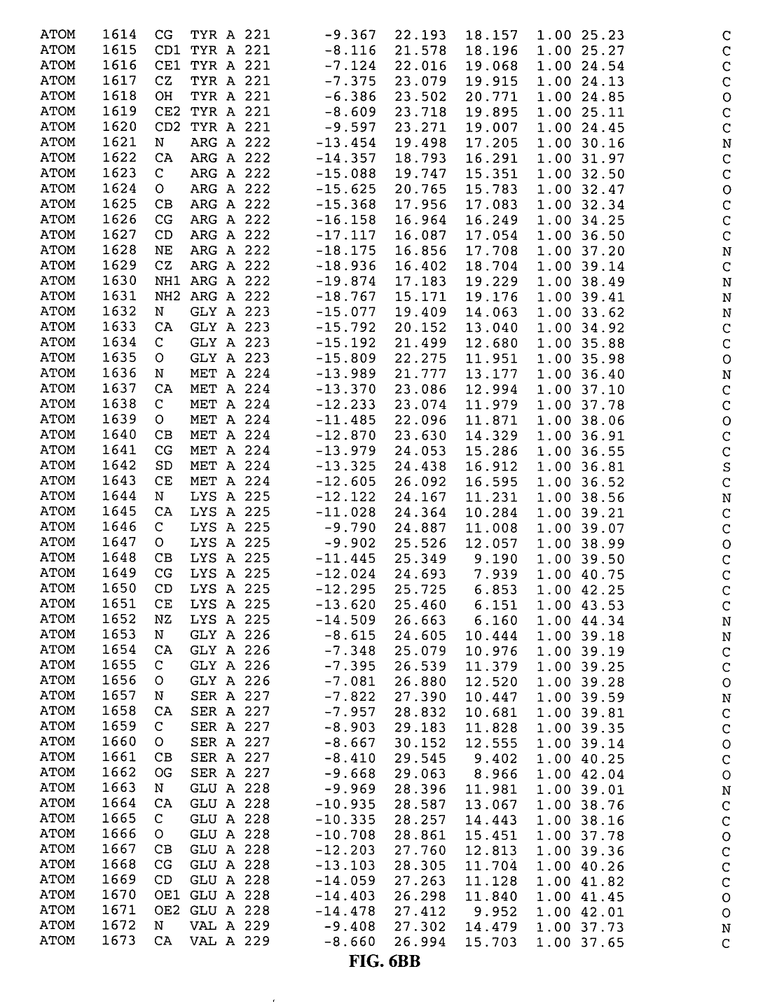

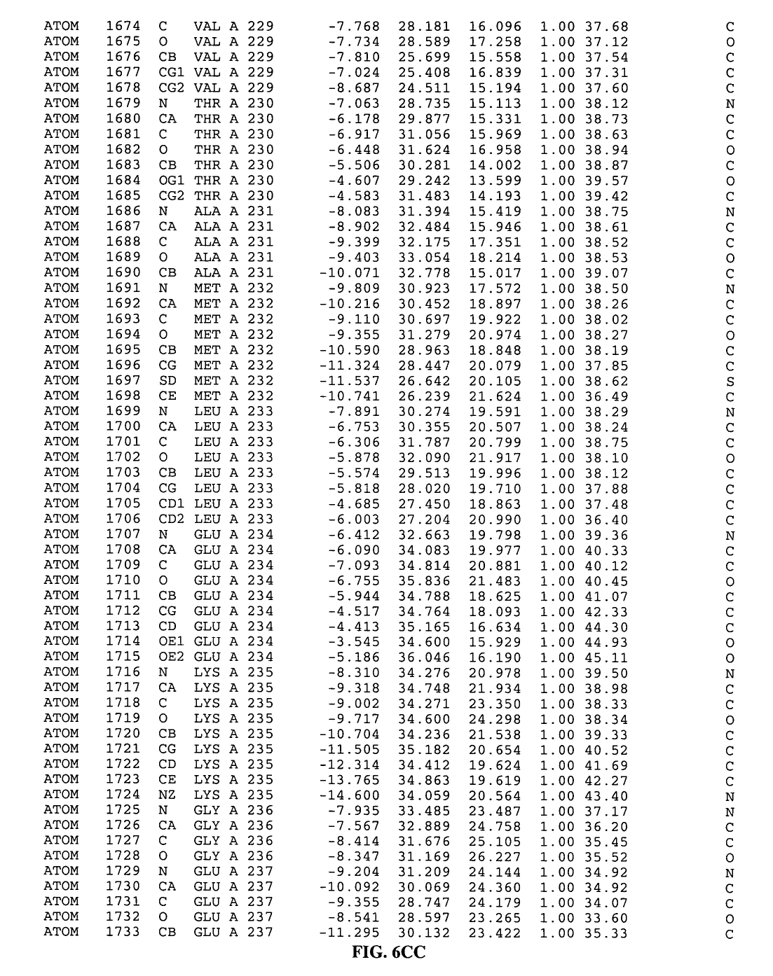

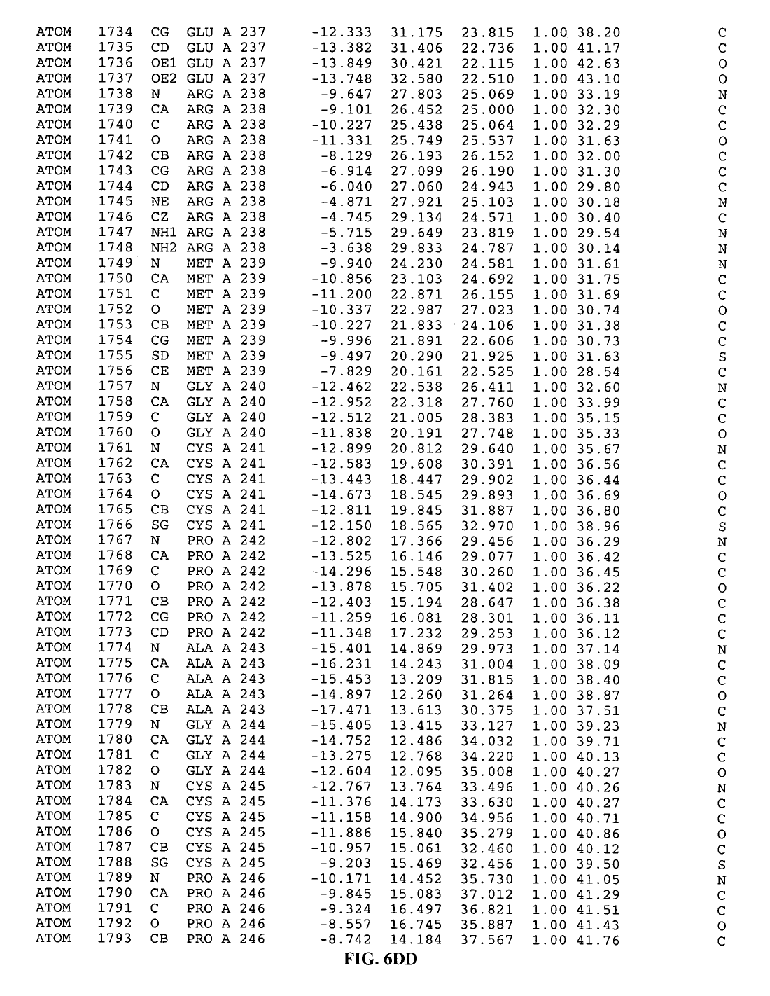

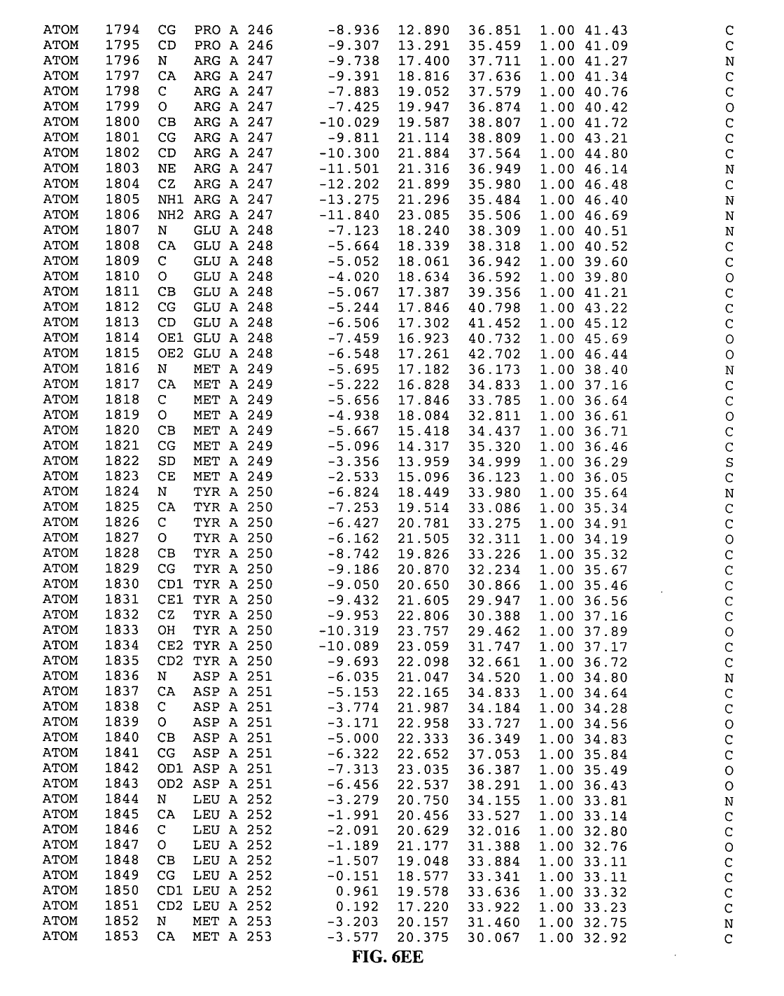

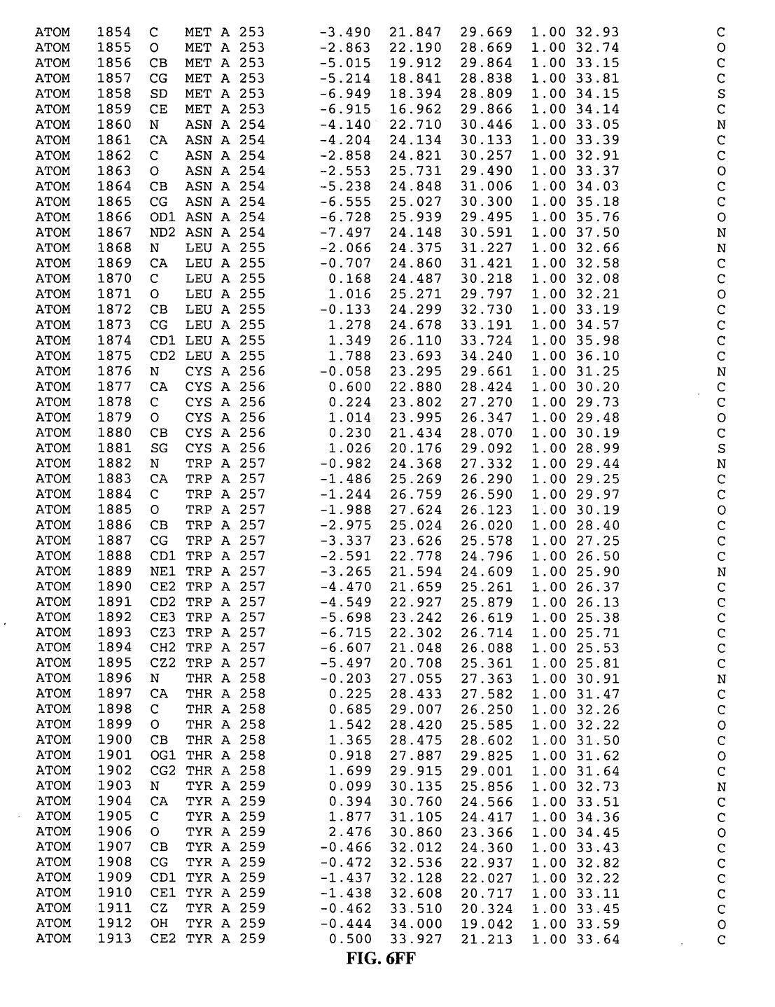

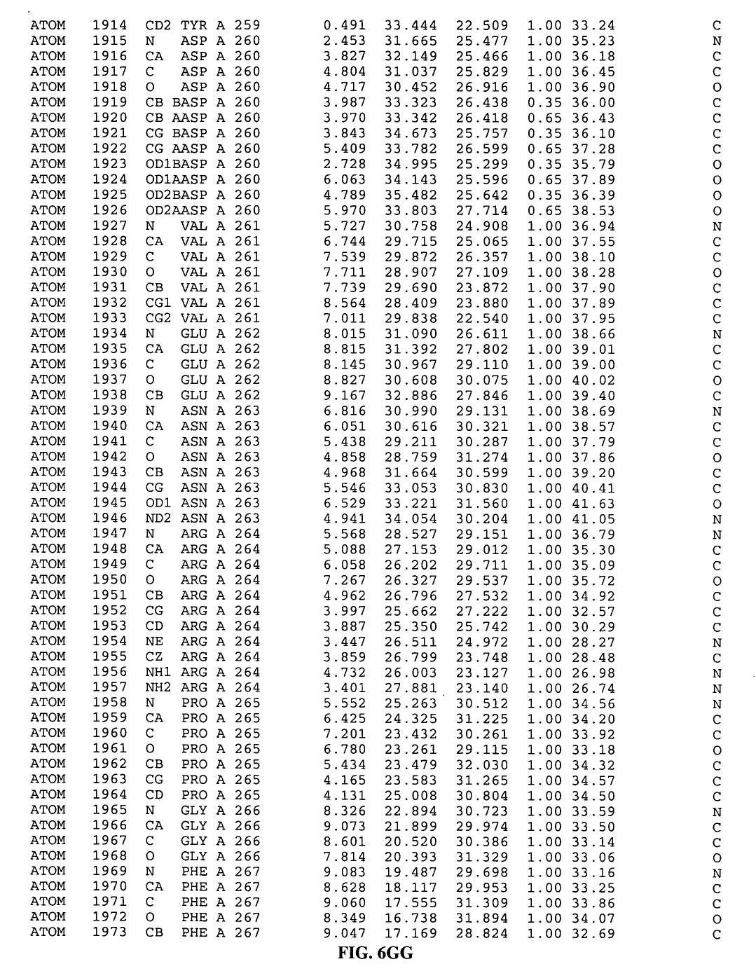

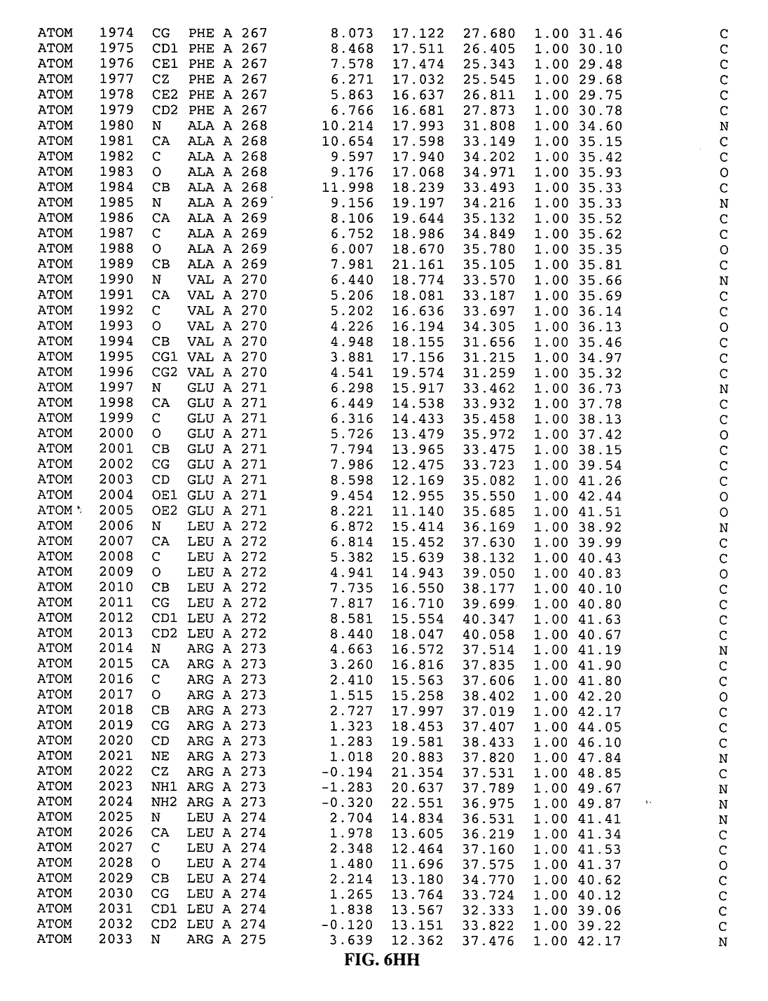

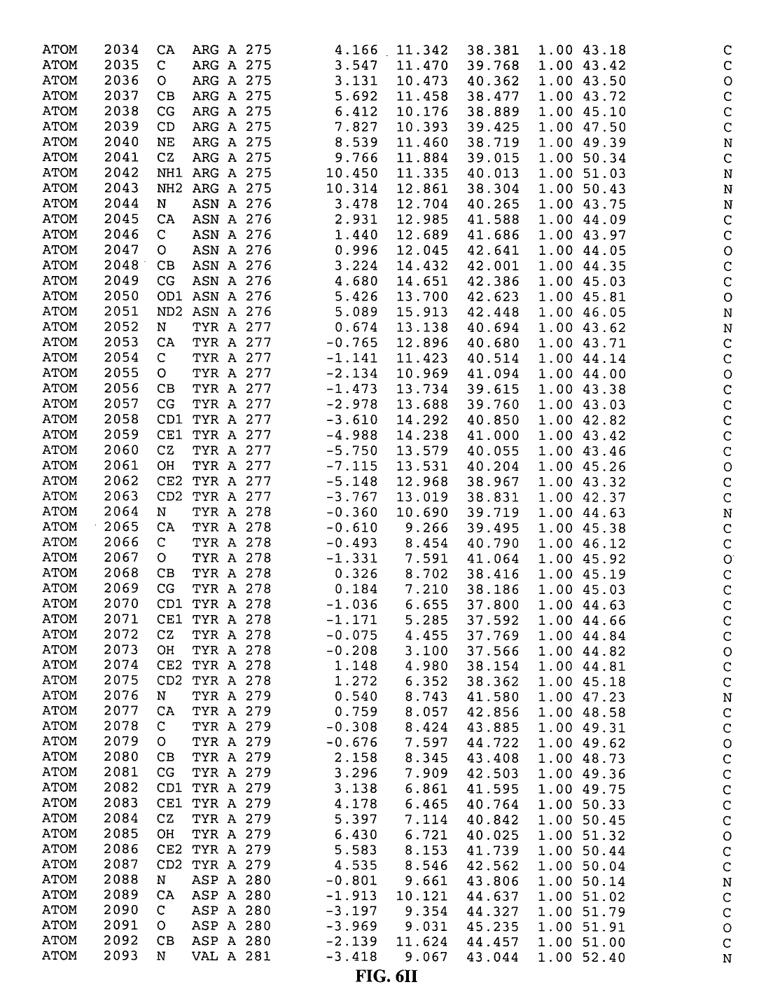

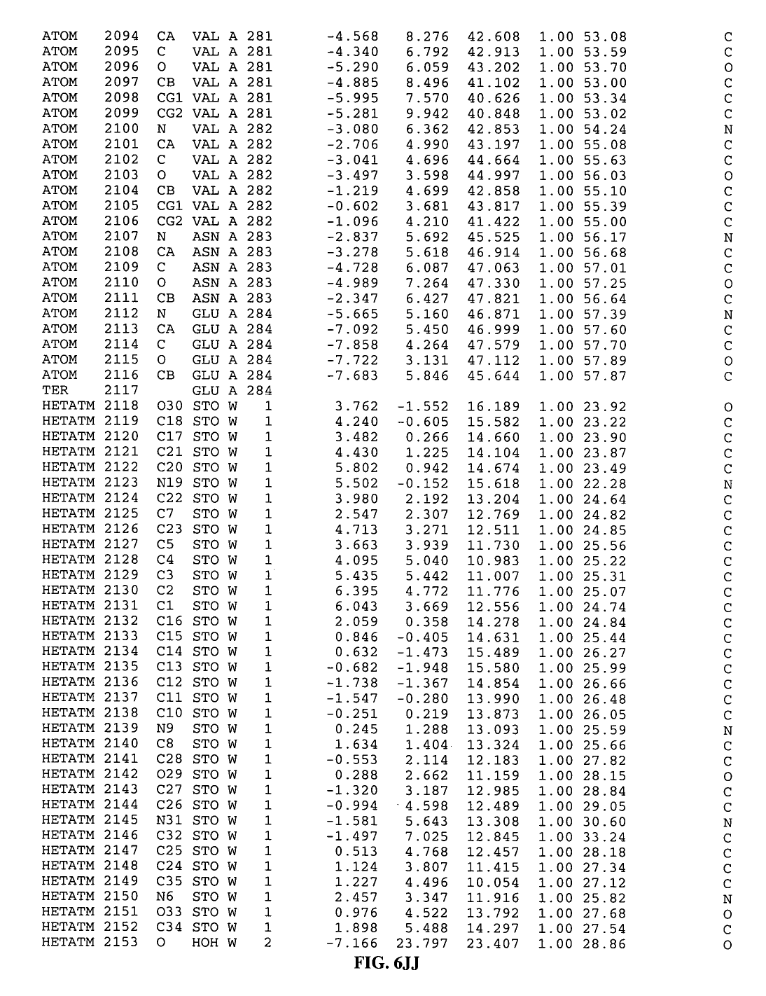

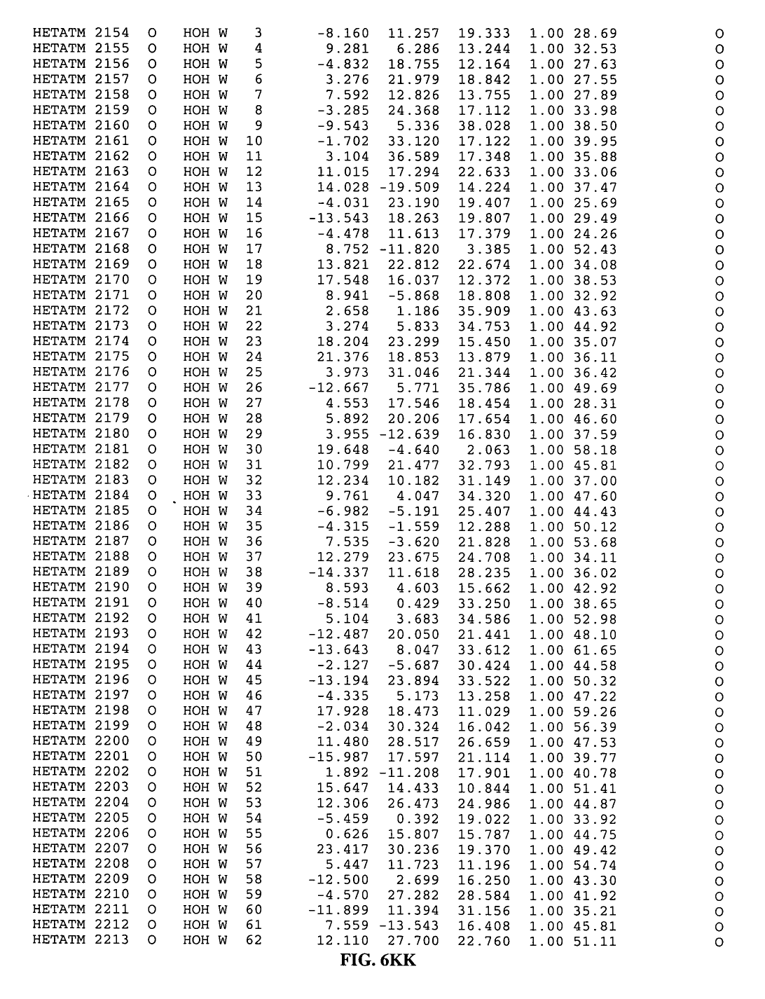

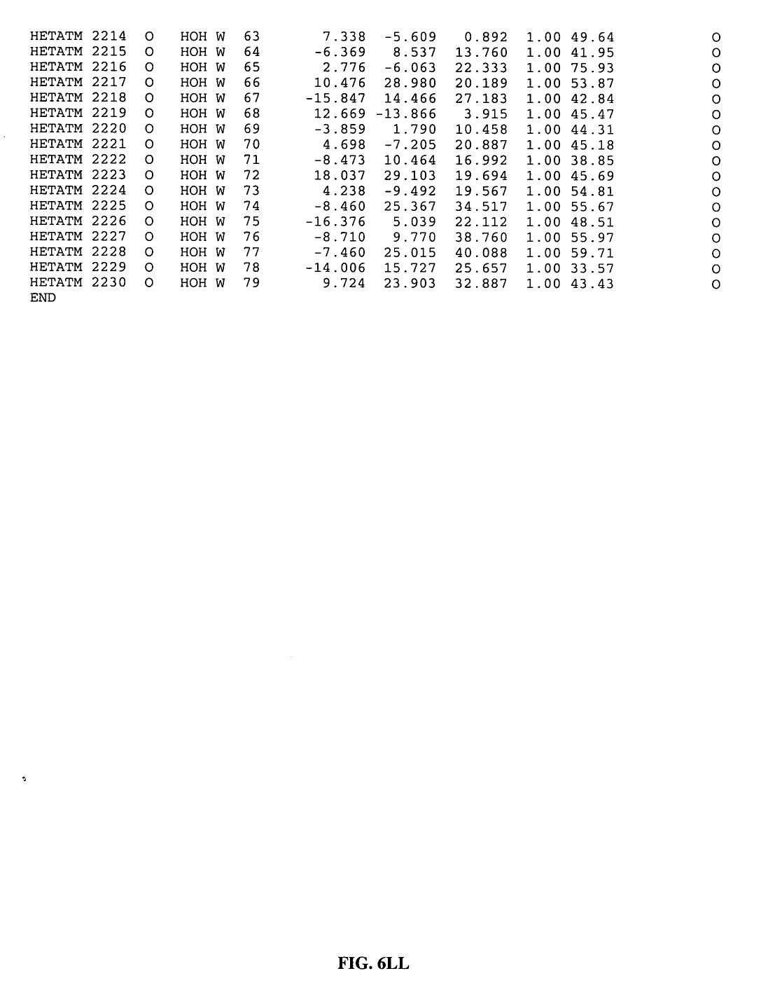

The PCR product (840 base pairs expected) was electrophoresed on a 1.2% E-gel ( Cat. #G5018-01, Invitrogen Corporation) and the appropriate size band was excised from the gel and eluted using a standard gel extraction kit. The eluted DNA was TOPO ligated into a GATEWAY™ (Invitrogen Corporation) adapted pcDNA6 AttB HisC vector which was custom TOPO adapted by Invitrogen Corporation. The resulting sequence of the gene after being TOPO ligated into the vector, from the start sequence through the stop site was as follows: ATG GCC CTT 3′[SYK]KD5′AA GGG CAT CAT CAC CAT CAC CAC TGA The SYKKD expressed using this vector has an N-terminal methionine, the kinase domain of SYKKD, and a C terminal 6 X His-tag. FIG. 6.

Plasmids containing TOPO ligated inserts were transformed into chemically competent TOP 10 cells (Invitrogen Corporation, Cat.#C4040-10). Colonies were then screened for inserts in the correct orientation and small DNA amounts were purified using a “miniprep” procedure from 2ml cultures, using a standard kit, following the manufacturer's instructions. For standard molecular biology protocols followed here, see also, for example, the techniques described in Sambrook et al., Molecular Cloning: A Laboratory Manual, Cold Spring Harbor Laboratory, NY, 2001, and Ausubel et al., Current Protocols in Molecular Biology, Greene Publishing Associates and Wiley Interscience, NY, 1989. The DNA that was in the “correct” orientation was then sequence verified.

A standard GATEWAY™ BP recombination was performed into pDONR201 (Invitrogen Corporation, Cat.#11798014. Gateway technology Cat.#1 1821014) and the recombination reaction was transformed into chemically competent TOP 10 cells (Invitrogen Corporation, Cat.#C4040-10), and plated on selective media. One colony was picked into a miniprep and DNA was obtained (the “entry vector”).

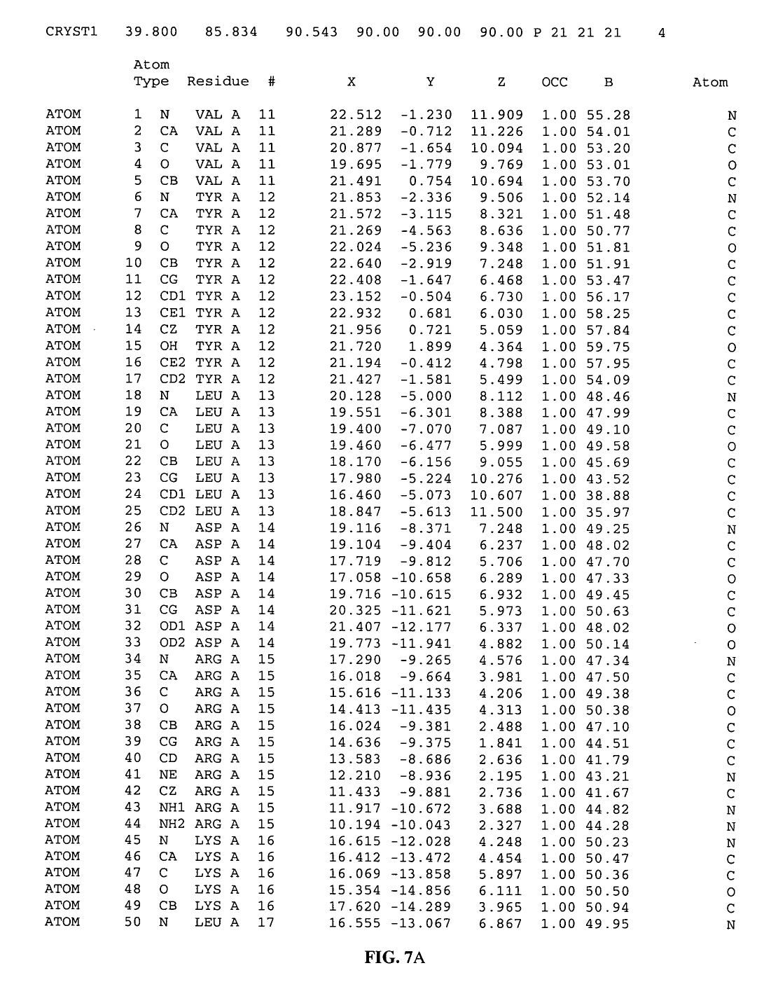

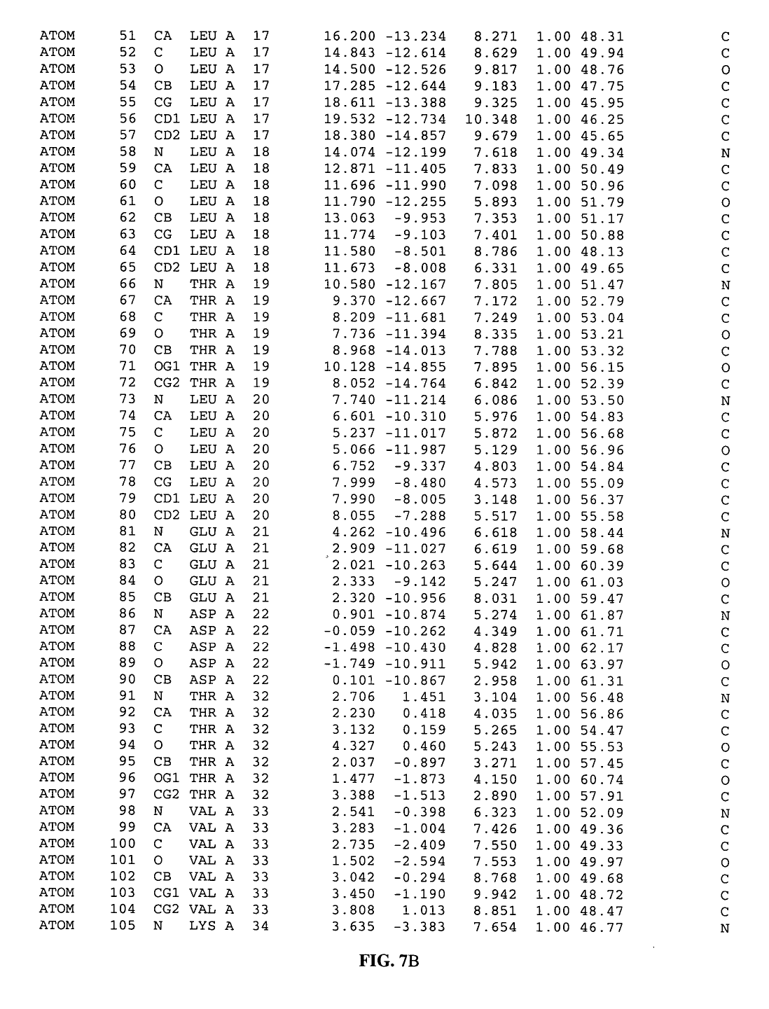

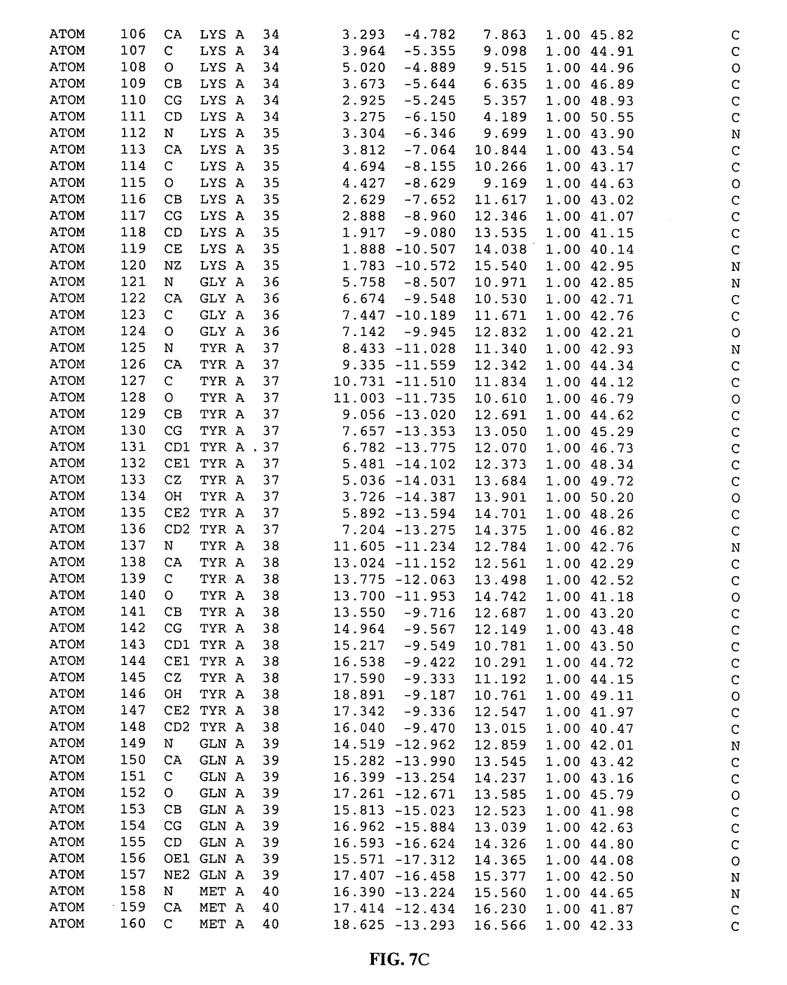

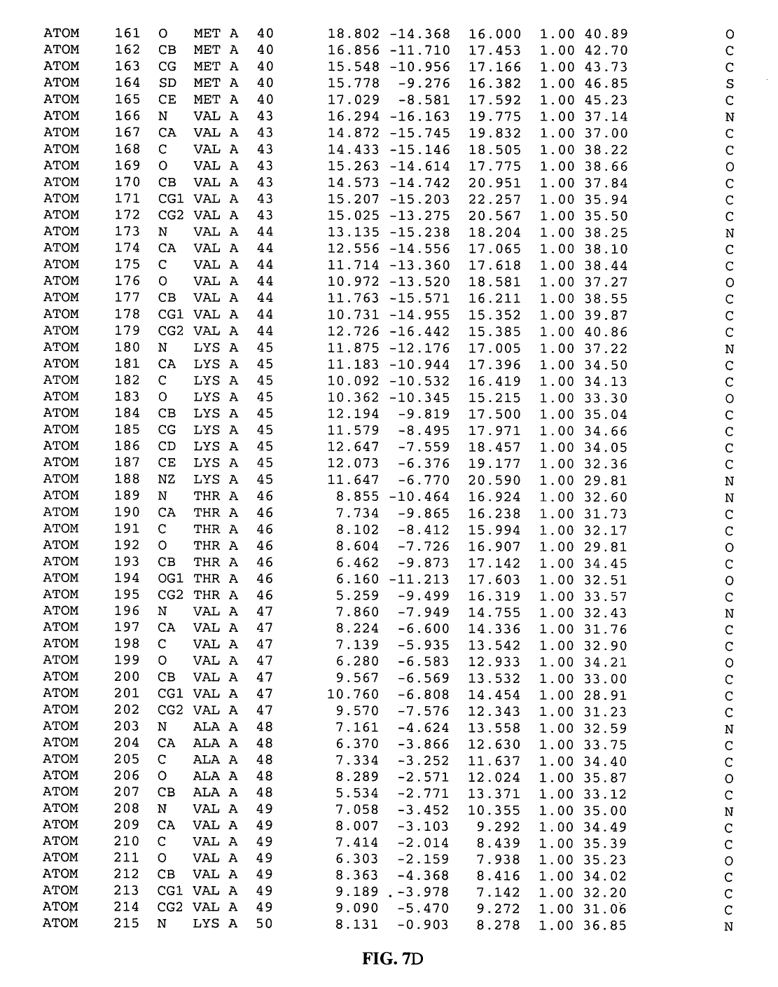

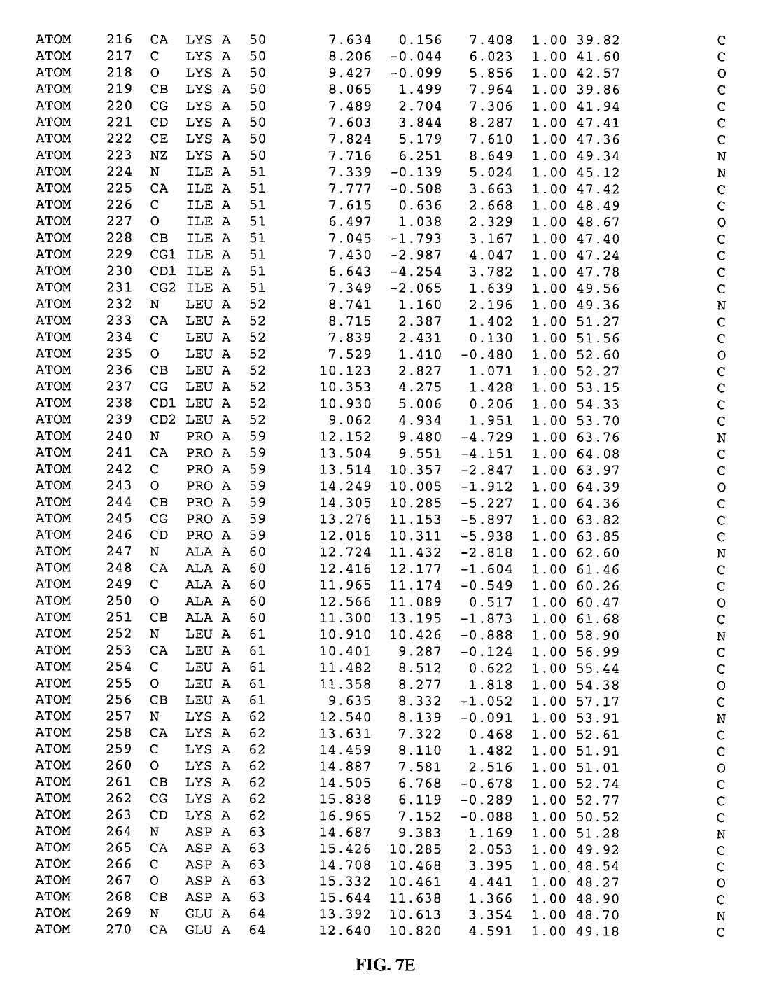

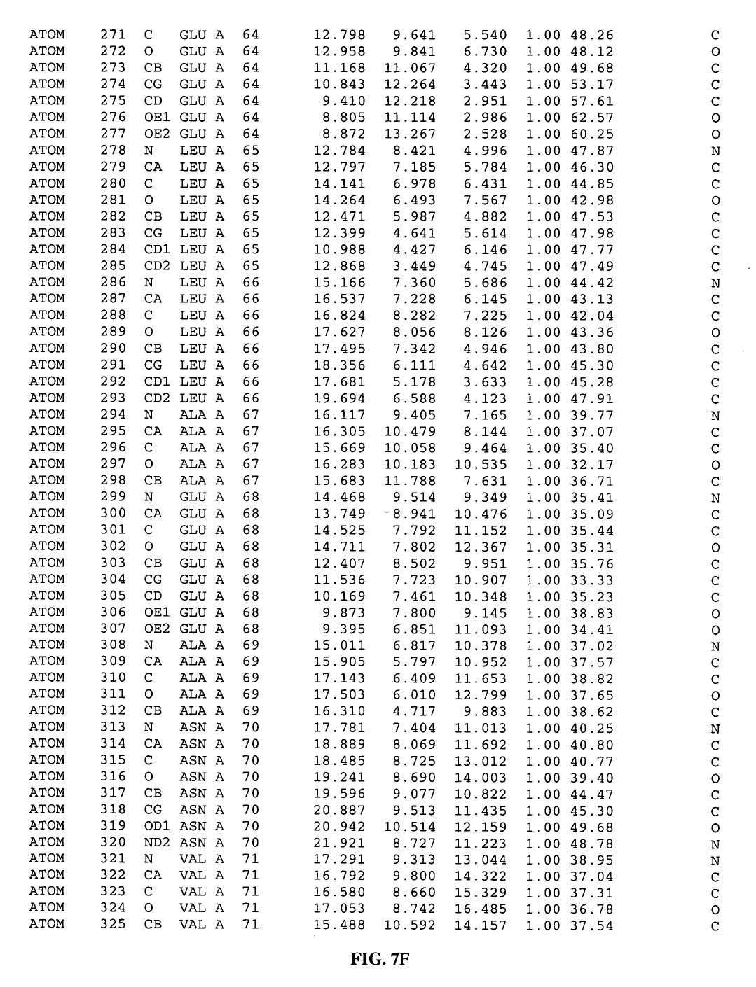

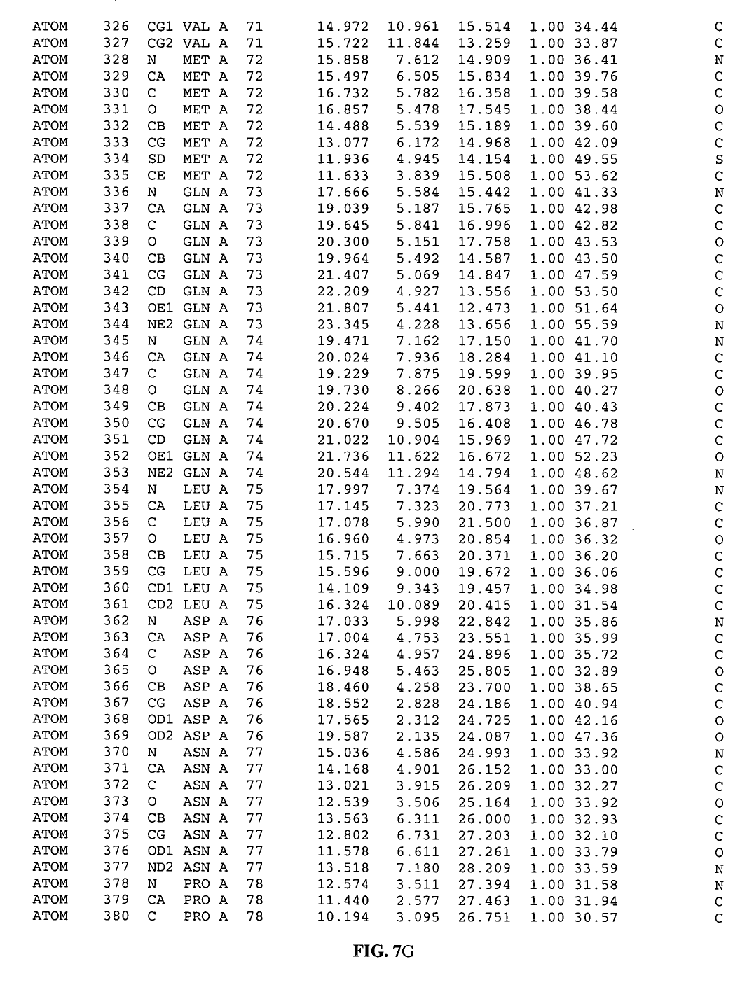

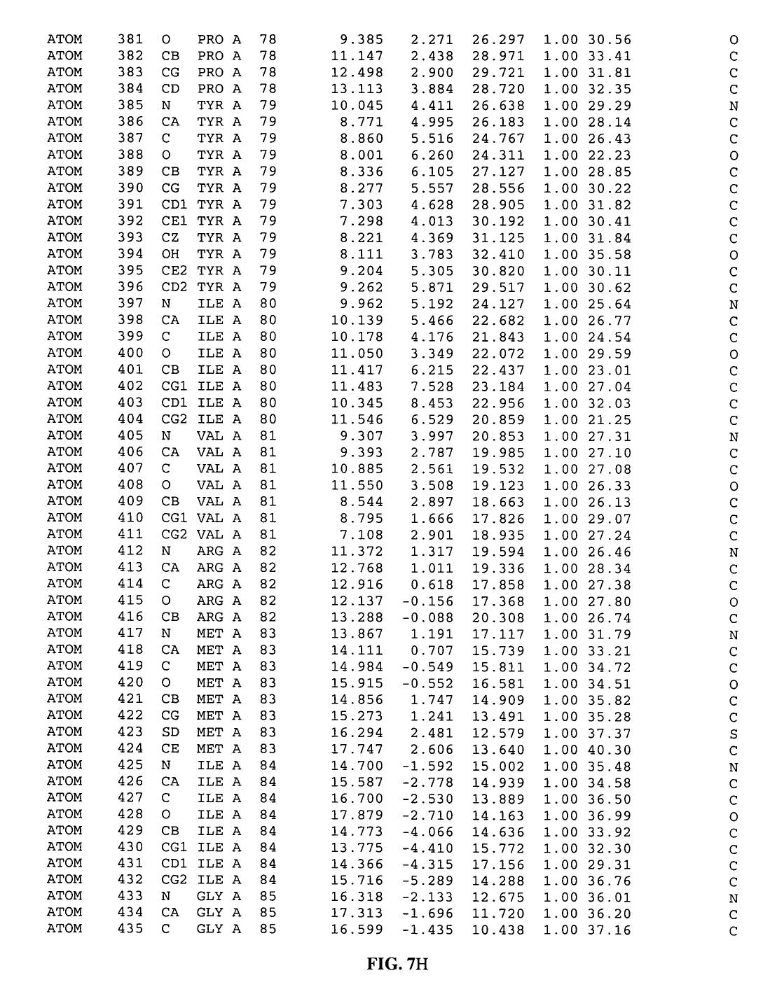

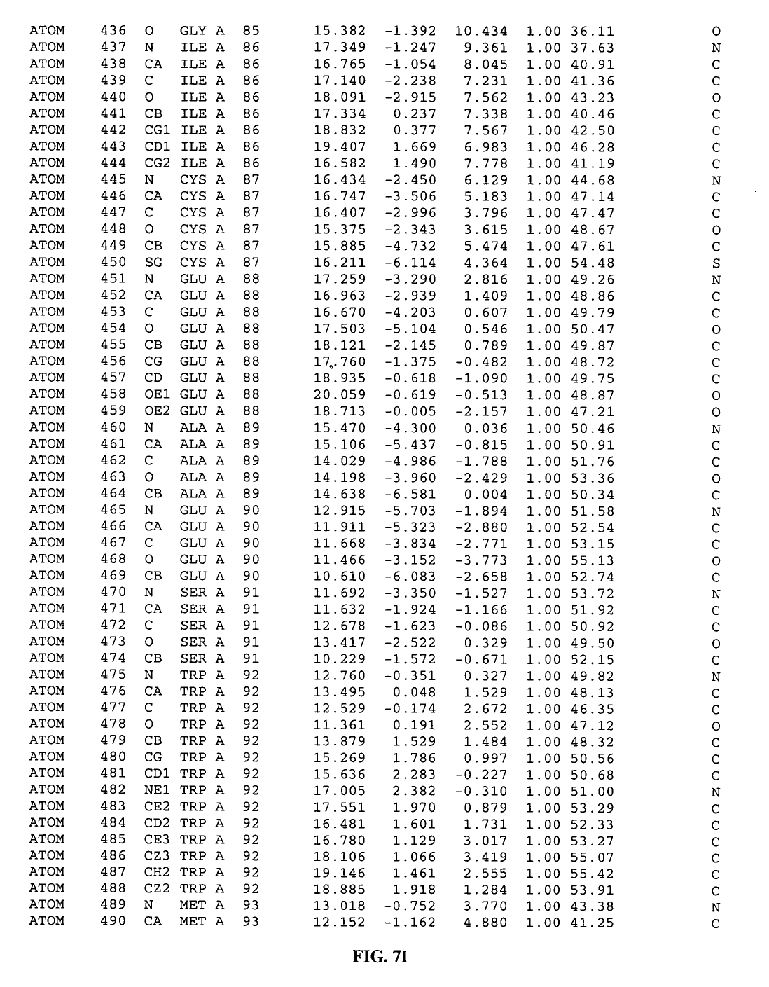

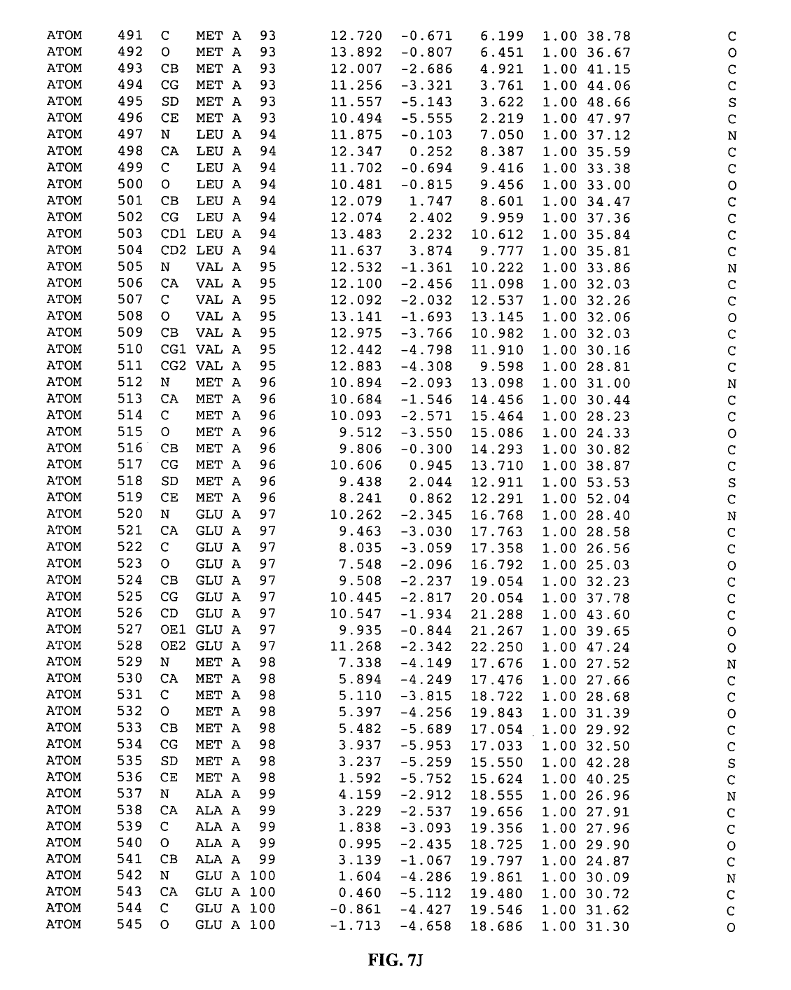

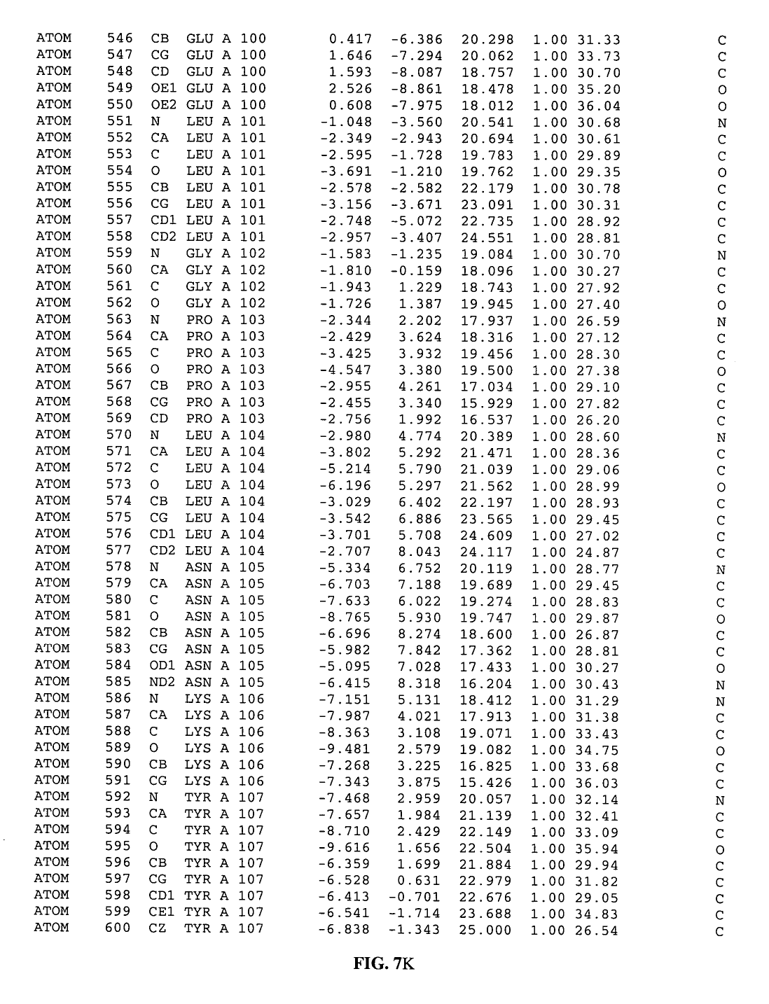

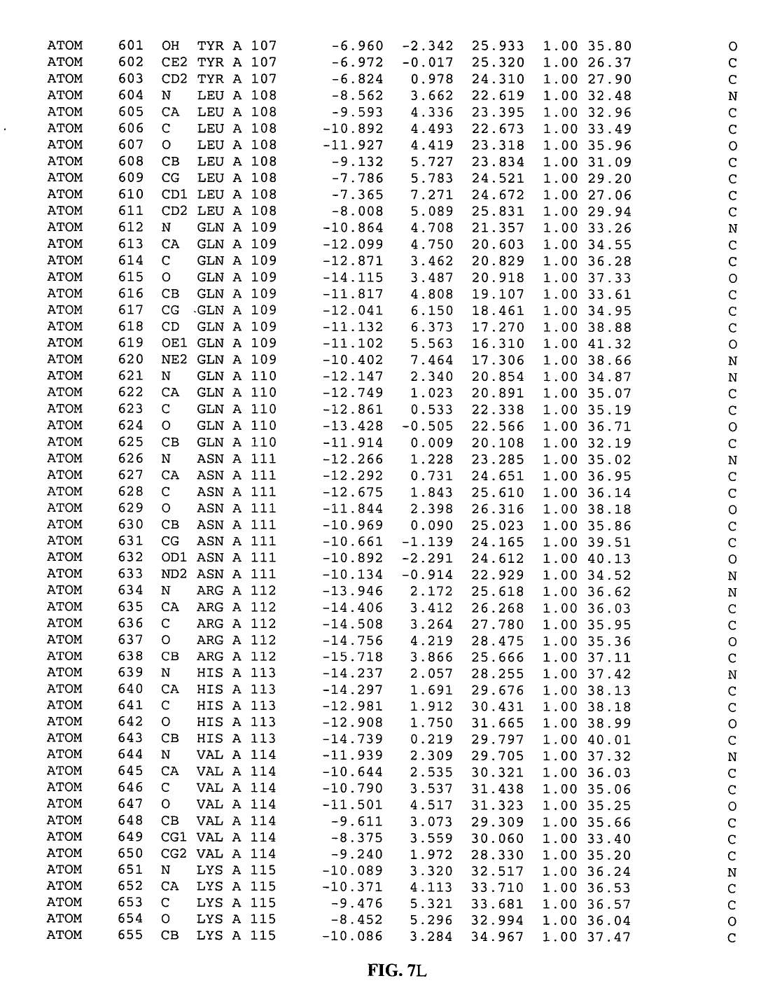

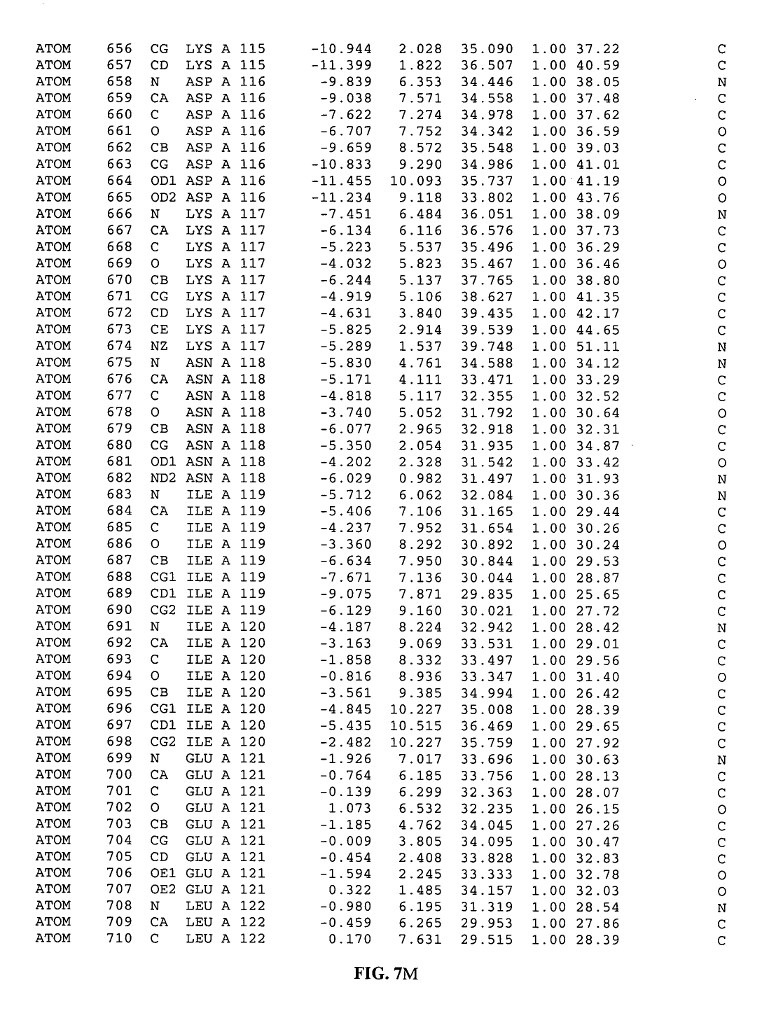

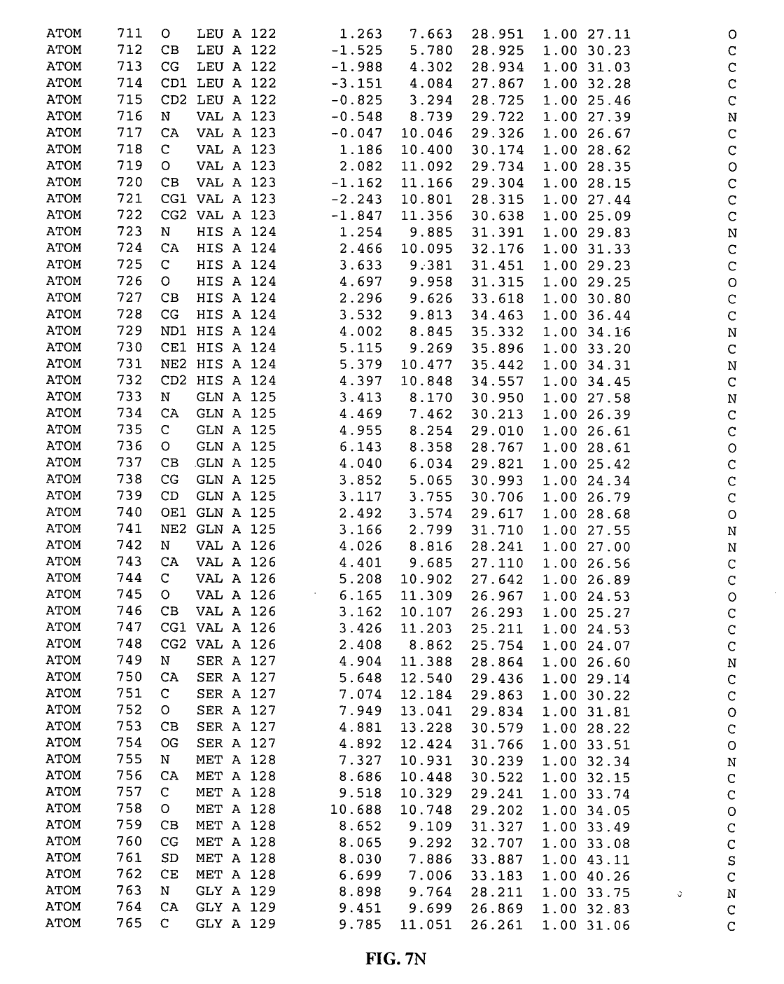

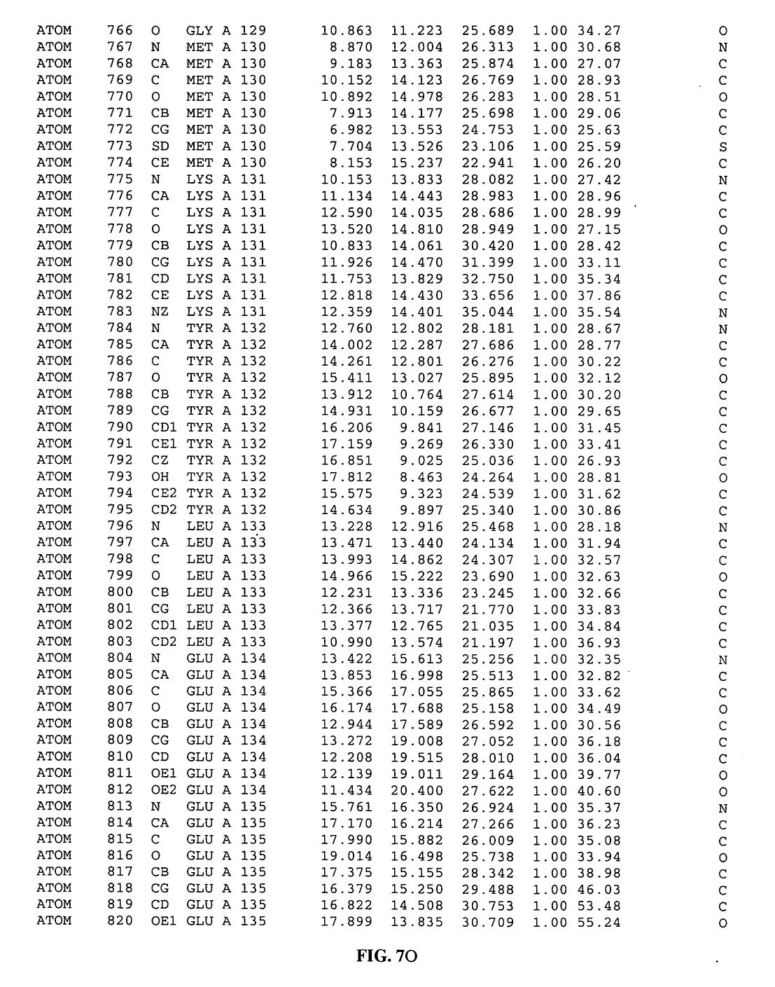

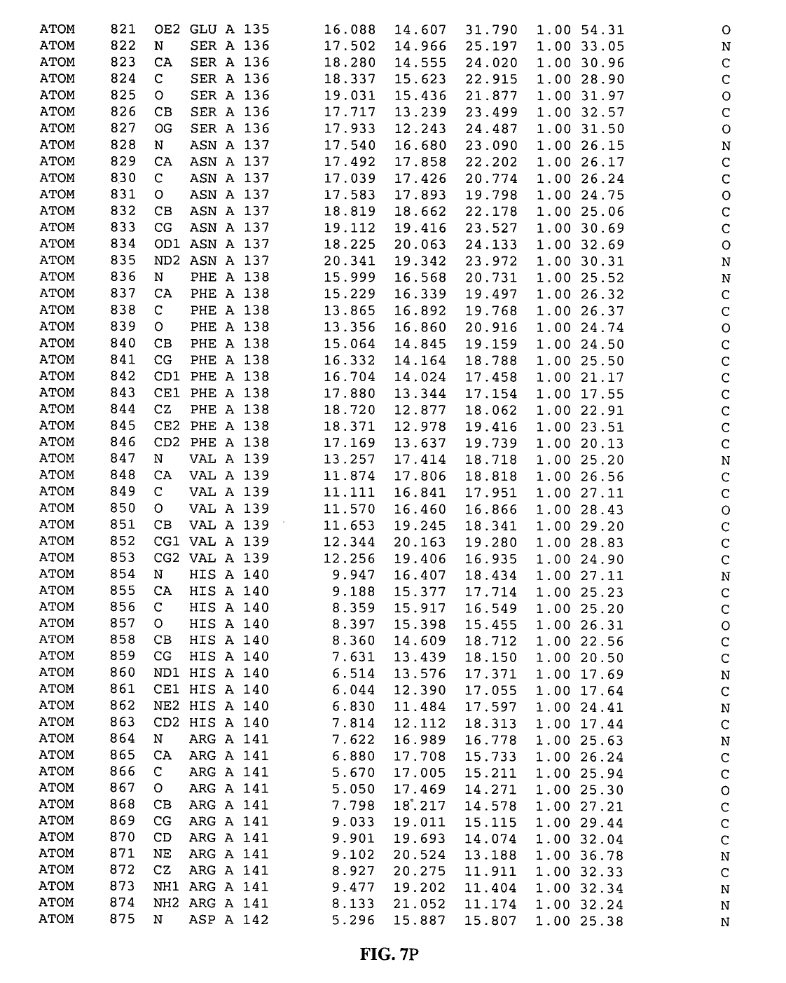

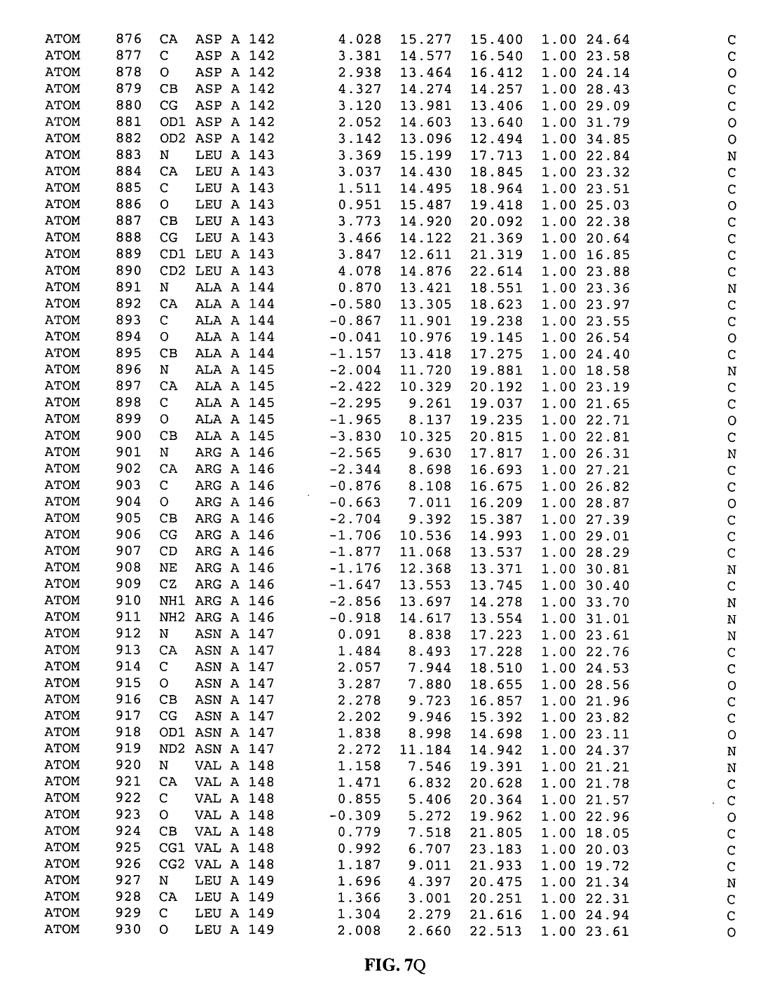

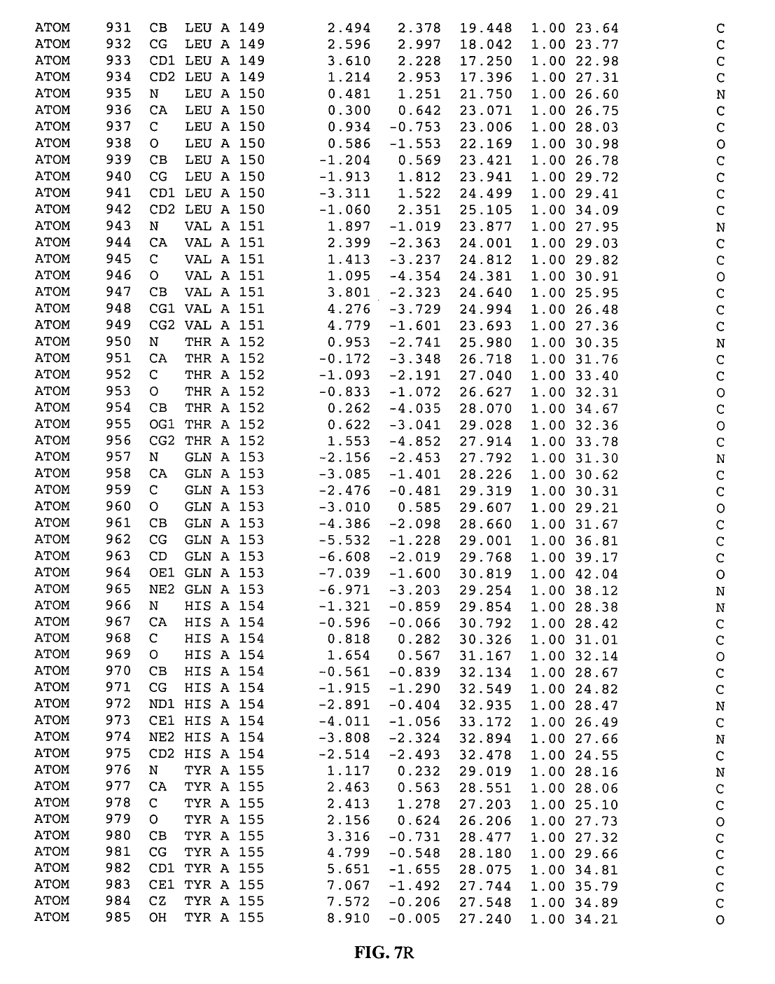

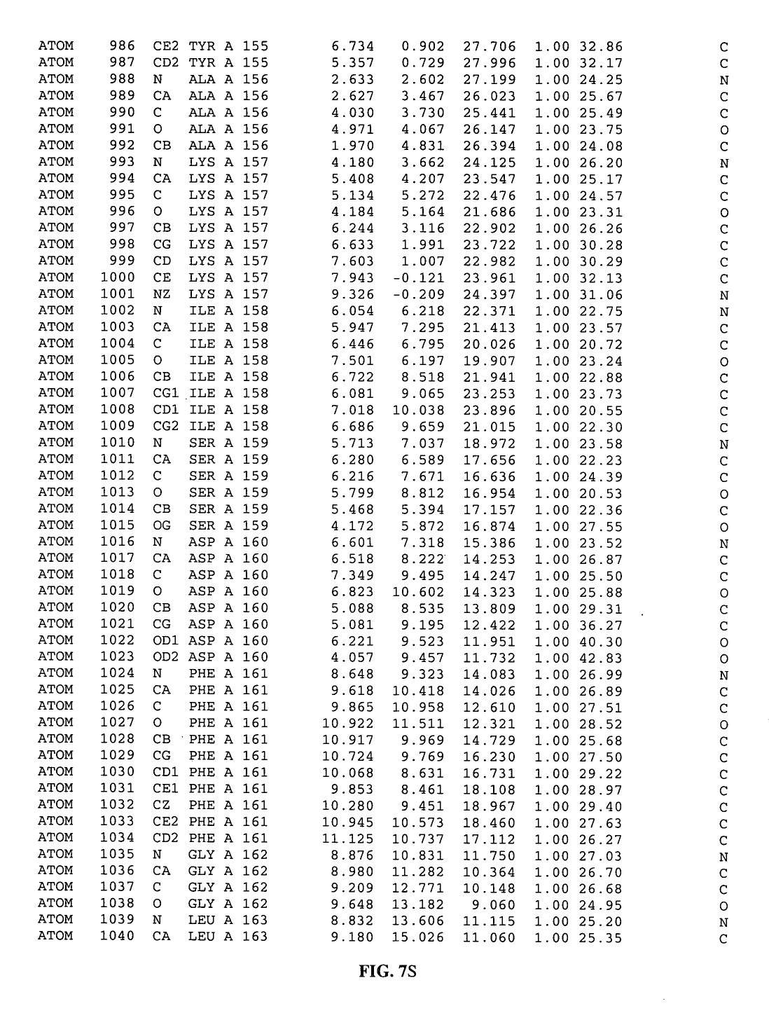

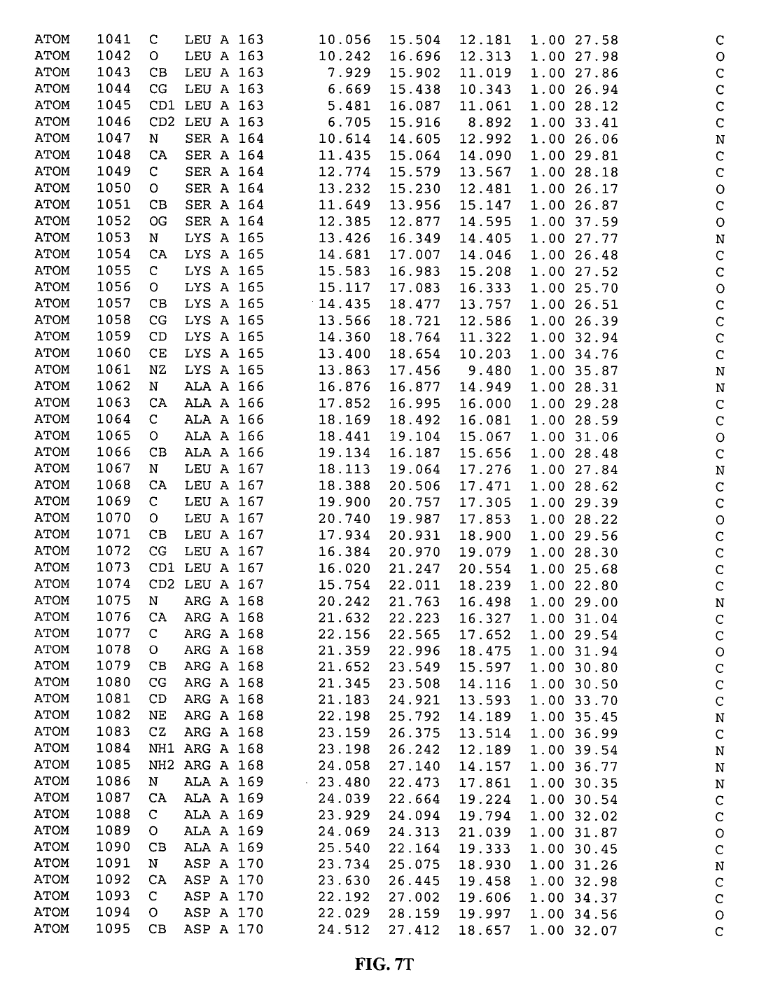

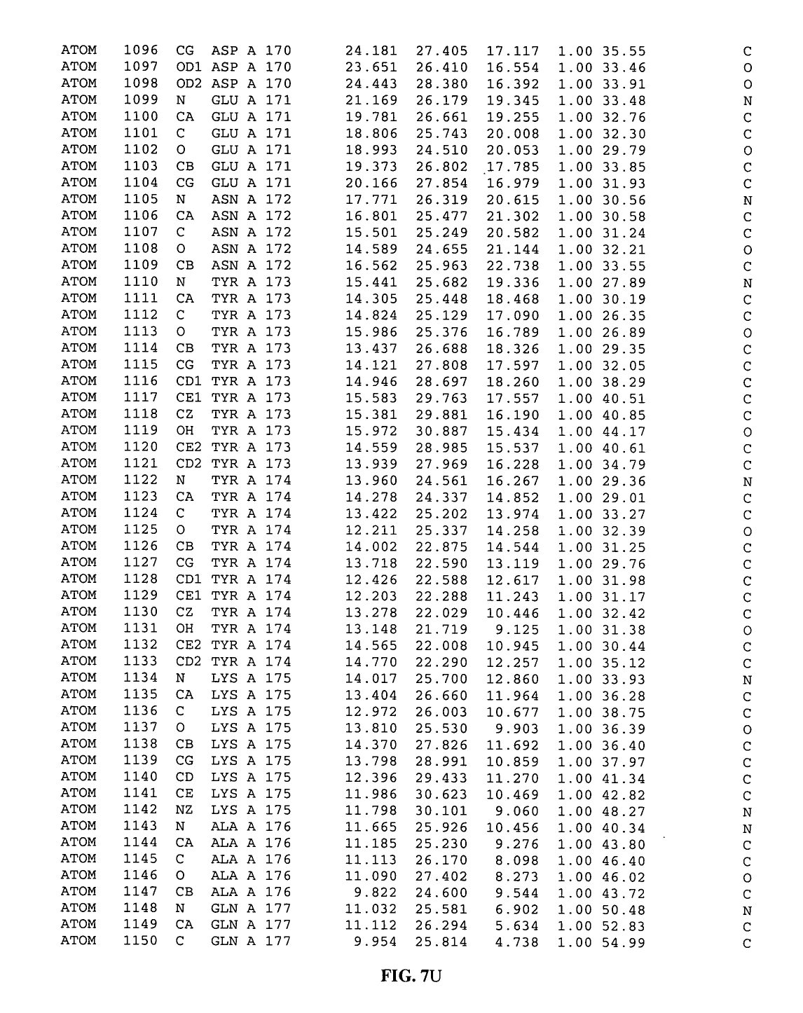

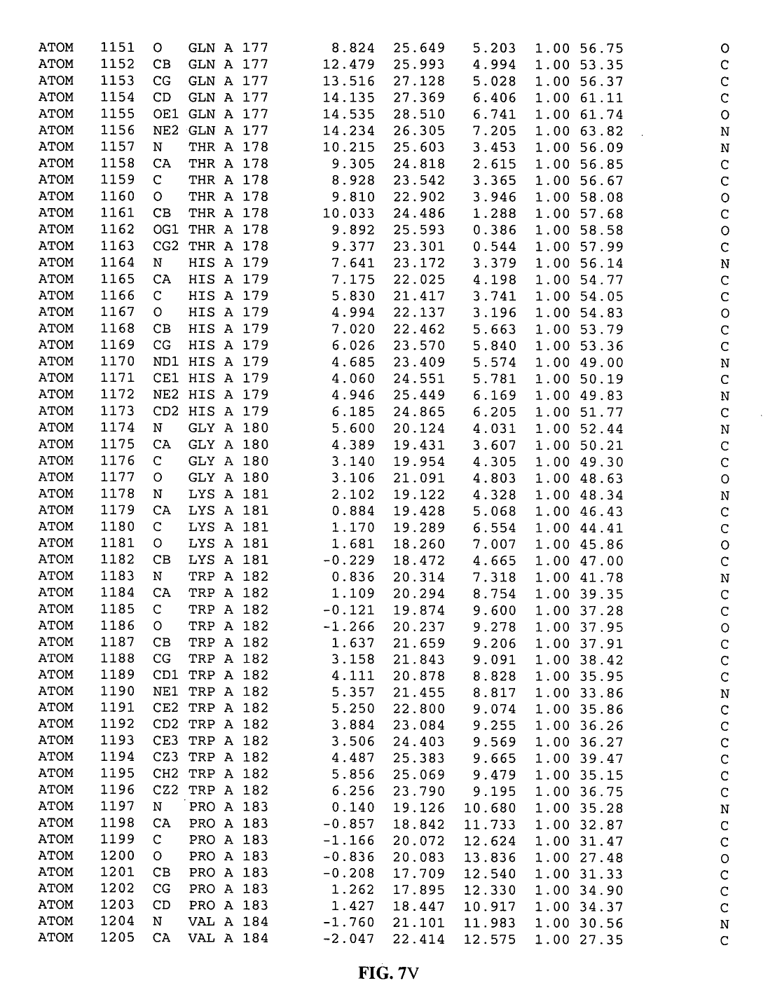

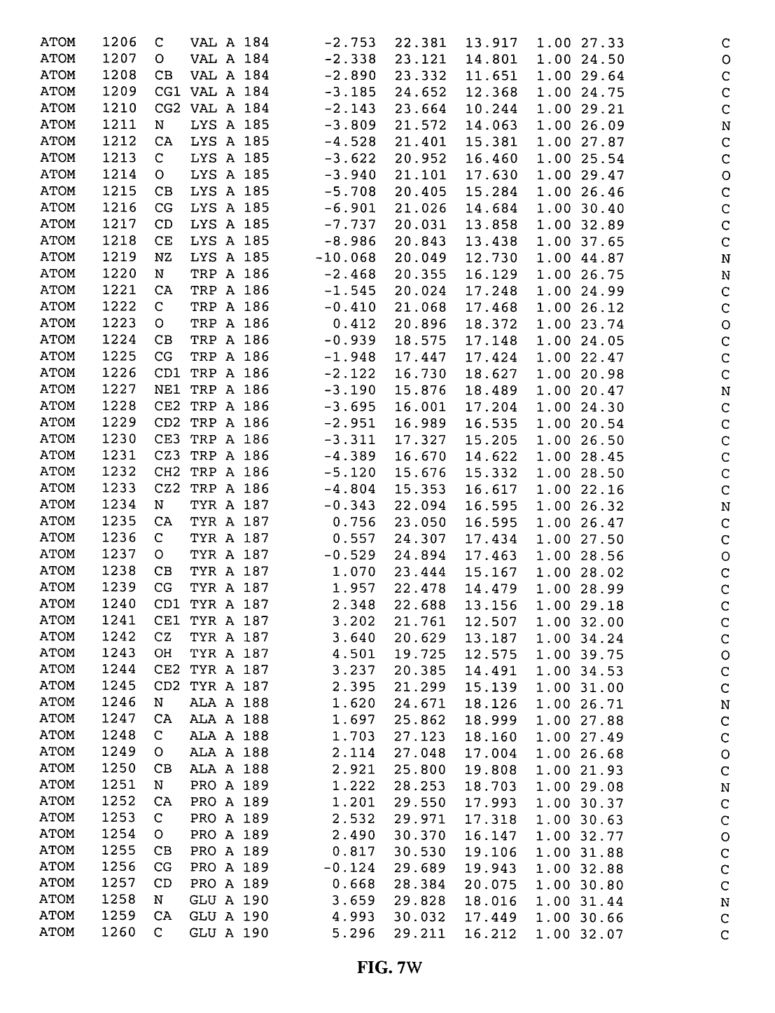

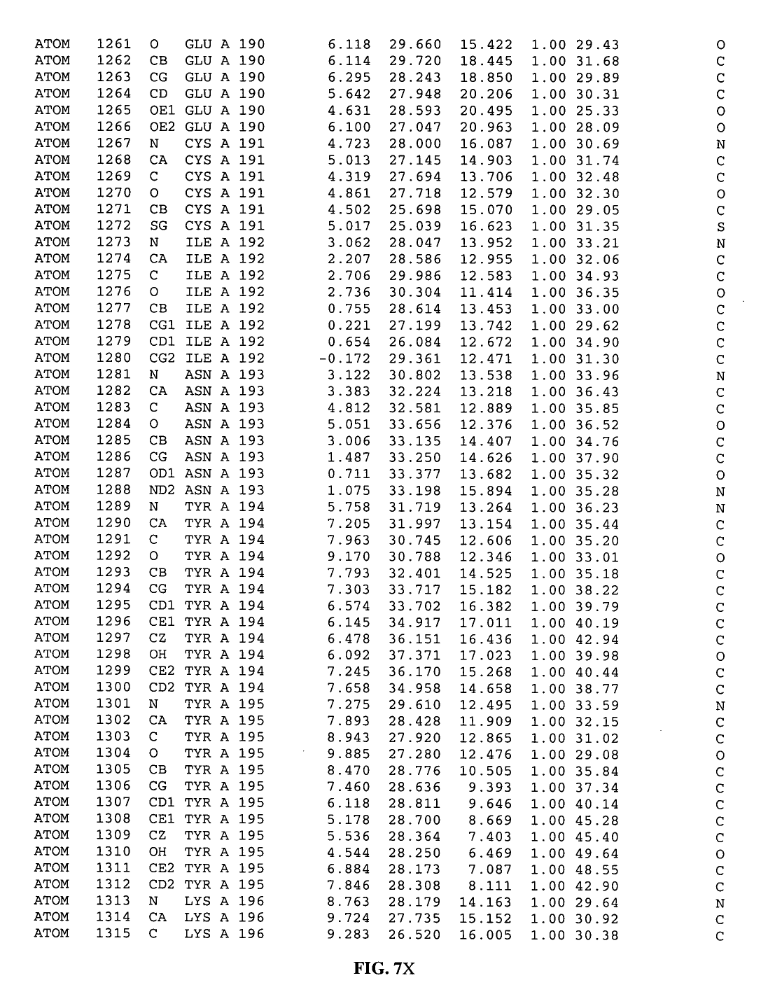

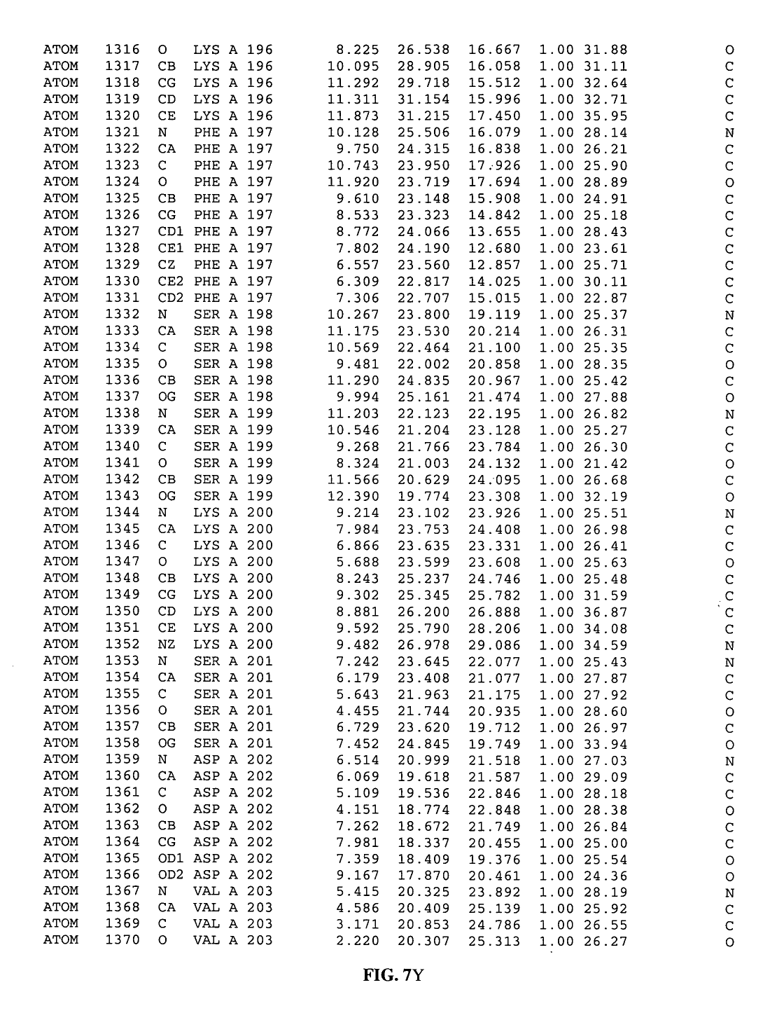

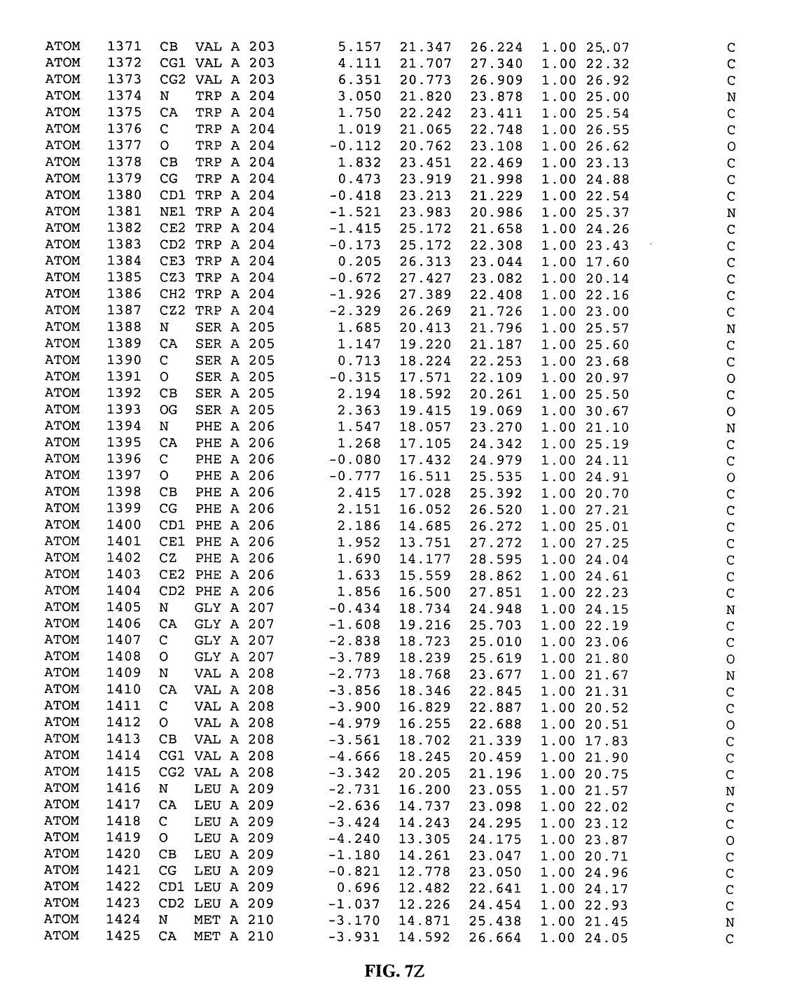

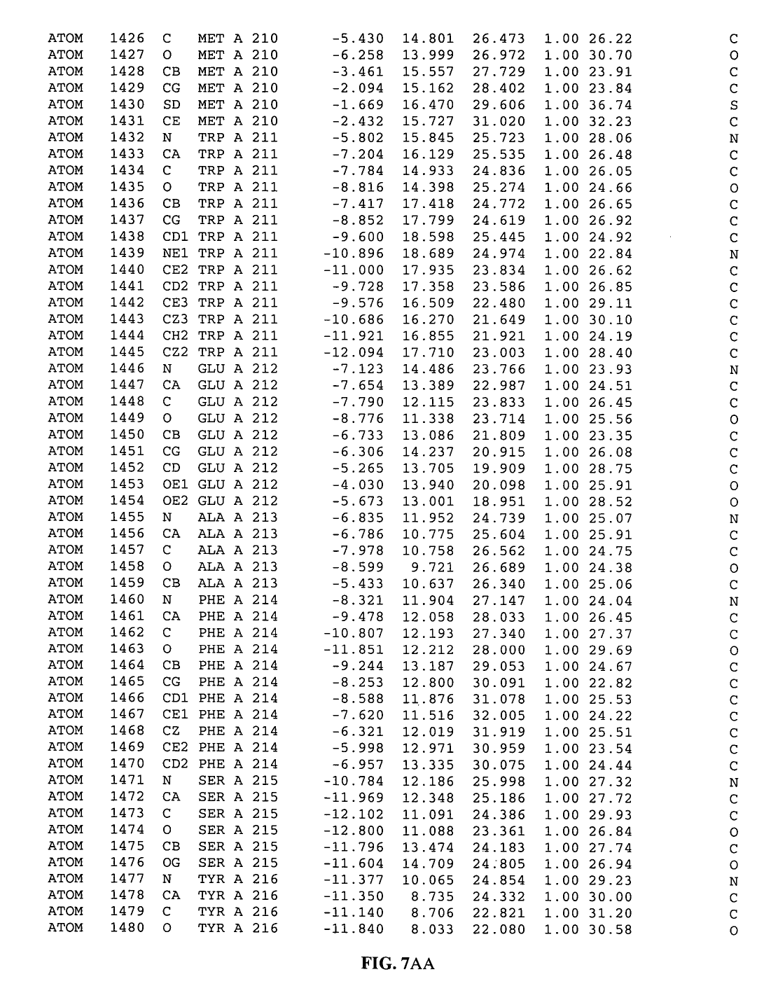

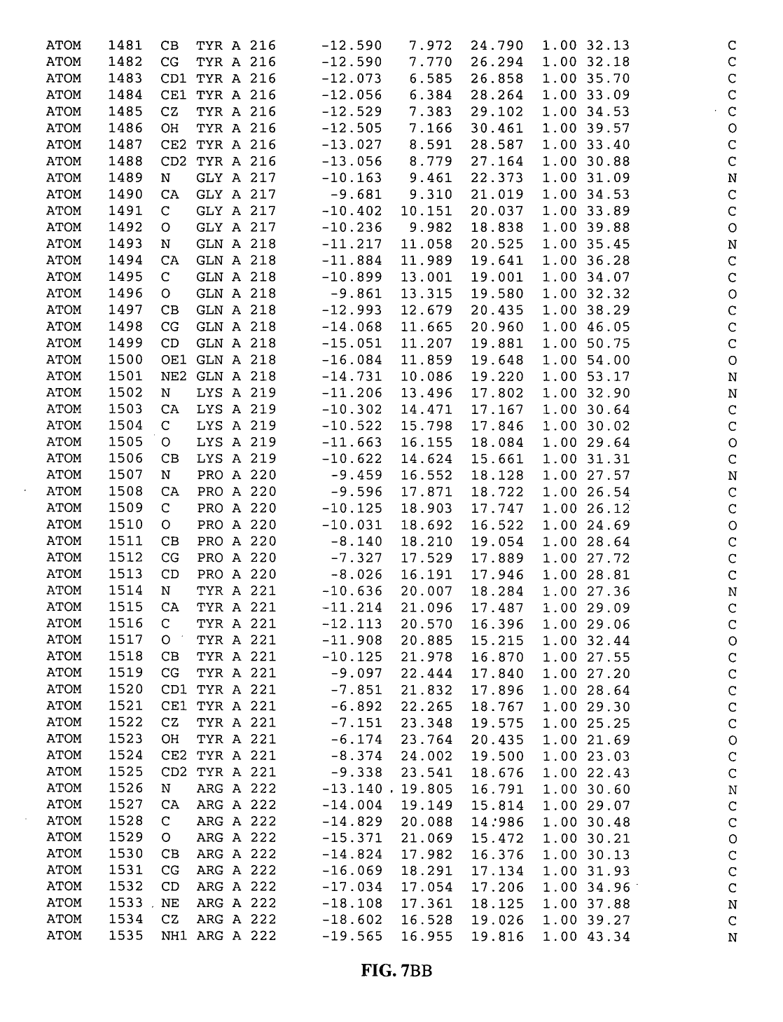

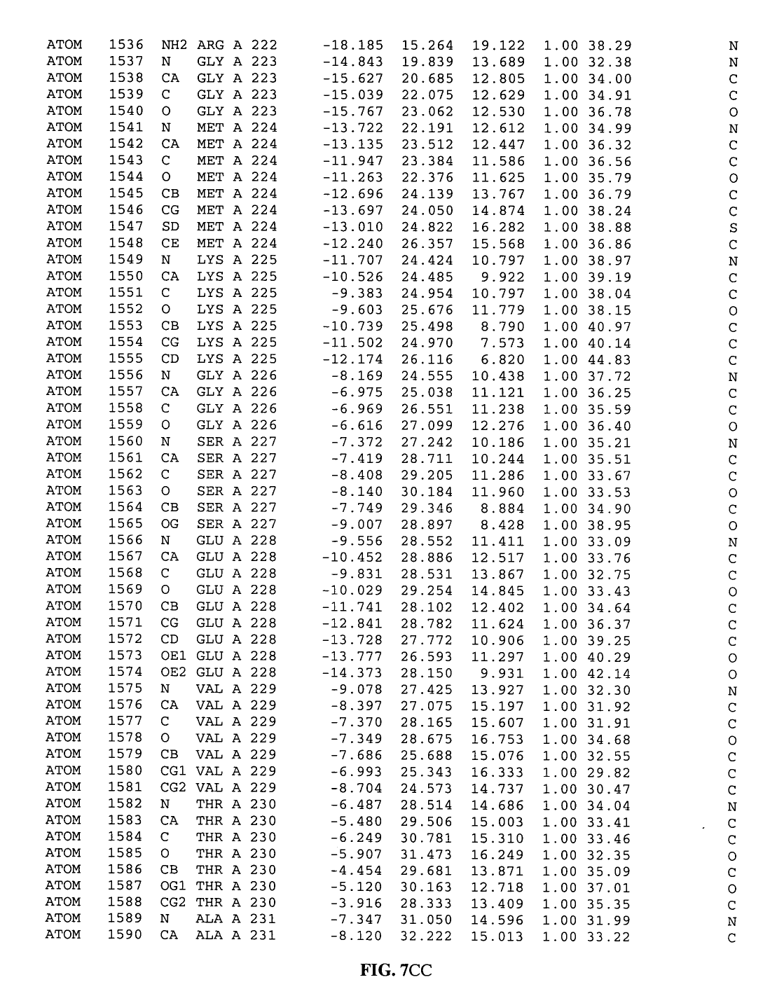

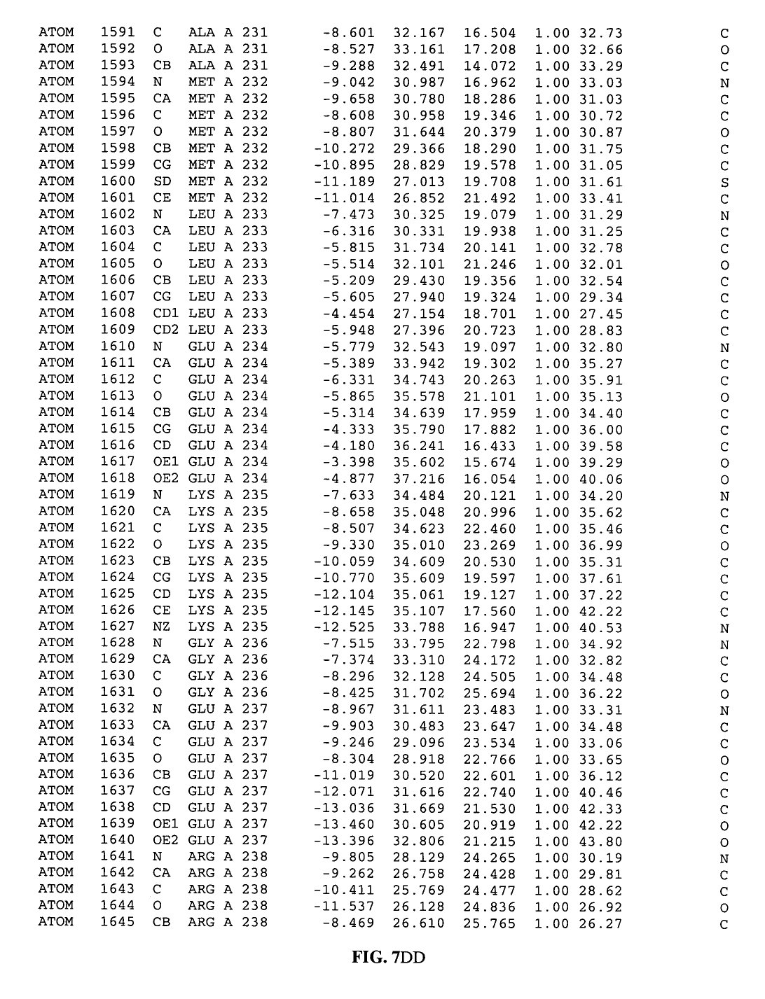

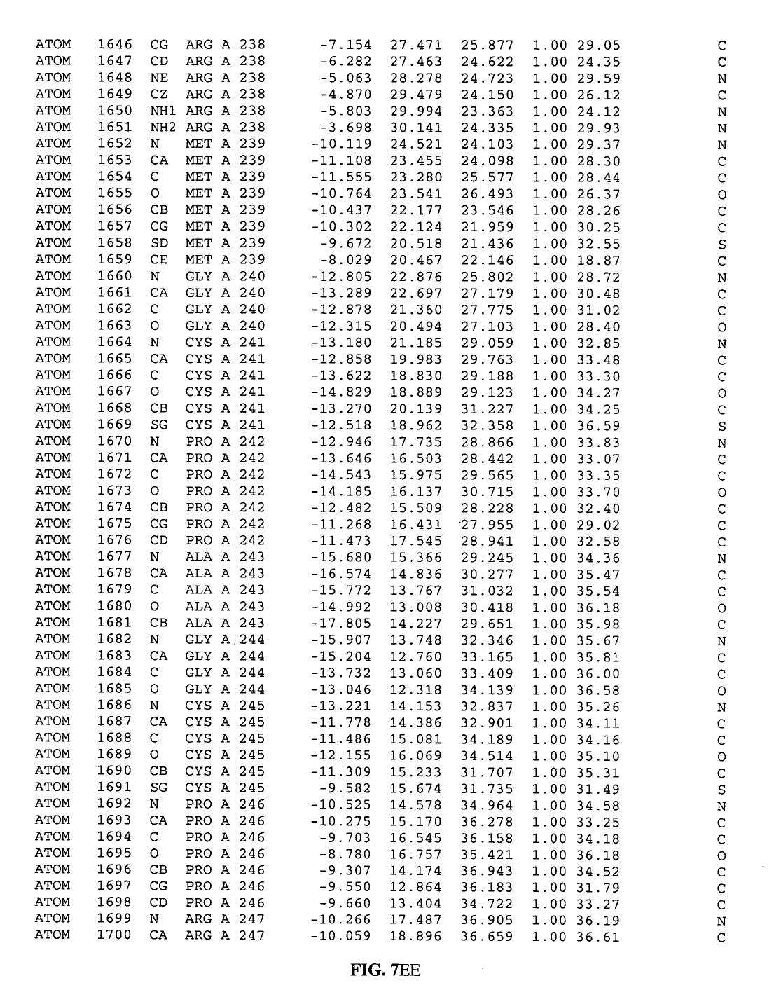

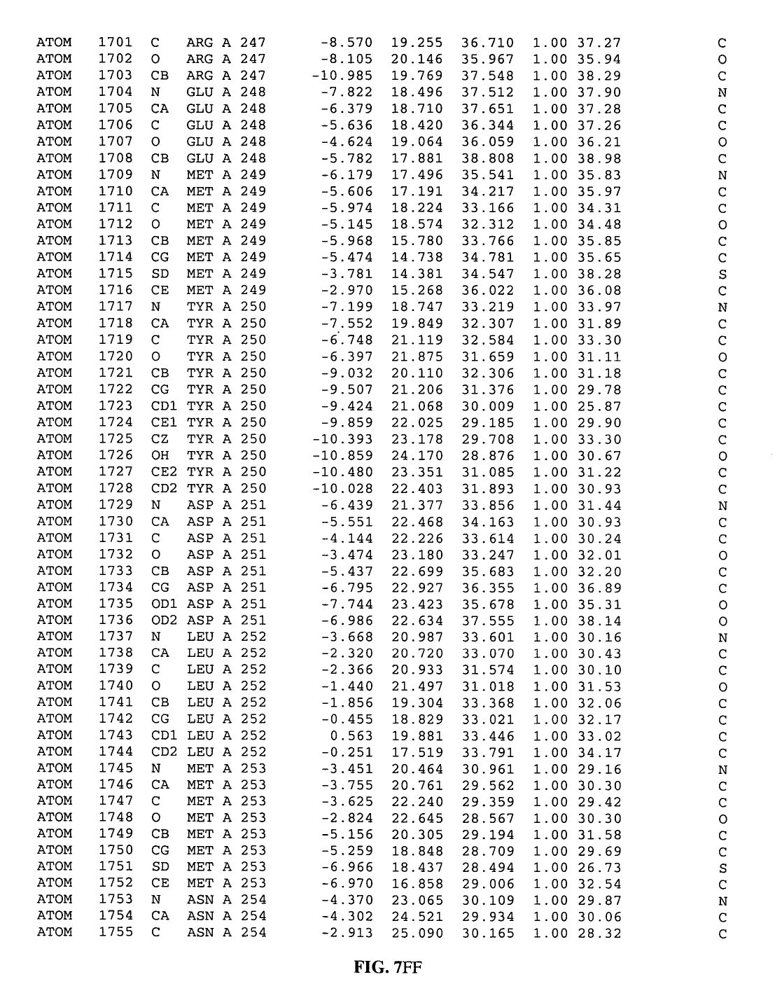

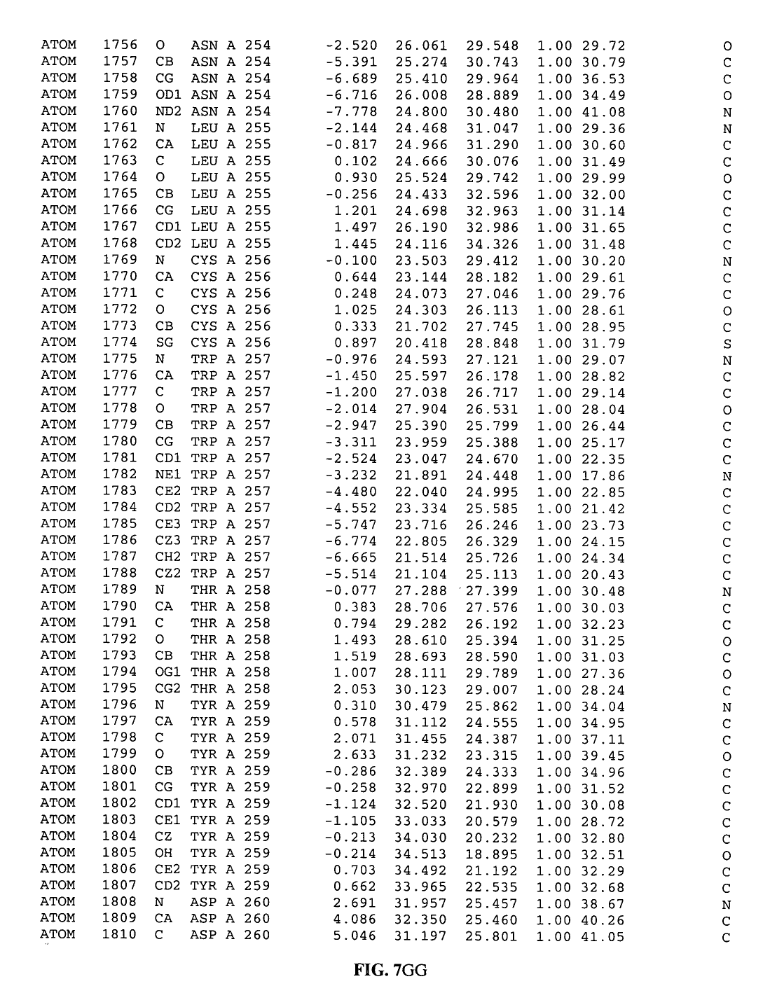

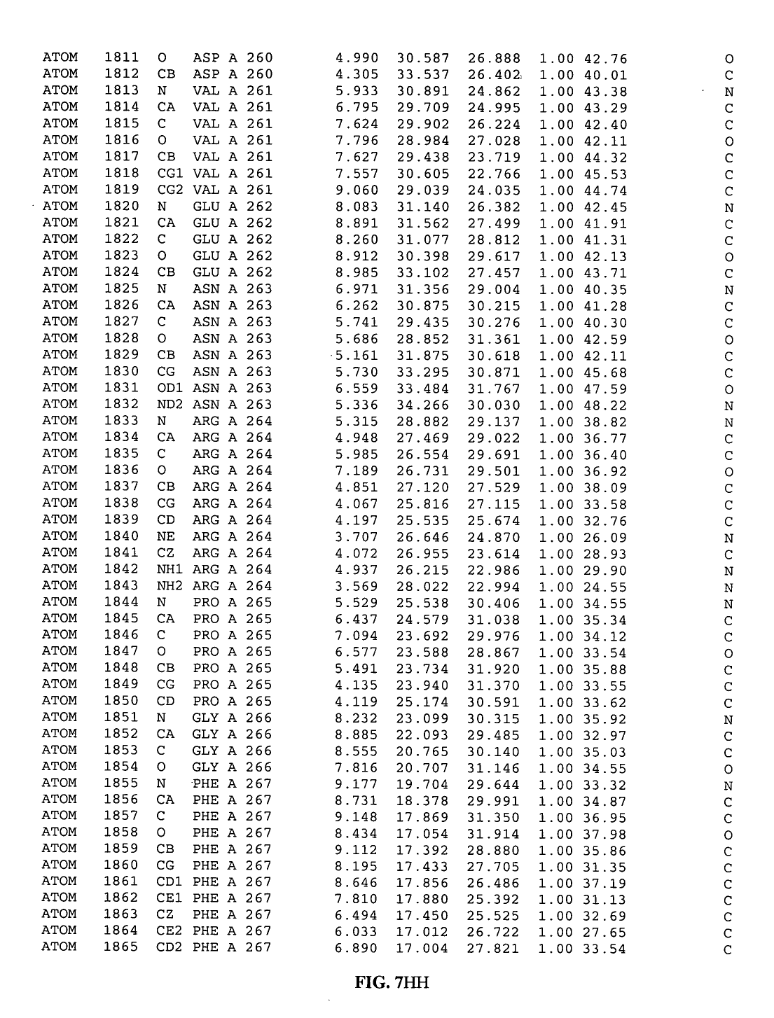

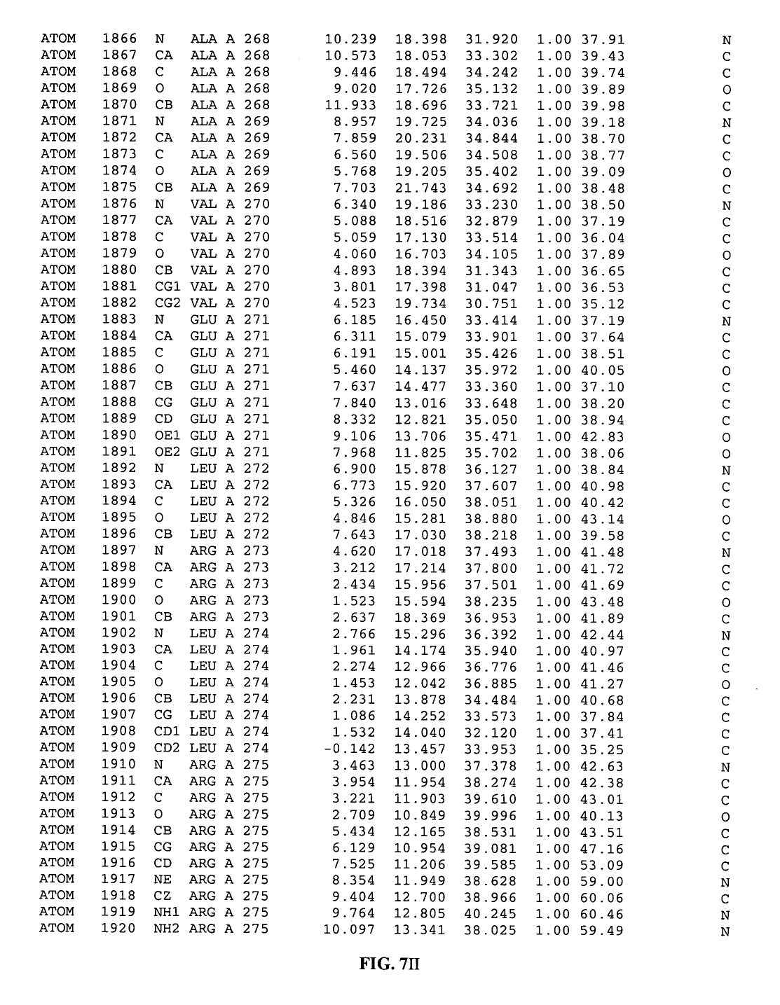

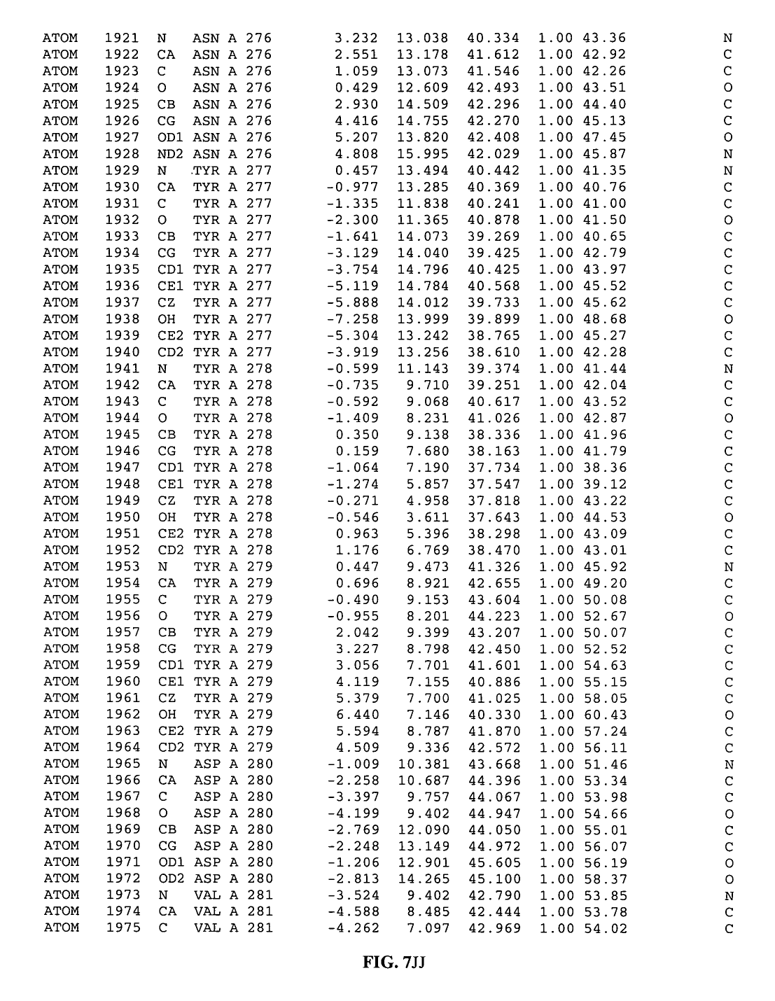

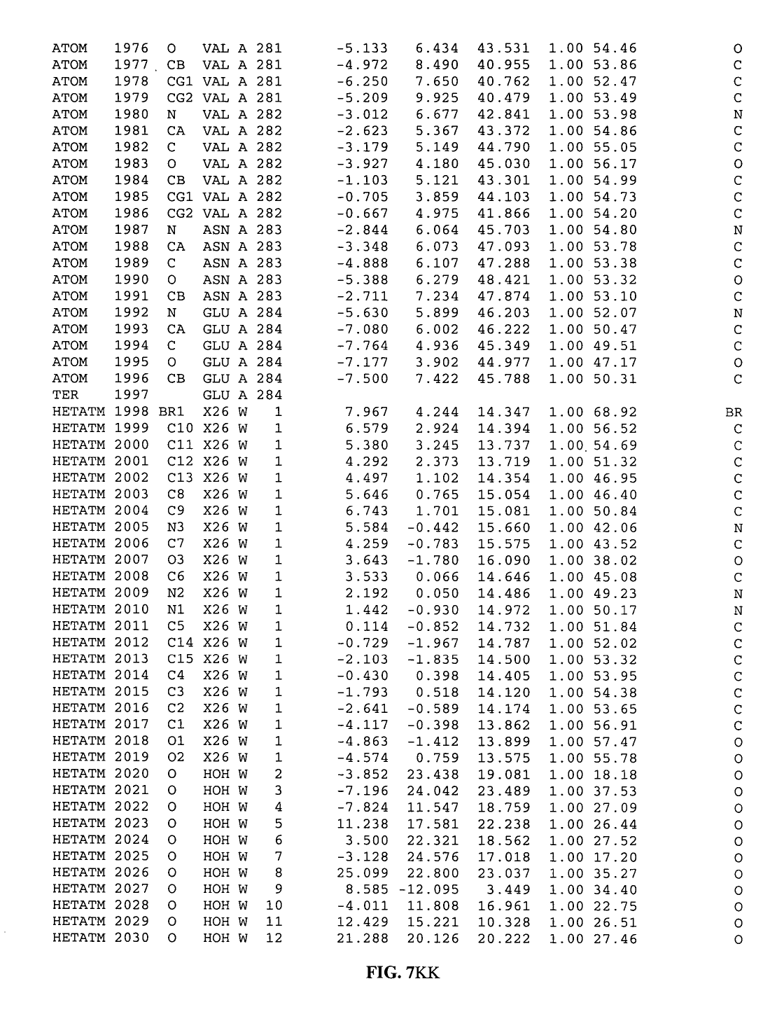

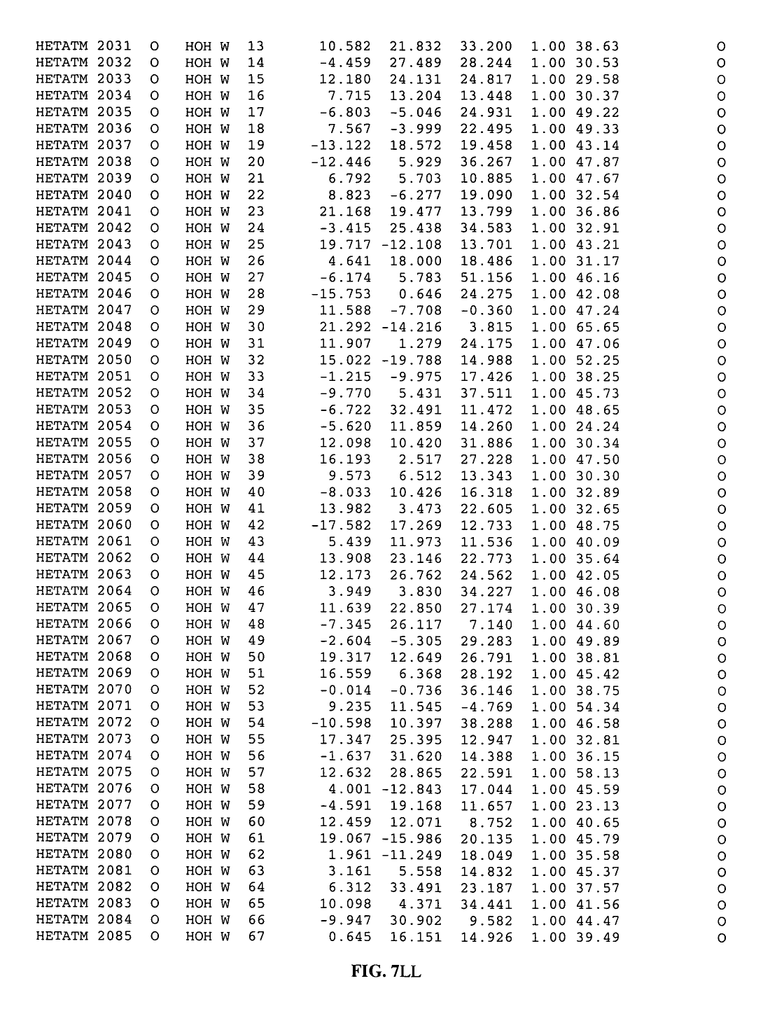

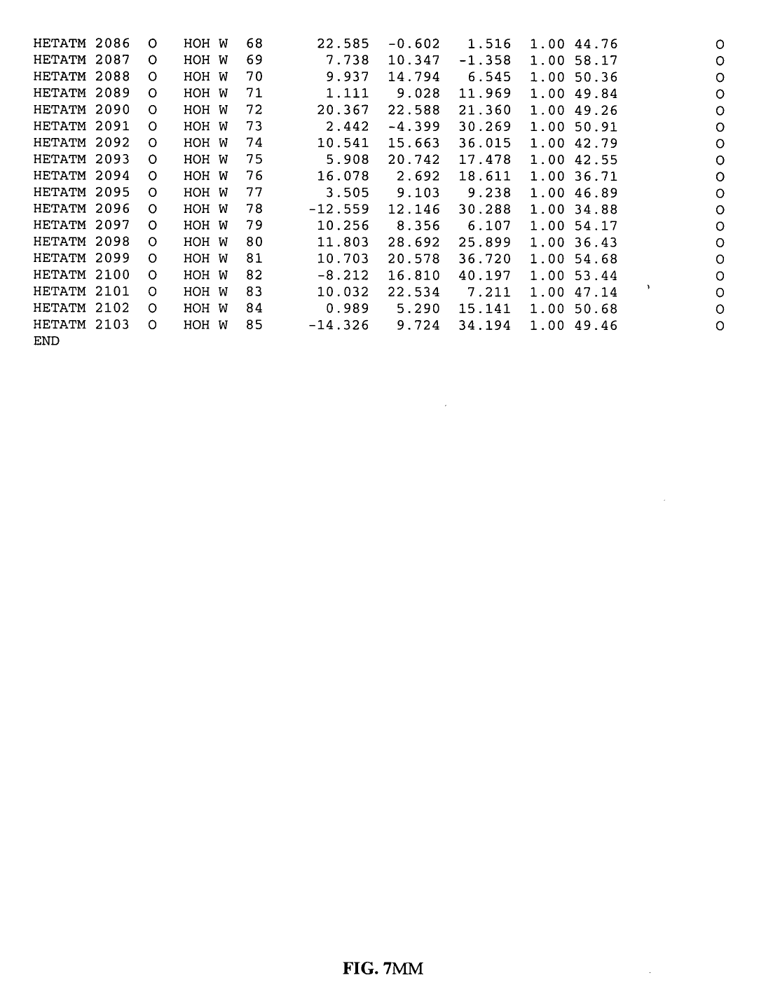

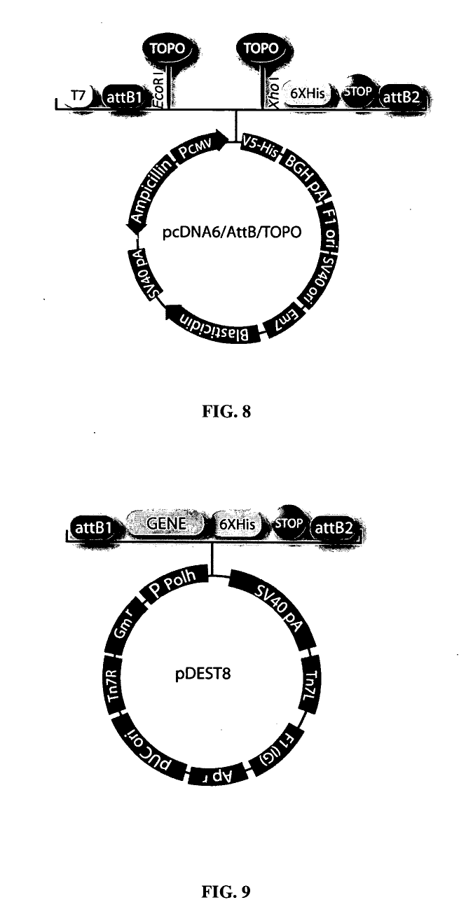

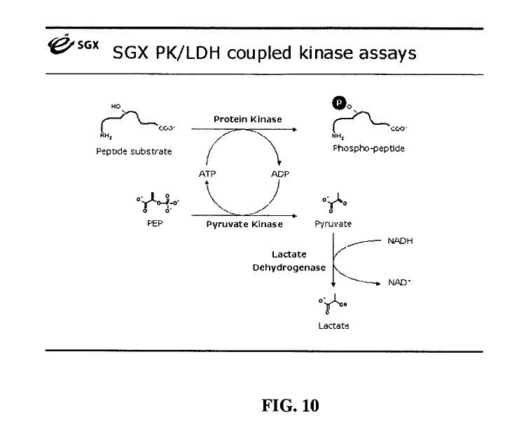

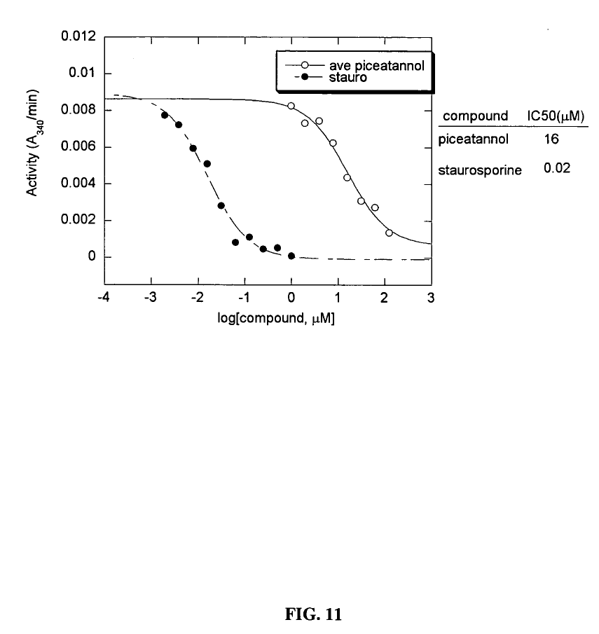

The “entry vector” DNA is used in a standard GATEWAY™ LR recombination with pDEST8™(Invitrogen Corporation, Cat.#11804010) and transformed into chemically competent TOP 10 cells (Invitrogen Corporation, Cat.#C4040-10), and plated on selective media. One colony was picked into a miniprep and DNA was obtained (the “destination vector”). FIG. 7.

The “destination vector” was then transformed into DH10 BAC chemically competent cells (Invitrogen Corporation, Cat#10361012) which uses site specific transposition to insert a foreign gene into a bacmid propogated in E. coli. The transformation was then plated on selective media. 1-2 colonies were picked into minipreps. The Nautilus Genomic miniprep kit (Active Motif, Cat.#50050) was used to purify the bacmid DNA. The bacmid was then verified by PCR.

The bacmid was transfected and expressed in SF9 cells using the following standard Bac to Bac protocol (Invitrogen Corporation, Cat.#10359-016)

Day 0

Seeded 9X10E5 cells per 35 mm well (of a 6 well plate) in 2 ml Sf-900II SFM (Cat. #10902-104, Invitrogen Corporation) containing 1% Penicillin/Streptomycin (Cat. #15140122, Invitrogen Corporation).

Allowed cells to attach at 27 C for 1 hour

In a Falcon 2059 polypropylene 12X75 mm tube prepared the following solutions. 1. Dilute 5 μl of SYK miniprep bacmid DNA (Nautilus Genomic DNA Mini Kit Cat. #50050, Active Motif) into 100 μl Sf-900II SFM without pen/strep.

2. Dilute 6 μl of CellFECTIN reagent (Cat. #10362-010 Invitrogen Corporation) in into 100 μl Sf-900II SFM without pen/strep.

Combined the 2 solutions together and incubated 30 minutes at room temperature.

Washed the cells once by aspirating old media and adding Sf-900II SFM without pen/strep.

Removed media and add 0.8 ml Sf-900II SFM without pen/strep to each well. Added lipid/DNA to well.

Incubated 5 hours in 27 C. incubator.

Removed media and replaced with 2 ml Sf-900II SFM containing Penicillin/Streptomycin.

Placed in 27 C. incubator.

Day 3, P1 to P2

In a T75 Tissue Culture Flask seeded 6X10E6 SF9 cells in a total volume of 14 ml Sf-900II SFM containing Penicillin/Streptomycin. Allowed to attach for 1 hour.

Using a 5 ml pipet removed supernatant containing infectious P1 SYK Baculovirus particles from the transfected well of the 6 well and transferred directly into T75 Flask.

Placed in 27 C. incubator.

Day 10,P2to P3

On Day 10 Harvested SYK Baculovirus supernatant and cells by vigorously pipeting the media to remove the cells from the flask wall.

Pipeted the media and cells into a 15 ml sterile conical tube and centrifuged the tube at ©2000 rpm at room temperature for 5 minutes. Saved supernatant (P2).

Cells were analyzed for protein expression by western blot.

P3 infection

Seeded SF21 cells in a 500 ml suspension flask at 2X10E6 cells per ml. In a total volume of 100 ml.

Added infectious SYK supernatant (14 ml) from P2 expression to suspension flask Incubated at 27 C., shaking at 120-130 rpm.

Expressed protein for 72 hours.

Harvested 1 ml cells and western blot to determine expression

Harvested P3 supernatant by centrifugation 3000 rpm for 15 minutes at room temperature.

Sterile filtered viral supernatant.

SYK Scale Up

Seeded 6 liters of SF21 cells at 2X10E6 cells per ml in 1 liter of cells in 2-liter suspension flasks. Infected cells with 15 ml of P3 SYK baculovirus per liter. Incubated at 27 C., shaking at 120-130 rpm.

Expressed protein for 48 hours. Harvested 1 ml cells from each liter and western blot to determine expression. Remaining cells were collected by centrifugation, and the pellets stored at −80° C.

After thawing at room temperature, cells were lysed in cracking buffer (50 mM Tris-HCl, pH 8.0; 200 mM arginine; 150 mM NaCl; 10% glycerol; 0.1% Igepal 630), and centrifuged to remove cell debris. The soluble fraction was purified over an IMAC column charged with nickel (Pharmacia, Uppsala, Sweden), and eluted under native conditions with a step gradient of 400 mM imidazole in 50 mM Tris pH7.8, 10mM methionine, 10% glycerol. The SYK protein was then purified by gel filtration using a Superdex 200 preparative grade column equilibrated in GF4 buffer (10 mM HEPES, pH 7.5, 10mM methionine, 500 mM NaCl, 5 mM DTT, and 10% glycerol). Fractions containing the purified SYK kinase domain were pooled and concentrated to 13.2 mg/ml. The protein obtained was >98% pure as judged by mass spectroscopic analysis. Mass spectroscopic analysis of the purified protein showed that it was not phosphorylated.

For crystals of Homo sapiens SYK from which the molecular structure coordinates of the invention are obtained, it has been found that a sitting drop containing 2 μ of SYK polypeptide (13.2 mg/ml) in 10 mM Hepes, pH 7.5, 10% glycerol, 150 mM NaCl, 5 mM DTT, and 10 mM methionine; and 1 μl reservoir solution: 25% (v/v) PEG 3350, and 100 mM Tris (pH 8.5), in a sealed container containing 100 μL reservoir solution, incubated overnight (12 hours) at 25° C. provides diffraction quality crystals. One hour before setting up the trays, 1 mM AMP-PNP and 2 mM MgCl₂ were added to the polypeptide.

Those of ordinary skill in the art recognize that the drop and reservoir volumes may be varied within certain biophysical conditions, up to about 10%, 25%, 40% or 50% greater or less than those stated here, and still allow crystallization.

### Example 1.2

### Crystal Diffraction Data Collection

The crystals were individually harvested from their trays and transferred to a cryoprotectant consisting of reservoir solution plus 15% glycerol. After about 2 minutes the crystal was collected and transferred into liquid nitrogen. The crystals were then transferred in liquid nitrogen to the Advanced Photon Source (Argonne National Laboratory).

### Example 1.3

### Structure Determination

X-ray diffraction data were indexed and integrated using the program MOSFLM (Collaborative Computational Project, Number 4, Acta. Cryst. D50, 760-63, 1994; www.ccp4.ac.uk/main.html) and then merged using the program SCALA (Collaborative Computational Project, Number 4, Acta. Cryst. D50, 760-63, 1994; www.ccp4.ac.uk/main.html). The subsequent conversion of intensity data to structure factor amplitudes was carried out using the program TRUNCATE (Collaborative Computational Project, Number 4, Acta. Cryst. D50, 760-763, 1994; www.ccp4.ac.uk/main.html). An initial model was obtained by molecular replacement using 1 IEP.pdb as a search model using the program MOLREP. (Collaborative Computational Project, Number 4, Acta. Cryst. D50, 760-63, 1994). The initial protein model was built into the resulting map using the program XTAL VIEW/XFIT (McRee, D.E. J. Structural Biology, 125:156-65, 1993; available from CCMS (San Diego Super Computer Center) CCMS-request@sdsc.edu.). This model was refined using the program REFMAC (Collaborative Computational Project, Number 4, Acta. Cryst. D50, 760-63, 1994; www.ccp4.ac.uk/main.html) with interactive refitting carried out using the program XTALVIEW/XFIT (McRee, D. E. J. Structural Biology, 125:156-65, 1993; available from CCMS (San Diego Super Computer Center) CCMS-request@sdsc.edu). The stereochemical quality of the atomic model was monitored using PROCHECK (Laskowski et al., J. Appl. Cryst. 26, 283-91, 1993) and WHATCHECK (Vriend, G., J. Mol. Graph 8:52-56, 1990; Hooft, R. W. W. et al., Nature 381:272, 1996) and the agreement of the model with the x-ray data was analyzed using SFCHECK (Collaborative Computational Project, Number 4, Acta. Cryst. D50, 760-63, 1994); www.ccp4.ac.uk/main.html).

TABLE 1
Data Collection Statistics

| Space group | P 1 21 1 |
|---|---|
| Cell dimensions | a = 38.82 Å |
| | b = 84.28 Å |
| | c = 40.25 Å |
| | α = 90° |
| | β = 99.57° |
| | γ = 90° |
| Wavelength λ | 0.9794 Å |
| Overall Resolution limits | 42.258 Å |
| | 2.1 Å |
| Number of reflections collected | 141328 |
| Number of unique reflections | 20850 |
| Overall Redundancy of data | 7 |
| Overall Completeness of data | 96.6% |
| Completeness of data in last data shell | 97.9% |
| Overall R_{SYM} | 0.088 |
| R_{SYM} in last resolved shell | 0.353 |
| Overall I/sigma(I) | 11.5 |
| I/sigma(I) in last shell | 5.5 |

TABLE 2
Model Refinement Statistics

| | | |
|---|---|---|
| Model | Total number of atoms | 2018 |
| | Number of water molecules | 48 |
| | Temperature factor for all atoms | 40.62 Å² |
| | Matthews coefficient | 2.30 |
| | Corresponding solvent content | 46.05% |
| Refinement | Resolution limits | 42.258 Å |
| | | 2.1 Å |
| | Number of reflections used | 14735 |
| | with I > 1 sigma(I) | 14718 |
| | with I > 3 sigma(I) | 11692 |
| | Completeness | 98.5% |
| | R-factor for all reflections | 0.2452 |
| | Correlation coefficient | 0.9193 |
| | Number of reflections above 2 | 13603 |
| | sigma(F) and resolution from 5.0 Å - | |
| | high resolution limit | |
| | used to calculate Rworking | 12918 |
| | used to calculate Rfree | 685 |
| | R-factor without free reflections | 0.229 |
| | R-factor for free reflections | 0.29 |
| | Error in coordinates estimated by | 0.2845 Å |
| | Luzzati plot | |
| Validation | Phi-Psi core region | 89.9% |
| | Phi-Psi violations | 0 |
| | Residues in disallowed regions: | |
| % bad | Short contact distances | 0.4 |
| contacts | | |
| | RMSD from ideal bond length | 0.011 Å |
| | RMSD from ideal bond angle | 1.21° |

### Example 1.4

### Structure Analyses

Atomic superpositions were performed with MOE (available from Chemical Computing Group, Inc., Montreal, Quebec, Canada). Per residue solvent accessible surface calculations were done with GRASP (Nicholls et al., “Protein folding and association: insights from the interfacial and thermodynamic properties of hydrocarbons,” Proteins, 11:281-96, 1991). The electrostatic surface was calculated using a probe radius of 1.4 Å.

### Example 2

### Determination of SYKKD Structure

The section below describes the production of a polypeptide comprising the Homo sapiens SYKKD, and the preparation and characterization of diffraction quality crystals and heavy-atom derivative crystals. Purified protein was obtained as in Example 1. Crystallization conditions were as in Example 1, with the exception that the crystals were obtained using a reservoir solution of 100 mM Hepes (pH 7.0), and 10% PEG 6 K, (v/v) incubated for seven days at 4° C. Crystals were harvested and, before data collection, crystals were soaked in 50 microliters of mother liquor after adding 0.5 microliters of a 0.01 mg/ml solution of staurosporin in dimethylsulfoxide.

The crystal data was collected as in Example 1, and the structure determination was essentially as in Example 1, with the exception that the example 1 model was used as the reference model for molecular replacement.

TABLE 3
Data Collection Statistics

| Space group | P 21 21 21 |
|---|---|
| Cell dimensions | a = 39.78 Å |
| | b = 84.72 Å |
| | c = 90.19 Å |
| | α = 90° |
| | β = 90° |
| | γ = 90° |
| Wavelength λ | 0.9794 Å |
| Overall Resolution limits | 30.861 Å |
| | 1.99 Å |
| Number of reflections collected | 140193 |
| Number of unique reflections | 21299 |
| Overall Redundancy of data | 6.5 |
| Overall Completeness of data | 98.2% |
| Completeness of data in last data shell | 87.8% |
| Overall R_{SYM} | 0.117 |
| R_{SYM} in last resolved shell | 0.866 |
| Overall I/sigma(I) | 9.0 |
| I/sigma(I) in last shell | 1.8 |

TABLE 4
Model Refinement Statistics

| | | |
|---|---|---|
| Model | Total number of atoms | 2136 |
| | Number of water molecules | 68 |
| | Temperature factor for all atoms | 35.36 Å² |
| | Matthews coefficient | 2.54 |
| | Corresponding solvent content | 51.22% |
| Refinement | Resolution limits | 30.861 Å |
| | | 1.99 Å |
| | Number of reflections used | 21299 |
| | with I > 1 sigma(I) | 21122 |
| | with I > 3 sigma(I) | 13069 |
| | Completeness | 98.7% |
| | R-factor for all reflections | 0.2399 |
| | Correlation coefficient | 0.9247 |
| | Number of reflections above 2 | 19654 |
| | sigma(F) and resolution from 5.0 Å - | |
| | high resolution limit | |
| | used to calculate Rworking | 18639 |
| | used to calculate Rfree | 1015 |
| | R-factor without free reflections | 0.221 |
| | R-factor for free reflections | 0.285 |
| | Error in coordinates estimated by | 0.2589 Å |
| | Luzzati plot | |
| Validation | Phi-Psi core region | 92.3% |
| | Phi-Psi violations | 0 |
| | Residues in disallowed regions: | |
| % bad | Short contact distances | 0 |
| contacts | | |
| | RMSD from ideal bond length | 0.017 Å |
| | RMSD from ideal bond angle | 1.58° |

### Example 2.1

### Structure Analyses

Atomic superpositions were performed with MOE (available from Chemical Computing Group, Inc., Montreal, Quebec, Canada). Per residue solvent accessible surface calculations were done with GRASP (Nicholls et al., “Protein folding and association: insights from the interfacial and thermodynamic properties of hydrocarbons,” Proteins, 11:281-96, 1991). The electrostatic surface was calculated using a probe radius of 1.4 Å.

The structure in both cases was that of a monomeric kinase domain with a subdomain structure typical for kinases, i.e. with two lobes, one largely consisting of alpha helix and one largely consisting of beta sheet with the active site sandwiched between these two subdomains.

### Example 3

### Determination of SYKKD Staurosporine Co-crystal Structures

The section below describes the production of a polypeptide comprising the Homo sapiens SYKKD, and the preparation and characterization of diffraction quality crystals and heavy-atom derivative crystals. Protein was expressed as in Example 1.

After thawing at room temperature, cells were resuspended in salt lysis buffer (50 mM Tris-Cl, pH 7.7; 250 mM NaCl) and 2.5 micro M Staurosporine. The cell suspension was stirred for 1 h at 4 degree C and then centrifuged to remove cell debris. The soluble fraction was purified over a 5 ml Nickel-NTA drip column (Qiagen) and eluted under native conditions in a batch mode with 500 mM imdiazole in 50mM Tris pH7.8, 10 mM methionine, 10% glycerol. The SYK protein was then purified by gel filtration using a Superdex 200 preparative grade column equilibrated in GF4 buffer (10 mM HEPES, pH 7.5, 10 mM methionine, 500 mM NaCl, 5 mM DTT, and 10% glycerol). Fractions were pooled and run over a 1 ml Nickel-NTA column. SYK was eluted with 500 mM imidazole in 50 mM Tris pH7.8, 10 mM methionine, 10% glycerol. Following, the elution fractions were diluted 1:10 into anion exchange chromatography buffer A (50 mM Tris-CI, pH 8.0; 5 mM DTT) and run on a 6 ml ResourceQ column (Pharmacia). The elution buffer (50 mM Tris-Ci, pH 8.0; 1 M NaCl; 5 mM DTT) was run with a 0-50% gradient over 20 column volumes. Syk does not bind to the column under these conditions and is recovered >98% pure in the flow through, as judged by mass spectroscopic analysis. The protein is concentrated to 15 mg/ml. The Staurosporine remains bound to SYK throughout the purification, as shown by fluorescence measurements.

Crystallization conditions were as in Example 1, with the exception that the crystals were obtained using a reservoir solution of 12% PEG 6K, 0.1M Citric Acid pH 5, and 600 mM LiCl, the initial protein concentration was 8 mg/ml, and the incubation was carried out at 21° C.

The crystal data was collected as in Example 1, and the structure determination was essentially as in Example 1, with the exception that the example 1 model was used as the reference model for molecular replacement.

TABLE 5
Data Collection Statistics

| Space group | P 21 21 21 |
|---|---|
| Cell dimensions | a = 40.32 Å |
| | b = 84.65 Å |
| | c = 90.9 Å |
| | α = 90° |
| | β = 90° |
| | γ = 90° |
| Wavelength λ | 0.9794 Å |
| Overall Resolution limits | 28.513 Å |
| | 1.99 Å |
| Number of reflections collected | 133339 |
| Number of unique reflections | 20908 |
| Overall Redundancy of data | 6.3 |
| Overall Completeness of data | 96% |
| Completeness of data in last data shell | 80.2% |
| Overall R_{SYM} | 0.112 |
| R_{SYM} in last resolved shell | 1.616 |
| Overall I/sigma(I) | 10.1 |
| I/sigma(I) in last shell | 1.6 |

TABLE 6
Model Refinement Statistics

| | | |
|---|---|---|
| Model | Total number of atoms | 2229 |
| | Number of water molecules | 78 |
| | Temperature factor for all atoms | 36.775 Å² |
| | Matthews coefficient | 2.49 |
| | Corresponding solvent content | 50.13% |
| Refinement | Resolution limits | 28.398 Å |
| | | 2.1 Å |
| | Number of reflections used | 18831 |
| | with I > 1 sigma(I) | 18400 |
| | with I > 3 sigma(I) | 13111 |
| | Completeness | 98.3% |
| | R-factor for all reflections | 0.247 |
| | Correlation coefficient | 0.915 |
| | Number of reflections above 2 | 16954 |
| | sigma(F) and resolution from 5.0 Å - | |
| | high resolution limit | |
| | used to calculate Rworking | 16054 |
| | used to calculate Rfree | 900 |
| | R-factor without free reflections | 0.241 |
| | R-factor for free reflections | 0.297 |
| | Error in coordinates estimated by | 0.3014 Å |
| | Luzzati plot | |
| Validation | Phi-Psi core region | 91.6% |
| | Phi-Psi violations | 0 |
| | Residues in disallowed regions: | |
| % bad | Short contact distances | 0 |
| contacts | | |
| | RMSD from ideal bond length | 0.006 Å |
| | RMSD from ideal bond angle | 1.55° |

### Example 4

### Determination of SYKKD Co-Crystal Structure

Crystallization conditions were as in Example 1, with the exception that the crystals were obtained using a reservoir solution of 10% PEG 6K, 1M LiCl, 100 mM MES pH 6, and the protein was at 13.2 mg/ml in 1 mM AMP-PNP and 2mM MgCl₂. The incubation was carried out at 90° C. The compound 4-N′-(6-Bromo-2-oxo-1,2-dihydro-indol-3-ylidene)-hydrazino]-benzoic acid is soaked into a crystal by adding the crystal and the compound (from a concentrated stock in DMSO) to the reservoir solution. The final concentration of compound is 1 mM, and the final concentration of DMSO is 1% in the reservoir solution. Soaking continues for three days. The experiment is conducted at 4° C.

TABLE 7
Data Collection Statistics

| Space group | P 21 21 21 |
|---|---|
| Cell dimensions | a = 39.8 Å |
| | b = 85.83 Å |
| | c = 90.54 Å |
| | α = 90° |
| | β = 90° |
| | γ = 90° |
| Wavelength λ | 0.9198 Å |
| Overall Resolution limits | 29.18 Å |
| | 1.97 Å |
| Number of reflections collected | 106777 |
| Number of unique reflections | 21118 |
| Overall Redundancy of data | 5.0 |
| Overall Completeness of data | 94.5% |
| Completeness of data in last data shell | 95.7% |
| Overall R_{SYM} | 0.082 |
| R_{SYM} in last resolved shell | 1.782 |
| Overall I/sigma(I) | 9.5 |
| I/sigma(I) in last shell | 0.7 |

TABLE 8
Model Refinement Statistics

| | | |
|---|---|---|
| Model | Total number of atoms | 2102 |
| | Number of water molecules | 84 |
| | Temperature factor for all atoms | 36.07 Å² |
| | Matthews coefficient | 2.63 |
| | Corresponding solvent content | 52.82% |
| Refinement | Resolution limits | 29.235 Å |
| | | 2.26 Å |
| | Number of reflections used | 13994 |
| | with I > 1 sigma(I) | 13811 |
| | with I > 3 sigma(I) | 10422 |
| | Completeness | 92.6% |
| | R-factor for all reflections | 0.2475 |
| | Correlation coefficient | 0.9044 |
| | Number of reflections above 2 | 12490 |
| | sigma(F) and resolution from 5.0 Å - | |
| | high resolution limit | |
| | used to calculate Rworking | 11832 |
| | used to calculate Rfree | 658 |
| | R-factor without free reflections | 0.233 |
| | R-factor for free reflections | 0.295 |
| | Error in coordinates estimated by | 0.3121 Å |
| | Luzzati plot | |
| Validation | Phi-Psi core region | 89% |
| | Phi-Psi violations | |
| | Residues in disallowed regions: | 0 |
| % bad | Short contact distances | 0.8 |
| contacts | | |
| | RMSD from ideal bond length | 0.021 Å |
| | RMSD from ideal bond angle | 2.04° |

### Example 5

### Use of SYKKD Coordinates for Inhibitor Design

The coordinates of the present invention, including the coordinates of molecules comprising the binding pocket residues of FIGS. 4-7, as well as coordinates of homologs having a rmsd of the backbone atoms of preferably less than 1.5 Å, more preferably less than 1.25 Å, more preferably less than 1 Å, more preferably less than 0.75 Å, and more preferably less than 0.5 Å from the coordinates of FIGS. 4-7, are used to design compounds, including inhibitory compounds, that associate with SYK, or homologs of SYK. Such compounds may associate with SYK at the active site, in a binding pocket, in an accessory binding pocket, or in parts or all of both regions.

The process may be aided by using a computer comprising a computer readable database, wherein the database comprises coordinates of an active site, binding pocket, or accessory binding pocket of the present invention. The computer may preferably be programmed with a set of machine-executable instructions, wherein the recorded instructions are capable of displaying a three-dimensional representation of SYK, or portions thereof. The computer is used according to the methods described herein to design compounds that associate with SYK, for example, at the active site or a binding pocket.

A chemical compound library is obtained. The library may be purchased from a publicly available source such as, for example, ChemBridge (San Diego, Calif., www.chembridge.com), Available Chemical Database, or Asinex (Moscow 123182, Russia, www.asinex.com). A filter is used to retain compounds in the library that satisfy the Lipinski rule of five, which states that compounds are likely to have good absorption and permeation in biological systems and are more likely to be successful drug candidates if they meet the following criteria: five or fewer hydrogen-bond donors, ten or fewer hydrogen-bond acceptors, molecular weight less than or equal to 500, and a calculated logP less than or equal to 5. (Lipinski, C.A., et al., Advanced Drug Delivery Reviews 23 3-25 (1996)).

This filter reduces the size of the compound library used to screen against the structure of the present invention. Docking programs described herein, such as, for example, DOCK, or GOLD, are used to identify compounds that bind to the active site and/or binding pocket. Compounds may be screened against more than one binding pocket of the protein structure, or more than one set of coordinates for the same protein, taking into account different molecular dynamic conformations of the protein. Consensus scoring is then used to identify the compounds that are the best fit for the protein (Charifson, P.S. et al., J. Med. Chem. 42:5100-9 (1999)). Data obtained from more than one protein molecule structure may also be scored according to the methods described in Klingler et al., U.S. Utility Application, filed May 3, 2002, entitled “Computer Systems and Methods for Virtual Screening of Compounds.” Compounds having the best fit are then obtained from the producer of the chemical library, or synthesized, and used in binding assays and bioassays.

The coordinates of the present invention are also used to determine pharmacophores. These pharmacophores may be designed after reviewing results from the use of a docking program, to determine the shape of the SYK pharmacophore. Alternatively, programs such as GRID are used to calculate the properties of a pharmacophore. Once the pharmacophore is determined, it is be used to screen chemical libraries for compounds that fit within the pharmacophore.

The coordinates of the present invention are also used to identify substructures that interact with various portions of an active site or binding pocket of SYK. Once a substructure, or set of substructures, is determined, it is used to screen a chemical library for compounds comprising the substructure or set of substructures. The identified compounds are then docked to, for example, the active site or binding pocket.

### Example 6

### Bioassay

The kinase assays may use various forms of SYKKD and SYK, including, for

example, SYKKD or the SYK molecule itself, or a portion thereof.

Materials

Substrate peptide=Poly (Glu, Tyr) 4:1=poly EY (Sigma P-0275)

βNADH (Sigma CAT#N-8129, FW=709.4)

2M MgCl₂

1M HEPES buffer, pH 7.5

Phosphoenolpyruvate=PEP (Sigma CAT#P-7002, FW=234)

Lactate dehydrogenase=LDH (Worthington Biochemical CAT#2756)

Pyruvate Kinase=PK (Sigma CAT#P-9136)

ATP (Sigma CAT#A-3377, FW=551)

Greiner 384-well UV star plate

Purified SYK kinase domain

Stock Solutions

10 mM NADH (7.09 mg/mL in miliQH₂O) make fresh daily

1 mg/ml Poly EY (in miliQH₂O) store at −20° C.

200 mM HEPES buffer, pH 7.5 (10 ml 1 M stock+40 ml miliQH₂O)

100 mM MgCl2 (5 mL+95 ml dH₂O)

100 mM PEP (23.4 mg/mL in dH₂O) store at −20° C.

10 mM ATP (5.51 mg/mL in dH₂O) store at −20° C. (dilute 50 Å into total of 10 mL

miliQH₂O daily=50 μM ATP working stock)

1000 U/ml PK (U/mg varies with lot) flash-freeze under liquid N2 and store at −80° C.

1000 U/ml LDH (U/mg varies with lot) flash-freeze under liquid N2 and store at −80° C.

Standard Assay Setup for 384-well format (50 μl reaction)

300 μM NADH

10 mM MgCl₂

2 mM PEP

45 U/ml PK

60U/ml LDH

1 mg/ml Poly EY

2.5 μl test compound (in DMSO)

1 g/ml SYK kinase domain

10 μM ATP*

100 mM HEPES buffer

QS with miliQ dH2O to 50 μl

Positive controls contain DMSO with no test compound. Negative controls contain

DMSO with no test compound and buffer in place of SYK kinase domain.

*The kinase reaction is initiated at time t=0 by the addition of ATP

Assay Progress Measurements

The activity is measured by following the time-dependent loss of NADH by absorbance spectroscopy at 340 nm. The linear portion of the resulting progress curve can then be analyzed by linear regression to get the activity in absorbance units/time, reported as the slope of that best fit line (moles/unit time can be calculated from using molar extiction coeffecient for NADH at 340 nm, 6250M⁻¹ cm⁻¹).

Data Analysis

Screening

Z′=1−[3*(θ₊+θ₋)/|μ₊−μ|]

Where μ denotes the mean and θ the standard deviation. The subscript designates positive or negative controls. The Z′ score for a robust screening assay should be >0.50.

The typical threshold =μ₊−3*θ₊

Any value that falls below the threshold is designated a “hit”.

Dose Response

y=min+{(max-min)/( 1+10^{[compound]-logIC50})}

Where y=the observed initial slope, max=the slope in the absence of inhibitor, min=the slope at infinite inhibitor, and the IC₅₀ is the [compound] that corresponds to ½ the total observed amplitude (Amplitude=max-min).

The IC₅₀ is related to the Ki by the following equation:

IC₅₀ =Kᵢ(1+[ATP]/Km)

To measure modulation, activation, or inhibition of SYKKD, a test compound is added to the assay at a range of concentrations. Inhibitors may inhibit SYKKD activity at an IC₅₀ in the nanomolar range, and, for example, in the subnanomolar range.

Binding Assay:

To measure the binding of a compound to SYK or SYKKD, a test kit manufactured by Discoverx (Fremont, Calif.), ED-Staurosporine NSIP™ Enzyme Binding Assay Kit (see U.S. Pat. No. 5,643,734) may be used.

### Example 7

### Formulation and Administration

Pharmaceutical compositions comprising SYK modulators, such as inhibitors, are useful, for example, for treating diseases and disorders in which SYK activity is implicated. Pharmaceutical compositions containing SYK effectors may also be used to modify the activity of human homologs of SYK.

In therapeutic and/or diagnostic applications, the compounds of the invention can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

The compounds according to the invention are effective over a wide dosage range. For example, in the treatment of adult humans, dosages from 0.01 to 1000 mg from 0.5 to 100 mg, and from 1 to 50 mg per day, from 5 to 40 mg per day are examples of dosages that may be used. A most preferable dosage is 10 to 30 mg per day. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Depending on the specific conditions being treated, such agents may be formulated into liquid or solid dosage forms and administered systemically or locally. The agents may be delivered, for example, in a timed- or sustained- low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intraperitoneal, intranasal, or intraocular injections.

For injection, the agents of the invention may be formulated in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art. Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross- linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

The present invention is not to be limited in scope by the exemplified embodiments, which are intended as illustrations of single aspects of the invention. Indeed, various modifications of the invention in addition to those described herein will become apparent to those having skill in the art from the foregoing description and accompanying drawings. Such modifications are intended to fall within the scope of the invention. References cited throughout this application are examples of the level of skill in the art and are hereby incorporated by reference herein in their entirety, whether previously specifically incorporated or not.

We claim:

1. A SYKKD protein, or a functional SYKKD protein subunit, in crystalline form.

2. The crystalline protein of claim 1, which is a heavy-atom derivative crystal.

3. The crystalline protein of claim 2, in which SYKKD protein is a mutant.

4. The crystalline protein of claim 3, which is characterized by a set of structural coordinates that is substantially similar to the set of structural coordinates of FIGS. 4, 5, 6, or 7.

5. A machine-readable medium embedded with information that corresponds to a three-dimensional structural representation of a crystal of claim 1.

6. The machine readable medium of claim 5, embedded with the molecular structural coordinates of FIGS. 4, 5, 6, or 7, or at least 50% of the coordinates thereof.

7. The machine-readable medium of claim 5, embedded with the molecular structural coordinates of FIGS. 4, 5, 6, or 7, or at least 80% of the coordinates thereof.

8. The machine-readable medium of claim 5, embedded with the molecular structural coordinates of a protein molecule comprising a SYKKD protein binding pocket, wherein said binding pocket comprises at least three amino acids selected from the group consisting of Glu97, Met98, Ala99, Ala48, Leu149, Val33, Lys50, Val81, Leu94, Met96, Glu100, Gly102, Pro103, Arg146, Asn147, and Ser159 having the structural coordinates of FIGS. 4, 5, 6, or 7, or by the structural coordinates of a binding pocket homolog, wherein said the root mean square deviation of the backbone atoms of the amino acid residues of said binding pocket and said binding pocket homolog is less than 2.0 Å.

9. The machine-readable medium of claim 5, wherein said binding pocket comprises Glu97, Met98, and Ala99 according to the sequence of FIGS. 4, 5, 6, or 7.

10. The machine-readable medium of claim 9, wherein said binding pocket further comprises Ala48 and Leu149 according to the sequence of FIGS. 4, 5, 6, or 7.

11. The machine-readable medium of claim 10, wherein said binding pocket further comprises Val33, Lys50, Val81, Leu94, Met96, Glu100, Gly102, Pro103, Arg146, Asn147, and Ser159 according to the sequence of FIGS. 4, 5, 6, or 7.

12. A method of electronically transmitting all or part of the information stored in the machine-readable medium of claim 5.

13. A method of producing a computer readable database comprising the three-dimensional molecular structural coordinates of a binding pocket of a SYKKD protein, said method comprising
a) obtaining three-dimensional structural coordinates defining said protein or a binding pocket of said protein, from a crystal of said protein; and
b) introducing said structural coordinates into a computer to produce a database containing the molecular structural coordinates of said protein or said binding pocket.

14. The method of claim 13, wherein said binding pocket of said protein is part of a co-complex with at least one compound.

15. The method of claim 13 wherein said binding pocket comprises amino acids Glu, Met, and Ala.

16. The method of claim 15 wherein said computer is capable of utilizing or displaying a three-dimensional molecular structure comprising said binding pocket using said structural coordinates.

17. The method of claim 15, wherein said binding pocket further comprises amino acids corresponding to Ala and Leu.

18. The method of claim 17 wherein said binding pocket further comprises amino acids corresponding to Val, Lys, Val, Leu, Met, Glu, Gly, Pro, Arg, Asn, and Ser.

19. The method of claim 13 wherein said binding pocket comprises a binding pocket defined by the structural coordinates of at least three amino acids selected from the group consisting of Glu97, Met98, Ala99, Ala48, Leu149, Val33, Lys50, Val81, Leu94, Met96, Glu100, Gly102 ,Pro103, Arg146, Asn147, and Ser159.

20. The method of claim 19 wherein said binding pocket comprises Glu97, Met98, and Ala99 according to the sequence of FIGS. 4, 5, 6, or 7.

21. The method of claim 20, wherein said binding pocket further comprises Ala48 and Leu149 according to the sequence of FIGS. 4, 5, 6, or 7.

22. The method of claim 21, wherein said binding pocket further comprises Val33, Lys50, Val81, Leu94, Met96, Glu100, Gly102, Pro103, Arg146, Asn147, and Ser159 according to the sequence of FIGS. 4, 5, 6, or 7.

23. The method of claim 13, wherein said binding pocket comprises an active site.

24. A computer readable database produced by claim 13.

25. A method comprising electronic transmission of all or part of the computer readable database produced by claim 13.

26. A method of producing a computer readable database comprising a representation of a compound capable of binding a binding pocket of a SYKKD protein, said method comprising
a) introducing into a computer program a computer readable database produced by claim 13;
b) generating a three-dimensional representation of a binding pocket of said SYKKD protein in said computer program;
c) superimposing a three-dimensional model of at least one binding test compound on said representation of the binding pocket;
d) assessing whether said test compound model fits spatially into the binding pocket of said SYKKD protein; and
e) storing a representation of a compound that fits into the binding pocket into a computer readable database.

27. The method of claim 26 wherein in e), said representation is stored in the database produced by claim 13.

28. The method of claim 26, wherein said representation is selected from the group consisting of the compound's name, a chemical or molecular formula of the compound, a chemical structure of the compound, an identifier for the compound, and three-dimensional molecular structural coordinates of the compound.

29. The method of claim 26, wherein said generating of a three-dimensional representation of the binding pocket comprises use of structural coordinates having a root mean square deviation of the backbone atoms of the amino acid residues of said binding pocket of less than 2.0 Å from the structural coordinates of the corresponding residues according to FIGS. 4, 5, 6, or 7.

30. The method of claim 26, wherein said at least one binding test compound is selected by a method selected from i) selecting a compound from a small molecule database, (ii) modifying a known inhibitor, substrate, reaction intermediate, or reaction product, or a portion thereof, of SYKKD, (iii) assembling chemical fragments or groups into a compound, and (iv) de novo ligand design of said compound.

31. The method of claim 26, wherein said assessing of whether a test compound model fits is by docking the model to said representation of said SYKKD binding pocket and/or performing energy minimization.

32. The method of claim 26 further comprising
f) preparing a binding test compound represented in said computer readable database;
g) contacting said compound in a binding assay with a protein comprising said SYKKD protein binding pocket;
h) determining whether said test compound binds to said protein in said assay; and
i) introducing a representation of a compound that binds to said protein in said assay into a computer readable database.

33. The method of claim 32 wherein in i), said representation is stored in the database produced by claim 26.

34. The method of claim 32, wherein said representation is selected from the group consisting of the compound's name, a chemical formula of the compound, a chemical structure of the compound, an identifier for the compound, and three-dimensional molecular structural coordinates of the compound.

35. A method of producing a computer readable database comprising a representation of a binding pocket of a SYKKD protein in a co-crystal with a compound, said method comprising
a) preparing a binding test compound represented in a computer readable database produced by claim 26;
b) forming a co-crystal of said compound with a protein comprising a binding pocket of a SYKKD protein;
c) obtaining the structural coordinates of said binding pocket in said co-crystal; and
d) introducing the structural coordinates of said binding pocket or said co-crystal into a computer-readable database.

36. The method of claim 35, further comprising introducing the structural coordinates of said compound in said co-crystal into said database.

37. The method of claim 26 wherein said binding pocket comprises amino acids Glu, Met, and Ala.

38. The method of claim 37 wherein said computer is capable of utilizing or displaying a three-dimensional molecular structure of said binding pocket using said structural coordinates.

39. The method of claim 37 wherein said binding pocket further comprises amino acids corresponding to Ala and Leu.

40. The method of claim 39 wherein said binding pocket further comprises amino acids corresponding to Val, Lys, Val, Leu, Met, Glu, Gly, Pro, Arg, Asn, and Ser.

41. The method of claim 26 wherein said binding pocket comprises a binding pocket defined by the structural coordinates of at least three amino acids selected from the group consisting of Glu97, Met98, Ala99, Ala48, Leu149, Val33, Lys50, Val81, Leu94, Met96, Glu100, Gly102, Pro103, Arg146, Asn147, and Ser159.

42. The method of claim 41 wherein said binding pocket comprises Glu97, Met98, and Ala99 according to the sequence of FIGS. 4-7.

43. The method of claim 42, wherein said binding pocket further comprises Ala48 and Leu149 according to the sequence of FIGS. 4, 5, 6, or 7.

44. The method of claim 43, wherein said binding pocket further comprises Val33, Lys50, Val81, Leu94, Met96, Glu100, Gly102, Pro103, Arg146, Asn147, and Ser159 according to the sequence of FIGS. 4, 5, 6, or 7.

45. The method of claim 26, wherein said binding pocket comprises an active site.

46. A computer readable database produced by claim 26.

47. A method comprising electronic transmission of all or part of the computer readable database produced by claim 26.

48. A method of modulating SYKKD protein activity comprising contacting said SYKKD with a compound, wherein said compound is represented in a database produced by the method of claim 26.

49. A method of producing a compound comprising a three-dimensional molecular structure represented by the coordinates contained in a computer readable database produced by claim 26 comprising synthesizing said compound wherein said compound fits a binding pocket of SYKKD protein.

50. A method of modulating SYKKD protein activity, comprising contacting said SYKKD protein with a compound produced by claim 49.

51. A method of identifying an activator or inhibitor of a protein that comprises a SYKKD active site or binding pocket, comprising
a) producing a compound according to claim 49;
b) contacting said compound with a protein that comprises a SYKKD active site or binding pocket; and
c) determining whether the potential modulator activates or inhibits the activity of said protein.

52. A method of producing an activator or inhibitor identified by claim 51.

53. A method of producing a computer readable database comprising a representation of a compound rationally designed to be capable of binding a binding pocket of a SYKKD protein, said method comprising
a) introducing into a computer program a computer readable database produced by claim 13;
b) generating a three-dimensional representation of the protein or a binding pocket of said SYKKD protein in said computer program;
c) designing a three-dimensional model of a compound that forms non-covalent bonds with amino acids of a binding pocket of said representation; and
d) storing a representation of said compound into a computer readable database.

54. The method of claim 53, wherein said representation is selected from the group consisting of the compound's name, a chemical or molecular formula of the compound, a chemical structure of the compound, an identifier for the compound, and three-dimensional structural coordinates of the compound.

55. The method of claim 53 further comprising
e) preparing a binding test compound comprising a three-dimensional molecular structure represented by the coordinates contained in said computer readable database;
f) contacting said compound in a binding assay with a protein comprising said binding pocket of a SYKKD protein;
g) determining whether said test compound binds to said protein in said assay; and
h) introducing a representation of a compound that binds to said protein in said assay into a computer-readable database.

56. The method of claim 55, wherein said representation is selected from the group consisting of the compound's name, a chemical or molecular formula of the compound, a chemical structure of the compound, an identifier for the compound, and three-dimensional structural coordinates of the compound.

57. A method of producing a computer readable database comprising a representation of a binding pocket of a SYKKD protein in a co-crystal with a compound rationally designed to be capable of binding said binding pocket, said method comprising
a) preparing a binding test compound represented in a computer readable database produced by claim 53;
b) forming a co-crystal of said compound with a protein comprising a binding pocket of a SYKKD protein;
c) obtaining the structural coordinates of said binding pocket in said co-crystal; and
d) introducing the structural coordinates of said binding pocket or said co-crystal into a computer-readable database.

58. The method of claim 57, further comprising introducing the structural coordinates of said compound in said co-crystal into said database.

59. The method of claim 53 wherein said binding pocket comprises amino acids Glu, Met, and Ala.

60. The method of claim 59 wherein said binding pocket further comprises amino acids corresponding to Ala and Leu.

61. The method of claim 60 wherein said binding pocket further comprises amino acids corresponding to Val, Lys, Val, Leu, Met, Glu, Gly, Pro, Arg, Asn, and Ser.

62. The method of claim 53 wherein said binding pocket comprises a binding pocket defined by the structural coordinates of at least three amino acids selected from the group consisting of Glu97, Met98, Ala99, Ala48, Leu149, Val33, Lys50, Val81, Leu94, Met96, Glu100, Gly102, Pro103, Arg146, Asn147, and Ser159.

63. The method of claim 62 wherein said binding pocket comprises Glu97, Met98, and Ala99 according to the sequence of FIGS. 4, 5, 6, or 7.

64. The method of claim 63, wherein said binding pocket further comprises Ala48 and Leu149 according to the sequence of FIGS. 4, 5, 6, or 7.

65. The method of claim 64, wherein said binding pocket further comprises Val33, Lys50, Val81, Leu94, Met96, Glu100, Gly102, Pro103, Arg146, Asn147, and Ser159 according to the sequence of FIGS. 4, 5, 6, or 7.

66. The method of claim 53, wherein said binding pocket comprises an active site.

67. A computer readable database produced by claim 53.

68. A method comprising electronic transmission of all or part of the computer readable database produced by claim 53.

69. A method of producing a computer readable database comprising structural information about a molecule or a molecular complex of unknown structure comprising:
a) generating an x-ray diffraction pattern from a crystallized form of said molecule or molecular complex;
b) using a molecular replacement method to interpret the structure of said molecule; wherein said molecular replacement method uses the structural coordinates of a crystalline protein of claim 1, or a subset thereof comprising a binding pocket, the structural coordinates of a binding pocket of FIGS. 4, 5, 6, or 7, or structural coordinates having a root mean square deviation for the alpha-carbon atoms of said structural coordinates of less than 2.0 Å; and
c) storing the coordinates of the resulting structure in a computer readable database.

70. The method of claim 69 wherein said binding pocket comprises a binding pocket defined by the structural coordinates of at least three amino acids selected from the group consisting of Glu97, Met98, Ala99, Ala48, Leu149, Val33, Lys50, Val81, Leu94, Met96, Glu100, Gly102, Pro103, Arg146, Asn147, and Ser159.

71. The method of claim 70 wherein said binding pocket comprises Glu97, Met98, and Ala99 according to the sequence of FIGS. 4, 5, 6, or 7.

72. The method of claim 71, wherein said binding pocket further comprises Ala48 and Leu149 according to the sequence of FIGS. 4, 5, 6, or 7.

73. The method of claim 72, wherein said binding pocket further comprises Val33, Lys50, Val81, Leu94, Met96, Glu100, Gly102, Pro103, Arg146, Asn147, and Ser159 according to the sequence of FIGS. 4, 5, 6, or 7.

74. The method of claim 69, wherein said binding pocket comprises an active site.

75. A computer readable database produced by claim 69.

76. A method comprising electronic transmission of all or part of the computer readable database produced by claim 69.

77. A method for homology modeling the structure of a SYKKD protein homolog comprising:
a) aligning the amino acid sequence of a SYKKD protein homolog with an amino acid sequence of SYKKD protein;
b) incorporating the sequence of the SYKKD protein homolog into a model of the structure of SYKKD protein, wherein said model has the same structural coordinates as the structural coordinates of a crystalline protein of claim 1, the structural coordinates of FIGS. 4, 5, 6, or 7, or wherein the structural coordinates of said model's alpha-carbon atoms have a root mean square deviation from the structural coordinates of FIGS. 4, 5, 6, or 7, of less than 2.0 Å to yield a preliminary model of said homolog;
c) subjecting the preliminary model to energy minimization to yield an energy minimized model; and
d) remodeling regions of the energy minimized model where stereochemistry restraints are violated to yield a final model of said homolog.

78. A method for identifying a compound that binds SYKKD protein comprising:
a) providing a computer modeling program with a set of structural coordinates or a three dimensional conformation for a molecule that comprises a binding pocket of a crystalline protein of claim 1, or a homolog thereof;
b) providing a said computer modeling program with a set of structural coordinates of a chemical entity;
c) using said computer modeling program to evaluate the potential binding or interfering interactions between the chemical entity and said binding pocket; and
d) determining whether said chemical entity potentially binds to or interferes with said protein or homolog.

79. The method of claim 78 further comprising the steps of:
e) computationally modifying the structural coordinates or three dimensional conformation of said chemical entity to improve the likelihood of binding to said binding pocket; and
b) determining whether said modified chemical entity potentially binds to or interferes with said protein or homolog.

80. The method of claim 78 wherein determining whether the chemical entity potentially binds to said molecule comprises performing a fitting operation between the chemical entity and a binding pocket of the protein or homolog; and computationally analyzing the results of the fitting operation to quantify the association between, or the interference with, the chemical entity and the binding pocket.

81. The method of claim 78 wherein a library of structural coordinates of chemical entities is used to identify a compound that binds.

82. A method for designing a compound that binds SYKKD protein comprising:
a) providing a computer modeling program with a set of structural coordinates, or a three dimensional conformation derived therefrom, for a molecule that comprises a binding pocket comprising the structural coordinates of a binding pocket of a crystalline protein of claim 1, or a homolog thereof;
b) computationally building a chemical entity represented by set of structural coordinates; and
c) determining whether the chemical entity is expected to bind to said molecule.

83. The method of claim 82, wherein determining whether the chemical entity potentially binds to said molecule comprises performing a fitting operation between the chemical entity and a binding pocket of the molecule; and
computationally analyzing the results of the fitting operation to quantify the association between the chemical entity and the binding pocket.

84. The method of claim 82 wherein said binding pocket comprises a binding pocket defined by the structural coordinates of at least three amino acids selected from the group consisting of Glu97, Met98, Ala99, Ala48, Leu149, Val33, Lys50, Val81, Leu94, Met96, Glu100, Gly102, Pro103, Arg146, Asn147, and Ser159.

85. The method of claim 84 wherein said binding pocket comprises Glu97, Met98, and Ala99 according to the sequence of FIGS. 4, 5, 6, or 7.

86. The method of claim 85, wherein said binding pocket further comprises Ala48 and Leu149 according to the sequence of FIGS. 4, 5, 6, or 7.

87. The method of claim 86, wherein said binding pocket further comprises Val33, Lys50, Val81, Leu94, Met96, Glu100, Gly102, Pro103, Arg146, Asn147, and Ser159 according to the sequence of FIGS. 4, 5, 6, or 7.

88. The method of claim 82, wherein said binding pocket comprises an active site.

89. A method of producing a mutant SYKKD protein, having an altered property relative to SYKKD protein, comprising,
a) constructing a three-dimensional structure of SYKKD protein having structural coordinates selected from the group consisting of the structural coordinates of a crystalline protein of claim 1, the structural coordinates of FIGS. 4-7, and the structural coordinates of a protein having a root mean square deviation of the alpha carbon atoms of said protein of less than 2.0 Å when compared to the structural coordinates of FIGS. 4-7;
b) using modeling methods to identify in the three-dimensional structure at least one structural part of the SYKKD protein molecule wherein an alteration in said structural part is predicted to result in said altered property;
c) providing a nucleic acid molecule coding for a SYKKD mutant protein having a modified sequence that encodes a deletion, insertion, or substitution of one or more amino acids at a position corresponding to said structural part; and
d) expressing said nucleic acid molecule to produce said mutant; wherein said mutant has at least one altered property relative to the parent.

90. A method of producing a mutant SYKKD protein, having an altered property relative to SYKKD protein, comprising,
a) constructing a three-dimensional structure of a molecule comprising a binding pocket, wherein said binding pocket comprises at least three amino acids selected from the group consisting of Glu97, Met98, Ala99, Ala48, Leu149, Val33, Lys50, Val81, Leu94, Met96, Glu100, Gly102, Pro103, Arg146, Asn147, and Ser159 having the structural coordinates of a crystalline protein of claim 1, the structural coordinates of FIGS. 4, 5, 6, or 7, or the structural coordinates of a binding pocket homolog, wherein said the root mean square deviation of the backbone atoms of the amino acid residues of said binding pocket and said binding pocket homolog is less than 2.0 Å;
b) using modeling methods to identify in the three-dimensional structure at least one portion of said binding pocket wherein an alteration in said portion is predicted to result in said altered property;
c) providing a nucleic acid molecule coding for a mutant SYKKD protein having a modified sequence that encodes a deletion, insertion, or substitution of one or more amino acids at a position corresponding to said portion; and
d) expressing said nucleic acid molecule to produce said mutant;
wherein said mutant has at least one altered property relative to the parent.

91. A method of producing a computer readable database containing the three-dimensional molecular structural coordinates of a compound capable of binding the active site or binding pocket of a protein molecule, said method comprising
a) introducing into a computer program a computer readable database produced by claim 13;
b) generating a three-dimensional representation of the active site or binding pocket of said SYKKD protein in said computer program;
c) superimposing a three-dimensional model of at least one binding test compound on said representation of the active site or binding pocket;
d) assessing whether said test compound model fits spatially into the active site or binding pocket of said SYKKD protein;
e) assessing whether a compound that fits will fit a three-dimensional model of another protein, the structural coordinates of which are also introduced into said computer program and used to generate a three-dimensional representation of the other protein; and
f) storing the three-dimensional molecular structural coordinates of a model that does not fit the other protein into a computer readable database.

92. A method for determining whether a compound binds SYKKD protein, comprising,
a) providing a computer modeling program with a set of structural coordinates or a three dimensional conformation for a molecule that comprises a binding pocket of a crystalline protein of claim 1, or a homolog thereof;
b) providing a said computer modeling program with a set of structural coordinates of a chemical entity;
c) using said computer modeling program to evaluate the potential binding or interfering interactions between the chemical entity and said binding pocket; and
d) determining whether said chemical entity potentially binds to or interferes with said protein or homolog.

93. A method of producing a computer readable database comprising a representation of a compound capable of binding a binding pocket of a SYKKD protein, said method comprising,
a) introducing into a computer program a computer readable database produced by claim 13;
b) determining a pharmacophore that fits within said binding pocket;
c) computationally screening a plurality of compounds to determine which compound(s) or portion(s) thereof fit said pharmacophore; and
d) storing a representation of said compound(s) or portion(s) thereof into a computer readable database.

94. The method of claim 93, wherein said representation is selected from the group consisting of the compound's name, a chemical or molecular formula of the compound, a chemical structure of the compound, an identifier for the compound, and three-dimensional molecular structural coordinates of the compound.

95. The method of claim 93 wherein said binding pocket comprises a binding pocket defined by the structural coordinates of at least three amino acids selected from the group consisting of Glu97, Met98, Ala99, Ala48, Leu149, Val33, Lys50, Val81, Leu94, Met96, Glu100, Gly102, Pro103, Arg146, Asn147, and Ser159.

96. The method of claim 95 wherein said binding pocket comprises Glu97, Met98, and Ala99 according to the sequence of FIGS. 4, 5, 6, or 7.

97. The method of claim 96, wherein said binding pocket further comprises Ala48 and Leu149 according to the sequence of FIGS. 4, 5, 6, or 7.

98. The method of claim 97, wherein said binding pocket further comprises Val33, Lys50, Val81, Leu94, Met96, Glu100, Gly102, Pro103, Arg146, Asn147, and Ser159 according to the sequence of FIGS. 4, 5, 6, or 7.

99. The method of claim 93, wherein said binding pocket comprises an active site.

100. A computer readable database produced by claim 93.

101. A method comprising electronic transmission of all or part of the computer readable database produced by claim 93.

102. A method of producing a computer readable database comprising a representation of a compound capable of binding a binding pocket of a SYKKD protein, said method comprising
a) introducing into a computer program a computer readable database produced by claim 13;
b) determining a chemical moiety that interacts with said binding pocket;
c) computationally screening a plurality of compounds to determine which compound(s) comprise said moiety as a substructure of said compound(s); and
d) storing a representation of said compound(s) that comprise said substructure into a computer readable database.

103. The method of claim 102, wherein said representation is selected from the group consisting of the compound's name, a chemical or molecular formula of the compound, a chemical structure of the compound, an identifier for the compound, and three-dimensional molecular structural coordinates of the compound.

104. The method of claim 102 wherein said binding pocket comprises a binding pocket defined by the structural coordinates of at least three amino acids selected from the group consisting of Glu97, Met98, Ala99, Ala48, Leu149, Val33, Lys50, Val81, Leu94, Met96, Glu100, Gly102, Pro103, Arg146, Asn147, and Ser159.

105. The method of claim 104 wherein said binding pocket comprises Glu97, Met98, and Ala99 according to the sequence of FIGS. 4, 5, 6, or 7.

106. The method of claim 105, wherein said binding pocket further comprises Ala48 and Leu149 according to the sequence of FIGS. 4, 5, 6, or 7.

107. The method of claim 106, wherein said binding pocket further comprises Val33, Lys50, Val81, Leu94, Met96, Glu100, Gly102, Pro103, Arg146, Asn147, and Ser159 according to the sequence of FIGS. 4, 5, 6, or 7.

108. The method of claim 102, wherein said binding pocket comprises an active site.

109. A computer readable database produced by claim 102.

110. A method comprising electronic transmission of all or part of the computer readable database produced by claim 102.

111. Purified SYKKD polypeptide.

112. The method of claim 111, wherein said polypeptide is 98% pure.

113. The method of claim 111, wherein said polypeptide is unphosphorylated.

114. A method of purifying SYKKD polypeptide, comprising expressing SYKKD insect cells;
obtaining a soluble protein fraction from said insect cells;
using a two column chromatograph procedure to obtain purified SYKKD.

115. An insect cell capable of expressing SYKKD.

116. The insect cell of claim 115, wherein said insect cell comprises a vector, wherein said vector comprises a nucleic acid sequence coding for SYKKD.
